(19) United States
(12) Patent Application Publication
(10) Pub. No.: US 2024/0400654 A1
(43) Pub. Date: Dec. 5, 2024

(54) ANTIGEN-BINDING MOLECULES THAT BIND TO PORPHYROMONAS GINGIVALIS

(71) Applicant: KEYSTONE BIO, INC., St. Louis, MO (US)

(72) Inventors: Peter L. Nara, Senecaville, OH (US); Daniel L. Sindelar, St. Louis, MO (US)

(21) Appl. No.: 18/251,526

(22) PCT Filed: Nov. 2, 2021
(86) PCT No.: PCT/US2021/057758

§ 371 Date: May 2, 2023

Related U.S. Application Data
(60) Provisional application No. 63/109,286, filed on Nov. 3, 2020.
(60) Provisional application No. 63/135,878, filed on Jan. 11, 2021.
(60) Provisional application No. 63/208,873, filed on Jun. 9, 2021.
(60) Provisional application No. 63/221,405, filed on Jul. 13, 2021.
(60) Provisional application No. 63/225,295, filed on Jul. 23, 2021.
(60) Provisional application No. 63/231,964, filed on Aug. 11, 2021.

(51) Int. Cl. C07K 16/12

(52) U.S. Cl. CPC C07K 16/1203; C07K 2317/24; C07K 2317/33; C07K 2317/92

(57) ABSTRACT

Antigen-binding molecules (ABMs) that bind to Porphyromonas gingivalis are described. The ABMs may be human or humanized ABMs. The ABMs find use in treating infections involving P. gingivalis, such as periodontal disease. Also provided are methods of treating or preventing a disorder or disease by administering the ABM.

### CROSS-REFERENCE TO RELATED APPLICATIONS

This application is a U.S. national phase of P.C.T. App. No. US2021/057758 filed Nov. 2, 2021 entitled “ANTIGEN-BINDING MOLECULES THAT BIND TO PORPHYROMONAS GINGIVALIS,” and claims priority to U.S. Prov. App. No. 63/109,286 filed Nov. 3, 2020 entitled “ANTIGEN-BINDING MOLECULES THAT BIND TO PORPHYROMONAS GINGIVALIS,” to U.S. Prov. App. No. 63/135,878 filed Jan. 11, 2021 entitled “ANTIGEN-BINDING MOLECULES THAT BIND TO PORPHYROMONAS GINGIVALIS,” to U.S. Prov. App. No. 63/208,873 filed Jun. 9, 2021 entitled “ANTIGEN-BINDING MOLECULES THAT BIND TO PORPHYROMONAS GINGIVALIS,” to U.S. Prov. App. No. 63/221,405 filed Jul. 13, 2021 entitled “ANTIGEN-BINDING MOLECULES THAT BIND TO PORPHYROMONAS GINGIVALIS,” to U.S. Prov. App. No. 63/225,295 filed Jul. 23, 2021 entitled “ANTIGEN-BINDING MOLECULES THAT BIND TO PORPHYROMONAS GINGIVALIS,” and to U.S. Prov. App. No. 63/231,964 filed Aug. 11, 2021 entitled “ANTIGEN-BINDING MOLECULES THAT BIND TO PORPHYROMONAS GINGIVALIS,” which are each incorporated by reference in their entirety.

### REFERENCE TO SEQUENCE LISTING

The present application is being filed along with a Sequence Listing in electronic format. The Sequence Listing is provided as a file entitled KEYBI.001NP.xml created on Sep. 12, 2023, which is 351,227 bytes in size. The information in the electronic format of the Sequence Listing is incorporated herein by reference in its entirety.

### BACKGROUND

### Field

The present disclosure generally relates to antigen-binding molecules, e.g., biomolecules, such as antibodies, that bind to Porphyromonas gingivalis, and the treatment and/or prevention of systemic diseases associated with chronic inflammation, multi-systems inflammation, and/or periodontal disease(s) associated with P. gingivalis infection and/or the continuous release of exo-toxins therefrom, using such P. gingivalis bacteria and exotoxin antigen-binding molecules, e.g., biomolecules. Periodontal disease, including Porphyromonas gingivalis infection, has been implicated in various conditions, disorders or diseases including, without limitation, vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); and longevity or age-related disorder, regenerative and stem cell dysfunction.

### Description of the Related Art

Porphyromonas gingivalis is a gram-negative anaerobic, asaccharolytic, red complex bacteria. P. gingivalis can infect and remain permanently in the oral cavity as a polymicrobial biofilm and/or translocate to other body cells/tissues. Upon infection, P. gingivalis can produce and excrete outer membrane vesicles (containing gingipains, hemagglutinin, adhesins and LPS) into the gingival sulcus space with its attending fluid, blood and lymphatic circulation. As disclosed herein, the regularly distributed polyclonal bio-film colonies of P. gingivalis are deeper in the sulcular tissues and extracellular portions of the oral cavity, while the OMVs produced by P. gingivalis are more diffusely spread to surrounding tissues and in the GCF/lymph and micro-vascular systems. P. gingivalis infection can lead to a state of oral and systemic dysbiosis (pathological and abnormal change from the normal oral flora/microbiota) and subsequent chronic local and systemic infection/disease(s), further leading to increased vascular and tissue inflammation throughout the entire body. Certain end organs, e.g., heart vessels, carotid arteries, vessels in the brain, liver, joints, lungs, pancreas, reproductive system, etc., are more affected than others. P. gingivalis-induced inflammation is implicated in diseases such as cardiovascular disease, heart attacks, atherosclerosis, stroke, various dementias, early and later neuro-cognitive decline, Alzheimer's disease, diabetes, NASH, rheumatoid arthritis, insulin resistance, etc.

### SUMMARY

Provided herein is a human or humanized antigen binding molecule (ABM) that binds to Porphyromonas gingivalis, wherein the ABM comprises: a heavy chain variable region (HVR) comprising: a complementarity determining region (HCDR) 1 of a HCDR1 of SEQ ID NO:9 or 37; a HCDR2 of a HCDR2 of SEQ ID NO:9 or 37; and a HCDR3 of a HCDR2 of SEQ ID NO:9 or 37; and a light chain variable region (LVR) comprising: a complementarity determining region (LCDR) 1 of a LCDR1 of SEQ ID NO:10 or 38; a LCDR2 of a LCDR2 of SEQ ID NO:10 or 38; and a LCDR3 of a LCDR2 of SEQ ID NO:10 or 38, wherein the ABM comprises at least one of: one or more HVR residues selected from L48, L67, K71, V78, and M92, as numbered according to the numbering as provided in SEQ ID NO:37, and one or more LVR residues selected from Q46, W48, A61, Y72, and T86, as numbered according to the numbering as provided in SEQ ID NO:38. Optionally, the HVR comprises one or more of a HFR1, HFR2, HFR3, and HFR4 of a HFR1, HFR2, HFR3, and HFR4 of SEQ ID NO:37, respectively. In some embodiments, the LVR comprises one or more of a LFR1, LFR2, LFR3, and LFR4 of a LFR1, LFR2, LFR3, and LFR4 of SEQ ID NO:38, respectively. In some embodiments, the HVR comprises an amino acid sequence at least 80% identical to one of SEQ ID NOS:29-32. In some embodiments, the LVR comprises an amino acid sequence at least 80% identical to one of SEQ ID NOS:33-36.

Also provided herein is a human or humanized antigen binding molecule (ABM) that binds to Porphyromonas gingivalis, wherein the ABM competes for binding to Porphyromonas gingivalis with H5, H7, or H14, wherein the ABM is not KB001. Optionally, the ABM comprises a heavy chain complementarity determining region (HCDR) 1 of SEQ ID NO:3. In some embodiments, the ABM comprises a HCDR2 of SEQ ID NO:4. In some embodiments, the ABM comprises a HCDR3 of SEQ ID NO:5. In some embodiments, the ABM comprises a LCDR1 of SEQ ID NO:6. In some embodiments, the ABM comprises a LCDR2 of SEQ ID NO:7. In some embodiments, the ABM comprises a LCDR3 of SEQ ID NO:8. In some embodiments, the ABM comprises a HVR of SEQ ID NO:9. In some embodiments, the ABM comprises a LVR of SEQ ID NO:10. In some embodiments, the ABM comprises a FR sequence of one or more of SEQ ID NOs: 11-18.

In some embodiments, the ABM binds to a same or overlapping epitope as KB001. In some embodiments, the ABM comprises the CDRs of the 6 CDRs in SEQ ID NO: 1 and 2. In some embodiments, the ABM binds to an epitope comprising GVSPKVCKDVTVEGSNEFAPVQNLT (SEQ ID NO:19) and/or YCVEVKYTAGVSPK (SEQ ID NO:59) found in the HagA repeat epitope hemagglutinin/gingipains/adhesin domain (HXHRE domain). In some embodiments, the ABM is resistant to protease cleavage. In some embodiments, the resistance is to cleavage by a bacterial protease. Optionally, the resistance is a resistance of 25-75%.

In some embodiments, the ABM binds to a gingipain and/or a haemagglutinin. In some embodiments, the gingipain is selected from the group consisting of: lys-gingipain (Kgp), arg-gingipains (Rgp) A and RgpB. In some embodiments, the gingipain comprises a sequence of SEQ ID NO:19. In some embodiments, the gingipain comprises a sequence of at least one of SEQ ID NOs:21-28.

In some embodiments, the ABM neutralizes the activity of the gingipain. In some embodiments, the activity is at least one of: a peptidase inhibitor, hemagglutination inhibitor, hemolysis inhibitor, and adhesin-inhibitor. In some embodiments, the ABM binds to a pro-peptide domain, a auto-catalytic domain and/or a C-terminal adhesion domain that needs to undergo auto-catalytic processing into other smaller poly-protein fragments needed by the bacteria for survival (FIGS. 19 A, B and 20).

In some embodiments, the ABM binds to budding outer membrane vesicles of P. gingivalis.

Also provided herein is a human or humanized antigen binding molecule (ABM) that binds to Porphyromonas gingivalis, wherein the ABM binds to budding outer membrane vesicles of P. gingivalis.

In some embodiments, the ABM is digested at a slower rate than a fully humanized antibody that specifically binds P. gingivalis.

In some embodiments, the ABM is a Fab, a diabody, Fab′, F(ab′)₂, Fv, single-chain antibody, nanobody, domain antibody, bivalent antibody, bispecific antibody, or peptibody.

In some embodiments, the antibody when administered to a subject's mouth reduces a P. gingivalis infection in the mouth by at least 80%.

In some embodiments, the ABM is of an IgG isotype.

Also provided is a nucleic acid encoding an ABM of the present disclosure. Also provided is a vector comprising the nucleic acid of the present disclosure. Further provided is a cell comprising the nucleic acid or the vector of the present disclosure.

Provided herein is a method of administering an ABM of the present disclosure, the method comprising sub-gingivally and numerous other oral methods of administering the ABM to a subject. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. Optionally, the ABM is administered at least two times. In some embodiments, the ABM is administered 10-16 days apart.

Also provided herein is a method of treating or preventing a vascular disease or symptoms thereof, comprising: identifying a subject in need of treating or preventing a vascular disease or symptoms thereof; and administering to the subject a therapeutically effective amount of the ABM of the present disclosure, thereby treating or preventing the vascular disease or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. Optionally, the vascular disease comprises cardiovascular disease, atherosclerosis, coronary artery disease, myocardial infarction, stroke, and myocardial hypertrophy.

In some embodiments, the method includes administering to the subject at least one other therapeutic agent for treating or preventing the vascular disease, or symptoms thereof. Optionally, the other therapeutic agent comprises a serum lipid lowering agent. In some embodiments, the other therapeutic agent is a statin.

Also provided is a method of treating or preventing a vascular disease or symptoms thereof, comprising: administering to a subject in need of treating or preventing a vascular disease, or symptoms thereof, a therapeutically effective amount of at least one therapeutic agent for treating or preventing the vascular disease, or symptoms thereof; and administering an effective amount of the ABM of the present disclosure, to thereby enhance the therapeutic effect of the at least one therapeutic agent. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. Optionally, the other therapeutic agent comprises a serum lipid lowering agent. Optionally, the other therapeutic agent is a statin.

Provided herein is a method of treating or preventing a systemic disease or symptoms thereof, comprising: identifying a subject in need of treating or preventing a systemic disease or symptoms thereof, wherein the systemic disease is one or more of type II diabetes, insulin resistance and metabolic syndrome; and administering to the subject a therapeutically effective amount of the ABM of the present disclosure, thereby treating or preventing the systemic disease or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1.

Also provided is a method of treating or preventing rheumatoid arthritis or symptoms thereof, comprising: identifying a subject in need of treating rheumatoid arthritis or symptoms thereof; and administering to the subject a therapeutically effective amount of the ABM of the present disclosure, thereby treating or preventing the rheumatoid arthritis or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1.

Provided herein is a method of treating or preventing cancer or symptoms thereof, comprising: identifying a subject in need of treating cancer or symptoms thereof; and administering to the subject a therapeutically effective amount of the ABM of the present disclosure, thereby treating or preventing the cancer or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. Optionally, the cancer is oral, gastrointestinal, lung or pancreatic cancer.

In some embodiments, the method includes administering to the subject at least one other therapeutic agent for treating or preventing the cancer, or symptoms thereof. Optionally, the other therapeutic agent comprises a small molecule drug or immunotherapeutic agent.

Also provided is a method of treating or preventing cancer or symptoms thereof, comprising: administering to a subject in need of treating or preventing cancer, or symptoms thereof, a therapeutically effective amount of at least one therapeutic agent for treating or preventing the cancer, or symptoms thereof; and administering an effective amount of the ABM of the present disclosure, to thereby enhance the therapeutic effect of the at least one therapeutic agent. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. Optionally, the at least one therapeutic agent comprises a small molecule drug or immunotherapeutic agent. In some embodiments, the cancer is oral, gastrointestinal, lung or pancreatic cancer.

Also provided herein is a method of treating or preventing a gut microbiome-related disorder or symptoms thereof, comprising: identifying a subject in need of treating a gut microbiome-related disorder or symptoms thereof; and administering to the subject a therapeutically effective amount of the ABM of the present disclosure, thereby treating or preventing the gut microbiome-related disorder or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. Optionally, the gut microbiome-related disorder comprises inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity.

Provided herein is a method of treating or preventing a cognitive disorder or symptoms thereof, comprising: identifying a subject in need of treating a cognitive disorder or symptoms thereof; and administering to the subject a therapeutically effective amount of the ABM of the present disclosure, thereby treating or preventing the cognitive disorder or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. Optionally, the cognitive disorder is Alzheimer's disease. In some embodiments, the cognitive disorder is early, middle or late dementia.

Also provided is a method of treating or preventing an age-related or longevity-related disorder, or symptoms thereof, comprising: identifying a subject in need of treating an age-related or longevity-related disorder; and administering to the subject a therapeutically effective amount of the ABM of the present disclosure, thereby treating or preventing the age-related or longevity-related disorder, or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1.

Provided herein is a method of treating or preventing a post event myocardial hypertrophy or symptoms thereof, comprising: identifying a subject in need of treating or preventing a post event myocardial hypertrophy or symptoms thereof; and administering to the subject a therapeutically effective amount of the ABM of the present disclosure, thereby treating or preventing the post event myocardial hypertrophy or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1.

Further provided herein is a method of treating a wound, comprising: identifying a subject in need of treating a wound; and administering to the subject a therapeutically effective amount of the ABM of the present disclosure, whereby closure of the wound is enhanced, thereby treating the wound. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1.

Also provided is a method of treating or preventing an age-related macular degeneration (AMD) or symptoms thereof, comprising: identifying a subject in need of treating or preventing AMD or symptoms thereof; and administering to the subject a therapeutically effective amount of the ABM of the present disclosure, thereby treating or preventing the AMD or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1.

Provided herein is a method of treating or preventing an aneurysm or symptoms thereof, comprising: identifying a subject in need of treating or preventing an aneurysm or symptoms thereof; and administering to the subject a therapeutically effective amount of the ABM of the present disclosure, thereby treating or preventing the aneurysm or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. In some embodiments, the aneurysm is a cerebral or abdominal aneurysm.

Provided herein is a method of treating or preventing a glioma or symptoms thereof, comprising: identifying a subject in need of treating or preventing a glioma or symptoms thereof; and administering to the subject a therapeutically effective amount of the ABM of the present disclosure, thereby treating or preventing the glioma or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1.

Also provided is a method of treating or preventing a large vessel stroke C-IMT or symptoms thereof, comprising: identifying a subject in need of treating or preventing a large vessel stroke, C-IMT or symptoms thereof; and administering to the subject a therapeutically effective amount of the ABM of the present disclosure, thereby treating or preventing the large vessel stroke C-IMT or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1.

Also provided is a method of treating or preventing a microvascular defects and associated dementia (e.g., Parkinson's), or symptoms thereof, comprising: identifying a subject in need of treating or preventing a microvascular defects and associated dementias (e.g., Parkinson's), or symptoms thereof; and administering to the subject a therapeutically effective amount of the ABM of the present disclosure, thereby treating or preventing the microvascular defects Parkinson's or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1.

Provided herein is a method of treating or preventing a peri-implantitis and/or periodontal disease and/or associated bone loss, or symptoms thereof, comprising: identifying a subject in need of treating or preventing a peri-implantitis and/or periodontal disease and/or associated bone loss, or symptoms thereof; and administering to the subject a therapeutically effective amount of the ABM of the present disclosure, thereby treating or preventing the peri-implantitis and/or periodontal disease and/or associated bone loss or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1.

Also provided is a method of treating or preventing a renal disease or symptoms thereof, comprising: identifying a subject in need of treating or preventing a renal disease or symptoms thereof; and administering to the subject a therapeutically effective amount of the ABM of the present disclosure, thereby treating or preventing the renal disease or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1.

Also provided is a method of treating or preventing a regenerative and stem cell dysfunction, or symptoms thereof, comprising: identifying a subject in need of treating or preventing a regenerative and stem cell dysfunction, or symptoms thereof; and administering to the subject a therapeutically effective amount of the ABM of the present disclosure, thereby treating or preventing the regenerative and stem cell dysfunction, thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. Provided herein is a method of treating or preventing a condition, disorder or disease associated with a P. gingivalis infection, or symptoms thereof, comprising: identifying a subject in need of treating or preventing a condition, disorder or disease associated with a P. gingivalis infection, or symptoms thereof; and administering to the subject a therapeutically effective amount of the ABM of the present disclosure, thereby treating or preventing the condition, disorder or disease associated with a P. gingivalis infection, or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. Optionally, the method includes administering the therapeutically effective amount of the ABM to treat the condition, disorder or disease associated with a P. gingivalis infection, or symptoms thereof. Optionally, the method includes administering the therapeutically effective amount of the ABM to prevent the condition, disorder or disease associated with a P. gingivalis infection, or symptoms thereof. In some embodiments, the condition, disorder or disease is associated with a local infection of P. gingivalis. In some embodiments, the condition, disorder or disease is associated with a systemic infection of P. gingivalis. In some embodiments, the condition, disorder or disease is associated with an oral infection of P. gingivalis. In some embodiments, the condition, disorder or disease is one or more of: vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); regenerative and stem cell dysfunction; and longevity or age-related disorder. In some embodiments, the condition, disorder, or disease is present in multiple systems, organs, or tissues. In some embodiments, treating or preventing the condition, disorder or disease associated with a P. gingivalis infection results in the decrease of CRISPR-Cas gene expression at one or more site of infection. In some embodiments, treating or preventing the condition, disorder or disease associated with a P. gingivalis infection results in a decrease of local inflammation. In some embodiments, the decrease of local inflammation is reduced activity or activation of inflammasomes, reduced cytokine levels, and/or lowered host cell death. In some embodiments, treating or preventing the condition, disorder or disease associated with a P. gingivalis infection results in a decrease of systemic inflammation. In some embodiments, the decrease of systemic inflammation is reduced proinflammatory mediators, and/or reduced chronic distant site inflammatory atherosclerosis.

Also provided herein is a method of targeting a P. gingivalis. In some embodiments, the method comprises identifying a subject with a P. gingivalis infection, or symptoms thereof; and administering to the subject a therapeutically effective amount of the ABM disclosed herein, thereby targeting the P. gingivalis, or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. In some embodiments, the P. gingivalis infection is in the mouth. In some embodiments, the P. gingivalis infection is in the gums. In some embodiments, the P. gingivalis infection is in the brain. In some embodiments, the P. gingivalis infection is across the blood brain barrier. In some embodiments, the targeting of the P. gingivalis infection further comprises administration of a small molecule, antibiotic, or drug affective against P. gingivalis. In some embodiments, the small molecule, antibiotic, or drug targets P. gingivalis virulence factors, thereby inhibiting/decreasing the production of the Lys and Arg-specific proteases needed by P. gingivalis, thus reducing P. gingivalis ability for heme degradation and iron uptake, reduces the access to needed amino acids from protein catalysis by P. gingivalis, leading to and/or enhancing bacterial cell death and loss of biofilm integrity for P. gingivalis.

Also disclosed herein is a method of targeting a bacterial infection in a subject. In some embodiments, this method comprises identifying the subject with a bacterial infection, or symptoms thereof; and administering to the subject a therapeutically effective amount of any of the ABMs disclosed herein, thereby targeting the bacterial infection, or symptoms thereof. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. In some embodiments, the bacterial infection is in the mouth. In some embodiments, the bacterial infection is in the gums. In some embodiments, the bacterial infection is in the brain. In some embodiments, the bacterial infection is in the gut. In some embodiments, the bacterial infection is across the blood brain barrier. In some embodiments, the bacterial infection is systemic, and/or in multiple tissues including lungs. In some embodiments, the bacterial infection comprises a P. gingivalis infection. In some embodiments, the bacterial infection comprises more than one bacterial infections. In some embodiments, the targeting of the bacterial infection further comprises administration of a small molecule, antibiotic, or drug. In some embodiments, the small molecule, antibiotic, or drug targets at least one virulence factors, increases the production of proteases, reduces bacterial nutrient uptake, alters bacterial protein and energy production, and/or enhances bacterial cell death.

In some embodiments, the administering comprises administering the ABM intravenously, sub-gingivally, intradermally, subcutaneously, intrathecally, or by nebulization.

Also provided herein is a use of an ABM of the present disclosure, for treatment of a disorder associated with, caused by or complicated by P. gingivalis. In some embodiments, the ABM is any one of the ABMs described in the present disclosure. In some embodiments, the ABM has a heavy chain variable region within SEQ ID NO: 1 and a light chain variable region within SEQ ID NO: 2. In some embodiments, the ABM has a LCDR1, a LCDR2, and a LCDR3 within SEQ ID NO: 2 and a HCDR1, a HCDR2, and a HCDR3 within SEQ ID NO: 1. In some embodiments, disorder associated with, caused by or complicated by P. gingivalis is one or more of: vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); neuroinflammatory diseases; regenerative and stem cell dysfunction; and longevity or age-related disorder.

In some of the embodiments provided herein, the ABM comprises a point mutation for cleavage resistance from Pg proteases, in a human or humanized FR context. In some embodiments, the ABM comprises an amino acid sequence with a point mutation at position 222. In some embodiments, the point mutation at position 222 is an alanine. In some embodiments, the ABM comprises an amino acid sequence at least 80% identical to SEQ ID NO: 84. In some embodiments, the HVR comprises an amino acid sequence at least 80% identical to one of SEQ ID NOS:85-86. In some embodiments, the LVR comprises an amino acid sequence at least 80% identical to one of SEQ ID NOS:87-90. In some embodiments, the ABM comprises an HVR amino acid sequence corresponding to a nucleic acid sequence that is at least 80% identical to one of SEQ ID NOS:91-92. In some embodiments, the ABM comprises an LVR amino acid sequence corresponding to a nucleic acid sequence that is at least 80% identical to one of SEQ ID NOS:93-97. In some embodiments, the ABM corresponds to a nucleic acid sequence that is at least 80% identical to one of SEQ ID NOS: 98-101. In some embodiments, the ABM further comprises at least one of an alanine at position 222, an amino acid sequence that is at least 80% identical to SEQ ID NO: 84, an HVR sequence comprising an amino acid sequence at least 80% identical to one of SEQ ID NOS:85-86, and/or an LVR sequence comprising an amino acid sequence at least 80% identical to one of SEQ ID NOS:87-90. In some embodiments, the ABM binds to a gingipain and/or a haemagluttinin with a KD that is less than about 2E-9 M, less than about 1E-9 M, less than about 9E-10 M, less than about 8E-10 M, less than about 6E-10 M, less than about 4E-10 M, less than about 2E-10 M, less than about 1E-10 M, less than about 9E-11 M, and/or less than about 7E-11 M.

In some embodiments, the ABM comprises at least one, two, three, or all four of: an alanine at position 222; an amino acid sequence that is at least 80% identical to SEQ ID NO: 84; an HVR sequence comprising an amino acid sequence at least 80% identical to one of SEQ ID NOS:85-86; and/or an LVR sequence comprising an amino acid sequence at least 80% identical to one of SEQ ID NOS:87-90. In some embodiments, the ABM comprises SEQ ID NO: 1 and/or SEQ ID NO: 2. In some embodiments, the ABM comprises a heavy chain sequence of SEQ ID NO: 30, a light chain sequence of SEQ ID NO: 33, except that the ABM comprises an alanine at position 222. In some embodiments, the ABM is H5 K22A.

Also disclosed herein is an ABM that is humanized or human. In some embodiments, ABM that is humanized or human.

Also disclosed herein is a method for treating a disorder driven by P. gingivalis. In some embodiments, the method comprises providing an antibody that binds to a P. gingivalis associated peptide to a subject; wherein the antibody is known to function to stop a P. gingivalis infection; wherein the antibody is a humanized or human antibody; and wherein position 222 of the antibody has been changed to an alanine.

Also disclosed herein is a nucleic acid that is at least 80% identical to one of SEQ ID NOS: 98-101. Also disclosed herein is a human or humanized antigen binding molecule (ABM) that binds to a protein complex, protein, peptide, or amino acid sequence comprising the sequence YTYTVYRDGTKIK (SEQ ID NO: 190).

Any of the embodiments provided herein can be directed to or substituted with ABM (including antibodies) that bind to the following sequence: YTYTVYRDGTKIK (SEQ ID NO: 190).

### BRIEF DESCRIPTION OF THE DRAWINGS

FIGS. 1A and 1B show the heavy and light chain amino acid sequences, respectively, of KB001 (which includes HC SEQ ID NO: 1 and LC SEQ ID NO: 2). The construct is a mouse construct, which can be used in any of the method embodiments provided herein.

FIG. 2A shows the amino acid sequence of a full length RgpA exotoxin from Porphyromonas gingivalis, strain W50.

FIG. 2B shows the amino acid sequence of a full length RgpA exotoxin from Porphyromonas gingivalis, strain HG66.

FIG. 3A shows the amino acid sequence of a full length RgpB exotoxin from P. gingivalis, strain W50.

FIG. 3B shows the amino acid sequence of a full length RgpB exotoxin from P. gingivalis, strain W83.

FIG. 4A shows the amino acid sequence of a full length Kgp exotoxin from Porphyromonas gingivalis, strain W83.

FIG. 4B shows the amino acid sequence of a full length Kgp exotoxin from Porphyromonas gingivalis, strain ATCC 33277.

FIG. 5A shows the amino acid sequence of a full length HagA from Porphyromonas gingivalis, strain W83.

FIG. 5B shows the amino acid sequence of a full length HagA from Porphyromonas gingivalis, strain 381.

FIG. 6A shows the response curves at antibody concentrations of 33.3 nM (E3), 100 nM (C3) and 200 nM (A3).

FIG. 6B shows the data aligned by the step baseline. The data was further fitted, as shown in FIGS. 6C and 6D. These graphs show the response curves for KB001 binding to whole P. gingivalis cells, at different concentrations of antibody, measured using surface plasmon resonance. Table 2.1 summarizes the results.

FIG. 7 is SEM imaging of KB-001 binding to the P. gingivalis. strain W83. The left panel shows the cell surface at 500 nm magnification, using gold labeling. The middle panel shows KB-001 localization at 500 nm magnification. The right panel shows KB-001 localization at 2 μm magnification.

FIG. 8 is a collection of images showing binding of KB001 to outer membrane vesicles (OMV) and OMV blebs of P. gingivalis, W 83, visualized using secondary gold-labeled anti-mouse antibody.

FIG. 9 is a Western blot of P. gingivalis Outer Membrane Vesicles (OMV) probed with KB001.

FIG. 10 is a phylogram of P. gingivalis strains, grouped by the presence or absence of accessory genes. The arrows mark the ten strains selected to represent the diversity of P.g. strains.

FIG. 11 is a collection of SEM images showing W83 immunogold labeling against KB001 (left panel) and 1A1 (right panel) primary antibody, single label.

FIG. 12 is a collection of SEM images showing the lack of KB001 binding to gingipain mutants of P. gingivalis. Left panel is a RgpA-/KgP-gingipain knockout strain, and right panel is a RgpB-/KgP-gingipain knockout strain.

FIG. 13 is a graph showing binding of KB001 to acetone precipitated gingipain.

FIG. 14A is a collection of images showing immunohistochemistry staining (IHC) of hippocampal tissue slices from the brain of a deceased Alzheimer's disease patient using KB001.

FIG. 14B shows imaging of AD brain tissue. The brain tissue is labeled for gingipain using binding by KB-001.

FIG. 14C shows immunohistochemistry staining of P. gingivalis using KB001 binding to intra-cellular accumulated gingipains located in a hippocampal tissue from the brain of a deceased Alzheimer's disease patient.

FIG. 14D is an image showing a P. gingivalis positive control human gum tissue used in brain IHC analysis.

FIG. 14E shows frontal lobe using immunohistochemistry staining with KB001.

FIG. 14F is an image showing human choroid plexus IHC stained section of AD brains using KB001 (20X-left panel and 40 X-right panel).

FIG. 15A shows the gingipain antibody signal intensity from frontal lobe immunostaining of subjects AMC3,3, AD3,3, and AD4,4.

FIG. 15B shows the gingipain antibody signal intensity from occipital lobe immunostaining of subjects AMC3,3, AD3,3, and AD4,4.

FIG. 15C shows the gingipain antibody signal intensity from cerebellum immunostaining of subjects AMC3,3, AD3,3, and AD4,4.

FIG. 15D shows the gingipain antibody signal intensity from hippocampus immunostaining of subjects AMC3,3, AD3,3, and AD4,4.

FIG. 16 is a gel image showing the sensitivity of a PCR-based liquid hybridization assay for detection of P. gingivalis.

FIG. 17 is a graph showing dose response titration binding of KB001 monoclonal antibodies from various hybridoma clones to isolated P. gingivalis gingipains.

FIG. 18 is a graph showing selection of various KB001 cloned murine monoclonal antibody cell hybridomas selected for the master cell bank.

FIG. 19A is an image of a Western blot showing HagA processing by gingipains Kgp/RgpA mix, with KB001 interfering/blocking its normal bacterial proteolytic processing, according to embodiments of the present disclosure.

FIG. 19B is an image of an SDS-PAGE showing uninhibited processing of HagA by gingipains Kgp/RgpA mixture.

FIG. 20 shows a Western Blot for KB-001 binding to Kgp/RgpA:HagA and RgpB:HagA complexes.

FIGS. 21A and 21B are images showing mapping of KB001 mouse monoclonal antibody target binding by N-term sequencing and mass spectrometry, which can be equated to the relevant AP sections, as disclosed herein.

FIGS. 22A, 22B, 22C, 22D, 22E, 22F, 22G, 22H, 22I, and 22J are mapped protein sequences from the P. gingivalis the repeat epitope in hemagglutinin/adhesion and HagA gingipains domain (RE-HagA) protein complex specific to binding of KB-001 and the preliminary linear amino acid sequence of the KB-001 antibody binding epitope, according to some embodiments of the present disclosure, which can be equated to the AP as provided herein.

FIGS. 23A and 23B show expression of human chimeric KB001 monoclonal antibodies, according to some embodiments of the present disclosure.

FIG. 24 is a collection of ELISA graphs showing identification of and down selection of human chimeric KB001 monoclonal antibodies that compete with KB001 and bind gingipains, according to some embodiments of the present disclosure.

FIGS. 25A and 25B are graphs showing ELISA results from competition binding assay of varying concentrations of the KB001 and a humanized variant, according to some embodiments of the present disclosure.

FIG. 26A shows non-limiting examples of the amino acid sequences of a CDR grafted ABM variable regions, according to some embodiments of the present disclosure.

FIG. 26B shows non-limiting examples of the amino acid sequences of KB001 variable regions.

FIG. 26C shows an alignment of KB001 heavy chain with structural template 1DVF.

FIG. 26D shows non-limiting examples of the amino acid sequences of KB001 variable regions.

FIG. 26E shows an alignment of the VH and VL amino acid sequences of KB001 with the grafted VH and VL sequences, respectively.

FIGS. 27A, 27B, 27C, and 27D show non-limiting examples of amino acid sequences of heavy chain variable regions of antigen binding molecules, according to some embodiments of the present disclosure.

FIGS. 28A, 28B, 28C, and 28D show non-limiting examples of amino acid sequences of light chain variable regions of antigen binding molecules, according to some embodiments of the present disclosure.

FIG. 29 shows non-limiting examples of amino acid sequences of human heavy chain and light chain constant regions, according to some embodiments of the present disclosure.

FIG. 30 shows non-limiting examples of amino acid sequences of heavy and light chain variable regions of antigen binding molecules, according to some embodiments of the present disclosure.

FIG. 31 shows the amino acid sequence of KB001, according to some embodiments of the present disclosure.

FIG. 32 shows an alignment of some antigen binding molecule heavy chain variable region sequences, according to some embodiments of the present disclosure.

FIGS. 33A, 33B, 33C, and 33D are non-limiting examples of grafted nucleic acid sequences encoding heavy chain variable regions of KB001 antigen binding molecules, according to some embodiments of the present disclosure.

FIGS. 34A, 34B, 34C, and 34D are non-limiting examples of grafted nucleic acid sequences encoding light chain variable regions of KB001 antigen binding molecules, according to some embodiments of the present disclosure.

FIGS. 35A and 35B are non-limiting examples of grafted nucleic acid sequences encoding heavy and light chain variable regions, respectively, of an KB001 antigen binding molecule, according to some embodiments of the present disclosure.

FIGS. 36A and 36B are non-limiting examples of grafted nucleic acid sequences encoding human heavy chain and light chain constant regions of KB001, according to some embodiments of the present disclosure.

FIGS. 37A, 37B, 37C, 37D show nucleotide sequences encoding heavy and light chains of KB001, and their translated amino acid sequences, according to some embodiments of the present disclosure.

FIG. 38 shows a schematic design of constructing Hu-chimeric antibodies from a mouse parent IgG1 (KB001), according to some embodiments of the present disclosure.

FIGS. 39A and 39B show SEM images from whole P. gingivalis bacterial cell gold-label binding assay of antigen binding molecules, according to some embodiments of the present disclosure.

FIG. 40A shows an amino acid sequence of hemagglutinin protein HagA from Porphyromonas gingivalis strain ATCC 33277. Proteolytic processing sites are marked with bold font.

FIG. 40B shows amino acid sequences of the repeated domains of HagA, RgpA, and Kgp, with sequences encompassing some of the putative epitopes of KB001 underlined, according to some embodiments of the present disclosure. The Hemoglobin Receptor (HbR) domain is boxed in a rectangle. Proteolytic processing sites are marked with bold font. For “Kgp_W83”, HA1 is in italic, and proteolytic processing of C-terminal HA part of Kgp W83 is not well defined. For “RgpA_W83”, sequence in italics before the boxed sequence shows HA1, sequence in italics at C-terminus shows HA4, and sequence between the boxed sequence and HA4 shows HA3.

FIG. 40C shows a multiple sequence alignment of HA domains of HagA from Porphyromonas gingivalis strains W83 and ATCC 33277. Putative epitope of KB001, according to some embodiments, is underlined.

FIG. 40D shows a multiple sequence alignment of RgpA, Kgp and HagA sequences.

FIG. 40E shows a multiple sequence alignment of RgpA, Kgp and HagA sequences.

FIG. 40F shows a multiple sequence alignment of putative sequence motifs in HagA (from W83 and ATCC 33277 strains) and RgpA and Kgp (from W83) encompassing the epitope recognized by KB001, according to some embodiments of the present disclosure.

FIG. 41 displays amino acid and DNA sequences of the GST-TEV-gingipain-His fusion protein used to produce recombinant gingipain fusion proteins in E. coli. Linker and TEV protease sequence is bold and underlined. Putative KB001 epitope is shown in bold. The linker between the fusion partners and a TEV protease site is shown bold and underlined. Immediately after this sequence starts the gingipain protein fragment which contains a single KB001 epitope. GST Fusion partner is at the beginning, followed by the linker peptide and the TEV protease site (bold and underlined), and then the gingipain fragment.

FIG. 42A is a sequence of rGP-2

FIG. 42B is a comparison between rGP-1 and rGP-2.

FIG. 42C is a hydrophobicity plot of rGP-2.

FIG. 43 shows the sequence for Kgp-8HSLA domain N-terminus from the W83 strain of P.g. In some embodiments, this sequence can be used for screening of binding of one or more of the antibody variants thereof provided in the present application.

FIG. 44 shows the sequence for HRgpA-6H domain N-terminus from P.g. In some embodiments, this sequence can be used for screening of binding of one or more of the antibody variants thereof provided in the present disclosure.

FIG. 45 shows the amino acid sequences of alternative heavy chain segments, alternative light chain segments, hIgG1CH, hIgG1CH K22A, and hIgkCL.

FIG. 46 shows the DNA sequences of alternative heavy chain segments, alternative light chain segments, hIgG1CH, hIgG1CH K22A, and hIgkCL.

FIG. 47 is a table of the heavy and light chain segments present in the H5, H6, H7, H8, and H14 sequences.

FIG. 48A shows the binding kinetics (or “sensor-grams”) of H8 to HRgpA-6H.

FIG. 48B shows the binding kinetics of H14 to HRgpA-6H.

FIG. 48C shows the binding kinetics of KB001 to HRgpA-6H.

FIG. 48D shows the binding kinetics of H5 to HRgpA-6H.

FIG. 48E shows the binding kinetics of H7 to HRgpA-6H.

FIG. 49 shows the sensor-grams of the parental mouse (KB001) Fab FASEBA supernatant to antigen in a low salt buffer.

FIG. 50 shows the sensor-grams of the parental mouse (KB001) Fab FASEBA supernatant to antigen in a high salt buffer.

FIG. 51A shows the read coverage and distribution of VH-CDRs across chimeric variants.

FIG. 51B shows the read coverage and distribution of VL-CDRs across chimeric variants.

FIG. 52A shows the Fab VH sequence of the parental mouse (KB001) construct.

FIG. 52B shows the Fab VH sequence of the parental mouse (KB001) construct.

### DETAILED DESCRIPTION

Provided herein are antigen binding molecules (ABMs), e.g., murine, human-chimeric, human or humanized ABMs, that bind to Porphyromonas gingivalis. The ABMs, e.g., antibodies, of the present disclosure can specifically bind to an epitope associated with P. gingivalis, including certain cell-surface epitopes.

As disclosed herein, the ABMs are clinically validated for eliminating P. gingivalis. In some embodiments, the antigenic peptides, proteins, and/or antibodies disrupt the later stages of the major protein surface processing machinery and/or prevent the maturation of the unique subunit toxin “XXX Epitope.” This subunit toxin is needed for both P. gingivalis survival, and the creation of P. gingivalis's secreted outer membrane vesicles (OMVs) that result in systemic multi-systems pathology. The “XXX Epitope” is a one-of-a-kind virulent subunit protein complex in neuro-anatomic strategic sites of AD brain tissues.

In some embodiments, the ABM is an antibody. For instance, the antibody KB-001 is a monoclonal antibody with unique binding to P. gingivalis and its virulence factors, In some embodiments, the ABM binds to an epitope comprising GVSPKVCKDVTVEGSNEFAPVQNLT (SEQ ID NO:19) and/or YCVEVKYTAGVSPK (SEQ ID NO:59) and/or YTYTVYRDGTKIK (SEQ ID NO: 190) found in the HagA repeat epitope hemagglutinin/gingipains/adhesin domain (HXHRE domain).

As demonstrated in the below examples, KB-001 was shown during clinical study to prevent the recolonization of P. gingivalis, thereby eliminating all of the virulence factors of P. gingivalis contributing to systematic and/or organ-based inflammation at their source. In some embodiments, Kbhu-007 is effective in treating, ameliorating, and/or preventing neurodegenerative disorders, Alzheimer's disease, Parkinson's disease, dementia, systemic wide inflammatory disease and/or cardiometabolic diseases. KBhu-07 and KBhu-0014 are humanized chimeric monoclonal antibody candidates with similar binding to P. gingivalis and its “XXX Epitope” as KB-001. In some embodiments, Kbhu-007 is effective in treating, ameliorating, and/or preventing neurodegenerative and/or systemic wide inflammatory disease. In some embodiments, Kbhu-014 is effective in treating, ameliorating, and/or preventing neurodegenerative and/or systemic wide inflammatory disease.

The KB-001 monoclonal antibody recognizes the proteinase/adhesin/hemagglutinating complex. As disclosed herein, the antibody recognized all 22 laboratory and 105 human clinical isolates strains and serotypes by IF. The immunogen used to generate the body was formalinized Porphyromonas gingivalis, strain W83 (full length protein). On a gel, KB-001 has multiple bands between 31 and 65 kDa, two bands around 14 kDa, and higher MW bands at around 113 kDa. It has a mouse isotype of IgG1, and is registered with the Entrez Gene ID 2552074 29256891 2551934.

The broader target activity of KB-001 is unusual with possible gene duplication(s) of critical accessory functions. The two arginine-specific gingipains, RgpA and RgpB, possess practically identical caspase-like catalytic domains and specifically cleave Arg-Xaa peptide bonds. RgpA, however, possesses a large C-terminal extension bearing a hemagglutinin-adhesion domain, which is absent from RgpB. The Rgp/Kgp/adhesion/hemagglutinins complex recognized by the antibody KB-001 include RgpA (Gingipain R1; also known as prpR1 or hemagglutinin HagA), Kgp (Lys-gingipain) and HagA (Hemagglutinin A) are responsible for the known major survival virulence factors that include colonization, agglutination hemagglutination/heme acquisition via RBC lysis, amino acids, adhesion complex, and host defenses against innate complement degradation/inactivation and acquired immunity (antibody cleavage). The activity of RgpA, Kgp, and HagA are mediated through the human IL-1B/NLRP3 pathway, and thus binding of RgpA. Kgp, and/or HagA to KB-001 may also block the advancement and interaction of this cytokine with its receptors and downstream pathways, such as systematic cellular inflammation, host defenses, and pre-oncogenic pathways. Booth et at showed that subgingical application of an anti-gingipain A1 adhesin monoclonal antibody could prevent recolonization of subgingival plaque by P. gingivalis. As disclosed herein, the KB-001 antibody was mapped, and the inventors found that P.g. infected periodontal patients made natural antibody responses directed to non-protective epitope(s) adjacent to the KB-001 monoclonal antibody mapped epitope. Thus, the KB-001 antibody targets a protective epitope(s) that humans do not make under natural infections. Patients who had naturally developed a specific IgG1 and/or response to the gingipains did not exhibit progressive disease, and appeared stable compared with those subjects with predominant IgG2/IgG3 responses.

In some embodiments, the ABM specifically binds a P. gingivalis gingipain and/or hemagglutinin/adhesin. In some embodiments, the ABM interferes/blocks/reduces a molecular function(s) of its surface binding, bacterial defense activities and/or metabolic activities, e.g., gingipains and/or a hemagglutinin/adhesin complex. In some embodiments, the ABM, e.g., human-chimeric ABM, competes for binding with an ABM provided herein. Also provided are methods of treating and/or preventing periodontal infection or local and systemic inflammation by targeting P. gingivalis, e.g., surface OMV structures of P. gingivalis, using an ABM as described herein. In some embodiments, vesicle production, assembly, and OMV structures are regulated in P. gingivalis. In some embodiments, normal disease progression from P. gingivalis involves the lipopolysaccharide of P. gingivalis (LPS-PG) being integrated into and transported via OMVs. These OMVs are then released into tissue. In our own studies of P. gingivalis in culture and depending on the strains, hundreds of OMVs can be observed emerging from the cell membrane at the same time and on most if not all cells, suggesting that at any relative time point 1.0×10\hat{ }9 CFUs of P. gingivalis can produce 1.0×10\hat{ }11 or greater OMVs. This contributes to the etiology of distant organ diseases; for example, chronic systemic exposure to the lipopolysaccharide of P. gingivalis induces the accumulation of amyloid beta (Aβ) in the brain of middle-aged mice (a hallmark of Alzheimer's disease). Furthermore, there is evidence that OMVs from periodontal pathogens cause AD via leaky gum. In some embodiments, the targeting of surface OMV structures of P. gingivalis by ABM reduces the onset of distant organ disease. In some embodiments, a method of the present disclosure includes identifying a subject in need of treating a condition, disorder or disease associated with Porphyromonas gingivalis, and administering to the subject a therapeutically effective amount of an ABM as disclosed herein, to inactivate and reduce/eliminate the bacteria and its toxic OMVs, thus treating the various conditions, disorders or diseases. In some embodiments, the condition, disorder or disease is, without limitation, one or more of vascular disease (e.g., cardiovascular disease, atherosclerosis, coronary artery disease, myocardial infarction, stroke, and cardiac hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD age related macro-degeneration, cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, or late dementia; Alzheimer's disease); regenerative and stem cell dysfunction; and age-related disorder.

In some embodiments, Pg OMV-mediated sporadic AD and Pg OMV-mediated oral-neurogenic driven diseases are major driving processes for systemic inflammatory diseases. P. gingivalis is the most powerful LF-degrading bacterium of several periodontal pathogens tested in vitro. P. gingivalis exists initially and possibly ultimately as a small population poly-microbial infection. P. gingivalis is a heme auxotroph, and many studies have highlighted the major influence the environmental concentration of heme has on P. gingivalis gene and protein expression as well as the growth and virulence capacity of the microorganism. Heme can be derived from host hemoproteins present in the saliva, gingival crevicular fluid, and erythrocytes in the oral cavity. In vivo concentrations of free heme have been found to be too low (10\hat{ }-24 M) to support bacterial growth without the help of specialized heme acquisition systems produced by the bacteria themselves. Depending on environmental signaling, iron from salivary Lf provide a heme excess environment for so (Phase 1). It is hypothesized that Pg OMVs at this stage have a unique molecular signature that is enriched in various adhesion molecules. These find their way through and around the interstitial spaces (lymphatics) and epithelium/basement membrane to nearby micro-vascular networks. Once there, they circulate to the brain and bind endothelial extravasation signaling molecules, through the BBB/meningeal lining cells, and finally into adjacent neural parenchymal cells. These can explain the early localization to the cholinergic neurons, basal forebrain and anterior hypothalamic regions and regions near ventricles and peripheral neurons, an early pathway to Pg OMV entry to brain (Beginning of Phase 2). Ultimately the brain inflammation in this region leads to a shift in the delicate balance of salivary Lf coming from the decreased production of the salivary glands, shifting the biofilm sensing system to a heme limited environment. It is remarkable that the levels of LF are increased in the brains of AD patients, at least initially, and the also reduced in their whole saliva. The latter scenario could aggravate the BBB and setup the brain for additional less invasive, oro-dontophlic bacteria and other non-specific microbial/viral infections. Phase 3 begins with Pg OMVs enriching their protein cargo for increased iron scavenging. OMVs now entering the brain bring in iron with them and possibly through other unknown endothelial signaling and or now a general breakdown of the BBB these Fe-loaded OMVs target the hippocampus and frontal-temporal lobes and neo-cortex. This is a more pathogenic period for the brain with the loss of the Lf protein protection system of the brain and the more incessant loading of iron a more later advanced stage of AD occurs. Sometime between Phase 2-3 there is a greater chance for the entry of either more Pg bacterial cells other non-specific bacteria, viruses and fungi to locate in the parenchyma. This being due to both the loss of BBB integrity and innate and acquired immune suppression. The early cognitive decline seen in the prodromal period is most likely occurring in Phase 2. The more progressive cognition and memory losses coming in the Phase 3 period when both the Lf protection system is failing and the iron dyshomeostasis is occurring through the iron loaded OMV mediated period.

The extent to which lower amounts of non-iron containing OMVs verses higher containing iron OMVs may be involved in switching the early cognitive-decline form of AD into a more aggressive form of neuropathology and progressing dementia is not known. However it is not unreasonable to think the shift now to a greater deposition of higher iron into the deep gray matter and total neocortex, and regionally in temporal and occipital lobes would not be seen as a poorer prognostic indicator for AD disease progression.

Also provided herein are methods of preventing any one of the conditions, disorders, or diseases, as disclosed herein, by administering to a subject, e.g., a subject at risk of developing the condition, disorder, or disease, an effective amount of an ABM of the present disclosure, to thereby prevent the condition, disorder, or disease or developing. As used herein, “prevent” includes reducing the likelihood of a future event occurring, or delaying the onset of a future event. In some embodiments, the ABM may be used preventatively within the oral subgingival cavity to create a barrier, retardant, and/or non-colonizing effect by P. gingivalis, thereby preventing the bacteria from gaining access to the oral cavity, or reducing the likelihood thereof.

In some embodiments, any of the methods provided herein can be used to target Pg and/or its toxins at its source.

In some embodiments, the methods provided in the application can be used for the treatment/prevention of chronic inflammation, including disorders such as: cardiometabolic disease, atherosclerosis, inflammatory cardiovascular disease, stroke, specific cancers (including pancreatic, oral-esophageal, lung), type 2 diabetes mellitus, and neurodegenerative conditions especially Alzheimer's disease.

In some embodiments, the antibodies provided herein can be used to target and/or reduce virulence factor(s) bacterial protein complex produced by Pg in the mouth and transported via the blood to the end organs like the brain and specific neuro-anatomic regions of AD brain tissues. The Pg bacterial toxic protein complex is secreted actively in large amounts by the bacteria, mostly in the mouth, for its own survival and eventually crosses the blood-brain barrier (BBB). Thus, it impacts the brain parenchyma in specific lysine and arginine rich neuro-anatomic locations within the brain explaining AD locations and hence clinical symptoms and associated pathology. This results in a chronic low-grade systemic bacterial toxemia that disrupts our immune system and spreads throughout the body. This discovery explains the large number of inflammatory based diseases mentioned earlier, while at the same time explaining the conundrum of the pathogen driven form of Sporadic Alzheimer's disease. In some embodiments, the Ab or methods provided in the present application can be used to treat the pathogen driven form of Sporadic Alzheimer's disease. In some embodiments, this can employ KB-001 or a variant thereof, which can inactivate and eliminate both the source and the secreted virulence factors. KB-001 disrupts the later stages of the bacteria's required major protein surface processing machinery.

In some embodiments, KB-001, a monoclonal antibody, or any variant thereof or any Ab provided herein, can be used to inactivate and eliminate both the source and the secreted virulence factors. KB-001 disrupts the later stages of the bacteria's required major protein surface processing machinery. In some embodiments, any humanized version can be used in this manner. In some embodiments, any variant of KB-001 provided herein can be used in this manner. In some embodiments KB-001 can be used (e.g. SEQ ID NO: 1 and SEQ ID NO:2).

In some embodiments, KB001 can be used to treat as a combination of aspects including: general dentist and a general and specialty internal medical practice s (e.g., cardiology, primary care). In some embodiments, KB001 can be used as an antibody, or a DNA sequence or RNA (or mRNA) sequence encoding the amino acid (or applicable part thereof) can be used to administer the Ab to the subject. In some embodiments, any nucleic acid encoding any of the Ab provided herein are contemplated a nucleic acid based therapeutics for effectively delivering the Ab. The construct can include a nucleic acid sequence for part or all of the heavy and/or light chains and/or CDRs noted herein, and then be part of or configured for a viral vector delivery system or other system for delivery to humans. In some embodiments, the nucleic acid system includes the mouse sequence (e.g., KB001 or CDRs thereof) and is configured to administration to a human subject directly and either DNA or m-RNA or via any of a number of other nucleic acid delivery systems and viral vector systems.

In some embodiments, KB-001 and/or any of the variants provided in the present application can be used to prevent recolonization for up to 1 year in patients given the antibody.

In some embodiments, therapeutic antibody is a human chimeric monoclonal antibodies, allowing for repeat systemic dosing.

In some embodiments, the therapeutic Ab, including optionally KB-001, or variants thereof, prevents Pg from synthesizing its secreted outer membrane vesicles (OMVs) containing virulence protein complexes, resulting in the bacteria shutting down its metabolic and host defense functions. KB-001 has the capability to treat Pg, eliminating it and all of its virulence factors.

In some embodiments, KB-001 (or a variant thereof) binds directly to a unique hetero-multimer repeat protein epitope involved in the bacterial cargo IX transporter secretion protein complex essential for bacterial survival.

In some embodiments, the antibody can be used to treat an adverse medical condition associated with Porphyromonas gingivalis (Pg) infection associated with the long term, oral, biofilm-associated colonization in humans and associated with a state of chronic systemic inflammation and multiple organ system diseases (e.g., atherosclerosis, cardiovascular, stroke, diabetes type 2/metabolic syndrome, cancer, multiple forms of cognitive dementias, Alzheimer, Parkinson etc.

In some embodiments, KB-001 (or a variant thereof) binds directly to a unique hetero-multimer antigen involved in the bacterial cargo IX transporter secretion protein complex through a high affinity bi-valent binding (kD 10⁻⁸⁻⁹).

In some embodiments, about 40-60 antibody molecules bind to emerging OMVs per bacterial. Isolated OMVs demonstrate binding to the outer and inner membranes. In some embodiments, the mechanism of action is that the antibody interferes with the proteolytic processing of the larger parent protein required for subsequent endo-peptidase activity and assembly. More specifically, the binding of antibody to this complex prevents the maturation of the gingipains/LPS endo-protease/peptidase system-needed for its absolute survival and the production of its secreted OMVs responsible for the majority of its systemic multi-systems pathology. The paratope binding domain from this murine Mab has been successfully grafted onto a human IgG1 framework thus creating a variant that is a human-chimeric, bio-therapeutic antibody.

In some embodiments, the ABM of the present disclosure has therapeutic properties as a medicament. In some embodiments, the ABM of the present disclosure can be effective for as a medicament for Alzheimer's disease and early, middle and late onset cognitive, frontotemporal Dementias, Parkinson's disease, and Orphan Drug indication for Downs Dementia. In some embodiments, the ABM of the present disclosure can be effective for as a medicament for NASH, Glioma, and myocardium hypertrophy. Furthermore, research disclosed herein indicates the role of Pg in the peripheral model of disease, in which toxic proteins are delivered from Pg into the blood and brain. Consequently, the ABM of the present disclosure can be effective in targeting Pg and its downstream toxins. In some embodiments, the ABM of the present disclosure can be effective against system wide inflammation, neurodegenerative disorders, and other diseases. Non-limiting examples of systemic inflammation that the ABM of the present disclosure can be effective against includes those that are mediated by C-RP, A1c, TNF-alpha, IL1b, NLRP3, Lp-PLA2, and MPO. Non-limiting examples of neurodegenerative disorders that the ABM of the present disclosure can be effective against includes those that are mediated by APP, amyloid beta, TNF-alpha, ApoE fragmentation, tau, iron dysbiosis, and salivary lactoferrin.

In some embodiments, the ABM of the present disclosure can be effective as an anti-inflammatory therapeutic. In some embodiments, the ABM of the present disclosure can be effective as an anti-inflammatory therapeutic for atherosclerosis, cardiovascular disease, type II diabetes, and cardio-metabolic diseases.

In some embodiments, the ABM of the present disclosure can be effective in chemotherapy. In some embodiments, the ABM of the present disclosure can be effective as an adjuvant chemotherapy for oncology, including treating such cancers as esophageal, pancreatic, oral, and non-smokers lung cancers.

Also disclosed herein is the mRNA and DNA encoding any one of the ABMs of the present disclosure. In some embodiments, the ABM is formatted for administration to a subject for use as a medicament. In some embodiments, the mRNA and/or DNA encoding the ABM is administered to a subject, tissue, cell, or cell line in order to express or otherwise produce the ABM in vivo. In some embodiments, the mRNA and/or DNA encoding the ABM is administered to a subject, tissue, cell, or cell line for therapeutic use. In some embodiments, the mRNA and/or DNA encoding the ABM is used to generate the ABM, which in turn is used in therapeutics. In some embodiments, the mRNA and/or DNA encoding the ABM is incorporated into a cell line, such that the cell line functions to express the ABM. In some embodiments, a viral construct comprises the mRNA and/or DNA encoding the ABM. In some embodiments, the viral construct is administered to a subject, tissue, cell, or cell line, such that the ABM is expressed in vivo. In some embodiments, the viral construct is administered to a subject, tissue, cell, or cell line as a medicament.

In some embodiments, the ABM of the present disclosure can be effective in preventing the periodontal growth or recolonization by P. gingivalis in a subject to which the ABM is administered. Without being bound to theory, the ABM, e.g., antibody, can bind to critical survival surface structures of the bacteria so as to interfere with the bacteria's ability to attach, stay attached to form a protective bio-film, derive metabolites/energy sources, and inactivate anti-bacterial defenses and thus survive. This can cause the bacteria to die and can destroy its biofilm, such destruction of the biofilm changing the nutrient support to other dysbiotic bacteria that may have formed around and have inter-dependence with P. gingivalis colonies. As a result, the bacterial molecules leading to active chronic inflammation and disease e.g. gingipains/LPS are no longer produced, thus reducing and/or eliminating local/systemic inflammation in the human host, leading to repair, healing and re-establishment of a more healthy oral microbiome.

In some embodiments, the ABM provided herein, while human or humanized, can be especially resistant to degradation when used orally. In some embodiments, this can be achieved by retaining primary amino acid sequence structure(s) that confer resistance to bacterial proteases or by engineering the sequences into the AMB constructs.

In some embodiments, the ABM binds to an epitope that includes a “Hag×repeat” section, which is a motif that is present in various proteins/peptides of interest for gingipains. The motif comprises: YTYTVYRDGTKIK (SEQ ID NO: 190) as a component of the epitope for KB001. The motif is present at least once in Pg, but in pre-processed forms of the protein, can be present multiple times (e.g., 2, 3, 4, 5, 6, 7, 8, 9 or 10, 11, 12, 13, 14, 15 times or more for various complexes). By using antibodies that target to this motif, numerous antibodies can bind to the target of interest in an enhanced manner. The motif can comprise longer sequences as well, such as: YTYTVYRDGTKIK (SEQ ID NO: 190). Depending on Pg strain this motif is repeated at least twice on Kgp, 3× on RgpA and up to 6× on HagA. In some embodiments, the epitope occurs at least 10 times on proteins associated with the Pg cell surface, making it superior for therapeutics. The use of such an ABM embodiment is contemplated for all compositions and methods provided herein.

In some embodiments, the methods can involve using one or more of the ABMs presented herein, such as KB001 (or any other variant thereof provided herein, including any one or more of those in Table 13.1), as a therapeutic for a disease and/or a disorder in a subject. In some embodiments, one or more of the ABMs presented herein (including any one or more of those in Table 13.1) is used an indication for an inflammatory disease in a subject. In some embodiments, one or more of the ABMs presented herein (including any one or more of those in Table 13.1) is used to treat an indication for one or more of a neurodegenerative disorder, Alzheimer's Disease, Parkinson's, and/or dementia in a subject. In some embodiments, one or more of the ABMs presented herein (including any one or more of those in Table 13.1) is used to treat an indication involving the presence of Porphyromonas gingivalis in a subject. In some embodiments, one or more of the ABMs presented herein (including any one or more of those in Table 13.1) is used to treat an indication for a Porphyromonas gingivalis-driven disease in a subject. In some embodiments, one or more of the ABMs presented herein (including any one or more of those in Table 13.1) is used an indication for the presence of toxins as a byproduct of Porphyromonas gingivalis in a subject. In some embodiments, one or more of the ABMs presented herein (including any one or more of those in Table 13.1) is used to treat the presence of toxins in blood and/or plasma as a byproduct of Porphyromonas gingivalis in a subject. In some embodiments, one or more of the ABMs presented herein (including any one or more of those in Table 13.1) is used to treat a cardiometabolic disease in a subject. In some embodiments, one or more of the ABMs presented herein (including any one or more of those in Table 13.1) is used to treat at least one of a neurodegenerative disease and/or systemic wide inflammatory disease in a subject. In some embodiments, one or more of the ABMs presented herein is used to treat Downs Dementia. In some embodiments, any of the methods provided herein can be applied to the above indications.

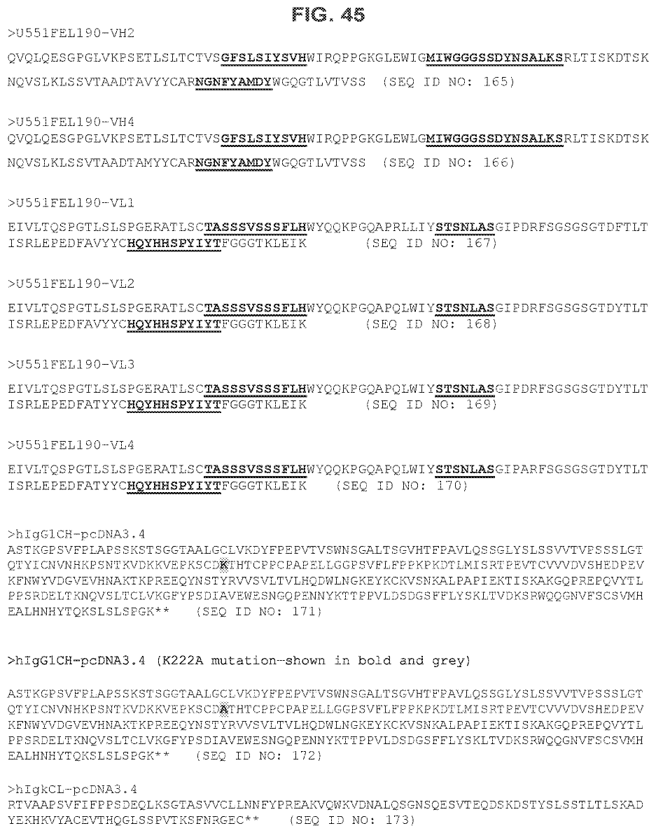

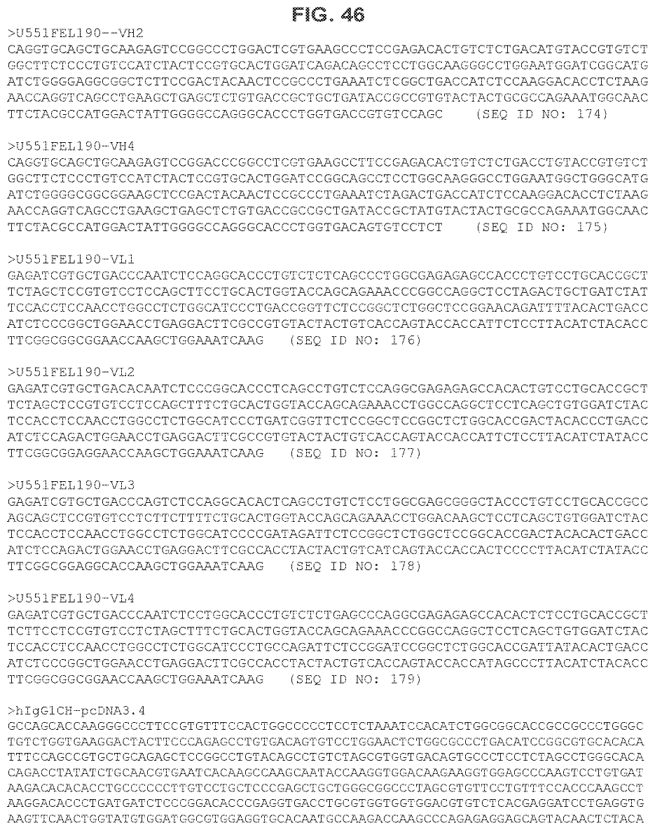

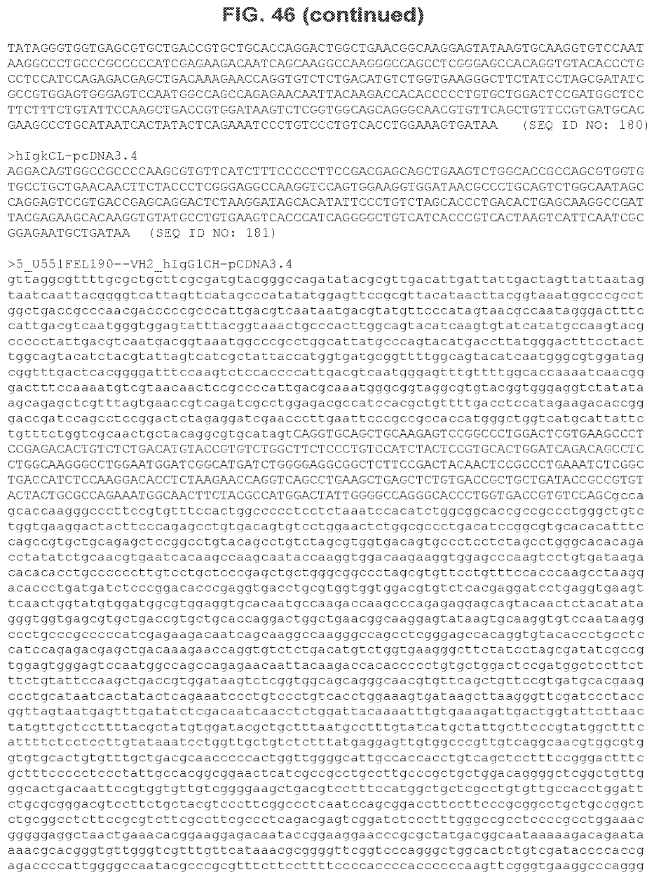

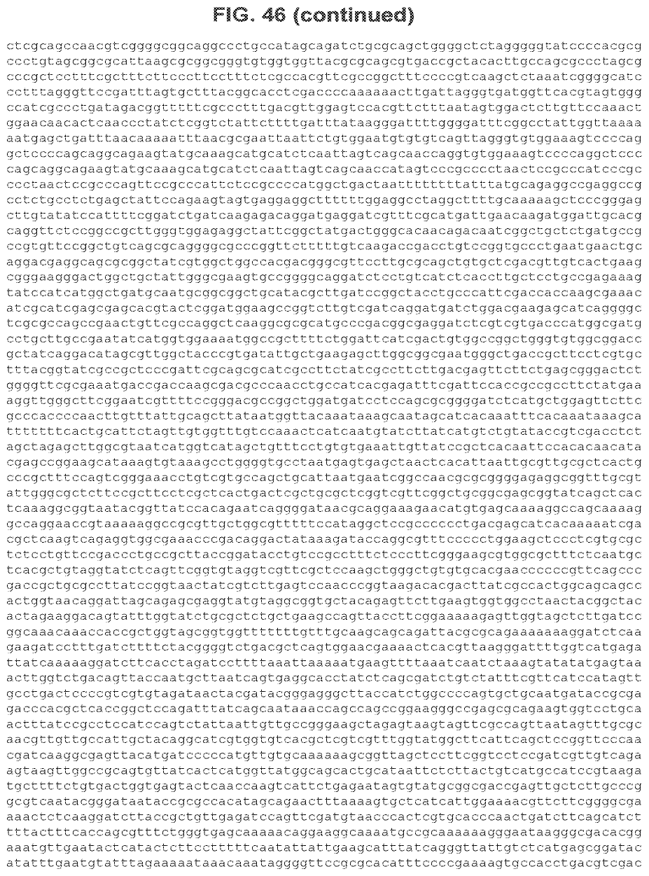

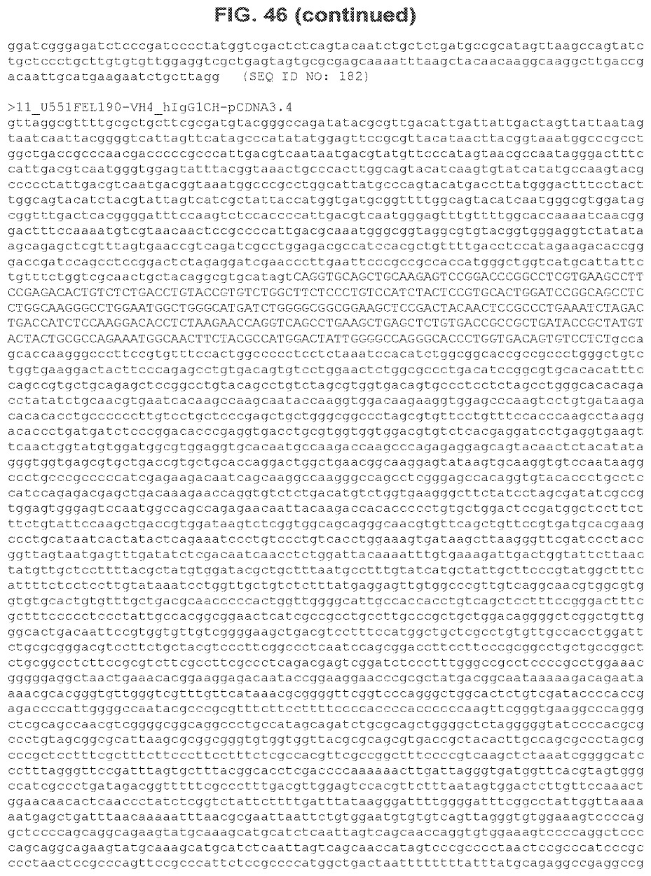

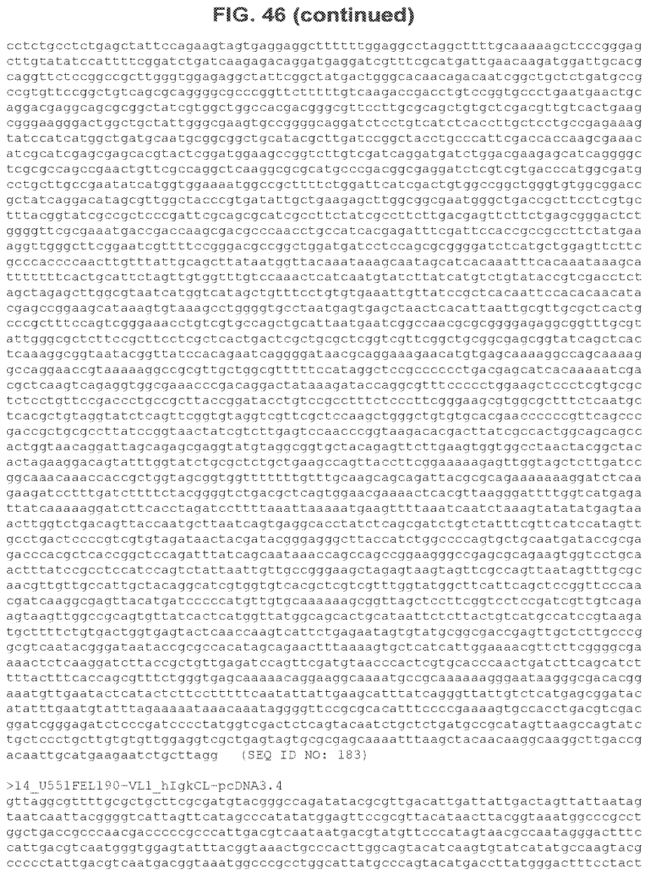

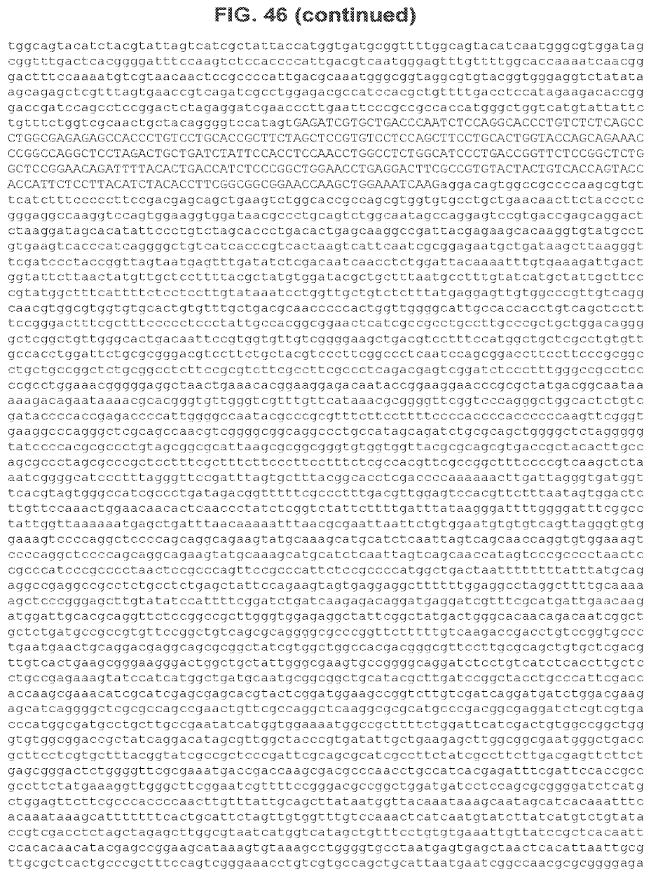

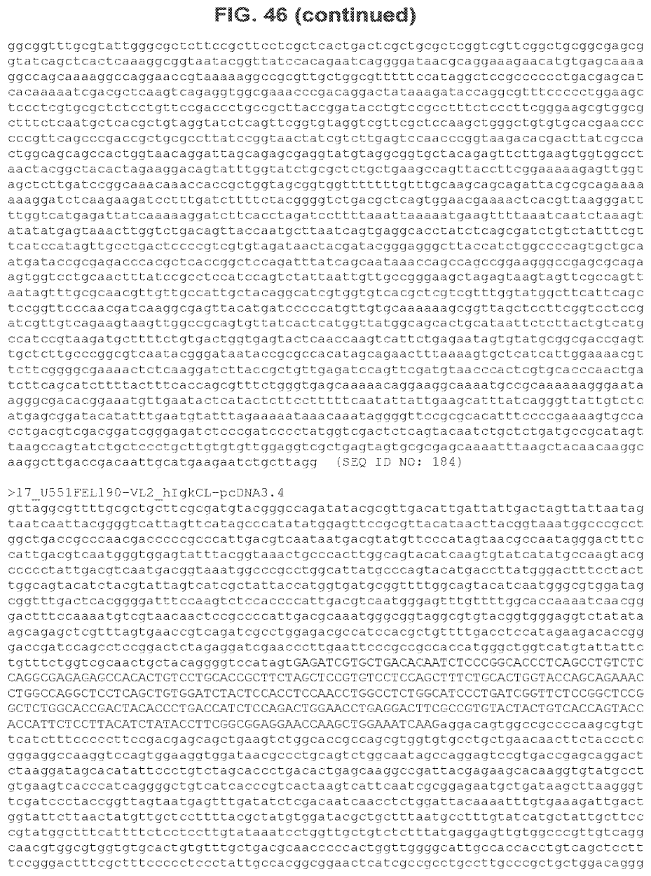

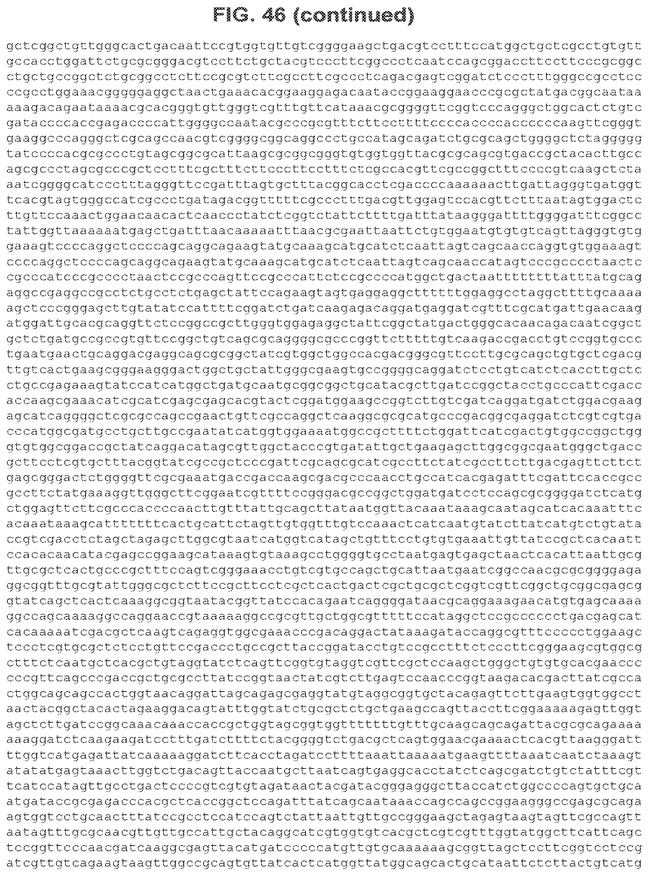

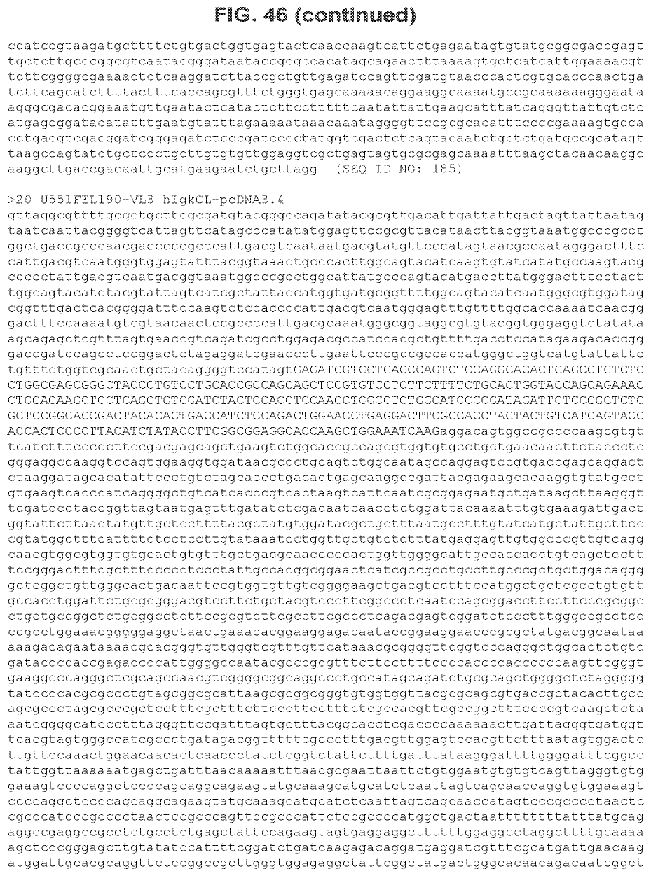

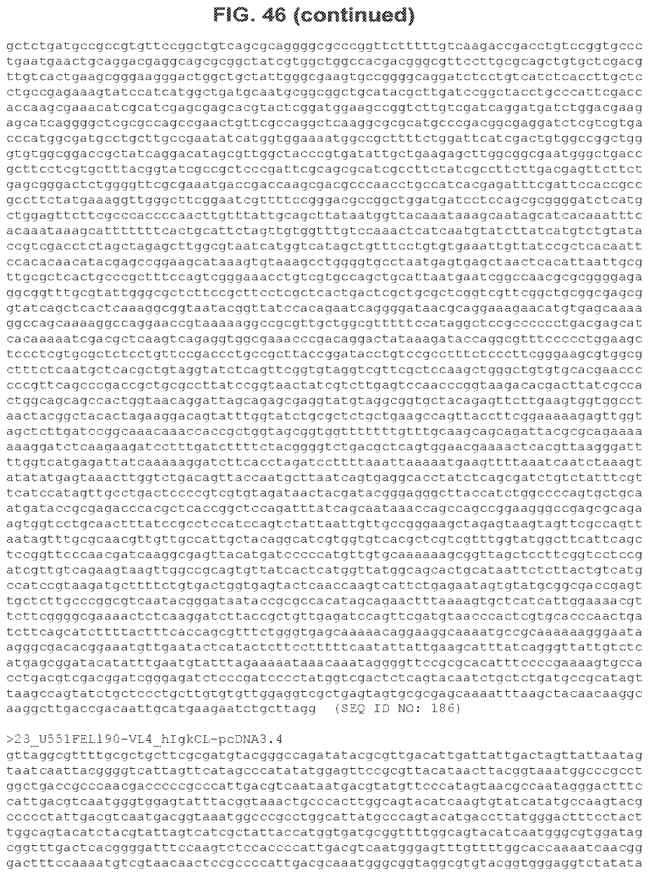

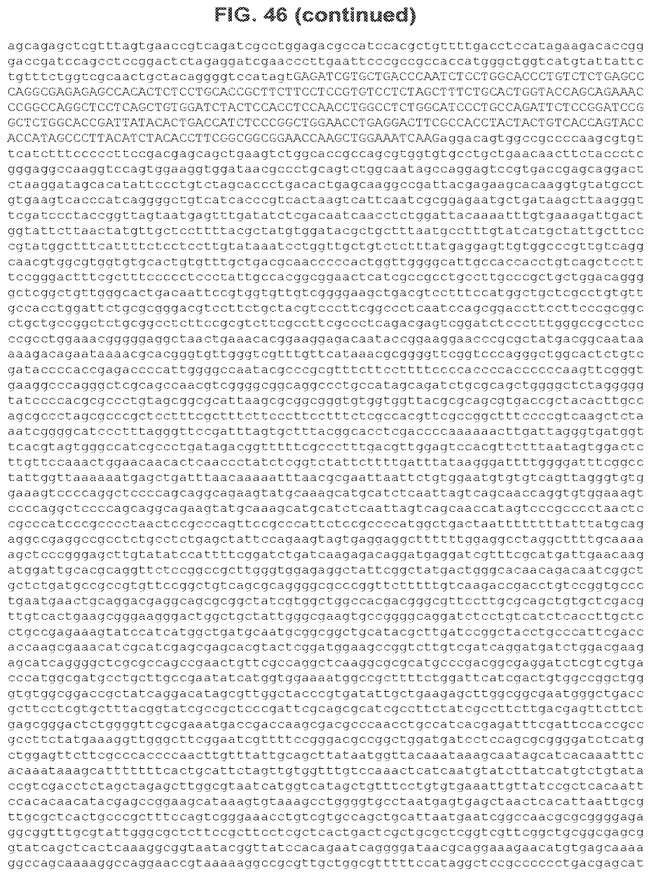

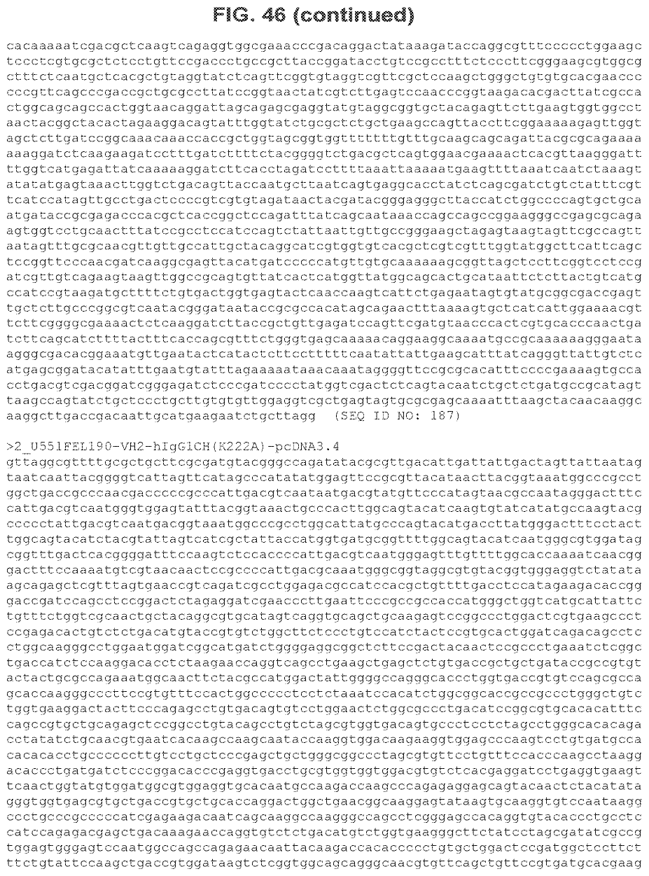

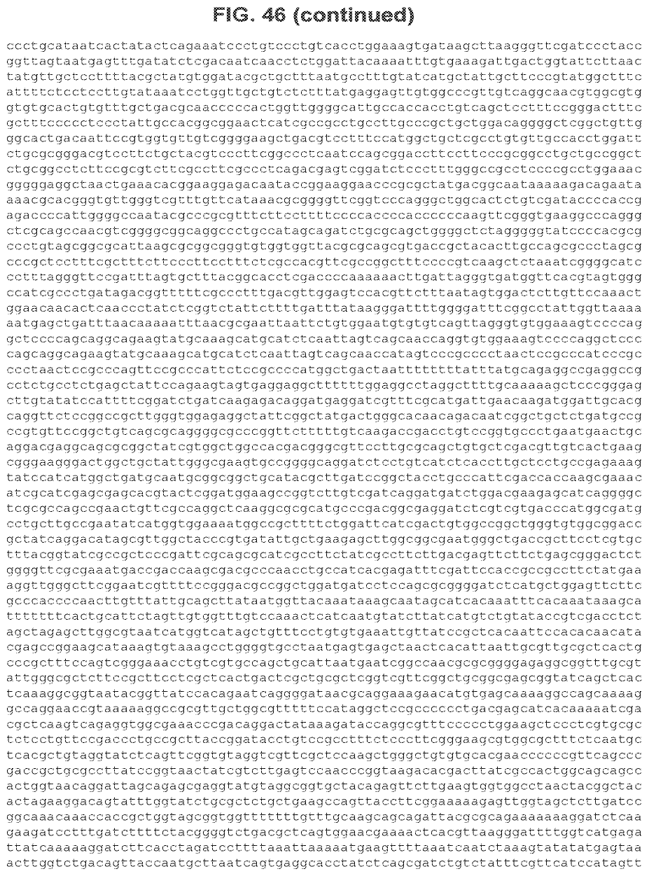

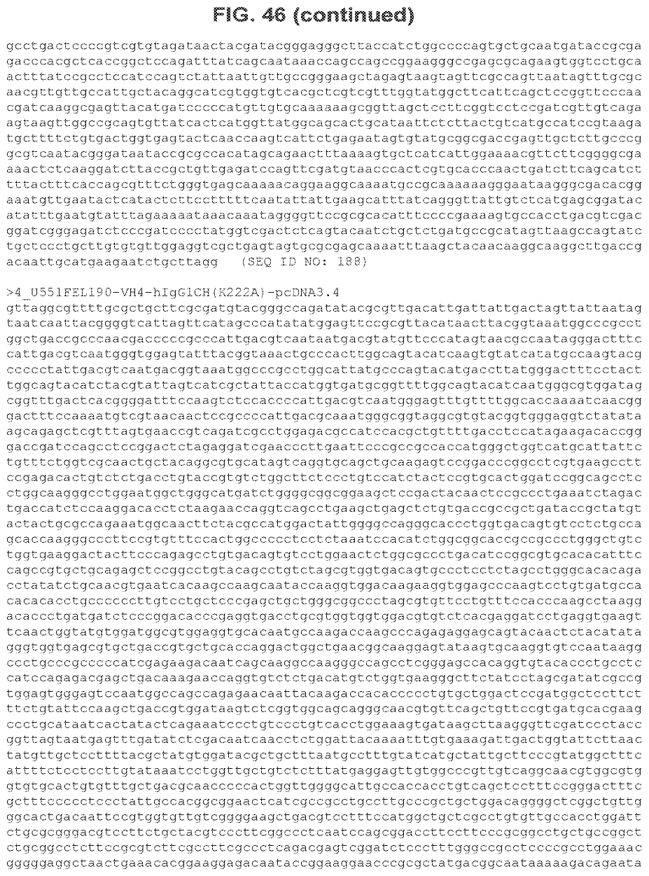

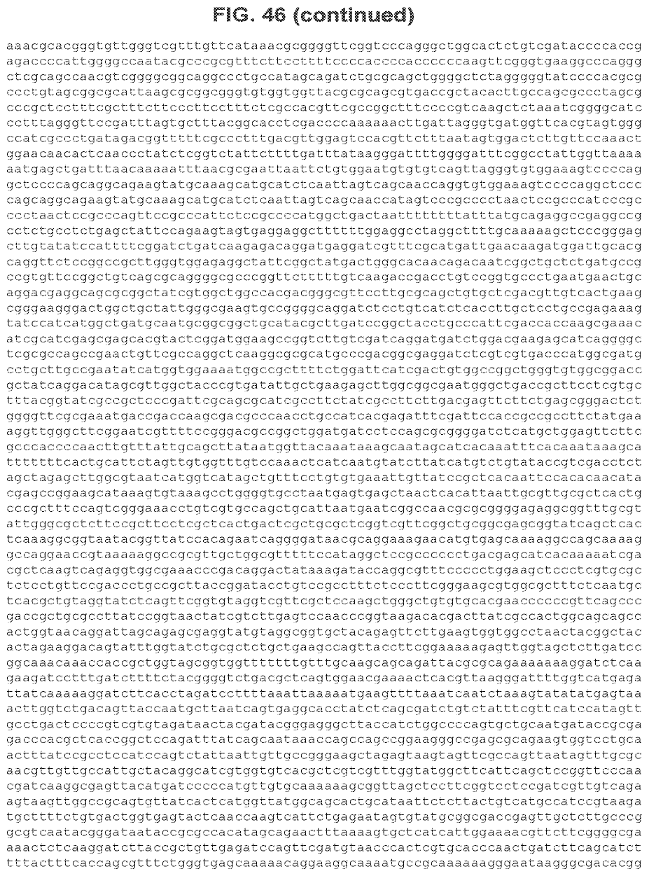

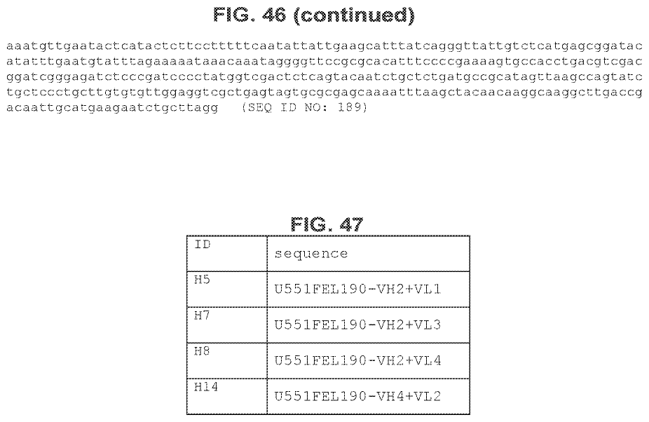

In some of the embodiments, the ABM has enhanced resistance against cleavage from P.g. proteases. In some embodiments, this enhanced resistance is conferred through the optimization of the sequence. In some embodiments, the enhanced resistance is at least partially due to a human chimeric sequence. In some embodiments, the enhanced resistance is at least partially due to a point mutation. In some embodiments, the point mutation is at position 222 in the amino acid sequence. In some embodiments, the point mutation at position 222 is an alanine. In some embodiments, position 222 can be with reference to SEQ ID NO: 172, in FIGS. 45 and 46. This denotes a confirmation of which residue position is designated 222 for reference to other ABM sequence (thus, the position corresponding in other ABMs to position 222 in SEQ ID NOs: 172 is what is being referred to when the phrase “position 222” or “222” or “K222A” is used herein.

In some embodiments, the ABM comprises an amino acid sequence at least about 70%, at least about 75%, at least about 80%, at least about 85%, at least about 90%, at least about 95%, at least about 99%, and/or at least about 100% identical to SEQ ID NO: 84. In some embodiments, the HVR comprises an amino acid sequence at least about 70%, at least about 75%, at least about 80%, at least about 85%, at least about 90%, at least about 95%, at least about 99%, and/or at least about 100% identical to one of SEQ ID NOS:85-86. In some embodiments, the LVR comprises an amino acid sequence at least about 70%, at least about 75%, at least about 80%, at least about 85%, at least about 90%, at least about 95%, at least about 99%, and/or at least about 100% identical to one of SEQ ID NOS:87-90. In some embodiments, the ABM comprises an HVR amino acid sequence corresponding to a nucleic acid sequence that is at least about 70%, at least about 75%, at least about 80%, at least about 85%, at least about 90%, at least about 95%, at least about 99%, and/or at least about 100% identical to one of SEQ ID NOS:91-92. In some embodiments, the ABM comprises an LVR amino acid sequence corresponding to a nucleic acid sequence that is at least about 70%, at least about 75%, at least about 80%, at least about 85%, at least about 90%, at least about 95%, at least about 99%, and/or at least about 100% identical to one of SEQ ID NOS:93-97. In some embodiments, the ABM corresponds to a nucleic acid sequence that is at least about 70%, at least about 75%, at least about 80%, at least about 85%, at least about 90%, at least about 95%, at least about 99%, and/or at least about 100% identical to one of SEQ ID NOS: 98-101. In some embodiments, the ABM further comprises at least one of an alanine at position 222, an amino acid sequence that is at least about 70%, at least about 75%, at least about 80%, at least about 85%, at least about 90%, at least about 95%, at least about 99%, and/or at least about 100% identical to SEQ ID NO: 84, an HVR sequence comprising an amino acid sequence at least about 70%, at least about 75%, at least about 80%, at least about 85%, at least about 90%, at least about 95%, at least about 99%, and/or at least about 100% identical to one of SEQ ID NOS:85-86, and/or an LVR sequence comprising an amino acid sequence at least about 70%, at least about 75%, at least about 80%, at least about 85%, at least about 90%, at least about 95%, at least about 99%, and/or at least about 100% identical to one of SEQ ID NOS:87-90. In some embodiments, the ABM binds to a gingipain and/or a haemagluttinin with a KD that is less than about 10E-9M, less than about 5E-9M, less than about 2.5E-9M, less than about 2E-9 M, less than about 1E-9 M, less than about 9E-10 M, less than about 8E-10 M, less than about 6E-10 M, less than about 4E-10 M, less than about 2E-10 M, less than about 1E-10 M, less than about 9E-11 M, less than about 7E-11 M, less than about 5E-11 M, less than about 3E-11 M, less than about 1E-11 M, less than about 1E-12 M, less than about 1E-13 M, less than about 1E-14 M, less than about 1E-15 M, and/or less than about 1E-20 M.

Also disclosed herein is a nucleic acid that is at least about 70%, at least about 75%, at least about 80%, at least about 85%, at least about 90%, at least about 95%, at least about 99%, and/or at least about 100% identical to one of SEQ ID NOS: 98-101. Also disclosed herein is a human or humanized antigen binding molecule (ABM) that binds to a protein complex, protein, peptide, or amino acid sequence comprising the sequence YTYTVYRDGTKIK (SEQ ID NO: 190). In some embodiments, the human or humanized antigen binding molecule (ABM) that binds to a protein complex, protein, peptide, or amino acid sequence comprises a sequence that is at least about 70%, at least about 75%, at least about 80%, at least about 85%, at least about 90%, at least about 95%, at least about 99%, and/or at least about 100% identical to the sequence YTYTVYRDGTKIK (SEQ ID NO: 190).

In some embodiments, the ABM comprises SEQ ID NO: 1. In some embodiments, the ABM comprises an amino acid sequence that is at least about 70%, at least about 75%, at least about 80%, at least about 85%, at least about 90%, at least about 95%, at least about 99%, and/or at least about 100% identical to SEQ ID NO: 1. In some embodiments, the ABM comprises SEQ ID NO: 2. In some embodiments, the ABM comprises an amino acid sequence that is at least about 70%, at least about 75%, at least about 80%, at least about 85%, at least about 90%, at least about 95%, at least about 99%, and/or at least about 100% identical to SEQ ID NO: 2. In some embodiments, the ABM comprises SEQ ID NO: 1 and SEQ ID NO: 2. In some embodiments, the ABM comprises an amino acid sequence that is at least about 70%, at least about 75%, at least about 80%, at least about 85%, at least about 90%, at least about 95%, at least about 99%, and/or at least about 100% identical to SEQ ID NO: 1, and an amino acid sequence that is at least about 70%, at least about 75%, at least about 80%, at least about 85%, at least about 90%, at least about 95%, at least about 99%, and/or at least about 100% identical to SEQ ID NO: 2. In some embodiments, the ABM is H5 K22A. In some embodiments, the ABM is at least about 70%, at least about 75%, at least about 80%, at least about 85%, at least about 90%, at least about 95%, at least about 99%, and/or at least about 100% identical to H5 K22A. In some embodiments, the ABM is humanized or human. In some embodiments, the ABM is murine. In some embodiments the ABM is chimeric and comprises human and/or mouse sequences. In some embodiments, the ABM comprises an alanine at position 222. In some embodiments, the ABM is human and comprises an alanine at position 222. In some embodiments, the ABM is murine and comprises an alanine at position 222. In some embodiments, the ABM is a human chimera and comprises an alanine at position 222. In some embodiments, the ABM is a murine chimera and comprises an alanine at position 222. In some embodiments, the ABM of the present disclosure comprises a heavy chain sequence of SEQ ID NO: 30, a light chain sequence of SEQ ID NO: 33, except that the ABM comprises an alanine at position 222. In some embodiments, the ABM of the present disclosure comprises a heavy chain sequence of SEQ ID NO: 30 and a light chain sequence of SEQ ID NO: 33. In some embodiments, the ABM of the present disclosure comprises a heavy chain sequence that is at least at least about 70%, at least about 75%, at least about 80%, at least about 85%, at least about 90%, at least about 95%, at least about 99%, and/or at least about 100% identical to SEQ ID NO: 30. In some embodiments, the ABM of the present disclosure comprises a light chain sequence that is at least about 70%, at least about 75%, at least about 80%, at least about 85%, at least about 90%, at least about 95%, at least about 99%, and/or at least about 100% identical to SEQ ID NO: 33.

Also disclosed herein is a method of treating a disorder driven or associated by P. gingivalis. As will be understood by one skilled in the art, the disorder may be any disease or disorder in a subject that has detectable levels of P. gingivalis in that subject's cell, cells, blood, plasma, tissue, fat deposits, gums, mouth, brain, brain cavity, organ, and/or organ system. In some embodiments, the method comprises providing an antibody that binds to a P. gingivalis associated peptide, to a subject. Optionally, the antibody is known to function to stop a P. gingivalis infection. In some embodiments, the antibody is a humanized or human antibody. In some embodiments, position 222 of the antibody has been changed to an alanine. As will be appreciated by one skilled in the art, the antibody may be administered alone or in an acceptable pharmaceutical composition, and at any concentration and/or route of administration that provides a therapeutic effect.

Any of the embodiments provided herein can be directed to or substituted with ABM (including antibodies) that bind to the following sequence: YTYTVYRDGTKIK (SEQ ID NO: 190).

P. gingivalis

Porphyromonas gingivalis is a keystone pathogen that converts the local and distant healthy microbiome of an individual into a disease-forming biofilm of both the mouth and gut. P. gingivalis has multiple survival mechanism, which creates a grossly undiagnosed chronic active/inactive infection in the host leading to a “silent” chronic state of systemic and end organ inflammation and ultimate failure.

Pg is unique in that it completely returns one week after regular dental cleaning and re-establishes its life-long bio-film 30 days after non-surgical periodontal treatment. It can even be present in a visually clean and healthy-looking mouth. This leads to a slow, low to high level of local and systematic damage that is mostly clinically silent and often without a person even noticing. In some embodiments, KB-001 prevents Pg from synthesizing its secreted outer membrane vesicles (OMVs) containing virulence protein complexes, resulting in the bacteria shutting down its metabolic and host defense functions. In some embodiments, KB-001 has the capability to treat Pg, eliminating it and all of its virulence factors.

The pathogen hypothesis for Alzheimer's disease has been met with new attention over the last 5 years, but the push back has been the Immune Privilege of the Brain and whether the suspected pathogen source is local or peripheral to the brain tissues. As disclosed herein, the inventors show that the effect of P. gingivalis in the brain is mostly if not entirely from an oral peripheral source. Second, the inventors have generated new data from the largest analysis of AD brain tissues to date showing no presence of P. gingivalis DNA in the brain. Thirdly, the inventors have identified and discovered a one-of-a-kind virulent subunit of the primary suspected pathogen in the strategic sites of AD brain tissues. It is a unique subunit toxin “XXX Epitope” domain of P. gingivalis, This virulent subunit toxin plays a massive role in disrupting the NLRP3 inflammasome and the IL-1b pathways. IL-1b and ubiquinone have been shown to trigger the pathogenesis and progression of Alzheimer's disease. This same virulent subunit toxin plays an equally large role in systemic inflammation, immune disruption, and has disease-causing effects on basic human cellular biology. The delivery of the virulent toxin to the brain appears to be primarily vascular, with possibly additional access through neuronal, all however, occurring from the oral source of P. gingivalis. The data described herein strongly suggests for the first time that the “XXX Epitope” and related material are coming to the brain in AD as secreted by outer membrane vesicles from the bacterial surface of oral cavities. Further research is currently being conducted by the inventors into the prevalence of, genotypes of, and relative amounts of the presence of P.g. and its associated secreted exotoxins (OMVs-gingipains and LPS) and anti-P.g./LPS antibodies in patients with increased markers of systemic vascular inflammation and overexpression of inflammasome pathways, as well as the prevalence of increased markers of vascular and gut inflammation in patients with and without P.g. infection.

### Definitions

As used herein, the term “antigen binding molecule” (ABM) refers to a polypeptide that includes one or more fragments of an antibody that retain the ability to specifically bind to an antigen, e.g., bacterial antigen (e.g., gingipain, adhesin hemagglutinin complex). ABM encompasses antigen-binding fragments of antibodies (e.g., single chain antibodies, Fab and Fab fragments, F(ab′)2, Fd fragments, Fv fragments, scFv, and domain antibodies (dAb) fragments (e.g., nanobodies) (see, e.g. de Wildt et al., Eur J. Immunol. 1996; 26(3):629-39; which is incorporated by reference herein in its entirety)) as well as complete antibodies. An ABM can include an antibody or a polypeptide containing an antigen-binding domain of an antibody. In some embodiments, an ABM can include a monoclonal antibody or a polypeptide containing an antigen-binding domain of a monoclonal antibody. For example, an ABM, e.g., antibody, can include a heavy (H) chain variable region (abbreviated herein as VH), and/or a light (L) chain variable region (abbreviated herein as VL). In another example, an ABM, e.g., antibody, includes two heavy (H) chain variable regions and/or two light (L) chain variable regions. An ABM, e.g., antibody, can have the structural features of IgA, IgG, IgE, IgD, IgM (as well as subtypes and combinations thereof). An ABM, e.g., antibody, can be from any source, including mouse, rabbit, pig, rat, and primate (human and non-human primate) and primatized (e.g., humanized) antibodies. ABM also include mini-bodies, humanized antibodies, chimeric antibodies, and the like, as well as nanobodies (single variable domain with two constant heavy domains) derived from Camelidae (camels and llamas) family. In addition they can be synthesized using protein synthetic chemistries ab initio.

As used herein an “antibody” refers to any immunoglobulin (Ig) molecule comprised of four polypeptide chains, two heavy (H) chains and two light (L) chains, interconnected by disulfide bonds or any functional fragment, mutant, variant, or derivation thereof, which retains the essential epitope binding features of an Ig molecule. The heavy chain constant region can include CH1, hinge, CH2, CH3, and, sometimes, CH4 regions. In some embodiments, for therapeutic purposes, the CH2 domain can be deleted or omitted. “Antibody” also refers to IgG, IgM, IgA, IgD or IgE molecules or antigen-specific antibody fragments thereof (including, but not limited to, a Fab, F(ab′)₂, Fv, disulfide linked Fv, scFv, single domain antibody, closed conformation multi-specific antibody, disulfide-linked scFv, diabody), whether derived from any species that naturally produces an antibody, or created by recombinant DNA technology; whether isolated from serum, B-cells, hybridomas, transfectomas, yeast or bacteria.

The VH and VL regions can be further subdivided into regions of hypervariability, termed “complementarity determining regions” (“CDR”), interspersed with regions that are more conserved, termed “framework regions” (“FR”). The extent of the framework region and CDRs has been defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4. In some embodiments, an ABM, e.g., antibody, includes 1, 2, 3, 4, 5, and/or 6 CDRs.

The terms “antigen-binding fragment” or “antigen-binding domain,” which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. Furthermore, the two domains of the Fv fragment, VL and VH, can be joined, using recombinant methods, by a synthetic linker that enables them to be made as a single protein chain in which the VL and VH regions pair form monovalent molecules known as single chain Fv (scFv). See e.g., U.S. Pat. Nos. 5,260,203, 4,946,778, and 4,881, 175; Bird et al. (1988) Science 242:423-426; and Huston et al. (1988) Proc. Natl. Acad. Sci. USA 85:5879-5883. Antibody fragments can be obtained using any appropriate technique.

The term “Fc region” refers to the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Specifically, in IgG, IgA and IgD types, the Fc region is composed of two identical protein fragments derived from CH2 and CH3 of the heavy chains. Fc regions of IgM and IgE contain three heavy chain constant domains, CH2, CH3, and CH4.

The term “monospecific antibody” refers to an antibody that displays a single binding specificity and affinity for a particular target, e.g., epitope. This term includes a “monoclonal antibody” or “mAb,” which as used herein refer to a preparation of antibodies or fragments thereof of single molecular composition, irrespective of how the antibody was generated. The monoclonal antibody can be obtained from a population of substantially homogeneous antibodies, i.e., the individual antibodies comprising the population are identical except for possible naturally occurring mutations that may be present in minor amounts. Monoclonal antibodies can be highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method. In an embodiment, the monoclonal antibody is produced by hybridoma technology.

The term “human antibody” or “human ABM” includes antibodies or ABMs having variable and constant regions corresponding to human germline immunoglobulin sequences as described by Kabat et al. (See Kabat, et al. (1991) Sequences of Proteins of Immunological Interest, Fifth Edition, U.S. Department of Health and Human Services, NIH Publication No. 91-3242) or Chothia, C. et al. (1987) J. Mol. Biol. 196:901-917; which are incorporated by reference herein in their entireties. The human antibodies or ABMs of the present disclosure may include amino acid residues not encoded by human germline immunoglobulin sequences (e.g., mutations introduced by random or site-specific mutagenesis in vitro or by somatic mutation in vivo), for example in the CDRs. Any suitable method for generating human or fully human antibodies or ABMs can be used, including but not limited to, EBV transformation of human B cells, selection of human or fully human antibodies from antibody libraries prepared by phage display, yeast display, mRNA display or other display technologies, and also from mice or other species that are transgenic for all or part of the human Ig locus comprising all or part of the heavy and light chain genomic regions defined further above. Selected human antibodies or ABMs may be affinity matured by art recognized methods including in vitro mutagenesis, preferably of CDR regions or adjacent residues, to enhance affinity for the intended target.

By “humanized antibody” or “humanized ABM” is meant an antibody or ABM that is composed partially or fully of amino acid sequences derived from a human antibody germline by altering the sequence of an antibody having non-human complementarity determining regions (CDR). A humanized antibody or ABM can include an antibody or ABM that comprises heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which non-human CDR sequences are introduced into human VH and VL sequences to replace the corresponding human CDR sequences. Also a “humanized antibody” is an antibody or a variant, derivative, analog or fragment thereof that specifically binds to an antigen of interest and which comprises a framework (FR) region having substantially the amino acid sequence of a human antibody and a CDR having substantially the amino acid sequence of a non-human antibody.

The term “chimeric antibody” refers to an antibody that comprises heavy and light chain variable region sequences from one species (e.g., mouse) and constant region sequences from another species (e.g., human), such as antibodies having murine heavy and light chain variable regions linked to human constant regions.

Traditionally, monoclonal antibodies have been produced as native molecules in murine hybridoma lines. In addition to that technology, the methods and compositions described herein provide for recombinant DNA expression of monoclonal antibodies. This allows the production of humanized antibodies as well as a spectrum of antibody derivatives and fusion proteins in a host species of choice. The production of antibodies in bacteria, yeast, transgenic animals and chicken eggs are also alternatives to hybridoma-based production systems.

As used herein, an “epitope” can be formed both from contiguous amino acids, or noncontiguous amino acids juxtaposed by folding of a protein. Epitopes formed from contiguous amino acids are typically retained on exposure to denaturing solvents, whereas epitopes formed by folding are typically lost on treatment with denaturing solvents. An epitope includes the unit of structure specifically bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein. In some embodiments, the epitope may have both linear and conformational sequence determinants and thus be derived from a single monomer, homo-dimer, homo trimer, etc., and/or hetero-dimers, hetero-trimers, etc.

The term “compete” as used herein in the context of antigen binding molecules (e.g., antibodies or antigen-binding fragments thereof) that compete for the same binding target, antigen, or epitope refers to competition between antigen binding molecules as determined by an assay in which the antigen binding molecule (e.g., antibody or immunologically functional fragment thereof) being tested prevents or inhibits (e.g., reduces) specific binding of a reference antigen binding molecule (e.g., a reference antibody) to a common antigen (e.g., P. gingivalis gingipain or a fragment thereof). Any suitable competitive binding assay can be used to determine if one antigen binding molecule competes with another, for example: solid phase direct or indirect radioimmunoassay (MA), solid phase direct or indirect enzyme immunoassay (EIA), sandwich competition assay, solid phase direct labeled assay, solid phase direct labeled sandwich assay, solid phase direct label MA using I-125 label, solid phase direct biotin-avidin EIA, and direct labeled MA. Typically, such an assay involves the use of purified antigen bound to a solid surface or cells bearing either of these, an unlabeled test antigen binding protein and a labeled reference antigen binding molecule. Competitive inhibition is measured by determining the amount of label bound to the solid surface or cells in the presence of the test antigen binding molecule. Usually the test antigen binding protein is present in excess. Antigen binding proteins identified by competition assay (competing antigen binding molecules) include antigen binding molecules binding to the same epitope as the reference antigen binding molecules and antigen binding molecules binding to an adjacent epitope sufficiently proximal to the epitope bound by the reference antigen binding molecule for steric hindrance to occur. Usually, when a competing antigen binding molecule is present in excess, it will inhibit (e.g., reduce) specific binding of a reference antigen binding molecule to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologues, paralogs, fragments and other equivalents, variants, fragments, and analogs of the foregoing.

Amino acid substitutions in a native protein sequence may be “conservative” or “non-conservative” and such substituted amino acid residues may or may not be one encoded by the genetic code. A “conservative amino acid substitution” is one in which the amino acid residue is replaced with an amino acid residue having a chemically similar side chain (i.e., replacing an amino acid possessing a basic side chain with another amino acid with a basic side chain). A “non-conservative amino acid substitution” is one in which the amino acid residue is replaced with an amino acid residue having a chemically different side chain (i.e., replacing an amino acid having a basic side chain with an amino acid having an aromatic side chain). The standard twenty amino acid “alphabet” is divided into chemical families based on chemical properties of their side chains. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and side chains having aromatic groups (e.g., tyrosine, phenylalanine, tryptophan, histidine).

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The nucleic acid may be double stranded, single stranded, or contain portions of both double stranded or single stranded sequence. As will be appreciated by those in the art, the depiction of a single strand (“Watson”) also defines the sequence of the other strand (“Crick”). By the term “recombinant nucleic acid” herein is meant nucleic acid, originally formed in vitro, in general, by the manipulation of nucleic acid by endonucleases, in a form not normally found in nature. Thus an isolated nucleic acid, in a linear form, or an expression vector formed in vitro by ligating DNA molecules that are not normally joined, are both considered recombinant for the purposes of this disclosure. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the disclosure.

As used herein, “sequence identity” or “identity” in the context of two nucleic acid sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and, therefore, do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Any suitable means for making this adjustment may be used. This may involve scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may include additions or deletions (i.e., gaps) as compared to the reference sequence (which does not include additions or deletions) for optimal alignment of the two sequences. The percentage can be calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Any suitable methods of alignment of sequences for comparison may be employed. Thus, the determination of percent identity between any two sequences can be accomplished using a mathematical algorithm. Preferred, non-limiting examples of such mathematical algorithms are the algorithm of Myers and Miller, CABIOS, 4:11 (1988), which is hereby incorporated by reference in its entirety; the local homology algorithm of Smith et al, Adv. Appl. Math., 2:482 (1981), which is hereby incorporated by reference in its entirety; the homology alignment algorithm of Needleman and Wunsch, JMB, 48:443 (1970), which is hereby incorporated by reference in its entirety; the search-for-similarity-method of Pearson and Lipman, Proc. Natl. Acad. Sci. USA, 85:2444 (1988), which is hereby incorporated by reference in its entirety; the algorithm of Karlin and Altschul, Proc. Natl. Acad. Sci. USA, 87:2264 (1990), which is hereby incorporated by reference in its entirety; modified as in Karlin and Altschul, Proc. Natl. Acad. Sci. USA, 90:5873 (1993), which is hereby incorporated by reference in its entirety.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237 (1988), Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994), which are hereby incorporated by reference in their entirety. The ALIGN program is based on the algorithm of Myers and Miller, supra. The BLAST programs of Altschul et al., JMB, 215:403 (1990); Nucl. Acids Res., 25:3389 (1990), which are hereby incorporated by reference in their entirety, are based on the algorithm of Karlin and Altschul supra.

As used herein, the terms “treat,” “treatment,” “treating,” or “amelioration” refer to therapeutic treatments, wherein the object is to reverse, alleviate, ameliorate, inhibit, slow down or stop the progression or severity of a condition, e.g., a chronic inflammatory condition, associated with a disease or disorder, e.g. arteriosclerosis, gingivitis, etc. The term “treating” includes reducing or alleviating at least one adverse effect or symptom of a condition, disease or disorder associated with, e.g., arteriosclerosis, gingivitis, etc. Treatment is generally “effective” if one or more local or systemic conditions, symptoms or clinical biomarkers of disease are reduced. Alternatively, treatment is “effective” if the progression of a disease is reduced or halted. That is, “treatment” includes not just the improvement of symptoms or biomarkers, but also a cessation of, or at least slowing of, progress or worsening of symptoms compared to what would be expected in the absence of treatment. Thus, a treatment is considered effective if one or more of the signs or symptoms of a condition described herein are altered in a beneficial manner, other clinically accepted symptoms are improved, or even ameliorated and/or reversed back to a more normal or normal state, or a desired response is induced e.g., by at least 10% following treatment according to the methods described herein. Beneficial or desired clinical results include, but are not limited to, alleviation of one or more symptom(s), diminishment of extent of disease, e.g., chronic inflammatory disease, stabilized (e.g., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

Efficacy of an agent, e.g., ABM, can be determined by assessing physical indicators of a condition or desired response, e.g. inflammation and/or infection. Efficacy can be assessed in animal models of a condition described herein, for example treatment of systemic chronic inflammatory diseases associated with an oral infection, e.g., periodontal disease. When using an experimental animal model, efficacy of treatment is evidenced when a statistically significant change occurs in one of a number of criteria, including a one or more biomarkers associated with inflammation following infection. In some embodiments, treatment according to the methods described herein can reduce the levels, and/or eliminate and/or prevent the colonization of the disease causing bacteria Porphyromonas gingivalis. In some embodiments, treatment according to the methods described herein can reduce the levels of a biomarker(s) or symptom(s) or the tissue pathology of a condition, e.g. infection or recolonization by at least 10%, at least 15%, at least 20%, at least 25%, at least 30%, at least 40%, at least 50%, at least 60%, at least 70%, at least 80%, at least 90% or more, at least 95% or more, at least 98% or more, at least 99% or more, or by about 100%.

The term “effective amount” as used herein refers to the amount of an active agent, e.g., ABM, or composition needed to alleviate at least one or more criteria listed above of the disease or disorder, and relates to a sufficient amount of active agent or pharmacological composition to provide the desired effect. The term “therapeutically effective amount” therefore refers to an amount of active agent or composition that is sufficient to provide a particular anti-bacterial or anti-recolonization effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease.

As used herein, “subject” means a human or animal. The animal can be a vertebrate, including a mammal, such as a primate, dog or rodent. Primates include human, chimpanzees, cynomolgus monkeys, spider monkeys, and macaques, e.g., Rhesus. Rodents include mice, rats, woodchucks, ferrets, rabbits and hamsters. Animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a primate, e.g., a human. The terms, “individual,” “patient” and “subject” are used interchangeably herein.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. Delivery and/or placement options include any suitable medicament delivery systems for intraoral, interproximal, intrasulcular, intra-periodontal pocket, intracanal, and intranasal. In some embodiments, a suitable delivery option includes any suitable mechanical and automated dental and medical syringes, including all calibrated and non-calibrated, all attachments, and all designs of tips including but not limited to blunt ended, and side port; Medicament delivery trays and systems including PerioProtect Trays; Medicament applicator delivery systems; Slow releasing medical preparation for intrasulcular drug delivery; Filler, oral packing, fiber, microparticles, films, gels, injectable gels, vesicular systems, strips compacts, chip, hydrogel, thermal gel, liquid, solid, including Actisite, Arestin, Atridox, Ossix Plus, Periochip, Periostat, Periofil; Injectable systems; Professional irrigation systems including piezoelectric and ultrasonic cavitron units with and without reservoir including Ora-Tec Viajet and Oral irrigation systems including Interplak, Waterpik, Hydrofloss, Viajet, Airfloss and Pro.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is used herein to indicate a non-limiting example. Thus, “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

### Antigen-Binding Molecules

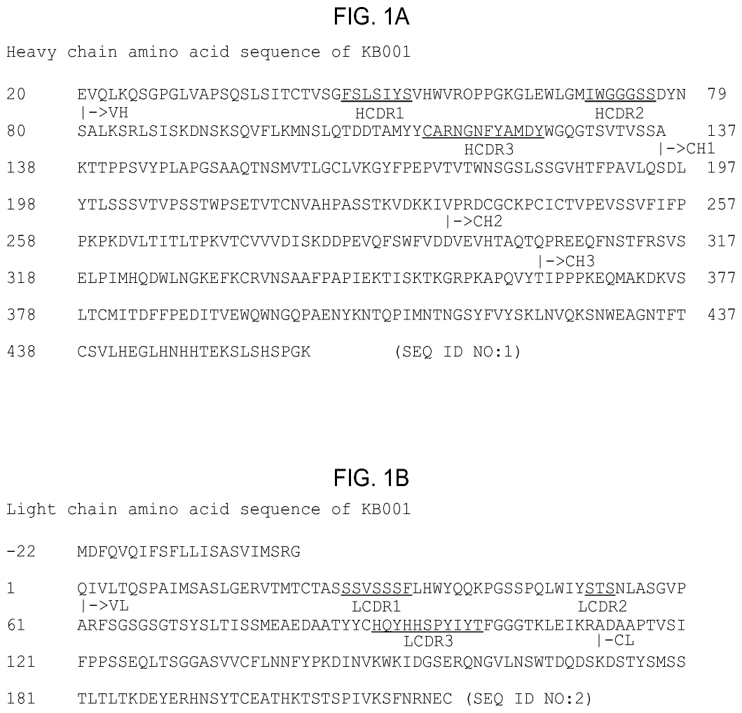

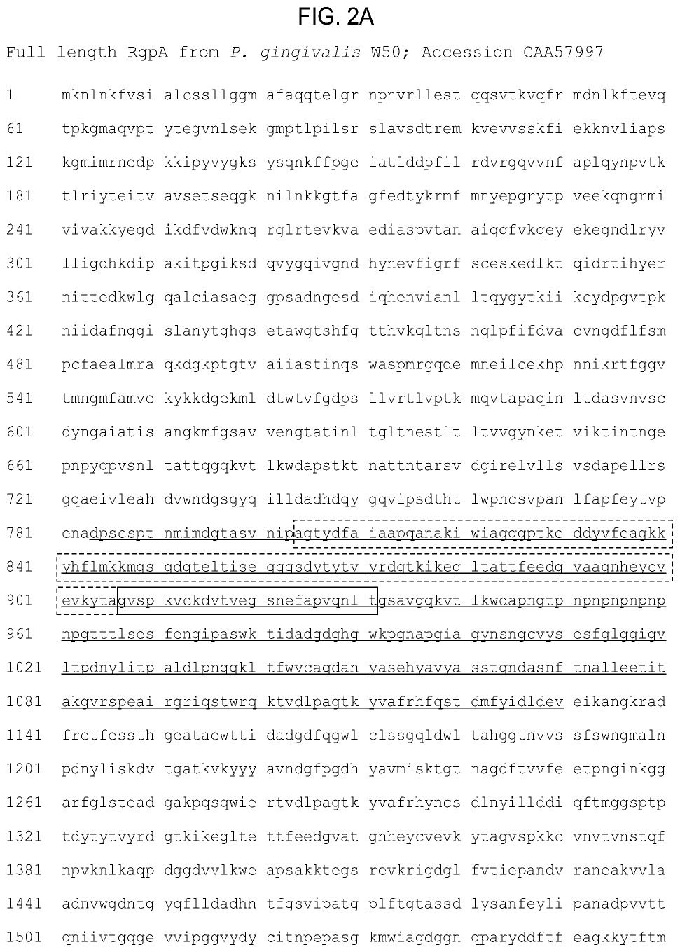

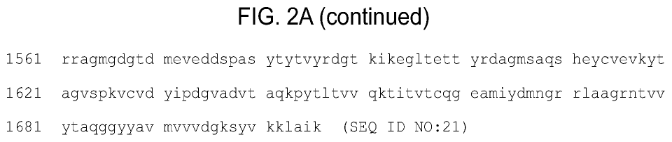

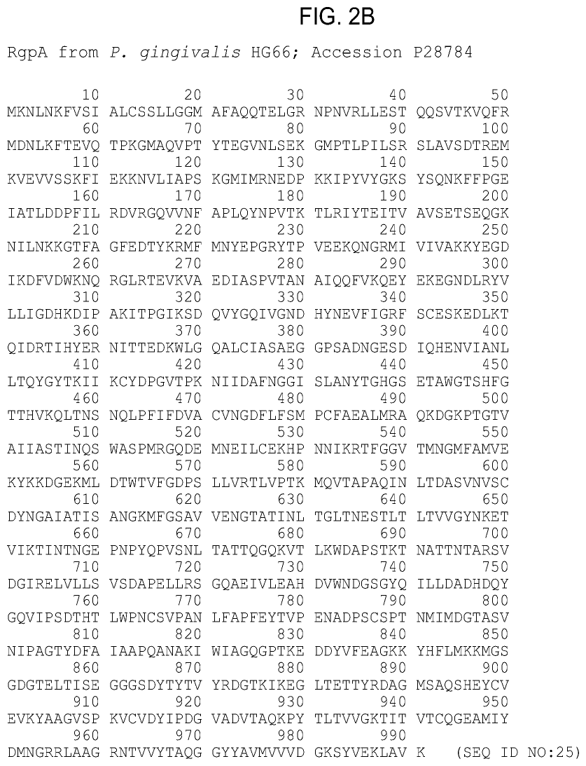

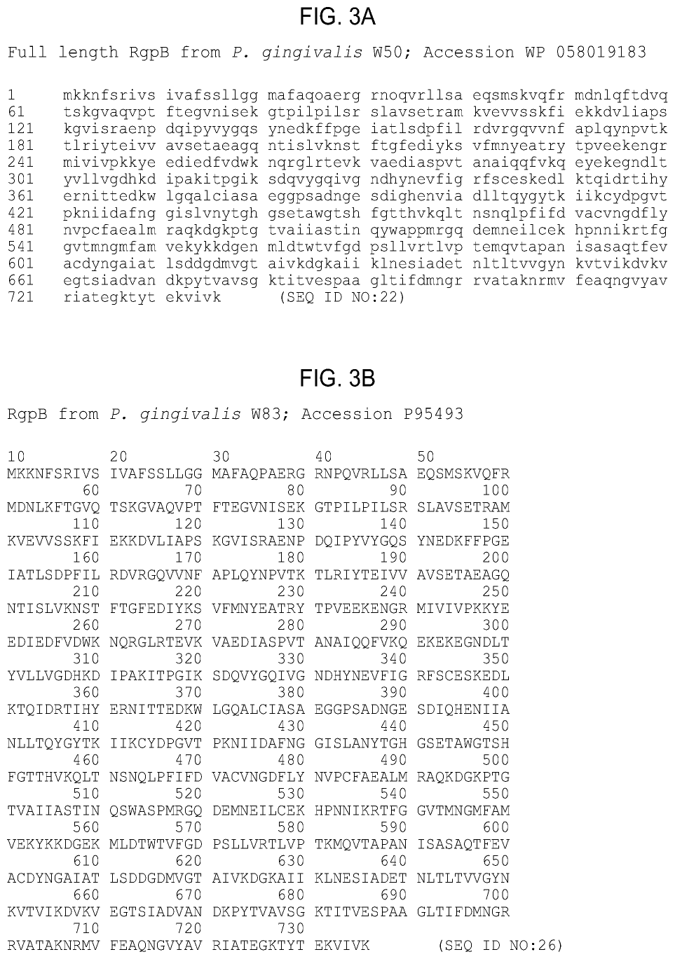

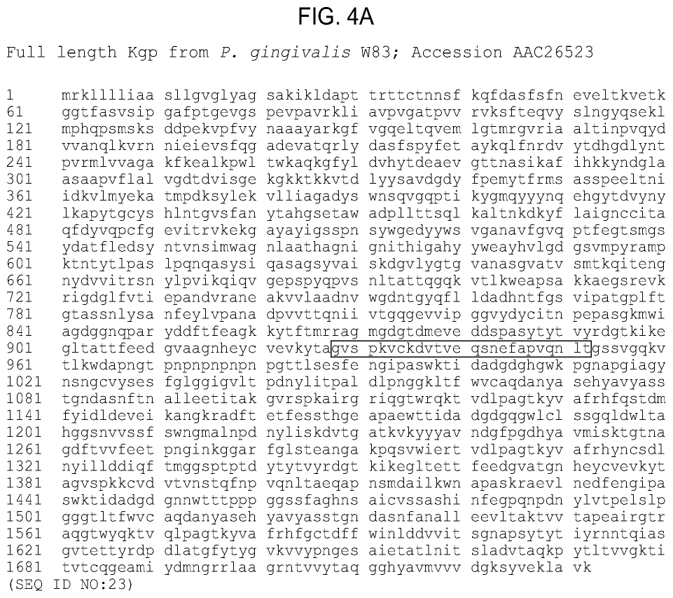

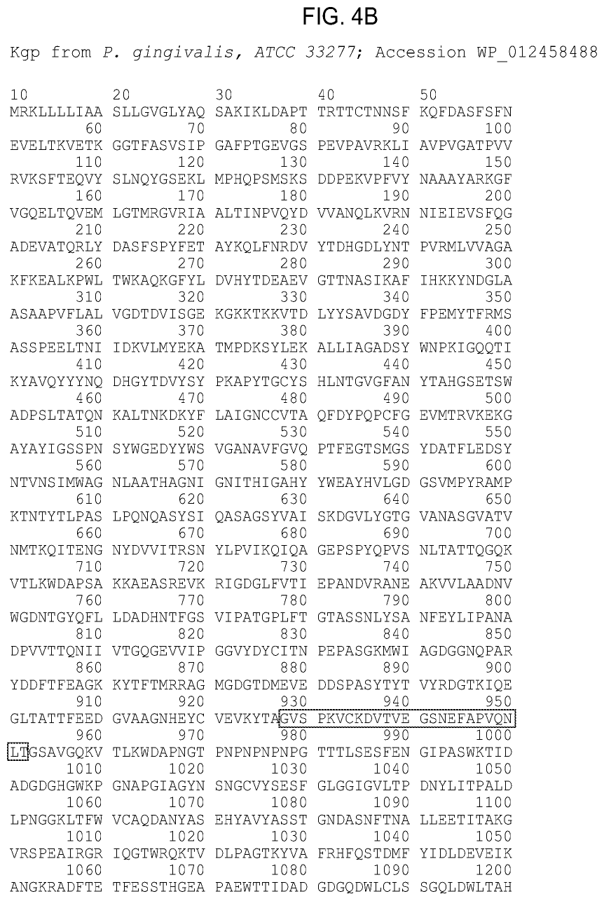

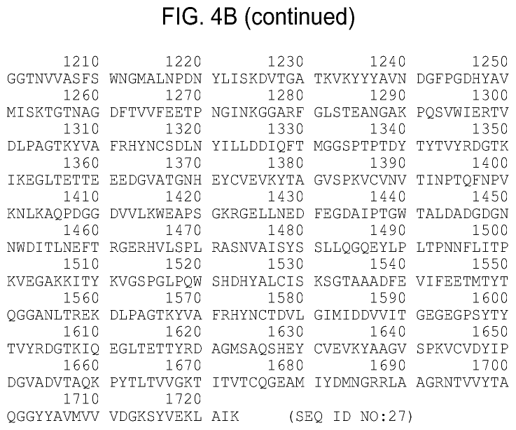

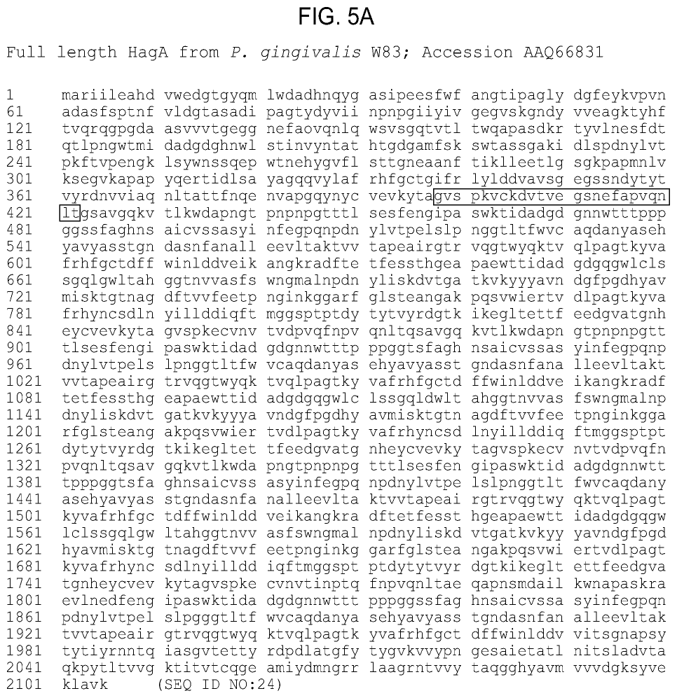

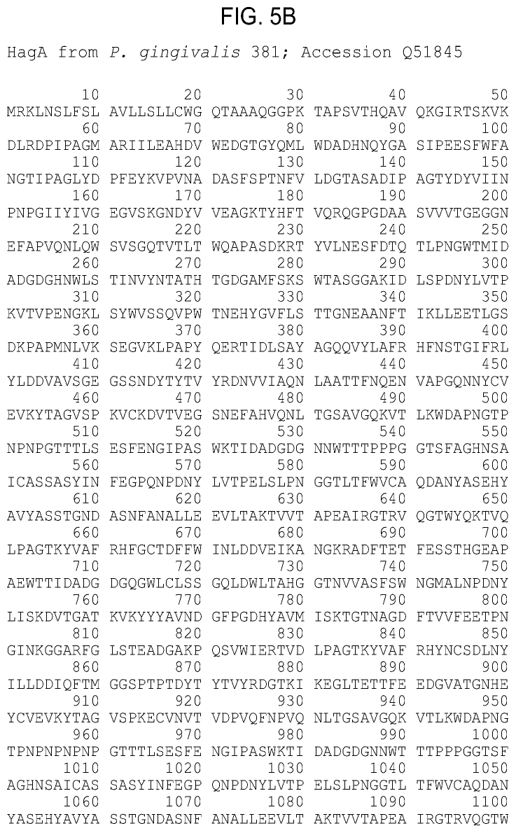

Antigen binding molecules (ABMs) that bind to Porphyromonas gingivalis (e.g. via its cell surface-associated and/or fully secreted outer membrane vesicles containing gingipains/hemaggultini/adhesin/LPS) are provided herein. In certain embodiments, the ABM is a human or humanized ABM. In several embodiments, the ABM is resistant to digestion or cleavage by a protease, e.g., a bacterial protease. In some embodiments, the CDRs are any 1, 2, 3, 4, 5, or 6 CDRs as provided in FIGS. 1A and 1B. In some embodiments, the CDRs are any 1, 2, 3, 4, 5 or 6 CDRs that are within SEQ ID NOS:1 and 2, per the Kabat or Chothia definitions of CDRs. In some embodiments, the CDRs are any 1, 2, 3, 4, 5 or 6 CDRs that are within SEQ ID NOS:9 and 10, per the Kabat or Chothia definitions of CDRs. In some embodiments, the CDRs are any 1, 2, 3, 4, 5 or 6 CDRs that are within SEQ ID NOS:37 and 38, per the Kabat or Chothia definitions of CDRs.

In some embodiments, the ABM, e.g., murine, human or humanized ABM, includes a heavy chain variable region (HVR). In some embodiments, the HVR includes one or more (e.g., 1, 2, or 3) heavy chain CDRs (HCDRs) corresponding to the HCDRs of a heavy chain variable region shown in Table 0.1, per the Kabat or Chothia definitions of CDRs. In some embodiments, the ABM, e.g., murine, human or humanized ABM, includes a light chain variable region (LVR). In some embodiments, the LVR includes one or more (e.g., 1, 2, or 3) light chain CDRs (LCDRs) corresponding to the LCDRs of a light chain variable region shown in Table 0.1, per the Kabat or Chothia definitions of CDRs. In some embodiments, the ABM includes an HVR having an amino acid sequence at least 80%, 85%, 90%, 95%, 97%, or 100% identical to SEQ ID NO:9. In some embodiments, the ABM includes an LVR having an amino acid sequence at least 80%, 85%, 90%, 95%, 97%, or 100% identical to SEQ ID NO:10. In some embodiments, the ABM includes a heavy chain having an amino acid sequence at least 80%, 85%, 90%, 95%, 97%, or 100% identical to SEQ ID NO:74. In some embodiments, the ABM includes a light chain having an amino acid sequence at least 80%, 85%, 90%, 95%, 97%, or 100% identical to SEQ ID NO:76.

TABLE 0.1

| Heavy chain variable | SEQ |
|---|---|
| region amino acid sequence | ID NO: |
| EVQLKQSGPGLVAPSQSLSITCTVSGFSLSIYSVHWVRQPPG | 9 |
| KGLEWLGMIWGGGSSDYNSALKSRLSISKDNSKSQVFLKMNS | |
| LQTDDTAMYYCARNGNFYAMDYWGQGTSVTVSS | |
| QVQLQESGPGLVKPSETLSLTCTVSGFSLSIYSVHWIRQPPG | 37 |
| KGLEWX₁GMIWGGGSSDYNSALKSRX₂TISX₃DTSKNQX₄SLK | |
| LSSVTAADTAX₅YYCARNGNFYAMDYWGQGTLVTVSS, | |
| where X₁ is I or L, X₂ is V or L, X₃ is V | |
| or K, X₄ is F or V, X₅ is V or M. | |
| Light chain variable | SEQ |
| region amino acid sequence | ID NO: |
| QIVLTQSPAIMSASLGERVTMTCTASSSVSSSFLHWYQQKPG | 10 |
| SSPQLWIYSTSNLASGVPARFSGSGSGTSYSLTISSMEAEDA | |
| ATYYCHQYHHSPYIYTFGGGTKLEIK | |
| EIVLTQSPGTLSLSPGERATLSCTASSSVSSSFLHWYQQKPG | 38 |
| QAPX₁LX₂IYSTSNLASGIPX₃RFSGSGSGTDX₄TLTISRLEP | |
| EDFAX₅YYCHQYHHSPYIYTFGGGTKLEIK, | |
| where X₁ is Q or R, X₂ is L or W, X₃ is D | |
| or A, X₄ is For Y, X₅ is V or T. | |

In some embodiments, the ABM, e.g., murine, human or humanized ABM, includes a heavy chain CDR1 (HCDR1) of the HCDR1 of SEQ ID NO:9 or 37; a HCDR2 of the HCDR2 of SEQ ID NO:9 or 37; and/or a HCDR3 of the HCDR3 of SEQ ID NO:9 or 37; and a light chain CDR1 (LCDR1) of the LCDR1 of SEQ ID NO:10 or 38; a LCDR2 of the LCDR2 of SEQ ID NO:10 or 38; and/or a LCDR3 of the LCDR3 of SEQ ID NO:10 or 38. In some embodiments, the HCDR1 of SEQ ID NO: 9 is FSLSIYS (SEQ ID NO:3), the HCDR2 of SEQ ID NO: 9 is IWGGGSS (SEQ ID NO:4), and the HCDR3 of SEQ ID NO:9 is ARNGNFYAMDY (SEQ ID NO:5). In some embodiments, the HCDR1 of SEQ ID NO: 37 is GFSLSIYSVH (SEQ ID NO:39), the HCDR2 of SEQ ID NO: 37 is MIWGGGSSDYNSALKS (SEQ ID NO:40), and the HCDR1 of SEQ ID NO: 37 is NGNFYAMDY (SEQ ID NO:41). In some embodiments, the LCDR1 of SEQ ID NO:10 is SSVSSSF (SEQ ID NO:6), the LCDR2 of SEQ ID NO:10 is STS (SEQ ID NO:7), and the LCDR3 of SEQ ID NO: 10 is HQYHHSPYIYT (SEQ ID NO:8). In some embodiments, the LCDR1 of SEQ ID NO:38 is TASSSVSSSFLH (SEQ ID NO:42), the LCDR2 of SEQ ID NO:38 is STSNLAS (SEQ ID NO:43), and the LCDR3 of SEQ ID NO:38 is HQYHHSPYIYT (SEQ ID NO:8).

In some embodiments, the ABM includes a HCDR1 having the amino acid sequence FSLSIYS (SEQ ID NO:3); a HCDR2 having the amino acid sequence IWGGGSS (SEQ ID NO:4); and/or a HCDR3 having the amino acid sequence ARNGNFYAMDY (SEQ ID NO:5); and/or a LCDR1 having the amino acid sequence SSVSSSF (SEQ ID NO:6); a LCDR2 having the amino acid sequence STS (SEQ ID NO:7); and/or a LCDR3 having the amino acid sequence HQYHHSPYIYT (SEQ ID NO:8). In some embodiments, the ABM includes 1, 2, 3, 4, 5, or 6 of the CDRs above.

In some embodiments, the ABM includes a HCDR1 having the amino acid sequence GFSLSIYSVH (SEQ ID NO:39); a HCDR2 having the amino acid sequence MIWGGGSSDYNSALKS (SEQ ID NO:40); and/or a HCDR3 having the amino acid sequence NGNFYAMDY (SEQ ID NO:41); and/or a LCDR1 having the amino acid sequence TASSSVSSSFLH (SEQ ID NO:42); a LCDR2 having the amino acid sequence STSNLAS (SEQ ID NO:43); and/or a LCDR3 having the amino acid sequence HQYHHSPYIYT (SEQ ID NO:8). In some embodiments, the ABM includes 1, 2, 3, 4, 5, or 6 of the CDRs above.

In some embodiments, the ABM, e.g., human or humanized ABM, includes at least one human framework region (FR). In some embodiments, the ABM includes at least one framework region having an amino acid sequence at least about 80%, e.g., at least about 85%, at least about 90%, at least about 95%, at least about 97%, at least about 99%, or 100% identical to a corresponding human framework region. In some embodiments, the ABM includes a HVR having at least one human FR. In some embodiments, the HVR includes at least one framework region having an amino acid sequence at least about 80%, e.g., at least about 85%, at least about 90%, at least about 95%, at least about 97%, at least about 99%, or 100% identical to a corresponding human HVR framework region. In some embodiments, the LVR includes at least one framework region having an amino acid sequence at least about 80%, e.g., at least about 85%, at least about 90%, at least about 95%, at least about 97%, at least about 99%, or 100% identical to a corresponding human LVR framework region.

In some embodiments, the ABM, e.g., human or humanized ABM, includes at least one of: the HVR residues selected from L48, L67, K71, V78, and M92, as numbered according to the numbering as provided in SEQ ID NO:37, and the LVR residues selected from Q46, W48, A61, Y72, and T86, as numbered according to the numbering as provided in SEQ ID NO:38. In some embodiments, the ABM includes 1, 2, 3, 4, 5, 6, 7, 8, 9 or all 10 of the HVR residues selected from L48, L67, K71, V78, and M92, as numbered according to the numbering as provided in SEQ ID NO:37, and the LVR residues selected from Q46, W48, A61, Y72, and T86, as numbered according to the numbering as provided in SEQ ID NO:38.

In some embodiments, the ABM, e.g., human or humanized ABM, includes a HVR having one or more residues selected from I48/L48, V67/L67, V71/K71, F78/V78, and V92/M92, as numbered according to the numbering as provided in SEQ ID NO:37; and a LVR having one or more residues selected from R46/Q46, L48/W48, D61/A61, F72/Y72, and V86/T86, as numbered according to the numbering as provided in SEQ ID NO:38. In some embodiments, the HVR includes 148, V67, V71, F78 and V92. In some embodiments, the HVR includes 148, L67, K71, V78 and V92. In some embodiments, the HVR includes L48, L67, V71, V78, and M92. In some embodiments, the HVR includes L48, L67, K71, V78, and M92. In some embodiments, the LVR includes Q46, W48, D61, F72 and V86. In some embodiments, the LVR includes Q46, W48, D61, Y72 and V86. In some embodiments, the LVR includes Q46, W48, D61, Y72, and T86. In some embodiments, the LVR includes Q46, W48, A61, Y72, and T86.

In some embodiments, the HVR includes 1, 2, or all 3 HCDRs of the HCDRs of SEQ ID NO:9 or 37, and one or more residues selected from I48/L48, V67/L67, V71/K71, F78/V78, and V92/M92, as numbered according to the numbering as provided in SEQ ID NO:37. In some embodiments, the HVR includes a HCDR1 of the HCDR1 of SEQ ID NO:9 or 37; a HCDR2 of the HCDR2 of SEQ ID NO:9 or 37; and a HCDR3 of the HCDR3 of SEQ ID NO:9 or 37, and one or more residues selected from I48/L48, V67/L67, V71/K71, F78/V78, and V92/M92 as numbered according to the numbering as provided in SEQ ID NO:37. In some embodiments, the HVR includes 148, V67, V71, F78 and V92. In some embodiments, the HVR includes 148, L67, K71, V78 and V92. In some embodiments, the HVR includes L48, L67, V71, V78, and M92. In some embodiments, the HVR includes L48, L67, K71, V78, and M92.

In some embodiments, the LVR includes 1, 2, or all 3 LCDRs of the LCDRs of SEQ ID NO:10 or 38, and one or more residues selected from R46/Q46, L48/W48, D61/A61, F72/Y72, and V86/T86, as numbered according to the numbering as provided in SEQ ID NO:38. In some embodiments, the LVR includes a LCDR 1 of the LCDR1 of SEQ ID NO:10 or 38; a LCDR2 of the LCDR2 of SEQ ID NO:10 or 38; and a LCDR3 of the LCDR3 of SEQ ID NO:10 or 38, and one or more residues selected from R46/Q46, L48/W48, D61/A61, F72/Y72, and V86/T86, as numbered according to the numbering as provided in SEQ ID NO:38. In some embodiments, the LVR includes Q46, W48, D61, F72 and V86. In some embodiments, the LVR includes Q46, W48, D61, Y72 and V86. In some embodiments, the LVR includes Q46, W48, D61, Y72, and T86. In some embodiments, the LVR includes Q46, W48, A61, Y72, and T86.

In some embodiments, the HVR includes an amino acid sequence at least about 80%, e.g., at least about 85%, at least about 90%, at least about 95%, at least about 97%, at least about 99%, or 100% identical to SEQ ID NO:37. In some embodiments, the HVR includes: a heavy chain CDR1 (HCDR1) of the HCDR1 of SEQ ID NO:9 or 37; a HCDR2 of the HCDR2 of SEQ ID NO:9 or 37; and/or a HCDR3 of the HCDR3 of SEQ ID NO:9 or 37; and an amino acid sequence at least about 80%, e.g., at least about 85%, at least about 90%, at least about 95%, at least about 97%, at least about 99%, or 100% identical to SEQ ID NO:37. In some embodiments, the HVR includes: a heavy chain CDR1 (HCDR1) of the HCDR1 of SEQ ID NO:9 or 37; a HCDR2 of the HCDR2 of SEQ ID NO:9 or 37; and a HCDR3 of the HCDR3 of SEQ ID NO:9 or 37; one or more residues selected from I48/L48, V67/L67, V71/K71, F78/V78, and V92/M92, as numbered according to the numbering as provided in SEQ ID NO:37; and an amino acid sequence at least about 80%, e.g., at least about 85%, at least about 90%, at least about 95%, at least about 97%, at least about 99%, or 100% identical to SEQ ID NO:37. In some embodiments, the HVR includes 148, V67, V71, F78 and V92. In some embodiments, the HVR includes 148, L67, K71, V78 and V92. In some embodiments, the HVR includes L48, L67, V71, V78, and M92. In some embodiments, the HVR includes L48, L67, K71, V78, and M92.

In some embodiments, the LVR includes an amino acid sequence at least about 80%, e.g., at least about 85%, at least about 90%, at least about 95%, at least about 97%, at least about 99%, including 100% identical to SEQ ID NO:38. In some embodiments, the LVR includes: a light chain CDR1 (LCDR1) of the LCDR1 of SEQ ID NO:10 or 38; a LCDR2 of the LCDR2 of SEQ ID NO:10 or 38; and/or a LCDR3 of the LCDR3 of SEQ ID NO:10 or 38; and an amino acid sequence at least about 80%, e.g., at least about 85%, at least about 90%, at least about 95%, at least about 97%, at least about 99%, including 100% identical to SEQ ID NO:38. In some embodiments, the LVR includes: a light chain CDR1 (LCDR1) of the LCDR1 of SEQ ID NO:10 or 38; a LCDR2 of the LCDR2 of SEQ ID NO: 10 or 38; and/or a LCDR3 of the LCDR3 of SEQ ID NO:10 or 38; one or more residues selected from R46/Q46, L48/W48, D61/A61, F72/Y72, and V86/T86, as numbered according to the numbering as provided in SEQ ID NO:38; and an amino acid sequence at least about 80%, e.g., at least about 85%, at least about 90%, at least about 95%, at least about 97%, or at least about 99% identical to SEQ ID NO:38. In some embodiments, the LVR includes Q46, W48, D61, F72 and V86. In some embodiments, the LVR includes Q46, W48, D61, Y72 and V86. In some embodiments, the LVR includes Q46, W48, D61, Y72, and T86. In some embodiments, the LVR includes Q46, W48, A61, Y72, and T86.

In some embodiments, the ABM, e.g., human or humanized ABM, includes a HVR having a heavy chain framework region 1 (HFR1) of the HFR1 in SEQ ID NO:37; a HFR2 of the HFR2 in SEQ ID NO:37; a HFR3 of the HFR3 in SEQ ID NO:37; and/or a HFR4 of the HFR4 in SEQ ID NO:37. In some embodiments, the ABM, e.g., human or humanized ABM, includes a LVR having a light chain framework region 1 (LFR1) of the LFR1 in SEQ ID NO:38; a LFR2 of the LFR2 in SEQ ID NO:38; a LFR3 of the LFR3 in SEQ ID NO:38; and/or a LFR4 of the LFR4 in SEQ ID NO:38. In some embodiments, the ABM, e.g., human or humanized ABM, includes a HVR having a heavy chain framework region 1 (HFR1) of the HFR1 in SEQ ID NO:37; a HFR2 of the HFR2 in SEQ ID NO:37; a HFR3 of the HFR3 in SEQ ID NO:37; and/or a HFR4 of the HFR4 in SEQ ID NO:37; and a LVR having a light chain framework region 1 (LFR1) of the LFR1 in SEQ ID NO:38; a LFR2 of the LFR2 in SEQ ID NO:38; a LFR3 of the LFR3 in SEQ ID NO:38; and/or a LFR4 of the LFR4 in SEQ ID NO:38.

In some embodiments, the HVR includes a heavy chain framework region 1 (HFR1) of the HFR1 in any one of SEQ ID NOS:29-32; a HFR2 of the HFR2 in any one of SEQ ID NOS:29-32; a HFR3 of the HFR3 in any one of SEQ ID NOS:29-32; and a HFR4 of the HFR4 in any one of SEQ ID NOS:29-32. In some embodiments, the LVR includes a light chain framework region 1 (LFR1) of the LFR1 in any one of SEQ ID NOS:33-36; a LFR2 of the LFR2 in any one of SEQ ID NOS:33-36; a LFR3 of the LFR3 in any one of SEQ ID NOS:33-36; and a LFR4 of the LFR4 in any one of SEQ ID NOS:33-36.

In some embodiments, the ABM, e.g., human or humanized ABM, includes a HVR having an amino acid sequence at least about 80%, e.g., at least about 85%, at least about 90%, at least about 95%, at least about 97%, at least about 99%, or 100% identical to any one of SEQ ID NOS: 29-32. In some embodiments, the ABM, e.g., human or humanized ABM, includes a LVR having an amino acid sequence at least about 80%, e.g., at least about 85%, at least about 90%, at least about 95%, at least about 97%, at least about 99%, or 100% identical to any one of SEQ ID NOS:33-36. In some embodiments, the ABM, e.g., human or humanized ABM, includes a HVR having an amino acid sequence at least about 80%, e.g., at least about 85%, at least about 90%, at least about 95%, at least about 97%, at least about 99%, or 100% identical to any one of SEQ ID NOS: 29-32; and a LVR having an amino acid sequence at least about 80%, e.g., at least about 85%, at least about 90%, at least about 95%, at least about 97%, at least about 99%, or 100% identical to any one of SEQ ID NOS:33-36. In some embodiments, the ABM, e.g., human or humanized ABM, includes a HVR having a HCDR1 of the HCDR1 of SEQ ID NO:9 or 37; a HCDR2 of the HCDR2 of SEQ ID NO:9 or 37; and a HCDR3 of the HCDR3 of SEQ ID NO:9 or 37; and an amino acid sequence at least about 80%, e.g., at least about 85%, at least about 90%, at least about 95%, at least about 97%, at least about 99%, or 100% identical to any one of SEQ ID NOS: 29-32; and a LVR having a LCDR1 of the LCDR1 of SEQ ID NO:9 or 37; a LCDR2 of the LCDR2 of SEQ ID NO:9 or 37; and a LCDR3 of the LCDR3 of SEQ ID NO:9 or 37; and an amino acid sequence at least about 80%, e.g., at least about 85%, at least about 90%, at least about 95%, at least about 97%, at least about 99%, or 100% identical to any one of SEQ ID NOS:33-36. In some embodiments, the HVR includes an amino acid sequence at least about 80%, e.g., at least about 85%, at least about 90%, at least about 95%, at least about 97%, at least about 99%, or 100% identical to SEQ ID NO:29; and the LVR includes an amino acid sequence at least about 80%, e.g., at least about 85%, at least about 90%, at least about 95%, at least about 97%, at least about 99%, or 100% identical to any one of SEQ ID NOS:33-36. In some embodiments, the HVR includes an amino acid sequence at least about 80%, e.g., at least about 85%, at least about 90%, at least about 95%, at least about 97%, at least about 99%, or 100% identical to SEQ ID NO:30; and the LVR includes an amino acid sequence at least about 80%, e.g., at least about 85%, at least about 90%, at least about 95%, at least about 97%, at least about 99%, or 100% identical to any one of SEQ ID NOS:33-36. In some embodiments, the HVR includes an amino acid sequence at least about 80%, e.g., at least about 85%, at least about 90%, at least about 95%, at least about 97%, at least about 99%, or 100% identical to SEQ ID NO:31 and the LVR includes an amino acid sequence at least about 80%, e.g., at least about 85%, at least about 90%, at least about 95%, at least about 97%, at least about 99%, or 100% identical to any one of SEQ ID NOS:33-36. In some embodiments, the HVR includes an amino acid sequence at least about 80%, e.g., at least about 85%, at least about 90%, at least about 95%, at least about 97%, at least about 99%, or 100% identical to SEQ ID NO:32; and the LVR includes an amino acid sequence at least about 80%, e.g., at least about 85%, at least about 90%, at least about 95%, at least about 97%, at least about 99%, or 100% identical to any one of SEQ ID NOS:33-36. In some embodiments, the HVR includes an amino acid sequence at least about 80%, e.g., at least about 85%, at least about 90%, at least about 95%, at least about 97%, at least about 99%, or 100% identical to any one of SEQ ID NOS:29-32; and the LVR includes an amino acid sequence at least about 80%, e.g., at least about 85%, at least about 90%, at least about 95%, at least about 97%, at least about 99%, or 100% identical to SEQ ID NO:33. In some embodiments, the HVR includes an amino acid sequence at least about 80%, e.g., at least about 85%, at least about 90%, at least about 95%, at least about 97%, at least about 99%, or 100% identical to any one of SEQ ID NOS:29-32; and the LVR includes an amino acid sequence at least about 80%, e.g., at least about 85%, at least about 90%, at least about 95%, at least about 97%, at least about 99%, or 100% identical to SEQ ID NO:34. In some embodiments, the HVR includes an amino acid sequence at least about 80%, e.g., at least about 85%, at least about 90%, at least about 95%, at least about 97%, at least about 99%, or 100% identical to any one of SEQ ID NOS:29-32; and the LVR includes an amino acid sequence at least about 80%, e.g., at least about 85%, at least about 90%, at least about 95%, at least about 97%, at least about 99%, or 100% identical to SEQ ID NO:35. In some embodiments, the HVR includes an amino acid sequence at least about 80%, e.g., at least about 85%, at least about 90%, at least about 95%, at least about 97%, at least about 99%, or 100% identical to any one of SEQ ID NOS:29-32; and the LVR includes an amino acid sequence at least about 80%, e.g., at least about 85%, at least about 90%, at least about 95%, at least about 97%, at least about 99%, or 100% identical to SEQ ID NO:36.

In some embodiments, the ABM, e.g., human or humanized ABM, includes a HVR having an amino acid sequence of any one of SEQ ID NOS: 29-32. In some embodiments, the ABM, e.g., human or humanized ABM, includes a LVR having an amino acid sequence of any one of SEQ ID NOS: 23-36. In some embodiments, the ABM, e.g., human or humanized ABM, includes a HVR having an amino acid sequence of any one of SEQ ID NOS: 29-32; and a LVR having an amino acid sequence of any one of SEQ ID NOS:33-36. The ABM can have any suitable combination of HVR and LVR, as provided above. In some embodiments, the ABM includes a HVR having an amino acid sequence of SEQ ID NO:29 and a LVR having an amino acid sequence of any one of SEQ ID NOS:33-36. In some embodiments, the ABM includes a HVR having an amino acid sequence of SEQ ID NO:30 and a LVR having an amino acid sequence of any one of SEQ ID NOS:33-36. In some embodiments, the ABM includes a HVR having an amino acid sequence of SEQ ID NO:31 and a LVR having an amino acid sequence of any one of SEQ ID NOS:33-36. In some embodiments, the ABM includes a HVR having an amino acid sequence of SEQ ID NO:32 and a LVR having an amino acid sequence of any one of SEQ ID NOS:33-36. In some embodiments, the ABM includes a HVR having an amino acid sequence of any one of SEQ ID NOS:29-32 and a LVR having an amino acid sequence of any one of SEQ ID NOS:33. In some embodiments, the ABM includes a HVR having an amino acid sequence of any one of SEQ ID NOS:29-32 and a LVR having an amino acid sequence of any one of SEQ ID NOS:34. In some embodiments, the ABM includes a HVR having an amino acid sequence of any one of SEQ ID NOS:29-32 and a LVR having an amino acid sequence of any one of SEQ ID NOS:35. In some embodiments, the ABM includes a HVR having an amino acid sequence of any one of SEQ ID NOS:29-32 and a LVR having an amino acid sequence of any one of SEQ ID NOS:36.

In some embodiments, an ABM of the present disclosure includes a heavy chain variable region having an amino acid sequence at least 80%, 90%, 95%, 97%, 98%, 99%, or 100% identity to SEQ ID NO:32, and a light chain variable region having an amino acid sequence at least 80%, 90%, 95%, 97%, 98%, 99%, or 100% identity to SEQ ID NO:34. In some embodiments, the ABM includes a HVR having a HCDR1 of the HCDR1 of SEQ ID NO:9 or 37; a HCDR2 of the HCDR2 of SEQ ID NO:9 or 37; and a HCDR3 of the HCDR3 of SEQ ID NO:9 or 37; and an amino acid sequence at least 80%, 90%, 95%, 97%, 98%, 99%, or 100% identity to SEQ ID NO:32; and a LCDR1 of the LCDR1 of SEQ ID NO:10 or 38; a LCDR2 of the LCDR2 of SEQ ID NO:10 or 38; and/or a LCDR3 of the LCDR3 of SEQ ID NO:10 or 38; and an amino acid sequence at least 80%, 90%, 95%, 97%, 98%, 99%, or 100% identity to SEQ ID NO:34.

In some embodiments, an ABM of the present disclosure includes a heavy chain variable region having an amino acid sequence at least 80%, 90%, 95%, 97%, 98%, 99%, or 100% identity to SEQ ID NO:30, and a light chain variable region having an amino acid sequence at least 80%, 90%, 95%, 97%, 98%, 99%, or 100% identity to SEQ ID NO:33. In some embodiments, the ABM includes a HVR having a HCDR1 of the HCDR1 of SEQ ID NO:9 or 37; a HCDR2 of the HCDR2 of SEQ ID NO:9 or 37; and a HCDR3 of the HCDR3 of SEQ ID NO:9 or 37; and an amino acid sequence at least 80%, 90%, 95%, 97%, 98%, 99%, or 100% identity to SEQ ID NO:30; and a LCDR1 of the LCDR1 of SEQ ID NO:10 or 38; a LCDR2 of the LCDR2 of SEQ ID NO:10 or 38; and/or a LCDR3 of the LCDR3 of SEQ ID NO:10 or 38; and an amino acid sequence at least 80%, 90%, 95%, 97%, 98%, 99%, or 100% identity to SEQ ID NO:33.

In some embodiments, an ABM of the present disclosure includes a heavy chain variable region having an amino acid sequence at least 80%, 90%, 95%, 97%, 98%, 99%, or 100% identity to SEQ ID NO:30, and a light chain variable region having an amino acid sequence at least 80%, 90%, 95%, 97%, 98%, 99%, or 100% identity to SEQ ID NO:35. In some embodiments, the ABM includes a HVR having a HCDR1 of the HCDR1 of SEQ ID NO:9 or 37; a HCDR2 of the HCDR2 of SEQ ID NO:9 or 37; and a HCDR3 of the HCDR3 of SEQ ID NO:9 or 37; and an amino acid sequence at least 80%, 90%, 95%, 97%, 98%, 99%, or 100% identity to SEQ ID NO:30; and a LCDR1 of the LCDR1 of SEQ ID NO:10 or 38; a LCDR2 of the LCDR2 of SEQ ID NO:10 or 38; and/or a LCDR3 of the LCDR3 of SEQ ID NO:10 or 38; and an amino acid sequence at least 80%, 90%, 95%, 97%, 98%, 99%, or 100% identity to SEQ ID NO:35.

In some embodiments, an ABM of the present disclosure includes a heavy chain variable region having an amino acid sequence at least 80%, 90%, 95%, 97%, 98%, 99%, or 100% identity to SEQ ID NO:30, and a light chain variable region having an amino acid sequence at least 80%, 90%, 95%, 97%, 98%, 99%, or 100% identity to SEQ ID NO:36. In some embodiments, the ABM includes a HVR having a HCDR1 of the HCDR1 of SEQ ID NO:9 or 37; a HCDR2 of the HCDR2 of SEQ ID NO:9 or 37; and a HCDR3 of the HCDR3 of SEQ ID NO:9 or 37; and an amino acid sequence at least 80%, 90%, 95%, 97%, 98%, 99%, or 100% identity to SEQ ID NO:30; and a LCDR1 of the LCDR1 of SEQ ID NO:10 or 38; a LCDR2 of the LCDR2 of SEQ ID NO:10 or 38; and/or a LCDR3 of the LCDR3 of SEQ ID NO:10 or 38; and an amino acid sequence at least 80%, 90%, 95%, 97%, 98%, 99%, or 100% identity to SEQ ID NO:36.

In some embodiments, an ABM of the present disclosure includes a heavy chain variable region having an amino acid sequence at least 80%, 90%, 95%, 97%, 98%, 99%, or 100% identity to SEQ ID NO:32, and a light chain variable region having an amino acid sequence at least 80%, 90%, 95%, 97%, 98%, 99%, or 100% identity to SEQ ID NO:35. In some embodiments, the ABM includes a HVR having a HCDR1 of the HCDR1 of SEQ ID NO:9 or 37; a HCDR2 of the HCDR2 of SEQ ID NO:9 or 37; and a HCDR3 of the HCDR3 of SEQ ID NO:9 or 37; and an amino acid sequence at least 80%, 90%, 95%, 97%, 98%, 99%, or 100% identity to SEQ ID NO:32; and a LCDR1 of the LCDR1 of SEQ ID NO:10 or 38; a LCDR2 of the LCDR2 of SEQ ID NO:10 or 38; and/or a LCDR3 of the LCDR3 of SEQ ID NO:10 or 38; and an amino acid sequence at least 80%, 90%, 95%, 97%, 98%, 99%, or 100% identity to SEQ ID NO:35.

In some embodiments, the ABM, e.g., human or humanized ABM, is an antibody. In some embodiments, the ABM includes a heavy chain constant region derived from human gamma, mu, alpha, delta, or epsilon heavy chain. In some embodiments, the ABM includes a light chain constant region derived from human lambda or kappa light chain. In some embodiments, the ABM is of a human IgG (e.g. IgG1, IgG2, IgG3 or IgG4), IgM, IgA, IgD, or IgE isotype. In some embodiments, the ABM is of an IgG isotype, e.g., human IgG isotype. In some embodiments, the ABM binds to an epitope within a polypeptide comprising an amino acid sequence of any one of SEQ ID NOs: 77-83.

The ABM, e.g., murine, human or humanized ABM, of the present disclosure generally binds to an antigen associated with, and/or expressed by, P. gingivalis. The ABM in certain embodiments binds to one or more strains of P. gingivalis. Strains of P. gingivalis to which the ABM binds can include, without limitation, strains W83, W12, W50, 381, A7A1-28, HG66 and ATCC33277. In some embodiments, the ABM binds to any one, two, three, four, five or all six of P. gingivalis strains W83, W12, W50, 381, A7A1-28, and/or ATCC33277. In some embodiments, the ABM binds to strains W83, W12, W50, 381, A7A1-28, and/or ATCC33277. In some embodiments, the ABM binds to clinically important (e.g., virulent and/or chronic inflammation-causing) strains of P. gingivalis. In some embodiments, the ABM binds to clinically isolated strains of P. gingivalis.

In some embodiments, the ABM, e.g., murine, human or humanized ABM, of the present disclosure specifically binds to a P. gingivalis cell-surface antigen. In some embodiments, the ABM of the present disclosure specifically binds to an antigen associated with outer membrane vesicles (OMVs) of P. gingivalis.

In some embodiments, the ABM, e.g., murine, human or humanized ABM, competes with KB001 for binding to P. gingivalis. In some embodiments, the ABM binds to the same or overlapping epitope as KB001. In some embodiments, the ABM comprises the CDRs of the 6 CDRs in SEQ ID NO: 1 and 2. In some embodiments, the ABM comprises at least one, two, three, four, five, or all 6 of the CDRs in SEQ ID NO: 1 and 2. In some embodiments, an ABM of the present disclosure, e.g., human or humanized ABM, competes for binding to P. gingivalis (e.g., P. gingivalis gingipain, hemagglutinin, and/or OMV or budding OMV) with an antibody having a heavy chain variable region containing an amino acid sequence of SEQ ID NO:37, as shown in Table 0.1, and a light chain variable region containing an amino acid sequence of SEQ ID NO:38, as shown in Table 0.1. In some embodiments, an ABM of the present disclosure, e.g., human or humanized ABM, competes for binding to P. gingivalis (e.g., P. gingivalis gingipain, hemagglutinin, and/or OMV or budding OMV) with an antibody having a heavy chain variable region containing an amino acid sequence of any one of SEQ ID NOS:29-32, and a light chain variable region containing an amino acid sequence of any one of SEQ ID NOS:33-36. In some embodiments, an ABM of the present disclosure, e.g., human or humanized ABM, competes for binding to P. gingivalis (e.g., P. gingivalis gingipain, hemagglutinin, and/or OMV or budding OMV) with an antibody having a heavy chain variable region containing an amino acid sequence of SEQ ID NO:30 and a light chain variable region containing an amino acid sequence of SEQ ID NO:33. In some embodiments, an ABM of the present disclosure, e.g., human or humanized ABM, competes for binding to P. gingivalis (e.g., P. gingivalis gingipain, hemagglutinin, and/or OMV or budding OMV) with an antibody having a heavy chain variable region containing an amino acid sequence of SEQ ID NO:30 and a light chain variable region containing an amino acid sequence of SEQ ID NO:35. In some embodiments, an ABM of the present disclosure, e.g., human or humanized ABM, competes for binding to P. gingivalis (e.g., P. gingivalis gingipain, hemagglutinin, and/or OMV or budding OMV) with an antibody having a heavy chain variable region containing an amino acid sequence of SEQ ID NO:32 and a light chain variable region containing an amino acid sequence of SEQ ID NO:34. In some embodiments, an ABM of the present disclosure, e.g., human or humanized ABM, competes for binding to P. gingivalis (e.g., P. gingivalis gingipain, hemagglutinin, and/or OMV or budding OMV) with an antibody having heavy chain and light chain variable regions as set forth in Table 13.1. In some embodiments, an ABM of the present disclosure, e.g., human or humanized ABM, competes for binding to P. gingivalis (e.g., P. gingivalis gingipain, hemagglutinin, and/or OMV or budding OMV) with H5, H7, or H14.

In some embodiments, the ABM specifically binds to an epitope that includes the amino acid sequence GVSPKVCKDVTVEGSNEFAPVQNLT (SEQ ID NO:19). In certain embodiments, the ABM specifically binds to a polypeptide that includes an amino acid sequence at least about 70%, e.g., at least about 75%, at least about 80%, at least about 85%, at least about 90%, at least about 95%, at least about 97%, at least about 99%, or 100% identical to the sequence AGTYDFAIAAPQANAKIWIAGQGPTKEDDYVFEAGKKYHFLMKKMGSGDGTELTIS EGGGSDYTYTVYRDGTKIKEGLTATTFEEDGVAAGNHEYCVEVKYTAGVSPKVCK DVTVEGSNEFAPVQNLT (SEQ ID NO:20). In certain embodiments, the ABM specifically binds to a polypeptide that includes an amino acid sequence at least about 70%, e.g., at least about 75%, at least about 80%, at least about 85%, at least about 90%, at least about 95%, at least about 97%, at least about 99%, or 100% identical to residues 64-129 of the sequence AGTYDFAIAAPQANAKIWIAGQGPTKEDDYVFEAGKKYHFLMKKMGSGDGTELTIS EGGGSDYTYTVYRDGTKIKEGLTATTFEEDGVAAGNHEYCVEVKYTAGVSPKVCK DVTVEGSNEFAPVQNLT (SEQ ID NO:20). In some embodiments, the ABM specifically binds to a polypeptide that includes an epitope having the amino acid sequence GVSPKVCKDVTVEGSNEFAPVQNLT (SEQ ID NO:19), and includes an amino acid sequence at least about 70%, e.g., at least about 75%, at least about 80%, at least about 85%, at least about 90%, at least about 95%, at least about 97%, at least about 99%, or 100% identical to the sequence AGTYDFAIAAPQANAKIWIAGQGPTKEDDYVFEAGKKYHFLMKKMGSGDGTELTIS EGGGSDYTYTVYRDGTKIKEGLTATTFEEDGVAAGNHEYCVEVKYTAGVSPKVCK DVTVEGSNEFAPVQNLT (SEQ ID NO:20). In some embodiments, the ABM specifically binds to a polypeptide that includes an epitope having the amino acid sequence GVSPKVCKDVTVEGSNEFAPVQNLT (SEQ ID NO:19), and includes an amino acid sequence at least about 70%, e.g., at least about 75%, at least about 80%, at least about 85%, at least about 90%, at least about 95%, at least about 97%, at least about 99%, or 100% identical to residues 64-129 of the sequence AGTYDFAIAAPQANAKIWIAGQGPTKEDDYVFEAGKKYHFLMKKMGSGDGTELTIS EGGGSDYTYTVYRDGTKIKEGLTATTFEEDGVAAGNHEYCVEVKYTAGVSPKVCK DVTVEGSNEFAPVQNLT (SEQ ID NO:20).

In some embodiments, the ABM specifically binds to an epitope that includes an amino acid sequence at least about 70%, e.g., at least about 75%, at least about 80%, at least about 85%, at least about 90%, at least about 95%, at least about 97%, at least about 99%, or 100% identical to residues 784 to 1130 of SEQ ID NO:21. In some embodiments, the ABM binds to an epitope within a polypeptide comprising an amino acid sequence that is at least about 70%, e.g., at least about 75%, at least about 80%, at least about 85%, at least about 90%, at least about 95%, at least about 97%, at least about 99%, or 100% identical of any one of SEQ ID NOs: 77-83.

In some embodiments, the ABM specifically binds to an epitope that includes the linear amino acid sequence YCVEVKYTAGVSPK (SEQ ID NO:59). In some embodiments, the ABM competes with an antibody (e.g., KB001) for binding to a polypeptide containing a linear epitope having the amino acid sequence YCVEVKYTAGVSPK (SEQ ID NO:59). In some embodiments, the ABM specifically binds to an epitope that includes the amino acid sequence YCVEVKYX₁AGVSPK (SEQ ID NO:60), where X₁ is T or A. In some embodiments, the ABM competes with an antibody (e.g., KB001) for binding to a polypeptide containing a linear epitope having the amino acid sequence YCVEVKYX₁AGVSPK (SEQ ID NO:60), where X₁ is T or A. In some embodiments, the ABM specifically binds to an epitope that includes the linear amino acid sequence GVSPK (SEQ ID NO:162). In some embodiments, the ABM competes with an antibody (e.g., KB001) for binding to a polypeptide containing a linear epitope having the amino acid sequence GVSPK (SEQ ID NO: 162).

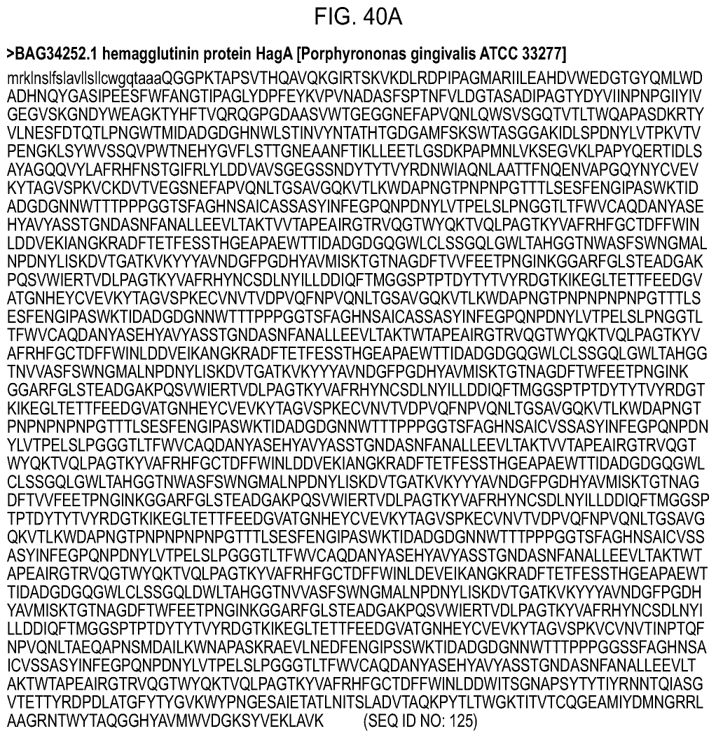

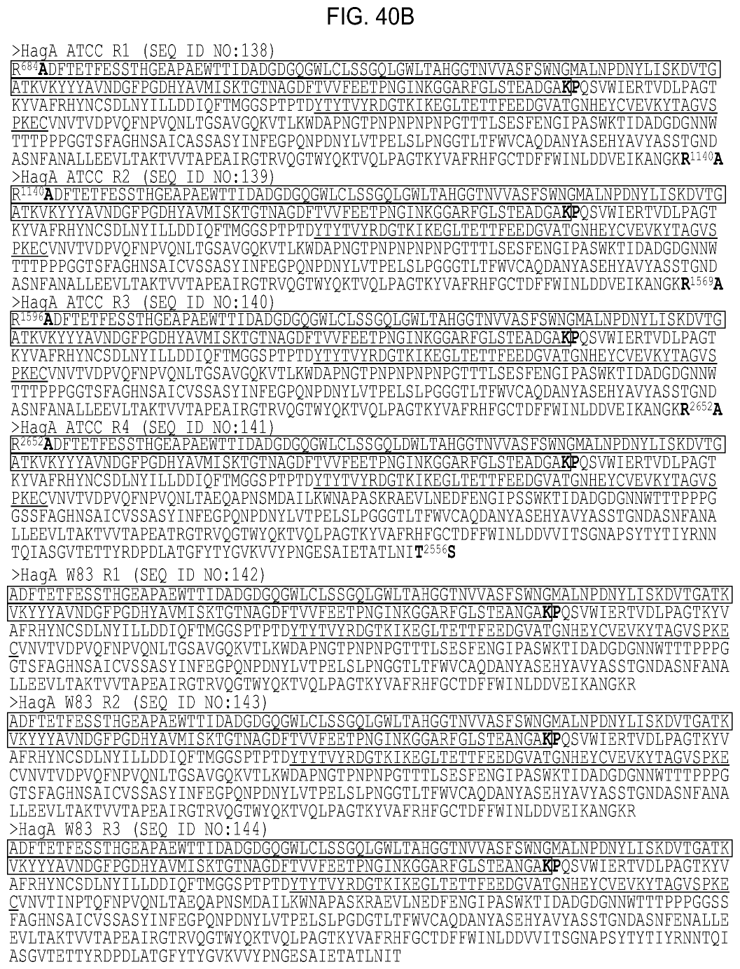

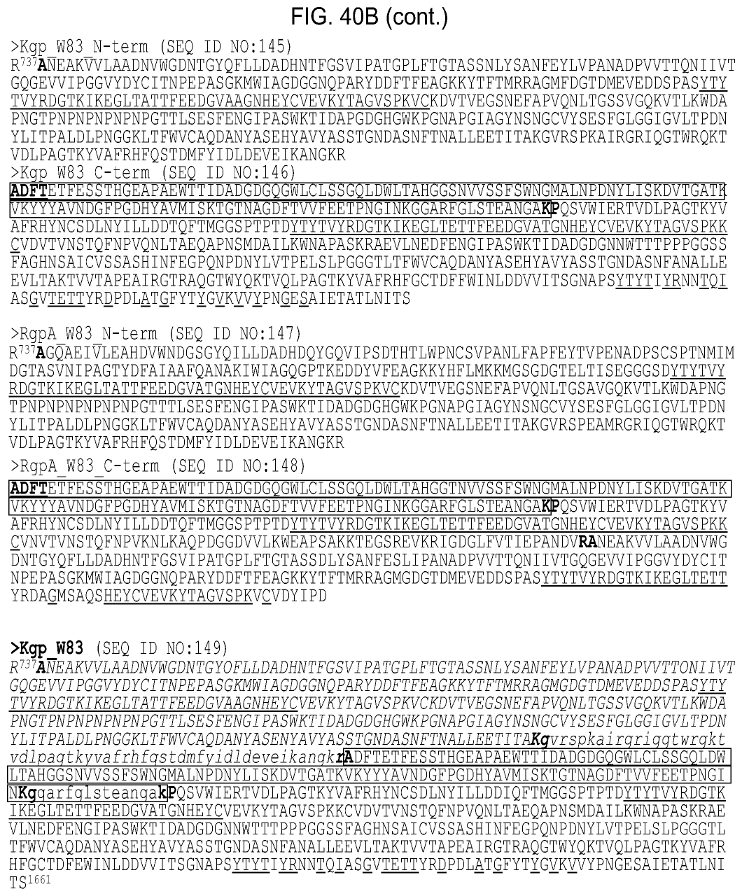

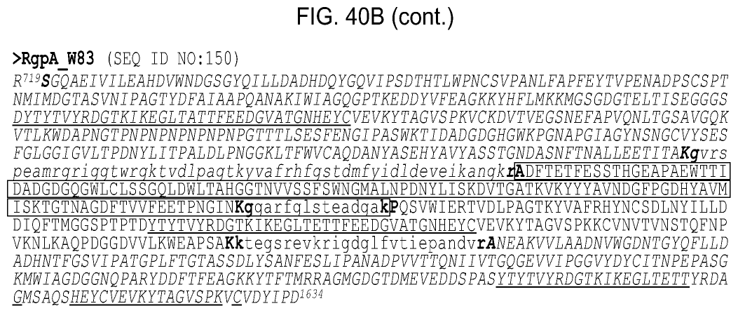

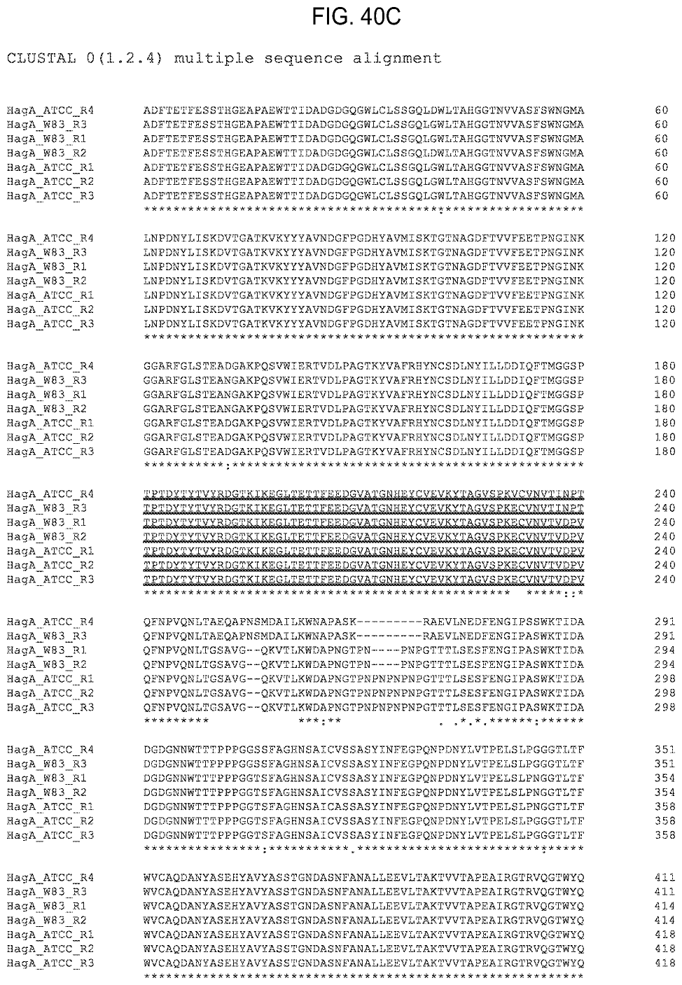

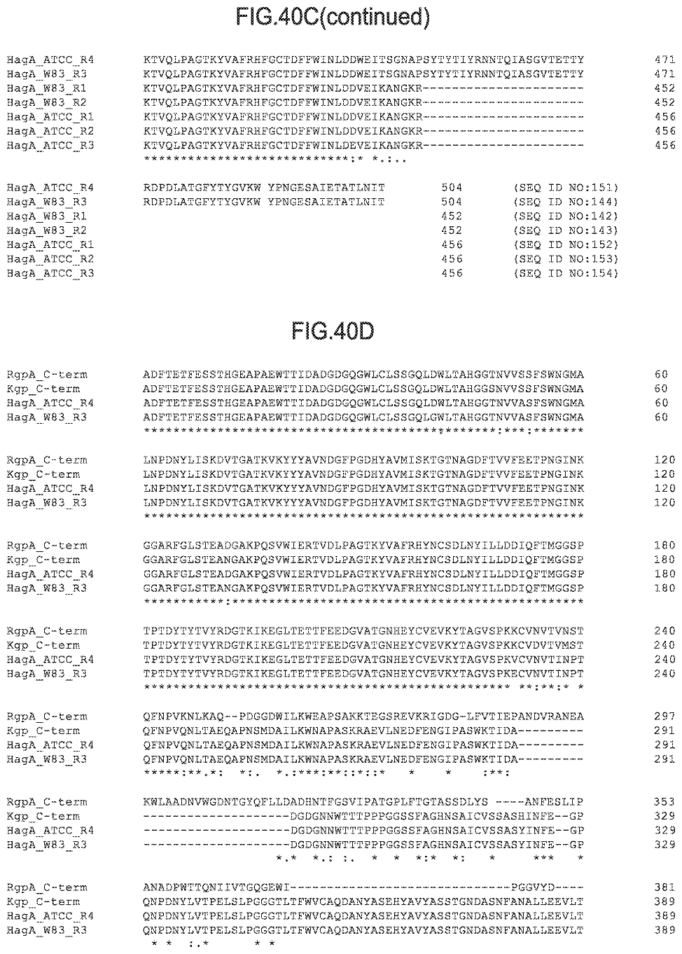

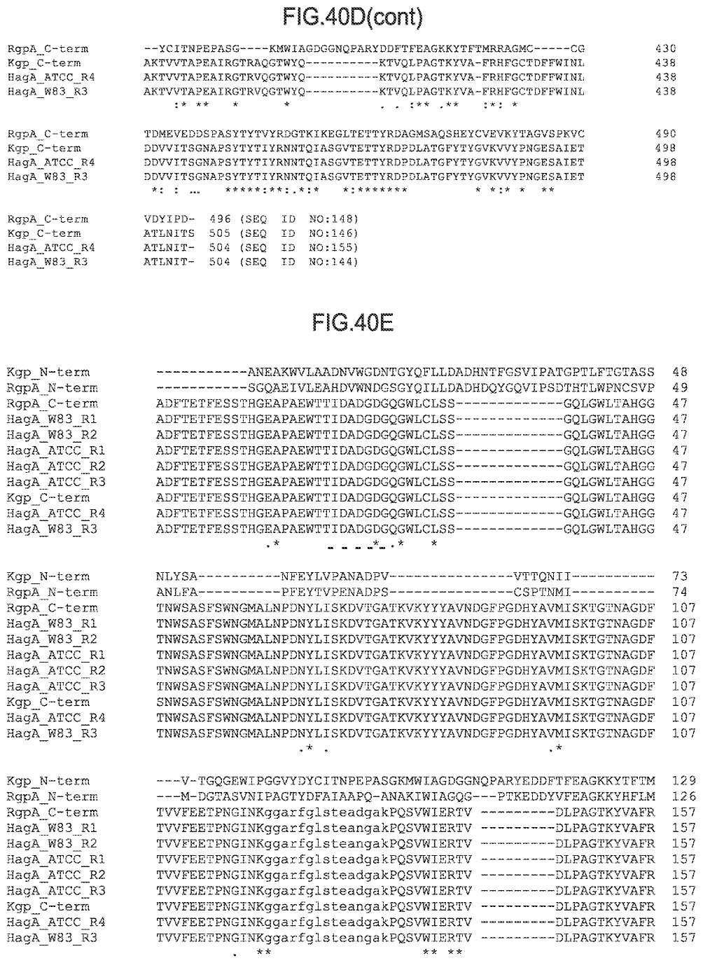

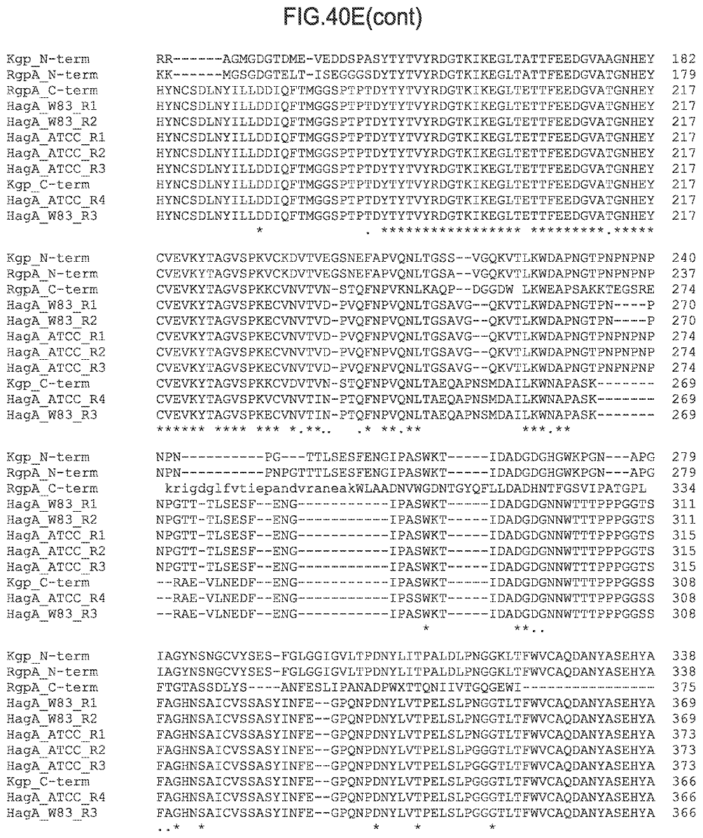

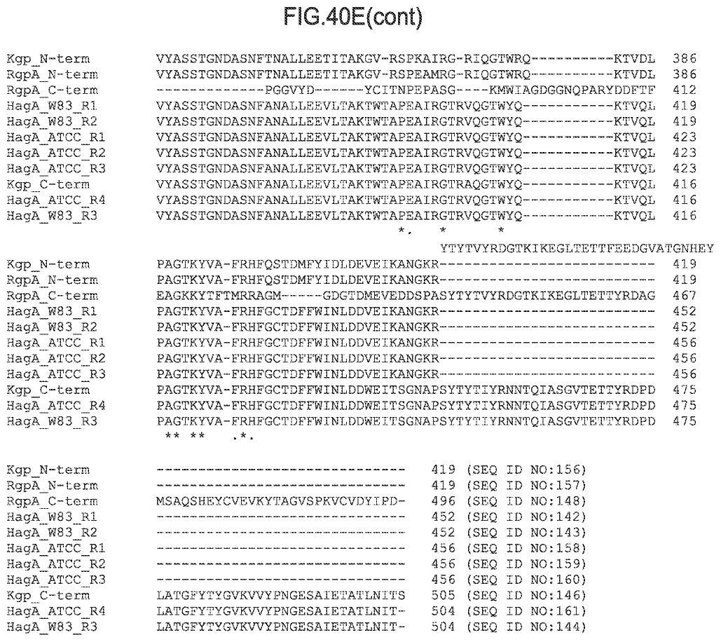

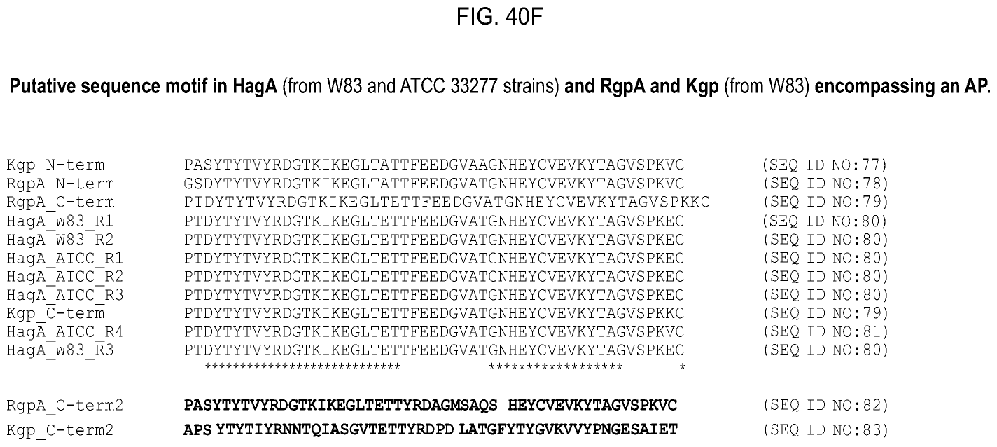

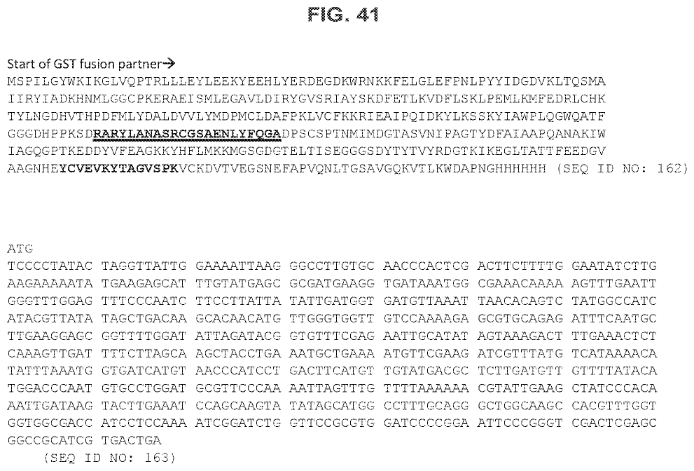

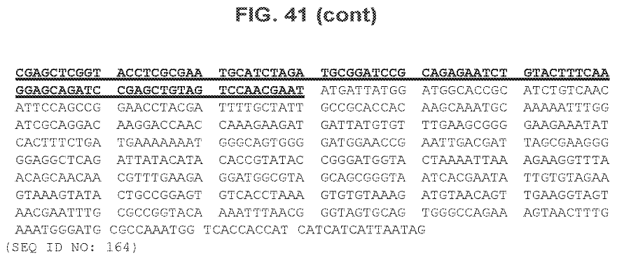

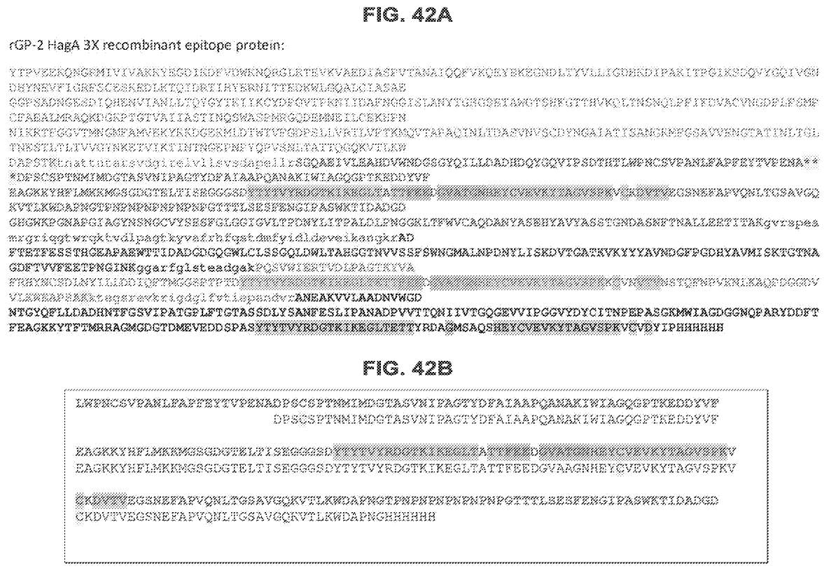

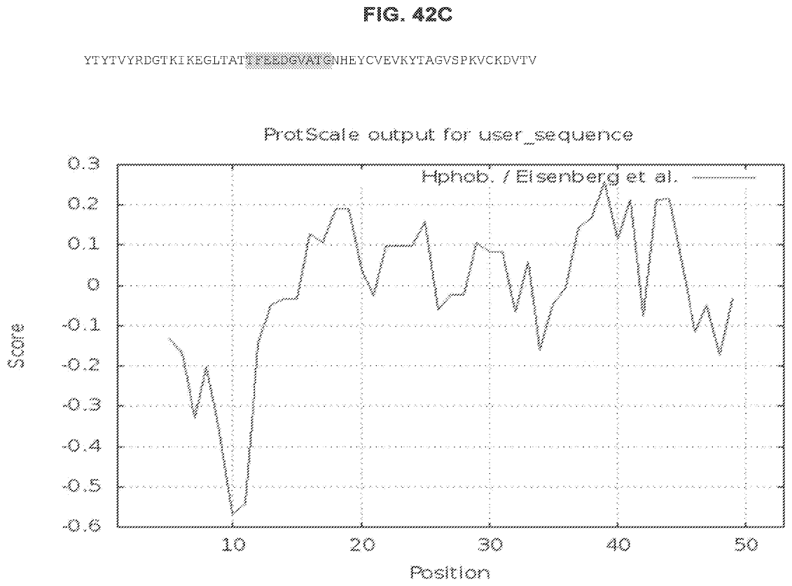

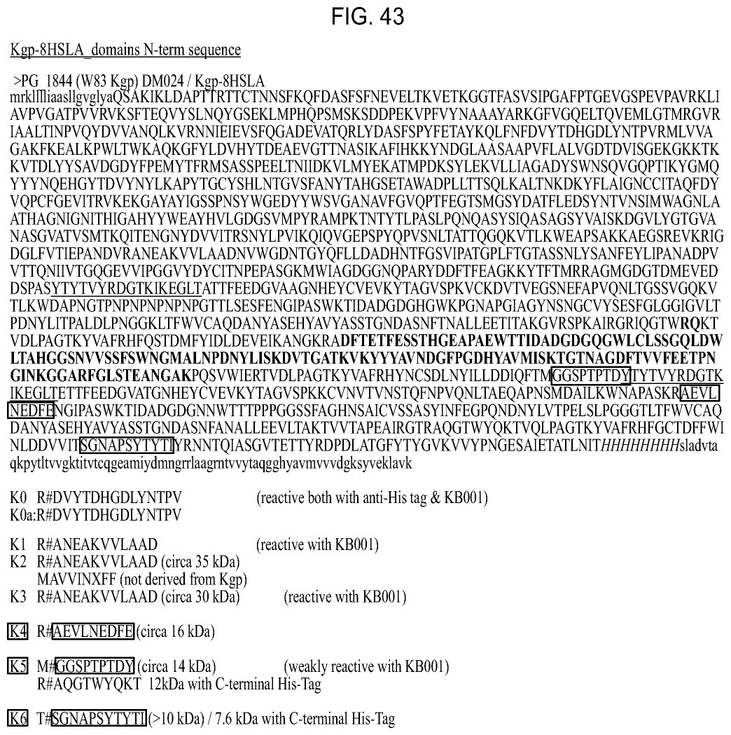

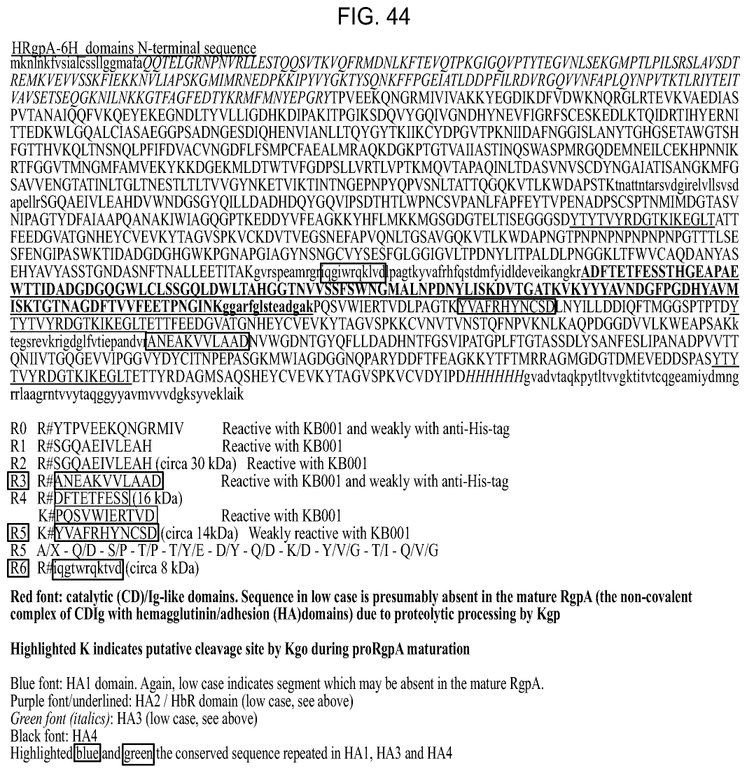

In some embodiments, the ABM binds an epitope in a sequence within a P. gingivalis gingipain (e.g., RgpA, Kgp) and/or hemagglutinin (e.g., HagA) from various strains. In some embodiments, the ABM binds an epitope within a sub-sequence of a P. gingivalis gingipain (e.g., RgpA, Kgp) and/or hemagglutinin (e.g., HagA) as shown in any one of FIGS. 40A-40F. FIG. 40B, provides non-limiting examples of amino acid sequences of the repeated domains of P. gingivalis gingipains and hemagglutinins (e.g., RgpA, Kgp, HagA) with sequences encompassing the putative epitope of an ABM of the present disclosure underlined. In some cases, the P. gingivalis gingipains (e.g., RgpA, Kgp) include an amino acid sequence that partially aligns with a sequence encompassing the putative epitope of an ABM of the present disclosure (e.g., broken underlining in C-terminal regions Kgp_W83_C-term, RgpA_W83_C-term, Kgp_W83, and RgpA_W83 in FIG. 40B). In FIG. 40B, the boxed portions indicate the HbR domain. Proteolytic processing sites are marked with bold font. In some embodiments, the ABM binds to an epitope within a repeated domain of a P. gingivalis gingipain (e.g., RgpA, Kgp) and/or hemagglutinin (e.g., HagA). In some embodiments, the repeated domain containing the epitope occurs at least 2, 3, 4 or more times within the P. gingivalis gingipain (e.g., RgpA, Kgp) and/or hemagglutinin (e.g., HagA). In some embodiments, HagA from W83 and ATCC33277, contains 3 and 4 nearly perfect repeats, respectively, of the sequence containing the putative epitope (FIGS. 40C, 40D, 40E, 40F). In some embodiments, the motif containing the putative epitope occurs twice in a gingipain structure (FIGS. 40D, 40E, 40F). In some embodiments, the third repeat is present in HA4 domain of RgpA but is degenerate in the Kgp (e.g., from W83 strain).

In some embodiments, the ABM binds to an epitope within any one of the amino acid sequences in Table 0.2. In some embodiments, the ABM binds to an epitope within an amino acid sequence at least about 70%, e.g., at least about 75%, at least about 80%, at least about 85%, at least about 90%, at least about 95%, at least about 97%, at least about 99%, or 100% identical to any one of the amino acid sequences in Table 0.2. In some embodiments, the ABM competes with an antibody (e.g., K1B001) for binding to a polypeptide containing any one or more of the amino acid sequences shown in Table 0.2. In some embodiments, the ABM competes with an antibody (e.g., K1B001) for binding to a polypeptide containing an amino acid sequence at least about 70%, e.g., at least about 75%, at least about 80%, at least about 85%, at least about 90%, at least about 95%, at least about 97%, at least about 99%, or 100% identical to any one of the amino acid sequences shown in Table 0.2.

TABLE 0.2
Putative sequence motifs in HagA, RgpA and
Kgp encompassing an epitope recognized by KB001

| Source (see | | SEQ |
|---|---|---|
| Example 12) | Sequence | ID NO: |
| Kgp_N-term | PASYTYTVYRDGTKIKEGLTATTFEEDGV | 77 |
| | AAGNHEYCVEVKYTAGVSPKVC | |
| RgpA_N-term | GSDYTYTVYRDGTKIKEGLTATTFEEDGV | 78 |
| | ATGNHEYCVEVKYTAGVSPKVC | |
| RgpA_C-term | PTDYTYTVYRDGTKIKEGLTETTFEEDGV | 79 |
| | ATGNHEYCVEVKYTAGVSPKKC | |
| HagA_W83_R1 | PTDYTYTVYRDGTKIKEGLTETTFEEDGV | 80 |
| | ATGNHEYCVEVKYTAGVSPKEC | |
| HagA_W83_R2 | PTDYTYTVYRDGTKIKEGLTETTFEEDGV | 80 |
| | ATGNHEYCVEVKYTAGVSPKEC | |
| HagA_ATCC_R1 | PTDYTYTVYRDGTKIKEGLTETTFEEDGV | 80 |
| | ATGNHEYCVEVKYTAGVSPKEC | |
| HagA_ATCC_R2 | PTDYTYTVYRDGTKIKEGLTETTFEEDGV | 80 |
| | ATGNHEYCVEVKYTAGVSPKEC | |
| HagA_ATCC_R3 | PTDYTYTVYRDGTKIKEGLTETTFEEDGV | 80 |
| | ATGNHEYCVEVKYTAGVSPKEC | |
| Kgp_C-term | PTDYTYTVYRDGTKIKEGLTETTFEEDGV | 79 |
| | ATGNHEYCVEVKYTAGVSPKKC | |
| HagA_ATCC_R4 | PTDYTYTVYRDGTKIKEGLTETTFEEDGV | 81 |
| | ATGNHEYCVEVKYTAGVSPKVC | |
| HagA_W83_R3 | PTDYTYTVYRDGTKIKEGLTETTFEEDGV | 80 |
| | ATGNHEYCVEVKYTAGVSPKEC | |
| RgpA_C-term2 | PASYTYTVYRDGTKIKEGLTETTYRDAGM | 82 |
| | SAQSHEYCVEVKYTAGVSPKVC | |
| Kgp_C-term2 | APSYTYTIYRNNTQIASGVTETTYRDPDL | 83 |
| | ATGFYTYGVKVVYPNGESAIET | |

In some embodiments, the ABM specifically binds to one or more P. gingivalis gingipains, where the gingipain is an arg-gingipain (Rgp) or a lys-gingipain (Kgp). In some embodiments, the ABM specifically binds to one or more Rgps selected from RgpA and RgpB. In some embodiments, the ABM specifically binds to RgpA having an amino acid sequence at least about 80%, e.g., at least about 85%, at least about 90%, at least about 95%, at least about 97%, at least about 99%, or 100% identical to SEQ ID NO:21. In some embodiments, the ABM specifically binds to RgpB having an amino acid sequence at least about 80%, e.g., at least about 85%, at least about 90%, at least about 95%, at least about 97%, at least about 99%, or 100% identical to SEQ ID NO:22. In some embodiments, the ABM specifically binds to Kgp having an amino acid sequence at least about 80%, e.g., at least about 85%, at least about 90%, at least about 95%, at least about 97%, at least about 99%, or 100% identical to SEQ ID NO:23. In some embodiments, the ABM specifically binds to a propeptide domain, a catalytic domain and/or a C-terminal adhesion domain of a gingipain. In some embodiments, the ABM specifically binds to a Rgp44 region of an RgpA adhesion domain, as described in, e.g., Li et al., Eur. J. Microbiol. Immunol., 2011, 1:41-58. In some embodiments, the ABM specifically binds to a Kgp39 region of a Kgp adhesion domain, as described in, e.g., Li et al., Eur. J. Microbiol. Immunol., 2011, 1:41-58.

In several embodiments, the ABM specifically binds to a P. gingivalis hemagglutinin/adhesin. In some embodiments, the hemagglutinin is HagA. In some embodiments, HagA has an amino acid sequence at least about 80%, e.g., at least about 85%, at least about 90%, at least about 95%, at least about 97%, at least about 99%, or 100% identical to SEQ ID NO:24. In some embodiments, the ABM specifically binds to an adhesion domain of HagA.

In some embodiments, an ABM of the present disclosure binds to emerging OMVs on P. gingivalis. In some embodiments, an ABM of the present disclosure includes a HVR having an amino acid sequence of SEQ ID NO:30 and a LVR having an amino acid sequence of SEQ ID NO:35. In some embodiments, an ABM of the present disclosure includes a HVR having an amino acid sequence of SEQ ID NO:32 and a LVR having an amino acid sequence of SEQ ID NO:34. In some embodiments, an ABM of the present disclosure includes a HVR having an amino acid sequence of SEQ ID NO:32 and a LVR having an amino acid sequence of SEQ ID NO:35. In some embodiments, an ABM of the present disclosure includes a HVR having an amino acid sequence of SEQ ID NO:30 and a LVR having an amino acid sequence of SEQ ID NO:33. In some embodiments, an ABM of the present disclosure includes a HVR having an amino acid sequence of SEQ ID NO:30 and a LVR having an amino acid sequence of SEQ ID NO:36. In some embodiments, the ABM is at least 80, 85, 90, 95, 96, 97, 98, 99, or 100% identical to one or both of the sequences in Table 13.1

TABLE 13.1

| Antibody | VH variant | VL variant |
|---|---|---|
| H1 | VH1 (SEQ ID NO: 29) | VL1 (SEQ ID NO: 33) |
| H2 | VH1 (SEQ ID NO: 29) | VL2 (SEQ ID NO: 34) |
| H3 | VH1 (SEQ ID NO: 29) | VL3 (SEQ ID NO: 35) |
| H4 | VH1 (SEQ ID NO: 29) | VL4 (SEQ ID NO: 36) |
| H5 | VH2 (SEQ ID NO: 30) | VL1 (SEQ ID NO: 33) |
| H6 | VH2 (SEQ ID NO: 30) | VL2 (SEQ ID NO: 34) |
| H7 | VH2 (SEQ ID NO: 30) | VL3 (SEQ ID NO: 35) |
| H8 | VH2 (SEQ ID NO: 30) | VL4 (SEQ ID NO: 36) |
| H9 | VH3 (SEQ ID NO: 31) | VL1 (SEQ ID NO: 33) |
| H10 | VH3 (SEQ ID NO: 31) | VL2 (SEQ ID NO: 34) |
| H11 | VH3 (SEQ ID NO: 31) | VL3 (SEQ ID NO: 35) |
| H12 | VH3 (SEQ ID NO: 31) | VL4 (SEQ ID NO: 36) |
| H13 | VH4 (SEQ ID NO: 32) | VL1 (SEQ ID NO: 33) |
| H14 | VH4 (SEQ ID NO: 32) | VL2 (SEQ ID NO: 34) |
| H15 | VH4 (SEQ ID NO: 32) | VL3 (SEQ ID NO: 35) |
| H16 | VH4 (SEQ ID NO: 32) | VL4 (SEQ ID NO: 36) |

In some embodiments, any of the ABMs from table 13.1 or the variants noted thereof above, can further include a point mutation at position 222, including the option of an alanine at position 222. In some embodiments, the ABM is H5 having an alanine at position 222, and can be a K222A substitution. Such a substitution will allow the humanized or human chimeric construct to be resistant to degradation.

### ABM Functionality/Properties for Some Embodiments

In some embodiments, the binding affinity (Kd) of the ABM to P. gingivalis is about 1×10⁻⁷ M or less, e.g., about 8×10⁻⁸ M or less, about 6×10⁻⁸ M or less, about 4×10⁻⁸ M or less, about 3×10⁻⁸ M or less, about 1×10⁻⁸ M or less, about 8×10⁻⁹ M or less, about 6×10⁻⁹ M or less, about 4×10⁻⁹ M or less, about 2×10⁻⁹ M or less, about 1×10⁻⁹ M or less, about 8×10⁻¹⁰ M or less, about 6×10⁻¹⁰ M or less, about 4×10⁻¹⁰ M or less, about 2×10⁻¹⁰ M or less, about 1×10⁻¹⁰ M or less, about 5×10⁻¹¹ M or less, about 2×10⁻¹¹ M or less, about 1×10⁻¹¹ M or less, about 5×10⁻¹² M or less, about 2×10⁻¹² M or less, about 1×10⁻¹² M or less, or a binding affinity in between any two of the preceding values. In some embodiments, the binding affinity (Kd) of the ABM to P. gingivalis is from about 1×10⁻⁷ M to about 1×10⁻¹² M, e.g., from about 1×10⁻⁸ M to about 1×10⁻¹² M, from about 1×10⁻⁸ M to about 1×10⁻¹¹ M, from about 1×10⁻⁹ M to about 1×10⁻¹¹ M, including from about 1×10⁻⁹ M to about 1×10⁻¹⁰ M. In certain embodiments, the ABM has a higher binding affinity (e.g., lower Kd) to P. gingivalis than KB001. In some embodiments, the ABM has a binding affinity to P. gingivalis that is about 1.2, 1.5, 2, 2.2, 2.5, 3, 3.2, 3.5, 4.0, 4.2, 4.5, 5, 6, 7, 8, 9, 10, 15, 20, 25, 30, 35, 40, 45, 50, 60, 70, 80, 90, 100 or more times, or any multiple in between those values listed, stronger than the binding affinity of KB001.

In some embodiments, the ABM prevents adhesion of P. gingivalis at a site of infection (e.g., oral site). In some embodiments, the ABM reduces survivability of P. gingivalis at a site of infection (e.g., oral site).

In some embodiments, the ABM binds to one or more virulence factors of P. gingivalis. In some embodiments, the one or more virulence factors are small (20-500 nm) proteo-liposomal membrane vesicles (OMVs) produced via the Type IX cargo secretion system that organizes and distributes macro and micro molecules through its cell membrane and into specific protein-lipo-protein structures. In some embodiments, the ABM binds to outer membrane vesicles (OMVs) of P. gingivalis. In some embodiments, the ABM binds to budding or emerging OMVs of P. gingivalis. In some embodiments, the ABM binds to one or more gingipains and/or hemagglutinins associated with OMVs, e.g., budding or emerging OMVs.

In some embodiments, the ABM binds to a P. gingivalis cell at a high density. In some embodiments, the ABM binds to a P. gingivalis cell surface at a density of at least about 1, 2, 3, 4, 5, 7, 10, 15, 20, 25, 30 35, 40, 45, 50, 55, 60, 65, 70, 75, 80, 85, 90, 95, 100, 110, 120, 130, 140, 150 μm⁻², or more, or at a density between any two of the preceding values. In some embodiments, the ABM shows increased binding to a P. gingivalis having a higher density of surface-associated OMVs and/or bleb-like structures than a P. gingivalis having a lower density. In some embodiments, clinical strains (e.g., clinically relevant strains) of P. gingivalis have a greater ability to secrete OMVs and/or produce a greater number of surface bleb-like structures than a non-clinically relevant strain, and the ABM has a greater affinity to the clinical strains.

In some embodiments, ABMs of the present disclosure find use in detecting P. gingivalis and/or associated exotoxins (e.g., one or more P. gingivalis gingipains) in a sample, e.g., a tissue sample. In some embodiments, an assay for detecting P. gingivalis and/or associated exotoxins in a sample using the ABM provides a sensitive assay. In some embodiments, the ABM provides for an assay for detecting P. gingivalis and/or associated exotoxins in a sample that is more sensitive than an assay based on detection of P. gingivalis nucleic acids, e.g., a PCR-based liquid hybridization assay. In some embodiments, the ABM has sufficient sensitivity to detect P. gingivalis and/or associated exotoxins in a sample where no P. gingivalis nucleic acids is detectably present, e.g., using a PCR-based liquid hybridization assay. In some embodiments, the sample is a brain or gum tissue sample.

In some embodiments, the ABM is resistant to digestion or cleavage, e.g., hydrolytic cleavage, by proteases. In some embodiments, the ABM is resistant to cleavage by a human protease, a bacterial protease and/or a fungal protease. In some embodiments, the ABM is resistant to cleavage by a serine protease, cysteine protease, and/or a metalloprotease. In some embodiments, the ABM is resistant to cleavage by a P. gingivalis protease, e.g., a P. gingivalis extracellular protease. In some embodiments, the ABM is resistant to cleavage by a P. gingivalis gingipain, e.g., RgpA, RgpB, and/or Kgp. In some embodiments, the ABM is resistant to cleavage by a protease as compared to the susceptibility to cleavage by the protease of a fully humanized antibody that specifically binds P. gingivalis, e.g., a fully humanized version of KB001. In some embodiments, the ABM is 10-20%, 20-30%, 30-40%, 40-50%, 50-60%, 60-70%, 70-80%, 80-90% or 90-100% or more resistant to proteolysis by the protease compared to the susceptibility to proteolysis by the protease of a fully humanized antibody that specifically binds P. gingivalis, e.g., a fully humanized version of KB001.

In some embodiments, the ABM is more resistant to cleavage when administered in vivo.

In some embodiments, the ABM inhibits or neutralizes one or more activities of the target protein to which it specifically binds. In some embodiments, the ABM inhibits or neutralizes an activity of the target protein to which it specifically binds by 10-20%, 20-30%, 30-40%, 40-50%, 50-60%, 60-70%, 70-80%, 80-90% or 90-100%. In some embodiments, the ABM inhibits or neutralizes one or more activities of a P. gingivalis. In some embodiments, the ABM inhibits or neutralizes an activity of P. gingivalis by 10-20%, 20-30%, 30-40%, 40-50%, 50-60%, 60-70%, 70-80%, 80-90% or 90-100%.

In some embodiments, the ABM inhibits or neutralizes one or more activities of P. gingivalis associated with one or more gingipains, e.g., RgpA, RgpB, and/or Kgp. In some embodiments, the ABM inhibits or neutralizes an extracellular protease activity of P. gingivalis. In some embodiments, the extracellular protease activity of P. gingivalis includes a protease activity of one or more gingipains, e.g., RgpA, RgpB, and/or Kgp. In some embodiments, the ABM inhibits or neutralizes full proteolysis of a substrate by one or more P. gingivalis gingipains, e.g., RgpA, RgpB, and/or Kgp. In some embodiments, the ABM inhibits, neutralizes, or reduces processing of a hemagglutinin domain-containing protein by one or more P. gingivalis gingipains, e.g., RgpA, RgpB, and/or Kgp. In some embodiments, the hemagglutinin domain-containing protein is P. gingivalis HagA. In some embodiments, the hemagglutinin domain-containing protein has an amino acid sequence at least 80%, at least 85%, at least 90%, at least 95%, at least 97%, or 100% identical to SEQ ID NO: 24. In some embodiments, the hemagglutinin domain-containing protein has an amino acid sequence at least 80%, at least 85%, at least 90%, at least 95%, at least 97%, or 100% identical to SEQ ID NO: 28. In some embodiments, the ABM inhibits the extracellular protease activity of P. gingivalis by 10-20%, 20-30%, 30-40%, 40-50%, 50-60%, 60-70%, 70-80%, 80-90% or 90-100%. In some embodiments, the ABM reduces processing of a hemagglutinin domain-containing protein by one or more P. gingivalis gingipains, e.g., RgpA, RgpB, and/or Kgp, by 10-20%, 20-30%, 30-40%, 40-50%, 50-60%, 60-70%, 70-80%, 80-90% or 90-100%.

In some embodiments, the ABM inhibits the extracellular protease activity of P. gingivalis with an IC₅₀ of about 10 μM or less, e.g., about 5 μM or less, about 2 μM or less, about 1 μM or less, about 0.5 μM or less, about 0.2 μM or less, about 0.1 μM or less, about 0.05 μM or less, about 0.02 μM or less, including about 0.01 μM or less, or an IC₅₀ in between any two of the preceding values. Inhibition of extracellular protease activity may be measured using, e.g., a culture plate assay, as described in, e.g., Grenier et al., Effect of Inactivation of the Arg- and/or Lys-Gingipain Gene on Selected Virulence and Physiological Properties of Porphyromonas gingivalis INFECTION AND IMMUNITY, August 2003, p. 4742-4748, which disclosure is incorporated herein by reference.

In some embodiments, the ABM inhibits the hemagglutination activity of P. gingivalis. In some embodiments, the hemagglutination activity of P. gingivalis includes a hemagglutination activity of one or more gingipains, e.g., RgpA, RgpB, and/or Kgp. In some embodiments, the hemagglutination activity of P. gingivalis includes a hemagglutination activity of an agglutinin, e.g., HagA. In some embodiments, the ABM inhibits the hemagglutination activity of P. gingivalis by 10-20%, 20-30%, 30-40%, 40-50%, 50-60%, 60-70%, 70-80%, 80-90% or 90-100%. Inhibition of hemagglutination activity may be measured using a hemagglutination inhibition assay, as described in, e.g., Booth et al., J. Periodont. 1997. 32:45-60, which disclosure is incorporated herein by reference.

In some embodiments, the ABM inhibits the hemolysis activity of P. gingivalis. In some embodiments, the hemolysis activity of P. gingivalis includes a hemolysis activity of one or more gingipains, e.g., RgpA, RgpB, and/or Kgp. In some embodiments, the ABM inhibits the hemolysis activity of P. gingivalis by 10-20%, 20-30%, 30-40%, 40-50%, 50-60%, 60-70%, 70-80%, 80-90% or 90-100%. Inhibition of hemolysis activity may be measured using a hemolysis assay, as described in Chu et al., Infect. Immun. 1991. 59:1932-1940, which disclosure is incorporated herein by reference.

### Compositions

Also provided herein is a composition that includes an antigen-binding molecule (ABM) that binds Porphyromonas gingivalis, as described herein. In some embodiments, a property of the ABM, e.g., level or glycosylation, is defined in the context of a population of ABM molecules in a composition. In some embodiments, the composition includes an ABM that includes a heavy chain having an amino acid sequence NST is glycosylated. In some embodiments, 0-10%, 10-20%, 20-30%, 30-40%, 40-50%, 50-60%, 60-70%, 70-80%, 80-90% or 90-100% of the ABM in the composition is glycosylated at the asparagine residue of the amino acid sequence NST in the heavy chain. In some embodiments, the composition includes an ABM that is not glycosylated at a position between MNT and YFVY within the heavy chain. In certain embodiments, at the most about 10%, e.g. at the most about 5%, at the most 4%, at the most 3%, at the most 2%, at the most 1%, at the most 0.5%, at the most 0.3%, at the most 0.2% of the ABM in the composition is glycosylated at a position between MNT and YFVY within the heavy chain.

In certain embodiments, the composition is for the topical, oral, and/or subgingival administration of the ABM, for treating a subject in need of treatment for a P. gingivalis infection, or in need of treatment of a condition, disorder or disease (e.g., vascular disease, systemic disease, rheumatoid arthritis, cancer, gut microbiome-related disorder, cognitive disorder, age-related disorder, etc.), as disclosed herein. Thus, in some embodiments, the composition is a pharmaceutical composition that includes an ABM and a pharmaceutically acceptable carrier or excipient. Pharmaceutically acceptable carriers and excipients include saline, aqueous buffer solutions, solvents and/or dispersion media. Some non-limiting examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, methylcellulose, ethyl cellulose, microcrystalline cellulose and cellulose acetate; powdered tragacanth; malt; gelatin; lubricating agents, such as magnesium stearate, sodium lauryl sulfate and talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol (PEG); esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; bulking agents, such as polypeptides and amino acids serum component, such as serum albumin, HDL and LDL; C2-C12 alcohols, such as ethanol; and other non-toxic compatible substances employed in pharmaceutical formulations. The terms such as “excipient,” “carrier,” “pharmaceutically acceptable carrier” or the like are used interchangeably herein. In some embodiments, the carrier inhibits the degradation of the active agent, e.g. an ABM as described herein.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient.

Suitable vehicles that can be used to provide parenteral dosage forms of compounds as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt can also be incorporated into the parenteral dosage forms of the disclosure, including conventional and controlled-release parenteral dosage forms.

### Nucleic Acids, Vectors and Transgenic Cells

Also provided herein are nucleic acids encoding one or more polypeptides of an ABM, as described herein. In some embodiments, the nucleic acid encoding one or more polypeptides of an ABM includes a nucleotide sequence of at least one of SEQ ID NO: 61-70, or a nucleotide sequence having at least about 80%, for example, e.g., at least about 85%, at least about 87%, at least about 90%, at least about 91%, at least about 92%, at least about 93%, at least about 94%, at least about 95%, at least about 96%, at least about 97%, at least about 98%, at least about 99% or greater identity thereto. In some embodiments, the nucleic acid sequence encodes any one or more of the amino acid sequences provided herein.

In some embodiments, a nucleic acid of the present disclosure encoding a variable heavy chain of an ABM as disclosed herein includes a nucleotide sequence at least about 80%, 85%, 90%, 95%, 97%, 98%, 99% or 100% identical to one of SEQ ID NOS:61-64. In some embodiments, a nucleic acid of the present disclosure encoding a variable heavy chain of an ABM as disclosed herein includes a nucleotide sequence at least about 80%, 85%, 90%, 95%, 97%, 98%, 99% or 100% identical to one of SEQ ID NO:69. In some embodiments, a nucleic acid of the present disclosure encoding a variable light chain of an ABM as disclosed herein includes a nucleotide sequence at least about 80%, 85%, 90%, 95%, 97%, 98%, 99% or 100% identical to one of SEQ ID NOS:65-68. In some embodiments, a nucleic acid of the present disclosure encoding a variable light chain of an ABM as disclosed herein includes a nucleotide sequence at least about 80%, 85%, 90%, 95%, 97%, 98%, 99% or 100% identical to one of SEQ ID NO:70.

Nucleic acid molecules encoding amino acid sequence of ABMs are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody. A nucleic acid sequence encoding at least one ABM, e.g., antibody, antigen-binding portion thereof, or polypeptide as described herein can be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Maniatis et al., Molecular Cloning, Lab. Manual (Cold Spring Harbor Lab. Press, NY, 1982 and 1989), and Ausubel, 1987, 1993, and can be used to construct nucleic acid sequences which encode an ABM, e.g., a monoclonal antibody molecule, or antigen binding region thereof. A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as peptides or antibody portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of an ABM, e.g., antibody, or antigen-binding portion thereof as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an ABM, e.g., antibody, or portion thereof as described herein with a specified amino terminus sequence. Moreover, problems associated with retention of initiation codon-derived methionine residues in direct yeast (or bacterial) expression may be avoided. Sabin et al., 7 Bio/Technol. 705 (1989); Miller et al., 7 Bio/Technol. 698 (1989). Any of a series of yeast gene expression systems incorporating promoter and termination elements from the actively expressed genes coding for glycolytic enzymes produced in large quantities when yeast are grown in media rich in glucose can be utilized to obtain recombinant ABMs, e.g., antibodies, or antigen-binding portions thereof. Known glycolytic genes can also provide very efficient transcriptional control signals. For example, the promoter and terminator signals of the phosphoglycerate kinase gene can be utilized.

Production of ABMs, e.g., antibodies, or antigen-binding portions thereof as described herein can be achieved in insects, for example, by infecting the insect host with a baculovirus engineered to express a transmembrane polypeptide by methods known to those of skill in the art. See Ausubel et al., 1987, 1993.

In some embodiments, the introduced nucleotide sequence is incorporated into a plasmid or viral vector capable of autonomous replication in the recipient host. Any of a wide variety of vectors can be employed for this purpose and are known and available to those of ordinary skill in the art. See, e.g., Ausubel et al., 1987, 1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Example prokaryotic vectors known in the art include plasmids such as those capable of replication in E. coli, for example. Other gene expression elements useful for the expression of cDNA encoding ABMs, e.g., antibodies, or antigen-binding portions thereof include, but are not limited to (a) viral transcription promoters and their enhancer elements, such as the SV40 early promoter (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin cDNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin genes comprised of part cDNA, part genomic DNA (Whittle et al., 1 Protein Engin. 499 (1987)), the transcriptional promoter can be human cytomegalovirus, the promoter enhancers can be cytomegalovirus and mouse/human immunoglobulin, and mRNA splicing and polyadenylation regions can be the native chromosomal immunoglobulin sequences.

In some embodiments, for expression of cDNA genes in rodent cells, the transcriptional promoter is a viral LTR sequence, the transcriptional promoter enhancers are either or both the mouse immunoglobulin heavy chain enhancer and the viral LTR enhancer, the splice region contains an intron of greater than 31 bp, and the polyadenylation and transcription termination regions are derived from the native chromosomal sequence corresponding to the immunoglobulin chain being synthesized. In other embodiments, cDNA sequences encoding other proteins are combined with the above-recited expression elements to achieve expression of the proteins in mammalian cells.

Each fused gene is assembled in, or inserted into, an expression vector. Recipient cells capable of expressing the chimeric immunoglobulin chain gene product are then transfected singly with an ABM (e.g., antibody), antigen-binding portion thereof, or chimeric H or chimeric L chain-encoding gene, or are co-transfected with a chimeric H and a chimeric L chain gene. The transfected recipient cells are cultured under conditions that permit expression of the incorporated genes and the expressed immunoglobulin chains or intact ABMs, e.g., antibodies, or fragments are recovered from the culture.

In some embodiments, the fused genes encoding the ABM (e.g., antibody) antigen-binding fragment thereof, or chimeric H and L chains, or portions thereof are assembled in separate expression vectors that are then used to co-transfect a recipient cell. Each vector can contain two selectable genes, a first selectable gene designed for selection in a bacterial system and a second selectable gene designed for selection in a eukaryotic system, wherein each vector has a different pair of genes. This strategy results in vectors which first direct the production, and permit amplification, of the fused genes in a bacterial system. The genes so produced and amplified in a bacterial host are subsequently used to co-transfect a eukaryotic cell, and allow selection of a co-transfected cell carrying the desired transfected genes. Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused genes encoding chimeric H and L chains can be assembled on the same expression vector.

For transfection of the expression vectors and production of the chimeric, humanized, or composite human ABMs, e.g., antibodies, described herein, the recipient cell line can be a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0 (ATCC #CRL 8287). SP2/0 cells produce only immunoglobulin encoded by the transfected genes. Myeloma cells can be grown in culture or in the peritoneal cavity of a mouse, where secreted immunoglobulin can be obtained from ascites fluid. Other suitable recipient cells include lymphoid cells such as B lymphocytes of human or non-human origin, hybridoma cells of human or non-human origin, or interspecies heterohybridoma cells.

An expression vector carrying a chimeric, humanized, or composite human ABM (e.g., antibody) construct, antibody, or antigen-binding portion thereof as described herein can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment. Johnston et al., 240 Science 1538 (1988), as known to one of ordinary skill in the art.

Yeast provides certain advantages over bacteria for the production of immunoglobulin H and L chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes peptides bearing leader sequences (i.e., pre-peptides). Hitzman et al., 1 lth Intl. Conf. Yeast, Genetics & Molec. Biol. (Montpelier, France, 1982).

Yeast gene expression systems can be routinely evaluated for the levels of production, secretion and the stability of ABMs, e.g., antibodies, and assembled chimeric, humanized, or composite human ABMs (e.g., antibodies), portions and regions thereof. Any of a series of yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. A number of approaches can be taken for evaluating optimal expression plasmids for the expression of cloned immunoglobulin cDNAs in yeast. See II DNA Cloning 45, (Glover, ed., IRL Press, 1985) and e.g., U.S. Publication No. US 2006/0270045.

Bacterial strains can also be utilized as hosts for the production of the ABM, e.g., antibody, molecules or peptides described herein. E. coli K12 strains such as E. coli W31 10 (ATCC 27325), Bacillus species, enterobacteria such as Salmonella typhimurium or Serratia marcescens, and various Pseudomonas species can be used. Plasmid vectors containing replicon and control sequences which are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of chimeric, humanized, or composite humanized ABMs, e.g., antibodies, and fragments thereof encoded by the cloned immunoglobulin cDNAs or CDRs in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin protein molecules including leader peptide removal, folding and assembly of H and L chains, glycosylation of the ABM, e.g., antibody, molecules, and secretion of functional ABM (e.g., antibody) protein.

In some embodiments, one or more ABMs (e.g., antibodies) as described herein can be produced in vivo in an animal that has been engineered or transfected with one or more nucleic acid molecules encoding the polypeptides, according to any suitable method.

In some embodiments, an ABM, e.g., antibody, as described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

Many vector systems are available for the expression of cloned H and L chain genes in mammalian cells (see Glover, 1985). Different approaches can be followed to obtain complete H₂L₂ antibodies. As discussed above, it is possible to co-express H and L chains in the same cells to achieve intracellular association and linkage of H and L chains into complete tetrameric H₂L₂ antibodies or antigen-binding portions thereof. The co-expression can occur by using either the same or different plasmids in the same host. Genes for both H and L chains or portions thereof can be placed into the same plasmid, which is then transfected into cells, thereby selecting directly for cells that express both chains. Alternatively, cells can be transfected first with a plasmid encoding one chain, for example the L chain, followed by transfection of the resulting cell line with an H chain plasmid containing a second selectable marker. Cell lines producing antibodies, antigen-binding portions thereof and/or H₂L₂ molecules via either route could be transfected with plasmids encoding additional copies of peptides, H, L, or H plus L chains in conjunction with additional selectable markers to generate cell lines with enhanced properties, such as higher production of assembled H₂L₂ antibody molecules or enhanced stability of the transfected cell lines.

Additionally, plants have emerged as a convenient, safe and economical alternative mainstream expression systems for recombinant ABM, e.g., antibody, production, which are based on large scale culture of microbes or animal cells. ABMs, e.g., antibodies, can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. No. 6,080,560; No. 6,512, 162; WO 0129242.

Mammalian cells are a preferred host for expressing nucleotide segments encoding immunoglobulins or fragments thereof. See Winnacker, From Genes to Clones, (VCH Publishers, NY, 1987), which is incorporated herein by reference in its entirety. A number of suitable host cell lines capable of secreting intact heterologous proteins have been developed in the art, and include CHO cell lines, various COS cell lines, HeLa cells, L cells and multiple myeloma cell lines. Expression vectors for these cells can include expression control sequences, such as an origin of replication, a promoter, an enhancer (Queen et al., “Cell-type Specific Regulation of a Kappa Immunoglobulin Gene by Promoter and Enhancer Elements,” Immunol Rev 89:49 (1986), incorporated herein by reference in its entirety), and necessary processing information sites, such as ribosome binding sites, RNA splice sites, polyadenylation sites, and transcriptional terminator sequences. Preferred expression control sequences are promoters substantially similar to a region of the endogenous genes, cytomegalovirus, SV40, adenovirus, bovine papillomavirus, and the like. See Co et al., “Chimeric and Humanized Antibodies with Specificity for the CD33 Antigen,” J Immunol 148: 1 149 (1992), which is incorporated herein by reference in its entirety.

Alternatively, ABM coding sequences can be incorporated in transgenes for introduction into the genome of a transgenic animal and subsequent expression in the milk of the transgenic animal (e.g., according to methods described in U.S. Pat. Nos. 5,741,957, 5,304,489, 5,849,992, all incorporated by reference herein in their entireties). Suitable transgenes include coding sequences for light and/or heavy chains in operable linkage with a promoter and enhancer from a mammary gland specific gene, such as casein or beta lactoglobulin. The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., supra, which is herein incorporated by reference in its entirety). For production of transgenic animals, transgenes can be microinjected into fertilized oocytes, or can be incorporated into the genome of embryonic stem cells, and the nuclei of such cells transferred into enucleated oocytes. Once expressed, ABMs, e.g., antibodies, can be purified according to standard procedures of the art, including HPLC purification, column chromatography, gel electrophoresis and the like (see generally, Scopes, Protein Purification (Springer-Verlag, NY, 1982), which is incorporated herein by reference in its entirety).

Once expressed, the whole ABMs (e.g., antibodies), their dimers, individual light and heavy chains, or other immunoglobulin forms of the present invention can be recovered and purified by known techniques, e.g., immunoabsorption or immunoaffinity chromatography, chromatographic methods such as HPLC (high performance liquid chromatography), ammonium sulfate precipitation, gel electrophoresis, or any combination of these. See generally, Scopes, PROTEIN PURIF. (Springer-Verlag, NY, 1982). Substantially pure immunoglobulins of at least about 90% to 95% homogeneity are advantageous, as are those with 98% to 99% or more homogeneity, particularly for pharmaceutical uses. Once purified, partially or to homogeneity as desired, a humanized or composite human ABM, e.g., antibody, can then be used therapeutically or in developing and performing assay procedures, immunofluorescent stainings, and the like. See generally, Vols. I & II Immunol. Meth. (Lefkovits & Pernis, eds., Acad. Press, NY, 1979 and 1981).

Additionally, and as described herein, a recombinant humanized ABM, e.g., antibody, can be further optimized to decrease potential immunogenicity, while maintaining functional activity, for therapy in humans. In this regard, functional activity means a polypeptide capable of displaying one or more known functional activities associated with a recombinant ABM, e.g., antibody, as described herein. Such functional activities include, e.g. the ability to bind to a cancer cell marker.

Chimeric, humanized and human ABMs, e.g., antibodies, are typically produced by recombinant expression. Recombinant polynucleotide constructs typically include an expression control sequence operably linked to the coding sequences of ABM, e.g., antibody, chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting ABMs, e.g., antibodies. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences. E. coli is one prokaryotic host particularly useful for cloning the DNA sequences. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

### Methods

Also provided herein are methods of using an antigen-binding molecule (ABM) that binds Porphyromonas gingivalis, as described herein, to treat a subject in need of treatment, e.g., for periodontal disease and/or acute/chronic systemic and organ inflammation. In some embodiments, the condition, disorder or disease is, without limitation, one or more of vascular disease (e.g., cardiovascular disease, atherosclerosis, coronary artery disease, myocardial infarction, stroke, and cardiac hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD age related macro-degeneration, cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); regenerative and stem cell dysfunction; and age-related disorder.

In general terms, the method includes administering a therapeutically effective amount of an ABM that binds P. gingivalis, as described herein, to a subject having an active and/or subclinical infection with or without periodontal disease or inflammation, e.g., gingivitis or periodontitis. In some embodiments, the method includes administering to the subject a therapeutically effective amount of an ABM that binds P. gingivalis, as described herein, to a subject having P. gingivalis localized in the sub-gingival gum line, either with or without gingivitis, and/or periodontal disease or inflammation. In some embodiments, the ABM for use in the present methods binds to P. gingivalis outer membrane forming vesicles and/or secreted outer membrane vesicles containing arg and Lys gingipains/adhesins/hemagglutinins/LPS. In some embodiments, the method includes administering to the subject a therapeutically effective amount of an ABM to a subject having P. gingivalis localized in the sub-gingival gum line and leaking or trans-migrating through epithelia cells and into local lymphatic drainage and the blood vascular system. In some embodiments, the method is a method for passive immunization of a subject against a periodontal infection (such as gingivitis or periodontitis) by administering the ABM, as described herein. In some embodiments, the method is a method for passive, topical oral passive administration of a subject against a periodontal infection (such as gingivitis or periodontitis) by administering the ABM, as described herein. In some embodiments, a method for administering an ABM (e.g., a therapeutically and/or preventative effective amount of an ABM) of the present disclosure includes subgingivally placing the ABM into a subject.

The ABM can be administered to subjects having or suffering from one or more of a variety of conditions, disorders or diseases in the present methods. In some embodiments, the subject has a local and/or systemic infection by P. gingivalis. In some embodiments, the subject has an oral infection of (e.g., colonization by) P. gingivalis. In some embodiments, the subject has an acute or prolonged or chronic P. gingivalis infection. In some embodiments, the subject has a subclinical P. gingivalis infection. In some embodiments, the subject has a condition, disorder or disease associated with a P. gingivalis infection (e.g., oral infection), or symptoms thereof. In some embodiments, the subject has periodontitis, e.g., early or advanced periodontitis. In some embodiments, the condition, disorder or disease is one or more of: vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); regenerative and stem cell dysfunction; and longevity or age-related disorder.

The ABM can be administered using any suitable route to treat the infection, e.g., periodontal infection. In some embodiments, the ABM is administered orally, subgingivally, subcutaneously, intradermally, or intravenously. In some embodiments, the infection is an infection of the gingiva (e.g. gingivitis or periodontitis), blood vessels, the lungs, heart, liver gastro-intestinal tract, brain, etc., and the method includes subgingivally placing a therapeutically effective amount of the ABM into the subject. The ABM may be placed subgingivally in any suitable manner to treat the periodontal infection. In several embodiments, the ABM is placed subgingivally at 1, 2, 3, 4, 5, or 6 or more sites around each tooth to be treated. In some embodiments, the ABM is placed subgingivally at or around each tooth in a subject's mouth. In some embodiments, the ABM is placed subgingivally at or around each of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32 teeth in a subject's mouth. In some embodiments, the ABM is placed subgingivally at or around one or more of the subject's incisor, canine, premolar and/or molar tooth. In some embodiments, the ABM is administered at about 0.001, 0.005, 0.01, 0.02, 0.05, 0.1, 0.2, 0.5, 1, 1.2, 1.5, 2, 2.2, 2.5, 3, 3.2, 3.5, 4, 4.2, 4.5, 5, 5.2, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 70, 80, 90, or 100 μg of the ABM per tooth, or an amount in between any two of the preceding values. In some embodiments, the ABM is administered at about 0.5-10 μg, about 1-8 μg, about 1.5-6 μg, or about 2-5 μg of the ABM per tooth in a treatment. In some embodiments, the ABM is administered at about 3 μg per tooth in a treatment. In some embodiments, the ABM is administered at about 10-400 μg, about 30-300 μg, about 50-200 μg, about 60-160 μg, about 70-140 μg of the ABM per a subject's mouth in a treatment. In some embodiments, the ABM is administered at about 96 μg per subject's mouth in a treatment.

In some embodiments, an ABM of the present disclosure is administered by administering one or more nucleic acids encoding the ABM to a subject in need thereof, as provided herein. Any suitable nucleic acid encoding the ABM can be administered to the subject. In some embodiments, the one or more nucleic acids encoding the ABM is configured to express the ABM when incorporated in a cell of the subject. In some embodiments, the nucleic acid is DNA or RNA. In some embodiments, the one or more nucleic acids is in one or more plasmids or viral vectors (e.g., an adenovirus-associated virus). In some embodiments, the nucleic acid is a mRNA. The nucleic acid encoding the ABM can be delivered to a cell of the subject using any suitable option. In some embodiments, the one or more nucleic acids is delivered to a cell of the subject via viral transduction. In some embodiments, the one or more nucleic acids is delivered to a cell of the subject by electroporation. In some embodiments, the one or more nucleic acids is delivered to a cell of the subject via a lipid nanoparticle. Suitable options for administering an ABM of the present disclosure to a subject is provided in, e.g., Patel et al. “In Vivo Delivery of Nucleic Acid-Encoded Monoclonal Antibodies.” BioDrugs (2020) 34:273-293.

In some embodiments, the method includes removing a microbial infection or preventing its re-colonization in a supra- and/or subgingival space of the subject, before administering the ABM. In certain embodiments, the method includes removing plaque from the supra- and/or subgingival space of the subject, before administering the ABM. In some embodiments, the ABM is placed subgingivally after removing plaque from the supra- and/or subgingival space of one or more teeth to be treated. Plaque can be removed using any suitable means. In some embodiments, the plaque is removed by cleaning and/or root planning. In some embodiments, the method includes administering one or more antibiotics to the subject to remove a microbial infection or colonization in a supra- and/or subgingival space of the subject.

In some embodiments, administration of the ABM prevents or prolongs the time before recolonization. “Recolonization” as used herein refers to detectable growth of P. gingivalis in a supra- and/or subgingival plaque after initial removal of P. gingivalis.

In some embodiments, methods of the present disclosure reduces or eliminates a P. gingivalis infection in the subject, e.g., in the subgingival space of the subject. In some embodiments, the P. gingivalis infection is reduced on average about 10% or more, e.g., 20% or more, 30% or more, 40% or more, 50% or more, 60% or more, 70% or more, 80% or more, 90% or more, 95% or more, including about 100%, compared to the pretreatment level of infection.

In some embodiments, methods of the present disclosure prevent recolonization and or initial colonization of the gingiva by P. gingivalis. Recolonization is inhibited when P. gingivalis growth is inhibited after initial removal of P. gingivalis from the gingival and/or subgingival space, e.g., by removal of plaque. Thus, the method in some embodiments includes removing P. gingivalis from a subgingival space of the subject before administering the ABM to the subject. In some embodiments, removing P. gingivalis from a subgingival space includes cleaning and/or root planing to thereby remove plaque from the subgingival space.

In some embodiments, recolonization is inhibited when P. gingivalis remains undetectable, or detectable at 5% or less, 3% or less, 2% or less, or 1% or less, in a subgingival plaque sample, after initial removal of P. gingivalis from the gingival and/or subgingival space. In some embodiments, recolonization is inhibited for about 1, 2, 3, 4, 5, 6, 7, 8, 9, 10, 11, 12, 13, 14, 15, 16, 17, 18, 19, 20, 21, 22, 23, 24 months or more, or for any period of time in between any two of the times listed above, after initial removal of P. gingivalis. P. gingivalis may be detected by, e.g., immunofluorescent staining of a plaque sample using KB001.

Also disclosed herein is a nucleic acid encoding any of the ABMs of the present disclosure. The nucleic acid may be RNA or DNA. The nucleic acid may also be inserted into a cell, tissue, and/or organism for expression of the ABM. As will be appreciated by one skilled in the art, the nucleic acid may be inserted into a host and used to express the ABM using any conventional method, including mutagenesis of the host DNA, viral vector insertion, CRISPR, resistance cassettes, genetic knock-ins, and electroporation with plasmids. Also disclosed herein is a cell expressing any one or more of the ABMs of the present disclosure. In some embodiments, the cell is mammalian. In some embodiments, the cell is human. In some embodiments, the cell is murine. In some embodiments, the cell is part of cell culture. In some embodiments, the cell is part of a tissue culture. In some embodiments, the cell is incorporated in an organism, such as a human.

In some embodiments, the ABM comprises a heavy chain variable region that is at least 80%, at least 85%, at least 90%, at least 95%, at least 97%, or 100% identical to SEQ ID NO: 1, and a light chain variable region that is at least 80%, at least 85%, at least 90%, at least 95%, at least 97%, or 100% identical to SEQ ID NO: 2. In some embodiments, the ABM comprises a heavy chain variable region that is at least 80%, at least 85%, at least 90%, at least 95%, at least 97%, or 100% identical to SEQ ID NO: 1. In some embodiments, the ABM comprises a light chain variable region that is at least 80%, at least 85%, at least 90%, at least 95%, at least 97%, or 100% identical to SEQ ID NO: 2. In some embodiments, the ABM comprises a heavy chain variable region that is within SEQ ID NO. 1. In some embodiments, the ABM comprises a light chain variable region that is within SEQ ID NO. 2. In some embodiments, the ABM comprises at least one, two, or all three of a LCDR1, a LCDR2, and a LCDR3 within SEQ ID NO: 2 and at least one, two, or all three of a HCDR1, a HCDR2, and a HCDR3 within SEQ ID NO: 1. In some embodiments, the ABM comprises at least one, two, or all three of a LCDR1, a LCDR2, and a LCDR3 within SEQ ID NO: 2. In some embodiments, the ABM comprises at least one, two, or all three of a HCDR1, a HCDR2, and a HCDR3 within SEQ ID NO: 1. In some embodiments, the ABM comprises at least one, two, or all three of a LCDR1, a LCDR2, and a LCDR3 that are at least 80%, at least 85%, at least 90%, at least 95%, at least 97%, or 100% identical to comprises at least one, two, or all three of the LCDR1, theLCDR2, and/or the LCDR3, respectively, of SEQ ID NO: 2. In some embodiments, the ABM comprises at least one, two, or all three of a HCDR1, a HCDR2, and/or a HCDR3 that are at least 80%, at least 85%, at least 90%, at least 95%, at least 97%, or 100% identical to comprises at least one, two, or all three of the HCDR1, the HCDR2, and/or the HCDR3, respectively, of SEQ ID NO: 1.

In some embodiments, the ABM binds to a sequence that is at least 80%, at least 85%, at least 90%, at least 95%, at least 97%, or 100% identical to YTYTVYRDGTKIK (SEQ ID NO: 190).

The ABM can be administered according to any suitable dosing regimen, depending on the embodiment. The dosing regimen may depend on, for example, the severity of periodontal disease (e.g., gingivitis or periodontitis), and/or the strain of P. gingivalis involved in the periodontal disease (e.g., the virulence of the strain, the amino acid sequence of the ABM target expressed by the strain, etc.). In some embodiments, an effective dose of the ABM can be administered once to a subject. In some embodiments, an effective dose of the ABM can be administered repeatedly to a subject, e.g., at least 2, 3, 4, 5, 6, 7, 8, 9, 10, 12, 15, 20, 25, 30, 40 or 50 times or more, or any number of times in between any two of the numbers listed above. In some embodiments, the method includes administering the ABM at an interval of about 1, 2, 3, 4, 5, 6, 7, 8, 9, 10, 11, 12, 13, 14, 15, 16, 17, 18, 19, 20, 25, 30, 40, or about 50 days between any two consecutive doses. In some embodiments, the method includes administering the ABM 1-5 days, 6-10 days, 10-16 days, 16-20 days, 20-25 days, 25-30 days, 30-35 days, 35-40 days, including 40-50 days between any two consecutive doses. In some embodiments, after an initial dosing regimen, the ABM can be administered on a less frequent basis. For example, after weekly or biweekly administration for three months, treatment can be repeated once per month, for six months or a year or longer.

For systemic administration, subjects can be administered a therapeutic amount of the ABM, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

The dosage of an ABM as described herein can be determined by a physician and adjusted, as necessary, to suit observed effects of the treatment. With respect to duration and frequency of treatment, depending on the embodiments, a skilled clinicians can monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the ABM. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more.

The dosage ranges for the administration of the ABMs described herein, according to the methods described herein depend upon, for example, the form of the ABM, its potency, and the desired outcome, e.g., the extent to which symptoms are to be reduced, level of markers, or other indicators of a condition, such as inhibition of recolonization. The dosage should not be so large as to cause adverse side effects. The dosage can vary with the age, condition, and sex of the patient and can be determined by one of skill in the art.

In some embodiments, the method includes administering (e.g., subgingivally) about 2-5 μg, or about 3 μg, per tooth of the ABM in a subject's mouth every 2-4 days for 1-2 weeks (e.g., on days 1, 3, 7, and 10) to prevent recolonization for at least 9 months, e.g., at least 12 months.

Administering the ABM may be done using any suitable option. In some embodiments, the ABM is administered using a syringe, e.g., a Hamilton syringe. In some embodiments, the ABM is administered using a syringe equipped with a suitable gauge needle. In some embodiments, the ABM is administered with a blunt small gauge needle attached to the syringe.

Any suitable delivery system for intraoral, interproximal, intrasulcular, intraperiodontal pocket, intracanal, and intranasal delivery of the ABM can be used to administer the ABM to an oral site. Suitable systems can be, without limitation, mechanical or automated, dental or medical syringes, calibrated or non-calibrated. In some embodiments, a delivery system includes one or more attachments. The delivery system can have any suitable tip, including, but not limited to, blunt ended, and side port. In some embodiments, the delivery system includes a medicament delivery tray and systems, including, without limitation, PerioProtect Trays. In some embodiments, the delivery system includes a medicament applicator delivery system. In some embodiments, the delivery system includes a slow releasing medical preparation, e.g., for intrasulcular drug delivery. In some embodiments, a delivery system includes, without limitation, a filler, oral packing, fiber, microparticles, films, gels, injectable gels, vesicular systems, strips compacts, chip, hydrogel, thermal gel, liquid, solid, including, but not limited to, Actisite, Arestin, Atridox, Ossix Plus, Periochip, Periostat, Periofil. In some embodiments, the delivery system is an injectable system. In some embodiments, the delivery system is an irrigation system including, but not limited to piezoelectric or ultrasonic cavitron units, with or without reservoir, including, without limitation, Ora-Tec Viajet and Oral irrigation systems, including, without limitation, Interplak, Waterpik, Hydrofloss, Viajet, Airfloss and Pro.

In some embodiments, a subject has been diagnosed with a condition or disease, e.g., a P. gingivalis infection, chronic inflammation, multi-system inflammation, Alzheimer's disease, etc., that may be treated with a method of the present disclosure. In some embodiments, the subject is diagnosed with a condition or disease using a kit for detecting the presence of P. gingivalis on the subject, e.g., at a site of infection. In some embodiments, the kit is configured to detect the presence of P. gingivalis in an oral environment of the subject. In some embodiments, the kit is configured to detect the presence of P. gingivalis in a gingival environment of the subject. In some embodiments, the kit includes instructions for using the kit and/or provide the subject with recommendations to seek treatment based on the result of the diagnosis.

### Additional Embodiments

In some embodiments, an ABM of the present disclosure when topically applied via a solution to the infected gums of patients with P. gingivalis binds specifically to the bacterial outer membrane surface, e.g., the molecular complex in the outer- and inner-membranes of the secreted vesicles (exomes) containing complex of toxins (LPS), gingipain proteases, and hemagglutinin. In some embodiments, the ABM binds to a repeating epitope present on multiple localities of the pre- and post-processed hetero-dimer/trimer. In some embodiments, the ABM find use in a prolonged topical oral setting, or intravenous, subcutaneous, intradermal, nebulized or intra-thecal administration. Without being bound to theory, P. gingivalis is thought to relocate into various other tissues/organs/end capillary beds throughout the body and cause local inflammation at these sites. In some embodiments, delivering an ABM of the present disclosure to local or primary site of infection (e.g., oral or subgingival infection) addresses the systemic infection or distant infections at one or more secondary sites. In some embodiments, an ABM that is a nanobody allows for deeper tissue penetration, e.g., to treat various P. gingivalis related cancers.

A variety of conditions, disorders or diseases may be treated through the use of an ABM of the present disclosure. Without being limited by theory, the use of the ABM of the present disclosure to eliminate and/or prevent re-colonization of P. gingivalis in the sub-gingival gum line can in some embodiments interrupt and/or block, or over express the host's inflammatory pathways, such as the inflammasome NLRP3/Interleukin-1l/IL-6 pathways, AIM2, C-reactive protein, the PCSK9 pathway, and the Interleukin-10 innate immunity pathway. In addition, the local and systemic secretion by the bacteria of tissue-damaging outer-membrane vesicles containing a potent mixture of toxins can be curtailed. The ABM of the present disclosure can, in certain embodiments, allow for specifically and locally targeting the P. gingivalis oral infection, which can be the root cause of a chronic active inflammation and toxemia throughout the host's body. In some embodiments, use of the ABM to specifically target and eliminate the disease-causing bacterial source, while sparing other existing oral bacterial strains, provides for treatment of the systemic inflammation without interrupting the complex host inflammation pathways. In some embodiments, used of ABM as disclosed herein avoids or reduces local and/or systemic side effects that may result from intervening in the disrupting/reducing/overexpressing inflammatory pathways such as but not limited to inflammasome NLRP3/Interleukin-10/IL-6 pathways, C-reactive protein, the PCSK9 pathway, and the Interleukin-10 innate immunity pathway for treating a disease.

In some embodiments, P. gingivalis infection occurs in the mouth, gum, teeth, oral cavity, brain, across the blood brain barrier, gut, blood, bone, and/or soft tissues. In some embodiments, P. gingivalis infection occurs in multiple organs. In some embodiments, P. gingivalis infection is local. In some embodiments, P. gingivalis infection is systemic. In some embodiments, P. gingivalis infection is one of several infections in a subject; non-limiting examples of which include Helicobacter pylori, Adenovirus, Acinetobacter spp., Actinomyces spp., Aeromonas hydrophila, Aggregatibacter actinomycetemcomitans, Ascaris lumbricoides, Astrovirus, Bacillus spp., Bacillus cereus, Bifidobacterium spp., Campylobacter spp., Campylobacter jejuni, Camplylobacter rectus, Candida albicans, Chlamydia trachomatis, Chlamydophila pneumoniae, Clostridium spp., Clostridium botulinum, Clostridium difficile, Clostridium perfringens, Clostridium tetanus, Coronaviridaea, Corynebacterium diphtheriae, Cryptococcus neoformans, Cryptosporidium parvum, Cyclospora cayetanensis, Eikenella corrodens, Entamoeba histolytica, Enterobacteriaceae spp., Enterobius vermicularis, Enterovirus, Escherichia coli, Eubacterium nodatum, Fusobacterium spp., Fusobacterium nucleatum, Giardia lamblia, Haemophilus influenzae, Hepatitis, Hymenolepis nana, Influenza, Klebsiella spp., Klebsiella pneumoniae, Lactobacillus casei, Listeria monocytogenes, Moraxella spp., Moraxella catarrhalis, Mycobacterium tuberculosis, Mycoplasma pneumoniae, Necator americanus, Neisseria gonorrhoeae, Neisseria meningitidis, Norovirus, Parvimonas micra, Pasteurella multocida, Peptostreptococcus, Prevotella intermedia, Prevotella nigrescens, Propionibacterium acne, Proteus mirabilis, Pseudomonas aeruginosa, Rotavirus, Salmonella typhi, Salmonella typhimurium, Serratia marcescens, Shigella dysenteriae, Shigella flexneri, Shigella sonnei, Staphylococcus aureus, Staphylococcus epidermidis, Streptococcus spp., Streptococcus agalactiae, Streptococcus enterococci, Streptococcus gordonii, Streptococcus intermedius, Streptococcus mitis, Streptococcus mutans, Streptococcus oralis, Streptococcus pneumoniae, Streptococcus pyogenes, Streptococcus sanquinis, Streptococcus sobrinus, Streptococcus viridans, Strongyloides stercoralis, Taenia saginata, Taenia solium, Tannerella forsythia, Treponema denticola, Vibrio cholerae, and Yersinia enterocolitica. In some embodiments, the at least one additional infection is bacterial, viral, and/or parasite. In some embodiments, the multiple infections form a community biofilm. These biofilms may form a combination of virulence factors, any of which may be targeted as part of subsequent treatment. In some embodiments, virulence factors from P. gingivalis may be targeted as part of treatment or therapy.

In some embodiments, a P. gingivalis infection at an oral site affects end organs, such as, without limitation, large and small vessels of the heart, carotid arteries, vessels in the brain, liver, joints, lungs, pancreas, reproductive system. In some embodiments, the condition, disorder or disease is, without limitation, one or more of vascular disease (e.g., cardiovascular disease, atherosclerosis, coronary artery disease, myocardial infarction, stroke, and cardiac hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD age related macro-degeneration, cerebral and abdominal aneurysms, glioma, large vessel stroke C-IMT, microvascular defects and associated dementias (e.g., Parkinson's), Peri-Implantitis, periodontal disease and/or associated bone loss, cognitive disorders (e.g., early middle late dementia Alzheimer's disease); regenerative and stem cell dysfunction; and age-related disorder. In some embodiments, the method involves any one of the above disorders, where the disorder is caused or complicated by P. gingivalis.

In some embodiments, the condition, disorder, disease, or complication is present in a single cell, organ, tissue, or organ system. In some embodiments, the condition, disorder, disease, or complication is present in multiple cells, organs, tissues, or organ systems.

As disclosed herein, there are many phenotypes that may occur during P. gingivalis infection. Non-limiting examples include an increase in CRISPR-Cas gene expression at the site of infection, an increase in local or systemic inflammation, an increase in the biofilm and/or presence of P. gingivalis, an increase in the activity or activation of inflammasomes, the diversion of oxygen, iron, and other nutrients to P. gingivalis, an increase in cytokine levels, increased host cell death, an increase in systemic inflammation, change of P. gingivalis protein expression, increased proinflammatory mediators, and enhanced chronic distant site inflammatory atherosclerosis. Subsequently, treatment by used of the present ABMs may inhibit, reduce, or eliminate any or multiple of the above phenotypes. In some embodiments, the P. gingivalis infection is in the mouth, gums, brain, gut/gastrointestinal system, blood brain barrier, bone, plasma/blood, soft tissue, or any combination thereof. In some embodiments, targeting the P. gingivalis infection further comprises administration of a small molecule, antibiotic, or drug affective against P. gingivalis. This will be understood to include any effective medicant that acts against P. gingivalis, including small molecules, antibiotics, or drugs that target P. gingivalis virulence factors, increases the production of proteases targeting P. gingivalis, reduces P. gingivalis oxygen, iron, and/or other nutrient uptake, alters protein production in P. gingivalis, alters bacterial metabolism, and/or enhances cell death for P. gingivalis.

Conditions, disorders or diseases treated by administration of an ABM of the present disclosure includes, without limitation, vascular disease (e.g., cardiovascular disease, atherosclerosis, coronary artery disease, myocardial infarction, stroke, and cardiac hypertrophy); systemic disease (e.g., type II diabetes, insulin resistance and metabolic syndrome); rheumatoid arthritis; cancer (e.g., oral squamous carcinomas, gastrointestinal cancer, pancreatic cancer, lung cancer, etc); gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); cognitive disorder (e.g., Alzheimer's disease); neuroinflammatory diseases; and longevity and/or age-related disorders. In general terms, the method includes identifying a subject in need of treating a condition, disorder or disease, as disclosed herein, and administering to the subject a therapeutically effective amount of the ABM of the present disclosure, to thereby treat the condition, disorder or disease.

In some embodiments, the condition, disorder or disease is a vascular disease. A variety of vascular diseases can be treated by use of the present ABMs. In some embodiments, the vascular disease is, without limitation, cardiovascular disease, atherosclerosis, coronary artery disease, myocardial infarction, stroke, or cardiac hypertrophy. Without being bound by theory, P. gingivalis and its virulence factors (e.g., outer membrane vesicles (OMVs), LPS, peptidylarginine deiminase (PPAD), gingipains, hemagglutinins, and fimbriae) are thought to disrupt the inflammatory pathways of heart and systemic vascular disease (CVD/Stroke), including the NLRP3/Interleukin-1β/IL-6 pathways, C-reactive protein (CRP) elevation, the PCSK9 pathway, and the suppression of adaptive immunity via reduction of regulatory T cells (Tregs). P. gingivalis infection can be associated with an increased risk of heart attack, and P. gingivalis is involved with forming oxidized LDL taken up by macrophages, leading to foam cell formation. These atherosclerotic lesions can develop a necrotic core, often forming a thrombus, leading to a downstream event (i.e. heart attack, stroke). Periodontal disease and/or P. gingivalis can be associated with elevated levels of systemic inflammatory markers, such as CRP, IL-6, and Lp-PLA2, Hb-A1c, IL-1b. P. gingivalis can play a major role in Abdominal Aortic Aneurysm development and salivary MPO enzyme activity. Periodontal therapy, as an intervention for improved oral health, can facilitate the management of thrombotic risk, and in the long term can contribute to the prevention of cardiovascular events in patients at risk.

In some cases, the development of atherosclerosis is due to systemic inflammation caused by severe periodontitis. Without being bound by theory, systemic inflammation induced by severe periodontitis, such as those associated with enhanced the secretion of pro-inflammatory cytokines from macrophages and increased the adhesion of monocytes to endothelial cells induce by P. gingivalis LPS, can exacerbate atherosclerosis via, in part, causing aberrant functions of vascular endothelial cells and the activation of macrophages. Further, patients with periodontitis can show higher serum pro-inflammatory cytokines such as tumor necrosis factor (TNF)-α, interleukin (IL)-1β, or IL-6. P. gingivalis can alter genes responsible for mitochondrial function and downregulate gene expression in the signaling pathway, which can lead to mitochondrial dysfunction and metabolic imbalance that promote the development of atherosclerosis. In some embodiments, P. gingivalis can prevent the regression of atherosclerotic plaques by interfering with reverse cholesterol transport. P. gingivalis can also promote atherosclerosis through alteration of gut microbiota, increased IL-1β, IL-18, and TNF-α production in peritoneal macrophages and gingival or aortic gene expression of the NOD-like receptor family, NLRP3, IL-1β, pro-IL-18 and pro-caspase-1, activation of the NLRP3 inflammasome, e.g., through CD36/SR-B2 and TLR2.

Chronic periodontitis (CP) can be associated with increased serum levels of HDL, Ox-LDL, hs-CRP, Hb-A1c, Lp-PLA2, MPO, LDH, troponins T & I, NT pro-BNP, and P selectin. Further, infection of type II P. gingivalis can cause prolonged cytokine response such as IL-1β, IL-8 and TNFα. Elevated cardiac markers found in periodontitis patients indicates that they may carry potential risks in developing cardiac lesions.

In some cases, P. gingivalis contribute to endothelial dysfunction and/or atherosclerotic cardiovascular disease. Without being limited by theory, P. gingivalis may cause vascular damage and increased endothelial permeability by degrading, via gingipain proteases, platelet endothelial cell adhesion molecule-1, and vascular endothelial cadherin, which play a role in endothelial junctional integrity. The vascular damage can increase endothelial permeability and initiate several processes implicated in atherosclerosis, including platelet aggregation, induction of proinflammatory cytokine release, and promotion of leukocyte extravasation to subendothelial regions.

Further, P. gingivalis promotes cardiac rupture after myocardial infarction (MI). Without being bound by theory, P. gingivalis is thought to invade the ischemic myocardium, promote cardiomyocyte apoptosis through activation of p18 Bax by gingipain, increase oxidative stress and MMP-9 protein level and activity, causing cardiac rupture. P. gingivalis-secreted factors can also promote cardiac hypertrophy, through activation of MEK/ERK signal pathways, Toll-like receptor-2 signaling. In some cases, mitogen-activated protein kinase kinase is involved in P. gingivalis-induced myocardial cell hypertrophy and apoptosis. In some cases, components of P. gingivalis spent culture medium increases total MEK-1 and ERK-1 protein products, but also causes increased cellular size, DNA fragmentation, and nuclear condensation in H9c2 cells. These three parameters, and the phosphorylated ERK-1 protein products of H9c2 cells treated with P. gingivalis medium, can be significantly reduced after pre-administration of U0126. The results indicate that P. gingivalis-secreted factors may initiate MEK/ERK signal pathways and lead to myocardial cell hypertrophy and apoptosis.

In some cases, P. gingivalis induces myocardial hypertrophy through Toll-like receptor-2 signaling in the isoproterenol-induced myocardial hypertrophy model. Regulation of chronic inflammation induced by periodontitis may have a key role in the treatment of myocardial hypertrophy. In some embodiments, P. gingivalis enhances myocardial vulnerability, thereby promoting post-infarct cardiac rupture. In some embodiments, Infection with Porphyromonas gingivalis (P.g.) promotes cardiac rupture after MI; P.g. invades the ischemic myocardium; Infection with P.g. promotes the accumulation of p18 Bax; Gingipains from P.g. activate Bax and promote cardiomyocyte apoptosis; Infection with P.g. promotes oxidative stress and MMP-9 protein level and activity.

In some embodiments, infection with periodontal pathogens can cause an adverse outcome after myocardial infarction (MI). C57BL/6J mice were inoculated with Porphyromonas gingivalis (P.g.), a major periodontal pathogen, or injected with phosphate-buffered saline (PBS) into a subcutaneously-implanted steelcoil chamber before and after coronary artery ligation. A significant increase in mortality, due to cardiac rupture, was observed in the P.g.-inoculated MI mice. Ultrastructural examinations revealed that P.g. invaded the ischemic myocardium of the P.g.-inoculated MI mice. The expression of p18 Bax, an active form of pro-apoptotic Bax protein, markedly increased in the P.g.-inoculated MI hearts. In vitro experiments demonstrated that gingipain, a protease uniquely secreted from P.g., cleaved wild type Bax at Arg34, as evidenced by the observation that the cleavage of Bax by gingipain was completely abolished by the Arg34Ala mutation in Bax. Treatment with immunoglobulin Y against gingipain significantly decreased the mortality of the P.g.-inoculated MI mice caused by cardiac rupture. Furthermore, inoculation of P.g. also resulted in an increase of MMP-9 activity in the post-MI myocardium by enhancing oxidative stress, possibly through impairing the selective autophagy-mediated clearance of damaged mitochondria. Without being bound by theory, infection with P.g. during MI can play a detrimental role in the healing process of the infarcted myocardium by invasion of P.g. into the myocardium, thereby promoting apoptosis and the MMP-9 activity of the myocardium, which, in turn, can cause cardiac rupture.

In some cases, P. gingivalis induces cellular hypertrophy and MMP-9 activity via different signaling pathways in H9c2 cardiomyoblast cells. P. gingivalis medium can elevate MMP-9 activity and induce cardiomyoblast hypertrophy. P. gingivalis-induced H9c2 cell hypertrophy was mediated through p38, ERK, PI3K, calcineurin, and JNK signaling pathways, which are in a totally different regulatory pathway from P. gingivalis-elevated MMP-9 activity. P. gingivalis infection activated multiple factors via different pathways to induce the development of hypertrophy of H9c2 cardiomyoblast cells.

In some cases, P. gingivalis deteriorates Isoproterenol-Induced myocardial remodeling in mice. In some situations, stronger cardiomyocyte hypertrophy can be observed in the ISO(+)/P.g.(+) mice compared with the ISO(+)/P.g.(−) mice. The total square of randomly selected cardiomyocytes was 23% larger in the ISO(+)/P.g.(+) mice than in the ISO(+)/P.g.(−) mice. A higher level of mRNA expression in Toll-like receptor 2 and NADPH oxidase 4 in the ISO(+)/P.g.(−) mice was detected compared with the control group. A periodontal pathogen affected ISO-induced cardiac hypertrophy via oxidative stress.

In some situations, P. gingivalis-related cardiac cell apoptosis can be co-activated by p38 and extracellular signal-regulated kinase pathways. In some situations, the development of cardiac cell apoptosis can be directly induced by P. gingivalis medium. Porphyromonas gingivalis-related H9c2 cell apoptosis was mainly co-activated by p38 and ERK pathways and may be involved in death receptor-dependent (caspase 8) and mitochondria (caspase 9)-dependent apoptotic pathways. Porphyromonas gingivalis-related cardiac cell apoptosis was also partially mediated by PI3K or calcineurin signaling pathways, whereas the JNK pathway might play a protective role in P. gingivalis-related cardiac cell apoptosis.

In some situations, the miRNA-212/132 family regulates both cardiac hypertrophy and cardiomyocyte autophagy. In some situations, miR-212/132 family has a key role in cardiac hypertrophy and heart failure development. Both miR-212 and miR-132 can target and negatively regulate the expression of the FoxO3 transcription factor, a powerful anti-hypertrophic and pro-autophagic factor in cardiomyocytes. The microRNA (miRNA)-212/132 family can regulate cardiac hypertrophy and autophagy in cardiomyocytes.

In some situations, Porphyromonas gingivalis-induced miR-132 regulates TNFα expression in THP-1 derived macrophages Live P. gingivalis infection induced miR-132 via TLR signaling and activation of NF-1B. Furthermore, inhibition of miR-132 expression strongly repressed the production of TNFα and increased NFE2L2 and NFAT5. Without being bound by theory, miR-132 modulates TNFα via inhibition of its target genes, which may provide a new window of opportunity to investigate therapeutic intervention for P. gingivalis-induced TNFα associated diseases such as periodontitis. Thus, ABMs of the present disclosure targeting P. gingivalis can be used to address these disorders, conditions or diseases in some embodiments.

In some embodiments, the condition, disorder or disease treated by the present methods is a wound. In some embodiments, administration of an ABM of the present disclosure promotes wound closure and/or prevents or reduces P. gingivalis-induced inhibition of wound closure. In some embodiments, a novel gingipain regulatory gene in Porphyromonas gingivalis mediates host cell detachment and inhibition of wound closure. In some situations, the pgn_0361 gene is involved in regulating gingipains. The PGN_0361-defective strain of P. gingivalis exhibited reduced virulence in terms of epithelial cell detachment and inhibition of wound closure. The culture supernatant of the mutant strain can highly inhibit wound closure, which may be due to high gingipain activity.

In some situations, the capsular polysaccharide and the Arg- and Lys-gingipains of P. gingivalis influences the capacity of P. gingivalis to hinder wound healing, while LPS and the major fimbriae may have no effect. In some situations, entry of Porphyromonas gingivalis Outer Membrane Vesicles into Epithelial Cells Causes Cellular Functional Impairment. Without being bound to theory, loss of intracellular TfR due to MVs causes serious impairment of cellular migration and proliferation. Fundamental cellular operations, including DNA synthesis and ATP generation, require iron, while transferrin-TfR complexes are internalized and ferric iron is released from transferrin at endosomal pH levels. TfR degradation by P. gingivalis can cause impairment of cellular functions, and it is notable that TfR is a target molecule of the bacterium. Thus, ABMs of the present disclosure targeting P. gingivalis can be used to address these disorders, conditions or diseases in some embodiments.

In some embodiments a balanced oral pathogenic bacteria and probiotics can promote wound healing via maintaining mesenchymal stem cell homeostasis. In some cases, P. gingivalis inhibits the functions of mesenchymal stem cells (MSCs) by activating NLRP3 inflammasome. LPS increase in P. gingivalis and thereby inhibits the functions of MSCs by activating NLRP3 inflammasome. Without being bound by theory, homeostasis of oral microbiomes can play a role in maintaining oral heath, provide options for the prevention and treatment of oral diseases, and have referential value for other systemic diseases caused by dysfunction of microbiota and MSCs. It is proposed that P. gingivalis lipopolysaccharide-treated human periodontal ligament stem cells (hPDLSCs) could used to study epigenetics modulations associated with periodontitis, which might be helpful to identify novel biomarkers linked to this oral inflammatory disease. Infection of hDFSCs with P. gingivalis can prolong the survival of neutrophils and increase their migration. These phenotypic changes can depend on direct cellular contacts and PPAD expression by P. gingivalis. Active JNK and ERK pathways in primed human dental follicle stem cells (hDFSCs) can be implicated in the phenotypic changes in neutrophils. In some cases, P. gingivalis can modify hDFSCs, thereby causing an immune imbalance and thus stem cell therapies may be improved and enhanced and protected by eliminating P.g. Thus, ABMs of the present disclosure targeting P. gingivalis can be used to address these disorders, conditions or diseases in some embodiments.

In some embodiments, the condition, disorder or disease is age-related macular degeneration (AMD). In some situations, P. gingivalis invades human retinal pigment epithelial cells, leading to vacuolar/cytosolic localization and autophagy dysfunction. In some situations, Periodontal disease (PD) is linked to age-related macular degeneration (AMD). Porphyromonas gingivalis(Pg), a keystone oral-pathobiont, can be causative of PD, and can efficiently invades human gingival epithelial and blood-dendritic cells. Live, but not heat-killed Pg-strains can adhere to and invade ARPEs. This involves early adhesion to ARPE cell membrane, internalization and localization of Pg within single-membrane vacuoles or cytosol, with some nuclear localization apparent. In infected human cells, Pg is found in vacuoles that contain undegraded ribosomes, where Pg ferments amino acids as an energy source. Co-localized ribosomes may provide a particularly digestible source of amino acids because of their enrichment for the positively charged residues that gingipains cleave. Cytosolically free Pg quickly localizes to the rough ER to form autophagosome-like vacuoles. Our model rather suggests that Pg OMVs entering the brain through the BBB are the more likely source of this diffuse toxic insult to the brain and not a direct infection by Pg. No degradation of Pg or localization inside double-membrane autophagosomes was evident, with dividing Pg suggesting a metabolically active state during invasion. Significant downregulation of autophagy-related genes particularly, autophagosome complex, can be observed. Antibiotic protection-based recovery assay further can confirm distinct processes of adhesion, invasion and amplification of Pg within ARPE cells. P. gingivalis can invade human-RPEs, begin to characterize intracellular localization and survive within these cells. The dysbiotic periodontal pathogen P. gingivalis can efficiently invade retinal epithelial cells in high levels, replicate and are sustained within them. This invasion and autophagy evasion by the keystone species may be one of the contributing elements in the pathogenesis of retinal degenerative diseases.

In some cases, invasion of RPE by Pg and mutants can elevate AMD-related genes involved in angiogenesis; immunosuppression and complement activation which might be the target molecules for both diseases. In some situations, infection of Porphyromonas gingivalis, A Keystone Bacterium in Periodontal Microbiota, is associated with a risk for diabetic retinopathy. In some situations, there is a significant association between a specific microbe in periodontal microbiota and DR, and oral microbiota play a role in retinal eye health.

In some situations, retinal blood flow and neurovascular are coupled in patients with Alzheimer's disease and mild cognitive impairment. In patients with MCI and AD, retinal blood flow and arterial vessel diameters can be reduced compared to healthy age- and sex-matched controls. No difference was found in flicker response between groups. This indicates alterations in retinal blood flow in patients with neurodegenerative disease. Thus, ABMs of the present disclosure targeting P. gingivalis can be used to address these disorders, conditions or diseases in some embodiments.

In some embodiments, the condition, disorder or disease is autism. In some situations, Autism spectrum disorder (ASD) is associated with several oropharyngeal abnormalities, including dysbiosis in the oral microbiota. Since the oral cavity is the start of the gastrointestinal tract, this strengthens and extends the notion of a microbial gut-brain axis in ASD and even raises the question whether a microbial oral-brain axis exists. It is clear that oral bacteria can find their way to the brain through a number of pathways following routine dental procedures. A connection between the oral microbiota and a number of other brain disorders has been reported.

In some situations, Clq as a regulator of brain development is implicated in autism spectrum disorders. Autism spectrum disorders (ASDs) represents a heterogeneous group of neurodevelopmental disorders with similar core features of social and communication impairments, restricted interests and repetitive behaviors. Early synaptic dysfunction due to neuroinflammatory insults may underpin the pathogenesis of abnormal brain development in some of individuals with ASDs. As a component of the innate immune response, the complement system can comprise both directly acting factors and factors that augment other components of the immune system. Beyond its involvement with innate immune responses in the brain, the complement system also plays important roles in neurodevelopment. Recent studies indicate involvement of complement component Clq in fundamental neurodevelopmental pathways and in maintenance and elimination of dendrites and synapses. The impact of aberrant complement system activity during critical windows of brain development may not only affect the local immune response but lead to atypical brain development. Thus, ABMs of the present disclosure targeting P. gingivalis can be used to address these disorders, conditions or diseases in some embodiments.

In some embodiments, the condition, disorder or disease is large vessel stroke, C-IMT (Carotid Intima-media Thickness). In some cases, periodontal treatment can have an effect on carotid intima-media thickness in patients with lifestyle-related diseases. At baseline, LDL-C (low-density lipoprotein cholesterol) levels and percentage (%) of mobile teeth can be positively related to plasma IgG (immunoglobulin) antibody titer against P. gingivalis. Corresponding to improvements in periodontal clinical parameters after treatment, right and left max IMT (maximum intima-media thickness) levels cam be decreased significantly after treatment (SPT-S: start of supportive periodontal therapy, SPT-1y: at 1 year under SPT, and SPT-3y: at 3 years under SPT). P. gingivalis infection can be positively associated with progression of atherosclerosis. Without being bound by theory, routine screening using plasma IgG antibody titer against P. gingivalis and periodontal treatment under collaborative with medical and dental care may prevent cardiovascular accidents caused by atherosclerosis.

P. gingivalis infection can be associated with LDL-C level, which facilitates atherosclerosis, and that periodontal treatment, in collaboration with medical care for atherosclerosis, may contribute to improvements in max carotid IMT. Plasma P. gingivalis IgG titer may be useful for the early detection of atherosclerosis. Finally, periodontal treatment is considered to be important for preventing the onset of cerebral and myocardial infarctions caused by atherosclerosis.

In some situations, overall periodontal bacterial burden can be related to carotid IMT. In some situations, changes in clinical and microbiological periodontal profiles relate to progression of carotid intima—media thickness. In some situations, improvement in periodontal status—defined both clinically and microbiologically—is associated with less progression in carotid atherosclerosis in a randomly selected population-based sample of men and women. Accelerated atherosclerotic progression can be a mechanistic explanation linking periodontal disease and clinical CVD. Thus, ABMs of the present disclosure targeting P. gingivalis can be used to address these disorders, conditions or diseases in some embodiments.

In some embodiments, the condition, disorder or disease is a systemic disease, e.g., a systemic metabolic disorder. A variety of systemic diseases can be treated by use of the present ABMs, as disclosed herein. In some embodiments, the systemic disease is, without limitation, type II diabetes, insulin resistance or metabolic syndrome. Without being bound by theory, P. gingivalis virulence factors can allow the pathogen's invasion to the periodontal tissue and subsequent dissemination into the systemic circulation, increasing the risk of systemic chronic diseases such as type 2 diabetes mellitus (T2DM), cardiovascular diseases, nonalcoholic fatty liver disease (NAFLD), rheumatoid arthritis, and Alzheimer disease. As used herein, “insulin resistance” refers to the reduction or loss of the response of the target organs and tissues to the biological effects of insulin, resulting in decreased efficiency of cell uptake and utilization of glucose and the occurrence of abnormal metabolism of glucose and lipids in cells. In some cases, P. gingivalis outer membrane vesicles (OMVs) can deliver gingipains to the liver, where gingipains can regulate hepatic glycogen synthesis by attenuating insulin sensitivity through the Akt/GSK-30 signaling pathway. Thus, P. gingivalis in the oral cavity can influence hepatic glucose metabolism by decreasing insulin sensitivity in the liver cells. Futher, P. gingivalis can induce insulin resistance through branched-chain amino acids (BCAA) biosynthesis. In addition, P. gingivalis/gingipain can translocate from the oral cavity to pancreatic islets and become localized primarily in 3-cells, and may be epigenetically influencing development of bihormonal cells. Thus, ABMs of the present disclosure targeting P. gingivalis can be used to address these disorders, conditions or diseases in some embodiments.

In some embodiments, the condition, disorder or disease is rheumatoid arthritis (RA). Without being bound by theory, antibodies against P. gingivalis have been found to be associated with RA and with anti-citrullinated protein antibodies (ACPA). Moreover, the DNA of P. gingivalis has been detected in the synovial fluid and plasma samples from patients with RA, and the coexistence of RA and periodontitis increased the probability of finding P. gingivalis DNA in these compartments. Clinical signs and symptoms of RA can improve after periodontal treatments and resolution of periodontitis. Thus, ABMs of the present disclosure targeting P. gingivalis can be used to address these disorders, conditions or diseases in some embodiments.

In some embodiments, the condition, disorder or disease is cancer. In some embodiments, the cancer is, without limitation, oral, gastrointestinal, or pancreatic cancer. In some embodiments, the cancer is, without limitation, esophageal squamous cell carcinoma, head and neck (larynx, throat, lip, mouth and salivary glands) carcinoma. Without being bound to theory, P. gingivalis can promote distant metastasis and chemoresistance to anti-cancer agents and accelerate proliferation of oral tumor cells by affecting gene expression of defensins, by peptidyl-arginine deiminase and noncanonical activation of β-catenin. In some cases, the pathogen can convert ethanol to the carcinogenic intermediate acetaldehyde. In addition, P. gingivalis can be implicated in precancerous gastric and colon lesions, esophageal squamous cell carcinoma, head and neck (larynx, throat, lip, mouth and salivary glands) carcinoma, and pancreatic cancer. P. gingivalis can have systemic tumorigenic effects in addition to the local effects in its native territory, the oral cavity. Thus, ABMs of the present disclosure targeting P. gingivalis can be used to address these disorders, conditions or diseases in some embodiments.

In some embodiments, an ABM of the present disclosure may be administered in conjunction with one or more cancer therapy agents, e.g., chemotherapeutic agent, to enhance the therapeutic effect of the cancer therapy agent. In some embodiments, the cancer therapy agent is a small molecule drug, or an immunotherapeutic agent. In some cases P. gingivalis, its OMVs and/or gingipains have been found to cause an overall immunosuppression of the host, suppressing the adaptive immune system and altering the innate immune system. Adjuvant therapy of eliminating P.g. for improved outcomes for current and future chemotherapies. In some cases, P. gingivalis can inhibitdrug induced apoptosis as well as necrosis (at least the LDH release) in the esophageal squamous cell carcinoma cell line EC0706. When the cancer cells are infected with P. gingivalis prior to the treatment with cisplatin, both apoptosis and necrosis is significantly reduced. Tumor xenografts composed of P. gingivalis-infected OSCC cells can exhibit a higher resistance to Taxol through Notch1 activation, as compared with uninfected cells. Furthermore, P. gingivalis-infected OSCC cells can form more metastatic foci in the lung than uninfected cells. Sustained infection with P. gingivalis, can promote distant metastasis of oral cancer, as well as its resistance to anti-cancer agents. Oral cancer cells sustainedly infected with Porphyromonas gingivalis can exhibit resistance to Taxol and have higher metastatic potential. Thus, in some embodiments, treating and eliminating P.g. with the ABMs improves multiple primary, secondary and adjuvant related cancer treatments.

In some embodiments, the condition, disorder or disease to be treated by the present methods is a lung disease, such as non-smokers lung cancer and aspiration pneumonia. In some embodiments, targeting inflammation with anti-inflammatory therapy can lead to a significantly lower rate of recurrent cardiovascular events independent of lipid-level lowering. There can be a substantial lowering of non-smokers lung cancer with anti-inflammatory therapy targeting the interleukin-1b innate immunity pathway leading to significantly lower cancer mortality consistent with experimental data relating to interleukin-1b.

In some situations, Porphyromonas gingivalis is the primary microbial pathogen as single source driver of inflammation and it's multiple NLRP3/IL-1β pathway mediated diseases including Atherosclerosis and Cardiovascular disease. In some situations, Infection with P. gingivalis can trigger the activation of NLRP3 and AIM2 inflammasomes via TLR2 and TLR4 signaling, leading to IL-1 secretion and pyroptic cell death. In addition, P. gingivalis-induced NLRP3 inflammasome activation can be dependent on ATP release, K+ efflux, and cathepsin B. In some embodiments, any of the ABM can be used to alter TLR4 signaling.

Without being bound by theory, the periodontopathogen Porphyromonas gingivalis has been shown to have several mechanisms of modulating innate immunity by limiting the activation of the NLRP3 inflammasome. The innate immune system can be the first line of defense against microbial pathogens. P. gingivalis can modify innate immunity by affecting inflammasome activity.

Wild type challenge of apolipoprotein E-deficient, spontaneously hyperlipidemic (ApoE) mice with P. gingivalis can increase IL-1β, IL-18, and TNF-α production in peritoneal macrophages and gingival or aortic gene expression of the NOD-like receptor family, NLRP3, IL-1β, pro-IL-1β and pro-caspase-1.

In some situations, outer membrane vesicles derived from Porphyromonas gingivalis can induce cell death with disruption of tight junctions in human lung epithelial cells. P. gingivalis OMVs can cause cell damage with cell membrane destruction in Human lung epithelial cell. P. gingivalis OMVs suppressed cell viability of Human lung epithelial cell by causing apoptosis. P. gingivalis OMVs translocated through oral cavity may be a trigger for inflammation of airway diseases. Thus, ABMs to this target can be used to address this in some embodiments.

In some situations, P. gingivalis OMVs can induce cell death by destroying the barrier system in lung epithelial cells. P. gingivalis OMVs may be a factor in the engagement of periodontitis with respiratory system diseases.

In some situations, Porphyromonas gingivalis is an aggravating factor for chronic obstructive pulmonary disease patients with periodontitis. The microbial analysis of sputum from COPD patients with CP to detect periodontal pathogen Porphyromonas gingivalis (P. gingivalis) both before and after nonsurgical periodontal therapy. A decrease in the count of P. gingivalis and decreased periodontal indices values can be observed in COPD patients with periodontitis after nonsurgical periodontal therapy. Lung function test (forced expiratory volume in the first/forced vital capacity) can be improved in COPD patients with periodontitis after nonsurgical periodontal therapy. In some embodiments, nonsurgical periodontal therapy can be a part of treatment protocol in COPD patients because it helps in reducing the P. gingivalis count and improves the lung function.

In some situations, gingipains are factors in the development of aspiration pneumonia caused by Porphyromonas gingivalis. Aspiration pneumonia can be a life-threatening infectious disease often caused by oral anaerobic and periodontal pathogens such as Porphyromonas gingivalis. This organism can produce proteolytic enzymes, known as gingipains, which can manipulate innate immune responses and promote chronic inflammation. P. gingivalis W83 gingipains can have a role in bronchopneumonia, lung abscess formation, and inflammatory responses. Gingipains can be important for clinical symptoms and infection-related mortality. Pathologies caused by wild-type (WT) P. gingivalis W83, including hemorrhage, necrosis, and neutrophil infiltration, can be absent from lungs infected with gingipain-null isogenic strains or WT bacteria preincubated with gingipain-specific inhibitors. Damage to lung tissue can be correlated with systemic inflammatory responses, as manifested by elevated levels of TNF, IL-6, IL-17, and C-reactive protein. These effects can be dependent on gingipain activity. Gingipain activity can also be implicated in the observed increase in IL-17 in lung tissues. Furthermore, gingipains can increase platelet counts in the blood and activated platelets in the lungs. Arginine-specific gingipains can make a greater contribution to P. gingivalis-related morbidity and mortality than lysine-specific gingipains. Thus, inhibition of gingipain may be a useful adjunct treatment for P. gingivalis-mediated aspiration pneumonia.

One of the pathogenic outcomes of P. gingivalis-triggered aspiration pneumonia can be thrombocytosis. Thrombocytosis can be associated with inflammatory disease, and the platelet count can be an acute-phase response to inflammation induced by P. gingivalis.

Animals challenged with WT P. gingivalis can show a sharp increase in TNF-α, IL-6, and MCP1 levels. The lungs from infected animals can show clear increases in MPO levels, which are indicative of neutrophil infiltration. The highest MPO concentrations can be detected in lung homogenates from animals infected with WT P. gingivalis, whereas those from mice infected with the ΔKgp and ΔRgp strains can show significantly lower MPO activity.

Intratracheal inoculation with either WT P. gingivalis or ΔKgp can lead to a significant increase in IL-17 expression in lung tissue and peripheral blood. Proteolytically active gingipains can modulate the course of P. gingivalis-associated aspiration pneumonia and aggravate the host immune response. P. gingivalis-derived enzymes can play an important role not only during chronic disease (e.g. periodontitis) but also during acute, life-threatening pneumonia. In some situations, TLR2 is implicated in Early Innate Immune Response to Acute Pulmonary Infection with Porphyromonas gingivalis in Mice. The periodontal pathogen Porphyromonas gingivalis is implicated in certain systemic diseases including atherosclerosis and aspiration pneumonia. This organism can induce innate responses predominantly through TLR2, which also mediates its ability to induce experimental periodontitis and accelerate atherosclerosis. TLR2-deficient mice can elicit reduced proinflammatory or antimicrobial responses (KC, MIP-1, TNF-, IL-6, IL-12p70, and NO) in the lung and exhibited impaired clearance of P. gingivalis compared with normal controls. However, the influx of polymorphonuclear leukocytes into the lung and the numbers of resident alveolar macrophages (AM) can be comparable between the two groups. TLR2 signaling can be important for in vitro killing of P. gingivalis by polymorphonuclear leukocytes or AM and, moreover, the AM bactericidal activity can require NO production. Strikingly, AM can be more potent than peritoneal or splenic macrophages in P. gingivalis killing, attributed to diminished AM expression of complement receptor-3 (CR3), which is exploited by P. gingivalis to promote its survival. Without being bound by theory, the selective expression of CR3 by tissue macrophages and the requirement of TLR2 inside-out signaling for CR3 exploitation by P. gingivalis indicates that the role of TLR2 in host protection may be contextual. In some embodiments, TLR2 may mediate destructive effects, as seen in models of experimental periodontitis and atherosclerosis, and the same receptor can confer protection against P. gingivalis in acute lung infection.

In some situations, periodontopathic anaerobes are involved in aspiration pneumonia. Porphyromonas gingivalis and Treponema denticola can coexist in chronic periodontitis lesions. In some situations, a mixed culture of P. gingivalis and T. denticola can be inoculated into the mouse trachea; and cause an infection inducing inflammatory cytokine production and pneumonia. In another series of investigations, professional oral health care (POHC), mainly cleansing administered by dental hygienists once a week for 24 months to elderly persons requiring daily care, can result in the reduction of the number of total anaerobes, Candida albicans, and Staphylococcus species and in the number of cases of fatal aspiration pneumonia. The POHC treatment of elderly persons for 6 months in the winter season can reduce the salivary levels of protease, trypsin-like activity, and neuraminidase and also can decrease the frequency of influenza cases.

In some embodiments, Porphyromonas gingivalis can induce inflammatory responses and promote apoptosis in lung epithelial cells infected with H1N1 via the Bcl-2/Bax/Caspase-3 signaling pathway. P. gingivalis may induce the production of a large number of inflammatory cytokines in lung epithelial cells. Lung epithelial cells infected with H1N1 and P. gingivalis can lead to the promoted production of inflammatory cytokines and the expression of iNOS, which may have also increased the accumulation of NO, resulting in an increased proportion of lung epithelial cells undergoing apoptosis via the Bcl-2/Bax/caspase-3 signaling pathway. Following BEAS-2B cell infection with P. gingivalis and H1N1, the concentrations of TNF-α, IL-1β and IL-6 in the supernatant can be significantly increased at each time point, compared with the H1N1 and P. gingivalis alone groups. These results demonstrated that lung epithelial cells infected with H1N1 and P. gingivalis can promote the production of inflammatory cytokines.

In some situations, Porphyromonas gingivalis modulates Pseudomonas aeruginosa-induced apoptosis of respiratory epithelial cells through the STAT3 signaling pathway. P. gingivalis invasion can transiently inhibit P. aeruginosa-induced apoptosis in respiratory epithelial cells via the signal transducer and activator of transcription 3 (STAT3) signaling pathway. The activated STAT3 can up-regulate the downstream anti-apoptotic moleculars survivin and B-cell leukemia-2 (bcl-2). This process can be accompanied by down-regulation of pro-apoptosis molecular Bcl-2-associated death promoter (bad) and caspase-3 activity inhibition. In addition, the activation of the STAT3 pathway can be affected by P. gingivalis in a dose-dependent manner. Finally, co-invasion of P. aeruginosa and P. gingivalis can lead to greater cell death compared with P. aeruginosa challenge alone. These results indicate that regulation of P. aeruginosa-induced apoptosis by P. gingivalis can contribute to the pathogenesis of respiratory disease. Thus, ABMs of the present disclosure targeting P. gingivalis can be used to address these disorders, conditions or diseases in some embodiments.

In some embodiments, oral cancer cells sustainedly infected with Porphyromonas gingivalis can exhibit resistance to Taxol and can have higher metastatic potential. Sustained infection with P. gingivalis, a major pathogen responsible for chronic periodontitis, can promote distant metastasis of oral cancer, as well as its resistance to anti-cancer agents. Thus, ABMs of the present disclosure targeting P. gingivalis can be used to address these disorders, conditions or diseases in some embodiments.

In some embodiments, the condition, disorder or disease treated by the present methods is Glioma. Without being bound by theory, Cathepsin B plays a critical role in inducing Alzheimer's Disease-like phenotypes following chronic systemic exposure to lipopolysaccharide from Porphyromonas gingivalis in mice. In some cases, systemic exposure to LPS from Porphyromonas gingivalis can induce AD-like phenotypes; Cathepsin B is implicated in inducing microglia-mediated neuroinflammation; Cathepsin B is implicated in inducing microglia-dependent A3 accumulation in neurons. In some situations, a strong association can exist between periodontitis and accelerated cognitive decline in Alzheimer's disease (AD). Cathepsin (Cat) B can play a critical role in the initiation of neuroinflammation and neural dysfunction following chronic systemic exposure to lipopolysaccharide from Porphyromonas gingivalis (PgLPS). Thus, ABMs of the present disclosure targeting P. gingivalis can be used to address these disorders, conditions or diseases in some embodiments.

In some embodiments, the condition, disorder or disease is a gut microbiome-related disorder. A variety of gut microbiome-related disorder can be treated by the ABMs of the present disclosure. In some embodiments, the gut microbiome-related disorder is an intestinal disorder such as, without limitation, inflammatory bowel disease, irritable bowel syndrome (IBS), coeliac disease. In some embodiments, the gut microbiome-related disorder is an extra-intestinal disorder such as, without limitation, allergy, asthma, metabolic syndrome, cardiovascular disease, and obesity. Without being limited by theory, endotoxemia that may cause metabolic disorders can be related to changes in the gut microbiota caused by oral bacteria, e.g., P. gingivalis. In some cases, periodontal inflammation can affect the mechanical and immune barrier functions of the gut. Orally administered P. gingivalis can cause composition shifts in the gut microbiota and increase serum endotoxin and inflammatory markers, and affect the gut immune system. In addition, P. gingivalis has been associated with NAFLD and non-alcoholic steatohepatitis (NASH). P. gingivalis can be detected in the gut of the NAFLD and NASH patients. Thus, ABMs of the present disclosure targeting P. gingivalis can be used to address these disorders, conditions or diseases in some embodiments.

In some embodiments, the condition, disorder or disease is a cognitive disorder. In some embodiments, the condition, disorder or disease is dementia associated with microvasculature defects. In some embodiments, the condition, disorder or disease is microvascular defects Parkinson's.

In some situations, cerebral oxidative stress and microvasculature defects are implicated in TNF-α Expressing Transgenic and Porphyromonas gingivalis-Infected ApoE−/− Mice. There can be a major difference in the hippocampi of P. gingivalis-infected and sham-infected ApoE−/− mice, in terms of increased protein carbonyl/oxidized protein content in the hippocampal micro-vasculature. Hippocampal microvascular structures and the homeostasis of the brain can be at risk from elevated oxidative stress and oxidative protein damage, following P. gingivalis infection. Without being bound by theory, following recurrent episodes of active periodontal disease, there exists a possibility for the development of a defective BBB, post neuroinflammation-mediated cerebral parenchymal tissue injury. The rising levels of intrinsic and extrinsic sources of cytokines, oxidative stress, and developing BBB defects may be implicated as early modifiers of neurodegenerative and disease severity leading to deteriorating memory. Infection with P. gingivalis can be interpreted as one of the plausible mechanisms by which a susceptible host can develop dementia.

A variety of cognitive disorders can be treated by the ABMs of the present disclosure. In some embodiments, the cognitive disorder is Alzheimer's disease (AD). Without being bound by theory, periodontitis has been shown to be a risk factor for AD and a more rapid cognitive decline. In some cases, genetic predisposition, P. gingivalis infection and microglia could promote neurodegeneration typical of that reported for AD. P. gingivalis specific cell free DNA can be detected in the cerebrospinal fluid of AD patients and the pathogen's protease virulence factors, arginine-gingipain (Rgp) and lysine-gingipain (Kgp), can be found in the brains of over 90% of AD patients and can correlate with tau and ubiquitin pathology. Concurrently, there is evidence of Pg OMVs either targeting and/or seeking out tissues higher in arginine and lysine amino acids P. gingivalis can invade and persist in mature neurons, which, once infected, can display signs of AD-like neuropathology, including the accumulation of autophagic vacuoles and multivesicular bodies, cytoskeleton disruption, an increase in phosphotau/tau ratio, and synapse loss. Gingipains of P. gingivalis can digest tau protein into peptide fragments, some of which include tau residues prone to phosphorylation and some of which include two of the four microtubule binding domains that form paired/straight helical filaments constituting neurofibrillary tangles (NFTs). In some cases, Gingipains have been found to be neurotoxic in vivo and in vitro, having detrimental effects on tau. P. gingivalis lipopolysaccharide (LPS) can activate the phosphoinositide 3-kinase/Akt (PI3K/AKT) pathway and increase expression of glycogen synthase kinases-3 beta (GSK-30), which can phosphorylate tau. P. gingivalis can invade and survive in neurons and generate intra-neuronal gingipains that are proteolytically active, leading to neurodegeneration associated with AD. This observation is consistent with studies looking at the neuro-anatomical analysis of Pg associated genes (gingipains) which mark cholinergic neurons, basal forebrain and anterior hypothalamic regions; regions near ventricles and peripheral neurons are also enriched, suggesting relevance to Pg brain entry. In addition to amyloid plaques and neurofibrillary tangles, functional studies suggest that hypothalamic dysfunction is a common event in AD, often early in the course of disease. Although there are evidences indicating that certain hypothalamic regions are also affected in Frontal temporal lobe dementia (FTD), specifically those that correlate with abnormal eating behaviors, they are different to those affected in AD. A possible explanation could be that the hypothalamic region, which controls body innate immunity, is affected in the earliest pro-domal stages of AD, but not in FTD. The apparently AD-specific salivary Lf reduction may thus not only be useful in the differential diagnosis but could also provide important insights into selective immune vulnerability in neurodegenerative diseases. As mentioned above the secretion of salivary proteins is controlled by cholinergic parasympathetic nerves that release acetylcholine, evoking the secretion of saliva by acinar cells in the salivary gland. These parasympathetic nerves are connected with the hypothalamus. We propose that early hypothalamic A3 accumulation is associated with Pg OMVs gingipains deposition found in postmortem brain tissue with the upregulation of ER translocation genes in the context of Alzheimer's disease. This could be an early switch that begins the loss of control and disrupt hypothalamic function affecting salivary gland regulation that ultimately results in reduced salivary Lf secretion. Pg is known to degrade Lf for its major early iron source in oral cavity. Should neural based impairment of the salivary glands produce a decline in the steady-state level of Lf, a major switch in an otherwise delicate balance between Pg and the oral cavity may ensue. More specifically, the diminishing oral salivary iron source would further signal to Pg the need for new iron source. In some embodiments, a subject with Down's syndrome is at increased risk of developing AD.

In some cases, P. gingivalis can induce migration of microglial cells to sites of infection in the brain, through activation of mitogen-activated protein kinase/extracellular signal-regulated kinase (ERK) kinase/ERK pathway. P. gingivalis can induce synthesis of matrix metalloproteinases (MMPs), which can have an important role in neuroinflammatory disorders including AD. Oral infection with P. gingivalis can result in the pathogen spreading to the brain and activating microglia. P. gingivalis can down-regulate TREM-2 expression in microglia. Lack of TREM-2 protein may accelerate aging processes, neuronal cell loss and reduce microglial activity leading to neuroinflammation. P. gingivalis can contribute to development of AD inflammatory pathology through mechanisms involving acute phase proteins, cytokines and the complement cascade where neurons would be attacked. Inappropriate complement activity can play a significant role in AD pathophysiology.

LPS, a virulence factor of P. gingivalis, in the brain can initiate neuroinflammation in the form of microglial cell activation, and the neuroinflammatory response can be stronger with age. Age-associated priming of microglia may have a role in exaggerated inflammation induced by activation of the peripheral immune system. In some cases, P. gingivalis can cause an imbalance in M1/M2 activation in macrophages, resulting in a hyperinflammatory environment that promotes the pathogenesis of periodontitis, and leptomeningeal cells can transduce inflammatory signals from peripheral macrophages to brain resident microglia exposed to P. gingivalis LPS. In microglia, P. gingivalis LPS can increase the production of cathepsin B and pro-forms of caspase-1 and IL-1β through activation of Toll-Like Receptor (TLR) 2/NF-kB signaling. Cathepsin B is implicated in in P. gingivalis LPS-induced AD-like pathology, and may be necessary for the induction of AD-like pathology following chronic systemic exposure to P. gingivalis LPS. In some cases, treating periodontitis can lead to improvements in cognition. A chronic infection of the brain with P. gingivalis can cause serious consequences for the BBB and subsequent mental health. Thus, ABMs of the present disclosure targeting P. gingivalis can be used to address these disorders, conditions or diseases in some embodiments.

In some embodiments, the condition, disorder or disease is an age-related disorder. Without being bound by theory, P. gingivalis can impact cellular biochemical pathways that are associated with improved longevity or shortened life spans, e.g., by regulating autophagy and apoptosis, modulating the mTORC1 pathway, or targeting cellular senescence by selectively eliminating senescent cells. Disrupted autophagy has been linked to numerous diseases including Parkinson's disease, and type 2 diabetes. In some cases, P. gingivalis minor (Mfa1) fimbriae can manipulate dendritic cell (DC) signaling to perturb both autophagy and apoptosis. Mfa1 can induce Akt nuclear localization and activation, and ultimately can induce mTOR in DCs. P. gingivalis can promote DC survival by increasing anti-apoptotic Bcl2 protein expression and decreasing pro-apoptotic proteins Bim, Bax and cleaved caspase-3. In some cases, lipophilic outer membrane vesicles (OMV) shed from P. gingivalis can promote monocyte unresponsiveness to live P. gingivalis. Full reactivity to P. gingivalis can be restored by inhibition of mTOR signaling, which can promote Toll-like receptor 2 and Toll-like receptor 4 (TLR2/4)-mediated tolerance in monocytes. Without being bound by theory, it is thought that P. gingivalis, a facultative intracellular microbe, may damage not only cell membranes but also the mitochondrion, triggering a bioenergetic crisis and NLRP3-induced cellular senescence. Moreover, age-related brain LPS elevation may trigger intracellular iron migration, an innate immune response to withhold iron from pathogens.

Without being bound by theory, the major surface glycoproteins of P. gingivalis—Pgm6 and Pgm7, also called outer membrane protein A-like proteins (OmpALPs)—mediate resistance to the bactericidal activity of human serum, and specifically protect P. gingivalis from the bactericidal activity of LL-37 and from innate immune recognition by TLR4. LL-37 proteolysis by P. gingivalis may provide neighboring dental plaque species with resistance to LL-37, which in turn can benefit P. gingivalis. Thus, ABMs of the present disclosure targeting P. gingivalis can be used to address these disorders, conditions or diseases in some embodiments.

In some embodiments, the condition, disorder or disease is an aneurysm, e.g., cerebral or abdominal aneurysm. In some cases, pro-inflammatory response elicited by Porphyromonas Gingivalis lipopolysaccharide exacerbates the rupture of experimental cerebral aneurysms. Porphyromonas gingivalis LPS can exacerbate vascular inflammation and can enhance the rupture of intracranial aneurysms.

In some situations, CPI can be significantly higher in patients with IAs than the controls (2.7 vs 1.9, p<0.05) and their DNA level of subgingival plaques and their plasma IgG titers of Pg can also be higher. Periodontal disease can be more severe and the plasma IgG titers of Pg can be higher in patients with ruptured—than unruptured IAs, suggesting that Pg is associated not only with the formation but also the rupture of IAs. Severe periodontal disease and Pg infection may be involved in the pathophysiology of IAs.

In some situations, the condition, disorder or disease is depression. Without being bound by theory, it is thought Porphyromonas gingivalis can induce depression via downregulating p75NTR-mediated BDNF maturation in astrocytes. In some embodiments, Pg-LPS decreases the level of astrocytic p75NTR and then downregulates BDNF maturation, leading to depression-like behavior in mice. Pg can be a modifiable risk factor for depression. In some embodiments, Porphyromonas gingivalis (Pg) can induce depression-like behaviors; Astrocytic p75NTR can be decreased in Pg-colonized mice; Overexpression of p75NTR in astrocytes can rescue depressive behaviors; Antibiotic therapy can ameliorate Pg-induced depressive behavior in mice. Thus, ABMs of the present disclosure targeting P. gingivalis can be used to address these disorders, conditions or diseases in some embodiments.

In some embodiments, the condition, disorder or disease is peri-implantitis. In some situations, oral infection with Porphyromonas gingivalis can induce peri-implantitis, and can be implicated in bone loss and the local inflammatory response. Porphyromonas gingivalis infection can induce greater bone loss around implants than around teeth. In non-infected animals, the presence of the implant can correlate with elevated expression of Il-10, Foxp3 and Rankl/Opg ratio, while Tnf-α levels can be decreased relative to tissue around teeth. Six weeks following infection, Tnf-α can be increased significantly while the expression of Foxp3 can be decreased in the tissue around the implants. Oral infection with P. gingivalis of mice with implants can induce bone loss and a shift in gingival cytokine expression. In some situations, thefimA type Ib genotype of P. gingivalis can play a role in the destruction of peri-implant tissue, indicating that it may be a distinct risk factor for peri-implantitis.

In some situations, biocorrosion of pure and SLA titanium surfaces is observed in the presence of Porphyromonas gingivalis and can have effects on osteoblast behavior. P. gingivalis can colonize on the pure and SLA titanium surfaces and weaken their surface properties, especially a decrease in the protective TiO2 film, which can induce the biocorrosion and further negatively affected the osteoblast behavior.

In some situations, titanium can have an influence on in vitro fibroblast-Porphyromonas gingivalis interaction in peri-implantitis. Higher doses of TiO₂ can be toxic to PIGFs and in sub-toxic doses, TiO₂ can cause an increase in gene expression of tumour necrosis factor (TNF)-A and increase protein production of TNF-α, interleukin (IL)-6 and IL-8. A challenge with P. gingivalis alone can induce gene expression of TNF-A, IL-1β, IL-6 and IL-8. A combined challenge with TiO₂ and P. gingivalis can cause a stronger increase in gene expression of TNF-A and protein production of TNF-α and MCP-1 than P. gingivalis alone. TiO₂ particles and P. gingivalis, individually, can induce pro-inflammatory responses in PIGFs. Furthermore, TiO₂ particles and viable P. gingivalis can further enhance gene expression and production of TNF-α by PIGFs. Without being bound by theory, Ti wear particles in the peri-implant tissues in combination with P. gingivalis infection may contribute to the pathogenesis of peri-implantitis by enhancing the inflammation in peri-implant tissues.

In some situations, cytokine and matrix metalloproteinase expression in fibroblasts from peri-implantitis lesions can be observed response to viable Porphyromonas gingivalis. Fibroblasts from peri-implantitis and periodontitis lesions can exhibit a more pronounced inflammatory response to the P. gingivalis challenge than fibroblasts from healthy donors. Without being bound by theory, they may therefore be involved in the development of inflammation in peri-implantitis and periodontitis. Moreover, the sustained upregulation of inflammatory mediators and MMP-1 in peri-implantitis fibroblasts may play a role in the pathogenesis of peri-implantitis.

In some embodiments, the condition, disorder or disease is bone loss or osteoporosis. In some cases periodontal disease and associated bone loss by Porphyromonas gingivalis Stimulates bone resorption by enhancing RANKL (Receptor Activator of NF-1B Ligand) through Activation of Toll-like Receptor 2 in Osteoblasts. LPS P. gingivalis and Pam2 can enhance osteoclast formation in periosteal/endosteal cell cultures by increasing RANKL. LPS P. gingivalis and Pam2 can also up-regulate RANKL and osteoclastic genes in vivo, resulting in an increased number of periosteal osteoclasts and immense bone loss in wild type mice but not in Tlr2-deficient mice. In some cases, LPS P. gingivalis can stimulate periosteal osteoclast formation and bone resorption by stimulating RANKL in osteoblasts via TLR2. Without being bound by theory, this effect might be important for periodontal bone loss and for the enhanced bone loss seen in rheumatoid arthritis patients with concomitant periodontal disease. In some situations, activation of TLR2 in osteoblasts by P. gingivalis increases RANKL production, osteoclast formation, and bone loss both ex vivo and in vivo. P. gingivalis can stimulate alveolar bone loss can cause a more severe loss of juxta-articular bone in RA. In some situations, TLR2, which is highly expressed in RA synovium, is not only activated by pathogen-associated molecular patterns such as P. gingivalis but also by endogenous ligands present in RA synovium such as gp96 and Snapin. There may be a role of endogenous ligands in the pathogenesis of RA bone erosions. Moreover, genetic or antibody-mediated inactivation of TLR2 can reduce cytokine production in P. gingivalis-stimulated neutrophils or macrophages, suggesting that TLR2 plays a non-redundant role in the host response to P. gingivalis. In the absence of MyD88, inflammatory TLR2 signaling in P. gingivalis-stimulated neutrophils or macrophages can depend upon PI3K. TLR2-PI3K signaling may be implicated in P. gingivalis evasion of killing by macrophages, since their ability to phagocytose this pathogen can be reduced in a TLR2 and PI3K-dependent manner. Moreover, within those cells that did phagocytose bacteria, TLR2-PI3K signaling can block phago-lysosomal maturation, thereby revealing a novel mechanism whereby P. gingivalis can enhance its intracellular survival. In some cases, P. gingivalis can uncouple inflammation from bactericidal activity by substituting TLR2-PI3K in place of TLR2-MyD88 signaling. P. gingivalis can be a keystone pathogen, which can manipulate the host inflammatory response in a way that promotes bone loss but not bacterial clearance. Without being bound by theory, modulation of these host response factors may be a therapeutic approach to improve outcomes in disease conditions associated with P. gingivalis.

In some cases, periodontal pathogenic bacteria as well as intestinal dysbiosis are involved in the determinism of bone mineral density BMD loss, and contribute to the onset and worsening of osteoporosis OP. Thus, ABMs of the present disclosure targeting P. gingivalis can be used to address these disorders, conditions or diseases in some embodiments.

In some situations, early host-microbe interaction is implicated in a peri-implant oral mucosa-biofilm model. In some situations, various factors (V. dispar, P. gingivalis, immune cells) could be involved in the disruption or maintenance of homeostasis. Thus, ABMs of the present disclosure targeting P. gingivalis can be used to address these disorders, conditions or diseases in some embodiments.

In some embodiments, a subject has been found to have detectable levels of gingipains associated with P. gingivalis such as Rgp and Kgp in the blood that may be eliminated with a method of the present disclosure in order to maintain wellness. In some embodiments, the wellness can be maintained through the optimization of the gut biome, prevention, initiation or progression of conditions such as vascular inflammation or other disease states to the point of clinical symptoms. In some embodiments, the method includes retreatment of the subject with the ABM. In some embodiments, the method includes obtaining one or more measures of blood borne gingipains associated with P. gingivalis to determine whether the subject requires retreatment with the ABM. Thus, ABMs of the present disclosure targeting P. gingivalis can be used to address these disorders, conditions or diseases in some embodiments.

In some embodiments methods of the resent disclosure include administering to the subject an ABM of the present disclosure in conjunction with one or more treatments of telomer length and/or prevention with various drugs and or natural supplements. Without being bound by theory, it has been shown that shorter telomere lengths are associated with a diagnosis of periodontitis and their measures correlate with the oxidative stress and severity of disease. Thus, ABMs of the present disclosure targeting P. gingivalis can be used to address these disorders, conditions or diseases in some embodiments.

Also provided herein are methods of preventing one or more conditions, disorders, or diseases, as disclosed herein, by administering to a subject, e.g., a subject at risk of developing the condition, disorder, or disease, an effective amount of an ABM of the present disclosure, to thereby prevent the condition, disorder, or disease or developing. In some embodiments, the subject is predisposed to developing the condition, disorder, or disease. In some embodiments, the subject has a past history of an P. gingivalis infection and/or condition or disease associated with a P. gingivalis infection, as disclosed herein. In some embodiments, the subject is genetically predisposed to develop the condition, disorder, or disease. In some embodiments, the method includes identifying a subject predisposed to developing any one or more of the conditions, disorders, or diseases, as disclosed herein, and administering to the subject an effective amount of an ABM of the present disclosure to thereby prevent, reduce the likelihood and/or delay the onset of the conditions, disorders, or diseases.

In any of the above methods, the ABM can be administered in conjunction with one or more additional therapeutic agents for treating or preventing the condition, disease or disorder. In some embodiments, a therapeutic agent for treating or preventing the condition, disease or disorder, as disclosed herein, can be administered to a subject in need thereof in at a therapeutically effective amount, and an effective amount of the ABM of the present disclosure can be administered to the subject. Administration of the ABM can in some embodiments improve or enhance the therapeutic effect of the other therapeutic agent. As used herein, a first agent administered in conjunction with administering a second agent can include administering the first agent before, after, or simultaneously as the second agent. In some embodiments, the first agent and second agent are administered within an interval such that the therapeutic effect of the first agent is present in the subject when the second agent is administered to the subject.

By way of non-limiting examples, the ABM can in some embodiments be administered in conjunction with one or more additional therapeutic agents for treating or preventing a vascular disease, as disclosed herein. In some embodiments, the other therapeutic agent includes a serum lipid lowering agent. Any suitable serum lipid lowering agent can be used. In some embodiments, the serum lipid lowering agent includes, without limitation, statins (e.g., atorvastatin, cerivastatin, fluvastatin, lovastatin, mevastatin, pitavastatin, pravastatin, rosuvastatin, simvastatin), Nicotinic acid (Niacin) (e.g., NIACOR, NIASPAN (slow release niacin), SLO-NIACIN (slow release niacin), CORDAPTIVE (laropiprant)), Fibric acid (e.g., LOPID (Gemfibrozil), TRICOR (fenofibrate), Bile acid sequestrants (e.g., QUESTRAN (cholestyramine), colesevelam (WELCHOL), colestipol (COLESTID)), Cholesterol absorption inhibitors (e.g., ZETIA (ezetimibe)), PPAR gamma agonsits, PPAR alpha/gamma agonists, squalene synthase inhibitors, CETP inhibitors, anti-hypertensives, anti-diabetic agents (such as sulphonyl ureas, insulin, GLP-1 analogs, DDPIV inhibitors, e.g., metaformin), ApoB modulators, such as mipomersan, MTP inhibitoris and/or arteriosclerosis obliterans treatments.

The ABM can in some embodiments be administered in conjunction with one or more additional therapeutic agents for treating or preventing cancer, as disclosed herein. In some embodiments, the other therapeutic agent includes an anti-cancer therapeutic that is a small molecule drug or immunotherapeutic agent. Any suitable small molecule drug or immunotherapeutic agent can be used.

In some embodiments, a dosing strategy for therapeutics can optimize the therapeutic outcome by minimizing adverse effects and maximizing efficacy across the target patient population. Multiple factors including pharmacokinetics, pharmacodynamics, exposure-response (efficacy/safety) relationships, disease burden, patient characteristics, compliance and pharmaco-economics can affect the decision on the clinical dose and dose regimen. In some embodiments, a consideration here is whether patients should be dosed based on body size, or whether body size-independent (fixed) dosing offers a viable alternative. The dosing strategy can vary. In some embodiments, body size based dosing (i.e. a dose proportional to the body size) can be used for mAbs. In some embodiments, this dosing approach can reduce inter-subject variability in drug exposure, and controlling for this pharmacokinetic variability in turn can significantly reduce variability in the response to drug treatment across the population. In some embodiments, mAbs are dosed based on body size. In some embodiments, body size-based dosing is used when there is a statistically significant body size effect on pharmacokinetic parameter(s) in the population pharmacokinetic analysis.

For systemic administration, subjects can be administered a therapeutic amount of the ABM, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more, or an amount in a range defined by any two of the preceding values.

### Kits

Also provided herein are kits that include an antigen-binding molecule (ABM) of the present disclosure. In several embodiments, the kit includes a pharmaceutically acceptable excipient or a buffer. In some embodiments, the kits of the present disclosure may be suitable for performing the methods of administering the ABM to a subject, as described herein. In some embodiments, components of the kit is packaged individually in vials or bottles or in combination in containers or multi-container units. In some embodiments, kits include instructions, in words, diagrams, or combinations thereof, for administering the ABMs, as described herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein, table numbering is assigned so as to provide a shorthand reference to the example, if any, that the table is discussed. Tables that are only discussed in the detailed description can be denoted by a sub 1 value (e.g., 0.1). This is not meant to limit the relevance or discussion or implications of the tables, but to serve as a quick reference guide.

The technology described herein is further illustrated by the following examples which in no way should be construed as being further limiting.

In some embodiments, an ABM of the present disclosure includes a heavy chain variable region having an amino acid sequence at least 80%, 90%, 95%, 97%, 98%, 99%, or 100% identity to SEQ ID NO:32, and a light chain variable region having an amino acid sequence at least 80%, 90%, 95%, 97%, 98%, 99%, or 100% identity to SEQ ID NO:34. In some embodiments, an ABM of the present disclosure includes a heavy chain variable region having an amino acid sequence at least 80%, 90%, 95%, 97%, 98%, 99%, or 100% identity to SEQ ID NO:30, and a light chain variable region having an amino acid sequence at least 80%, 90%, 95%, 97%, 98%, 99%, or 100% identity to SEQ ID NO:33. In some embodiments, an ABM of the present disclosure includes a heavy chain variable region having an amino acid sequence at least 80%, 90%, 95%, 97%, 98%, 99%, or 100% identity to SEQ ID NO:30, and a light chain variable region having an amino acid sequence at least 80%, 90%, 95%, 97%, 98%, 99%, or 100% identity to SEQ ID NO:35. In some embodiments, an ABM of the present disclosure includes a heavy chain variable region having an amino acid sequence at least 80%, 90%, 95%, 97%, 98%, 99%, or 100% identity to SEQ ID NO:30, and a light chain variable region having an amino acid sequence at least 80%, 90%, 95%, 97%, 98%, 99%, or 100% identity to SEQ ID NO:36. In some embodiments, an ABM of the present disclosure includes a heavy chain variable region having an amino acid sequence at least 80%, 90%, 95%, 97%, 98%, 99%, or 100% identity to SEQ ID NO:32, and a light chain variable region having an amino acid sequence at least 80%, 90%, 95%, 97%, 98%, 99%, or 100% identity to SEQ ID NO:35. In some embodiments, an ABM of the present disclosure competes with KB001 for binding to a P. gingivalis gingipain, e.g., RgpA. In some embodiments, any one of these sequences can further include a point mutation at position 222, such as to an alanine.

In some embodiments, an ABM of the present disclosure detects P. gingivalis gingipain/hemagglutinin in a biological sample which does not include detectable P. gingivalis genomic DNA. In some embodiments, an ABM of the present disclosure detects P. gingivalis gingipain/hemagglutinin in a brain tissue sample which does not include detectable P. gingivalis genomic DNA.

### EXAMPLES

### Example 1: Amino Acid Sequence of the Heavy and Light Chains of KB001 Antibody

Generation of purified mouse IgG1 monoclonal antibody: Hybridoma mAb03 was obtained and propagated in HyClone ADCF-MAb media supplemented with penicillin and streptomycin. The doubling time of the cells was approximately 36 hours.

Purification of monoclonal antibody: IgG from approximately 100 mL of conditioned media was purified using a standard Protein A column to confirm that the cell line produced antibody. Approximately 100 micrograms of antibody was purified. IgG from approximately 750 mL of conditioned media was processed to generate approximately 4 milligrams of IgG. It was estimated the hybridoma produced approximately 8 mg of antibody per Liter.

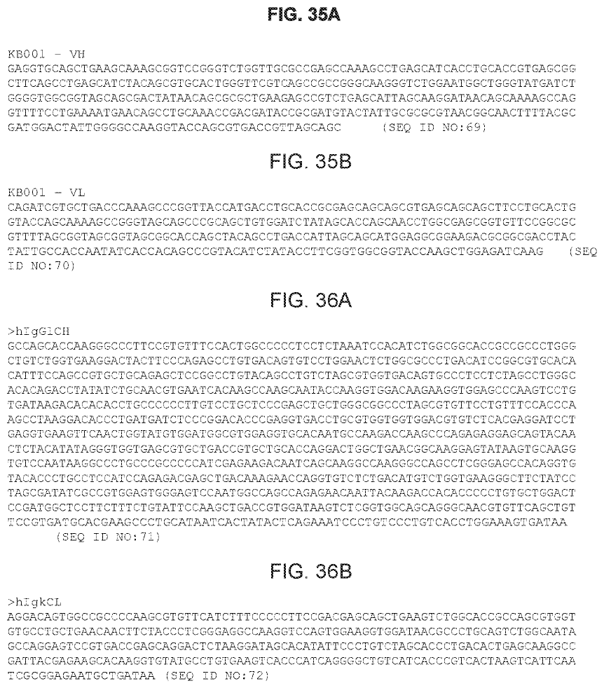

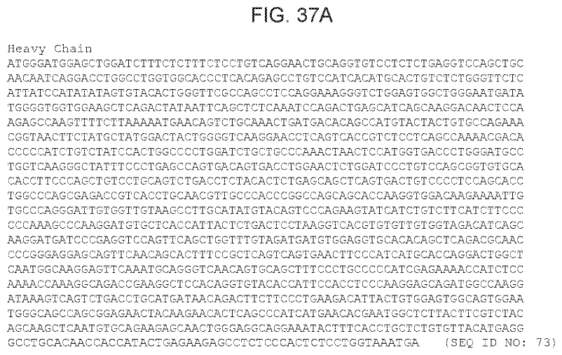

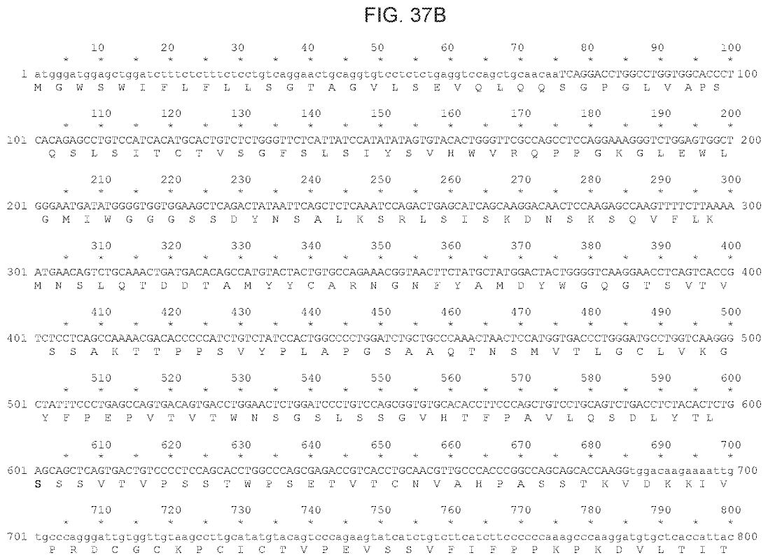

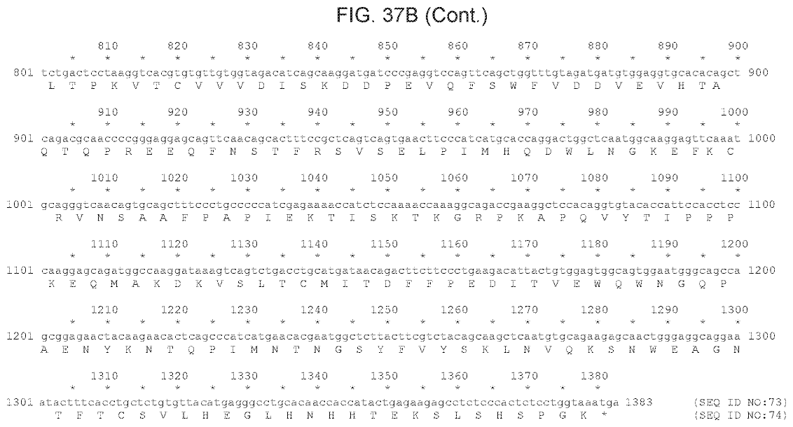

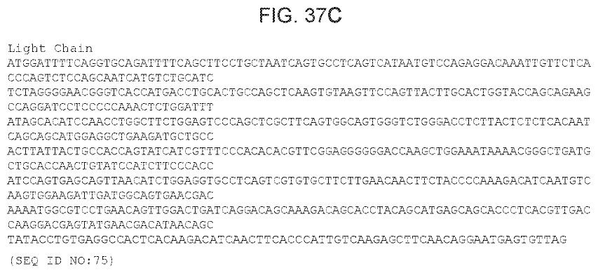

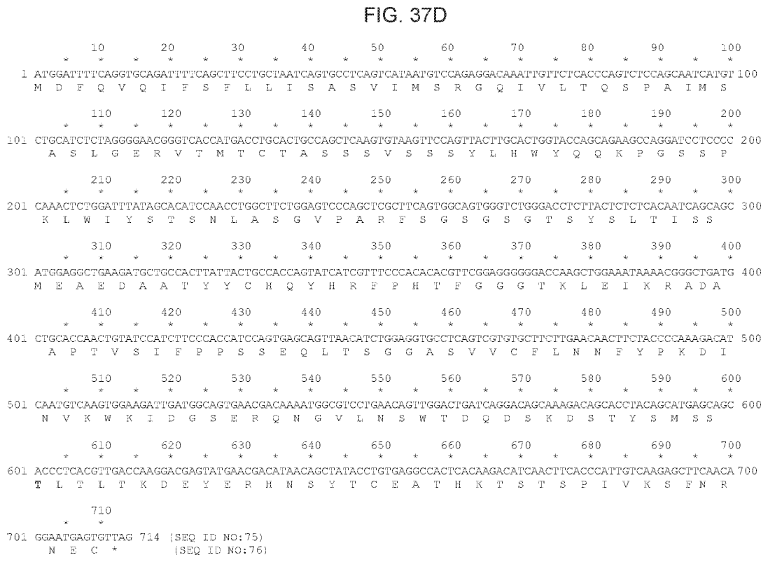

Sequencing the antibody: RNA from cultured cells was prepared using the RNAzol method. cDNA was synthesized using both random hexamer and oligo(dT) primers. Degenerative primers were designed to amplify conserved, constant regions of the Heavy and Light chains. Due to uncertainties of the sequence, approximately 24 primers were used. PCR fragments were synthesized and sent for sequence analysis. Initial efforts yielded the sequences of the hypervariable regions. Additional efforts were required to derive the sequences of the remaining regions. Preliminary plans called for grafting the hypervariable regions onto constant domains in silico. However, the IgG eluted from Protein A resin at a higher pH than normal (4.7 vs. 3.7) and suggested the constant regions may have some variation from conserved sequences. The presence of variant sequences was confirmed by the unusually rigorous efforts that were required to amplify and sequence the cDNA fragments. The nucleotide sequence data were used to create contiguous sequences and then translated to putative amino acid sequences for analysis. The nucleotide sequences encoding the heavy and light chains, including the signal peptide, are depicted in FIGS. 37A and 37C, respectively. The nucleotide sequences encoding the heavy and light chain variable regions are depicted in FIGS. 35A and 35B, respectively.

The amino acid sequences of the heavy and light chains, of KB001 is shown in FIGS. 1A and 1B, respectively.

The translated amino acid sequences were analyzed by BLAST to align with the nearest neighbor for the purpose of identifying antibody domains. The heavy chain aligned most closely with IgG1 heavy chains. The light chain aligned most closely with Lambda light chains.

### Example 2: Epitope Mapping of KB-001 Antibody

This non-limiting example shows a procedure for tryptic digest and mass spectrometry (MS) analysis of gingipains for epitope mapping of KB-001. Such epitopes can be used to define various APs.

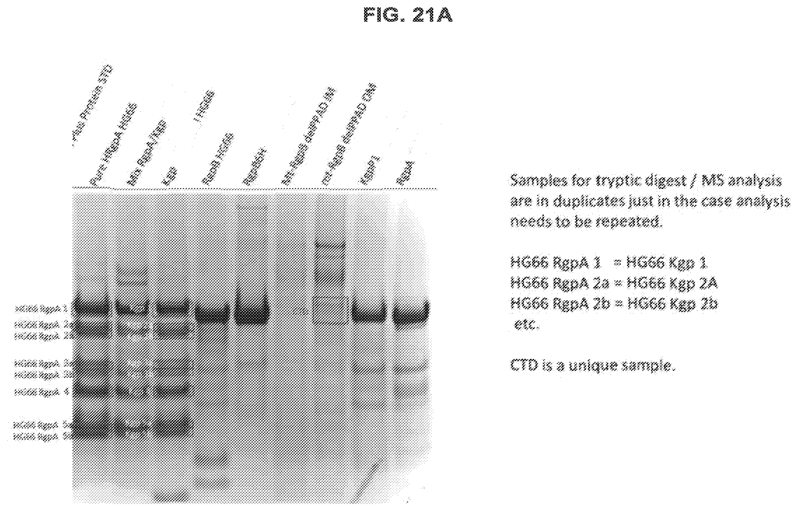

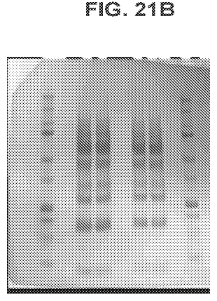

To determine viable APs, one can first identify the epitope on P. gingivalis target proteins of KB-001, gingipains (RgpA, Kgp) and hemagglutinin from various P. gingivalis strains were digested with trypsin and the tryptic digests were probed for KB-001 binding (FIGS. 21A and 21B). Peptides fragments binding to KB-001 were analyzed by MS and N-terminal sequencing.

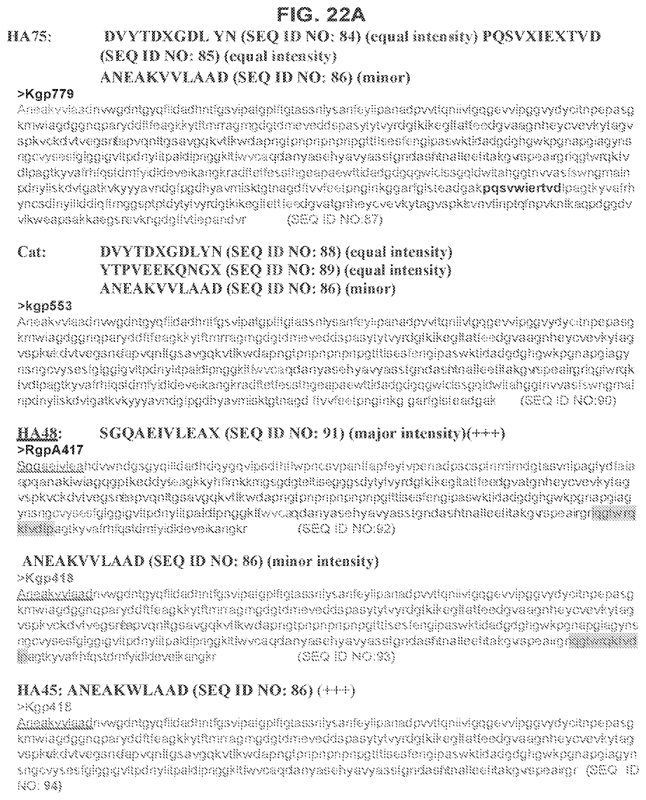

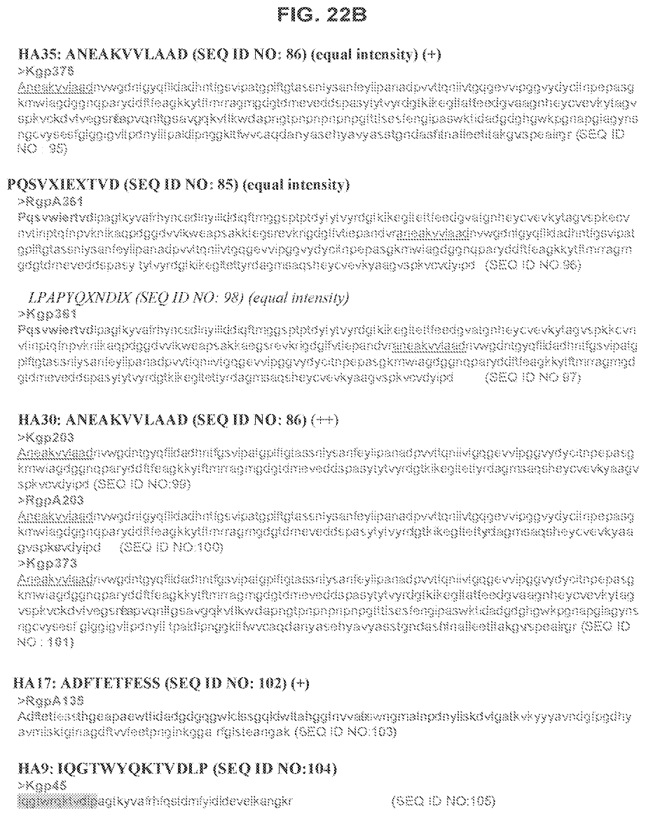

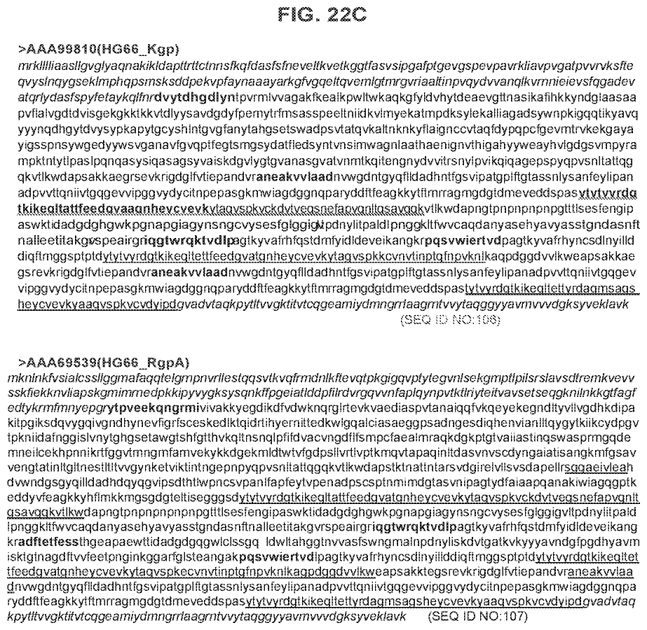

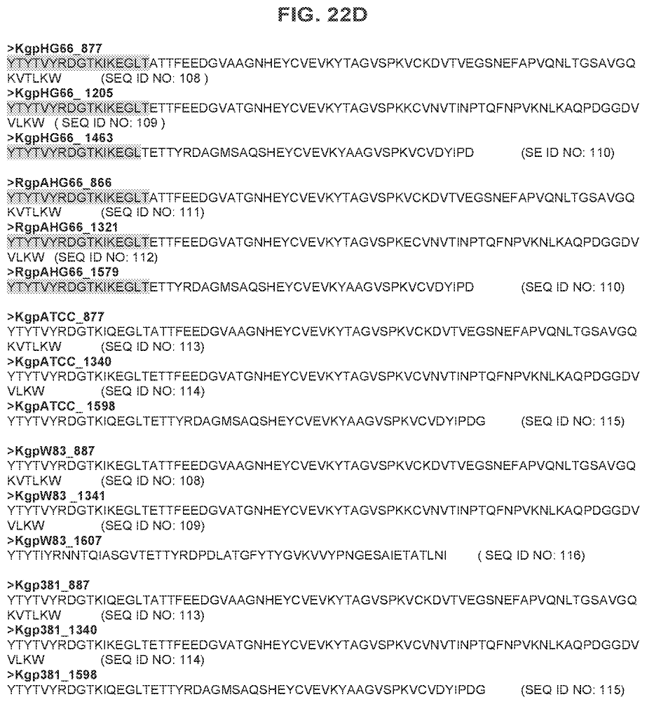

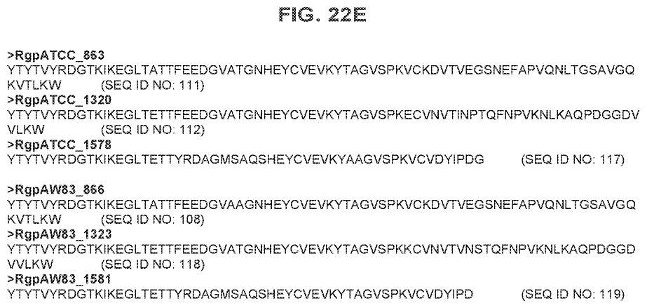

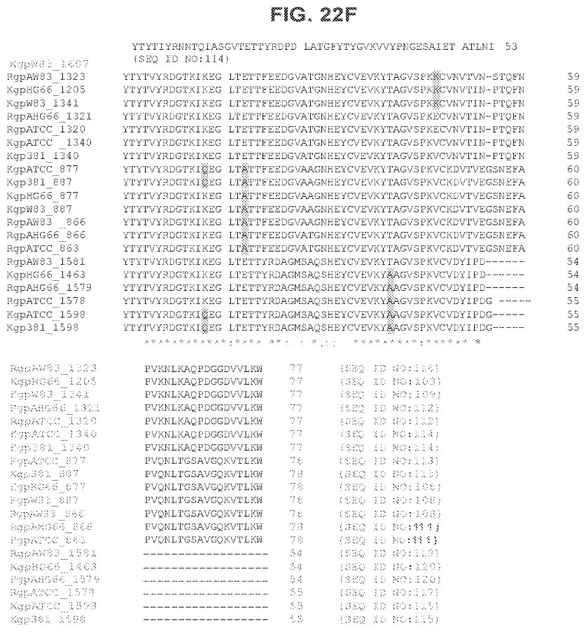

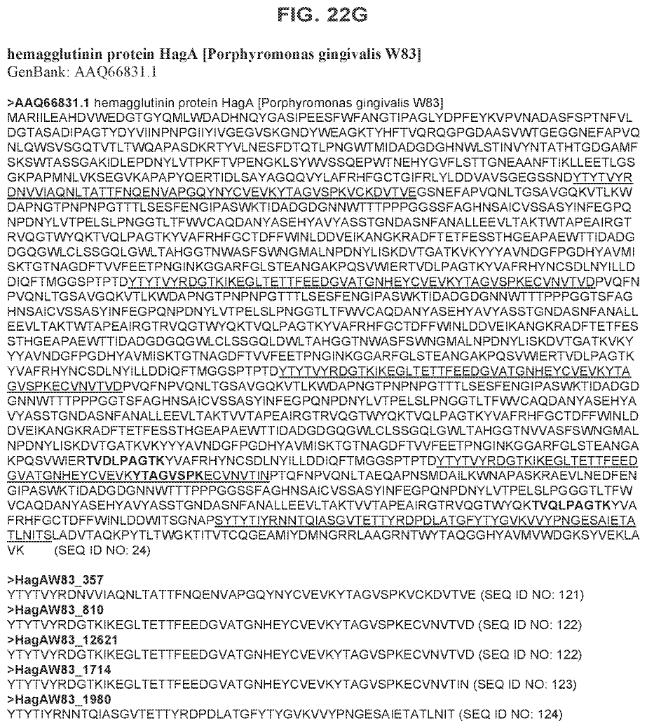

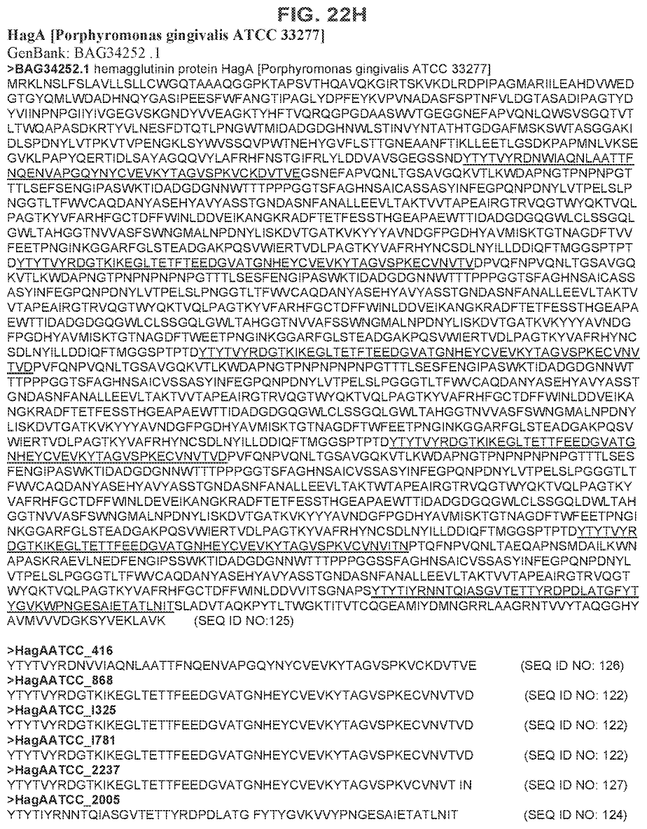

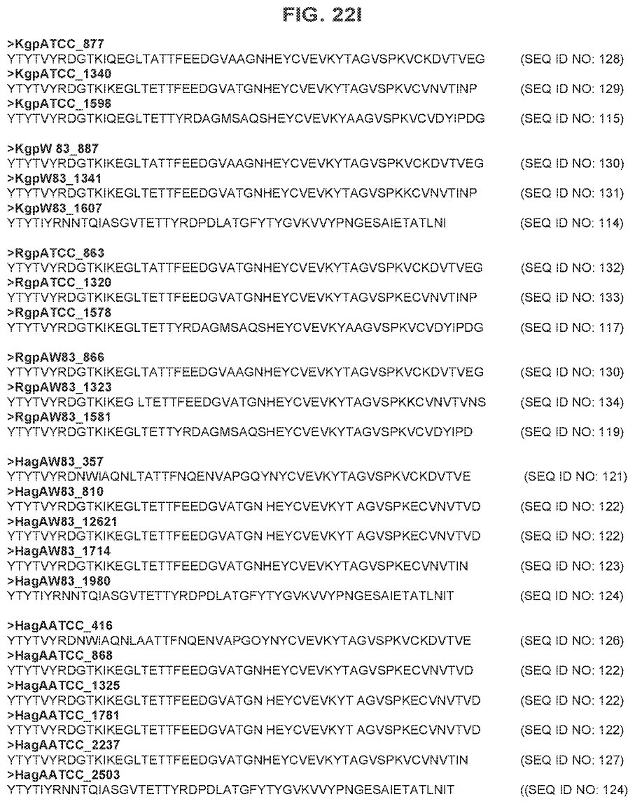

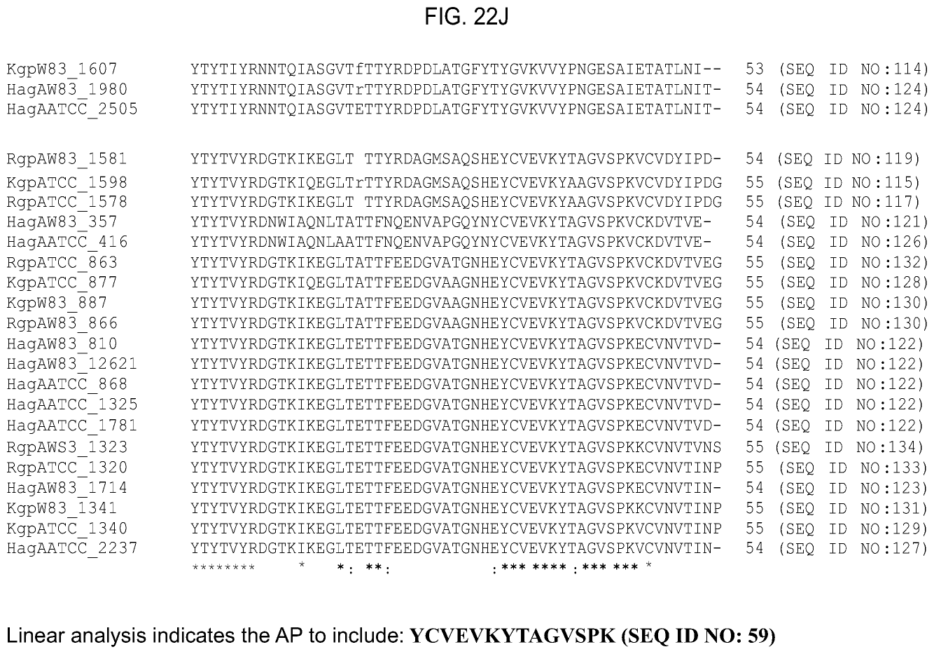

The deduced sequences of linear portion KB-001-binding fragments and the position of these sequences in the full protein are listed in FIGS. 22A-22J. Linear analysis indicated that the binding epitope to include: YCVEVKYTAGVSPK. Thus, a viable AP would include, in some embodiments, this sequence.

Sequences within gingipains (RgpA, Kgp) and hemagglutinin (HagA) from various P. gingivalis strains that encompass the putative linear portion of the epitope sequence recognized by KB-001 are indicated in FIGS. 40A-40F. HagA from W83 and ATCC33277 contain 3 and 4 nearly perfect repeats, respectively, of the sequence containing the putative epitope (FIGS. 40C, 40D, 40E, 40F). As a nearly perfect repeat the motif occurs twice in gingipain structure (FIGS. 40D, 40E, 40F). The third repeat is present in HA4 domain of RgpA but is degenerate in the Kgp (from W83 strain). The presence of the epitope within the sequences shown in FIG. 40F was verified by WB analysis of mAbs reactivity with different domains of RgpA and Kgp.

Based on the above example, in some embodiments, an AP of the present disclosure includes any one or more of the following sequences:

PASYTYTVYRDGTKIKEGLTATTFEEDGVAAGNHEYCVEVKYTAG
VSPKVC;
GSDYTYTVYRDGTKIKEGLTATTFEEDGVATGNHEYCVEVKYTA
GVSPKVC
PTDYTYTVYRDGTKIKEGLTETTFEEDGVATGNHEYCVEVKYTAG
VSPKKC
PTDYTYTVYRDGTKIKEGLTETTFEEDGVATGNHEYCVEVKYTAG
VSPKEC
PTDYTYTVYRDGTKIKEGLTETTFEEDGVATGNHEYCVEVKYTAG
VSPKVC
PASYTYTVYRDGTKIKEGLTETTYRDAGMSAQSHEYCVEVKYTA
GVSPKVC
APSYTYTIYRNNTQIASGVTETTYRDPDLATGFYTYGVKVVYPNG
ESAIET

### Example 3: Binding Analysis of the KB001 Antibody to Porphyromonas gingivalis

As disclosed herein, a GST-TEV-gingipain-His fusion protein was used to produce recombinant gingipain fusion proteins in E. coli (FIG. 41).

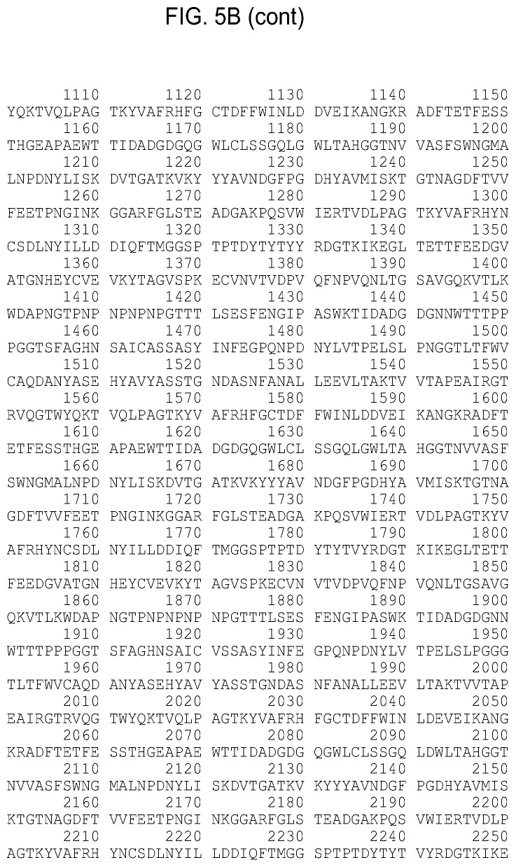

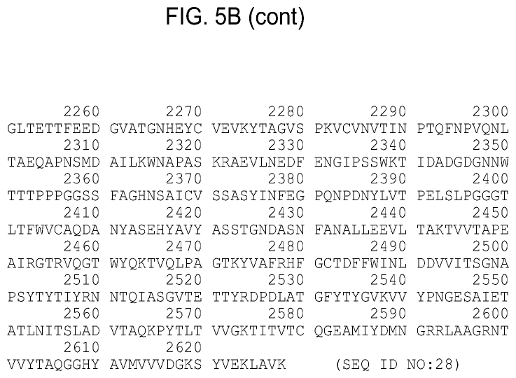

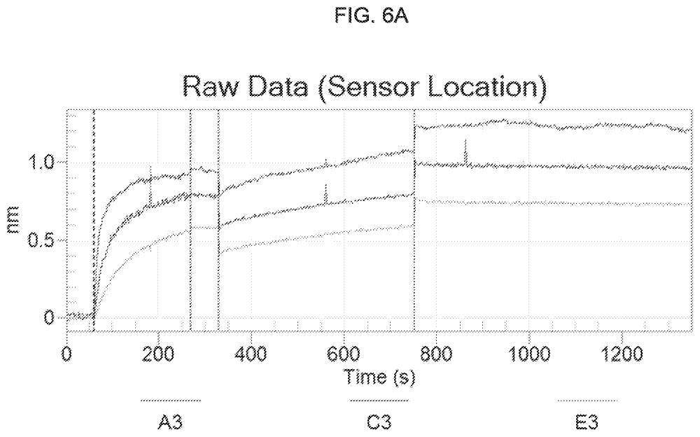

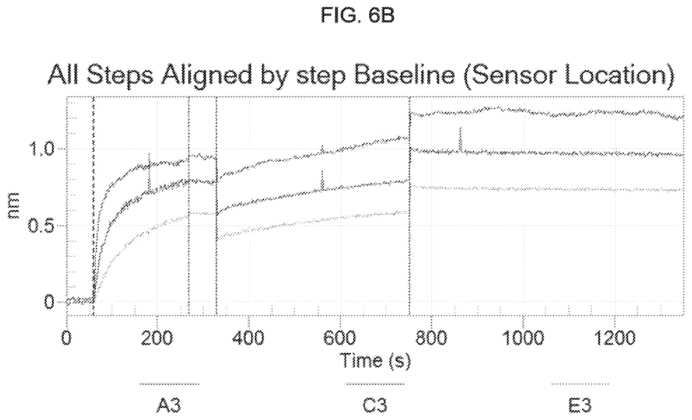

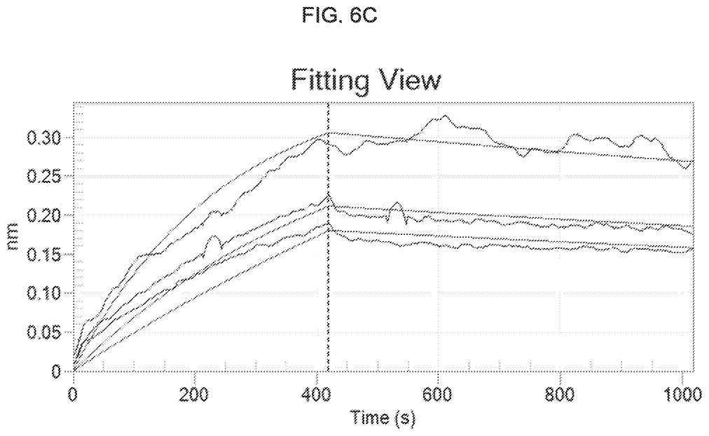

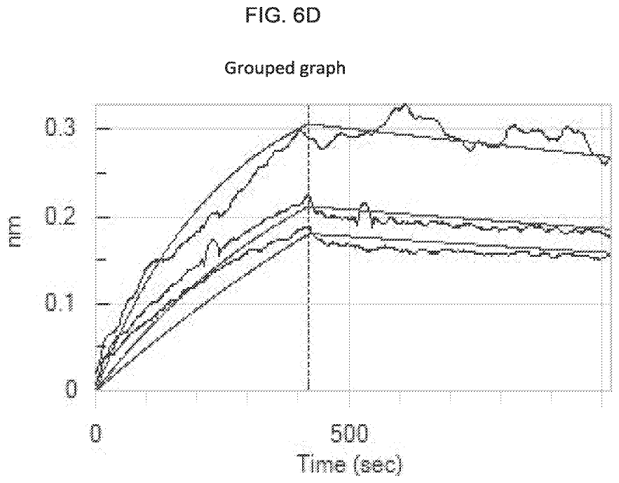

The binding affinity of KB001 for whole P. gingivalis cells (strain W83) was measured using surface plasmon resonance. The response curves at antibody concentrations of 33.3 nM (E3), 100 nM (C3) and 200 nM (A3) are show in FIG. 6A. FIG. 6B shows the data aligned by the step baseline. The data was further fitted, as shown in FIGS. 6C and 6D. Analysis of the rate of association, dissociation and the binding affinity are shown in Table 2.1. The data showed KB001 binds to whole P. gingivalis cells with an apparent Kd in the nanomolar range. In further analysis, KB-001 recognized all 22 laboratory strains and serotypes of P.g. tested as well as 105 human clinical isolates (data not shown).

TABLE 2.1

| Conc. | | KD | KD | | kon | | kdis |
|---|---|---|---|---|---|---|---|
| (nM) | Response | (M) | Error | kon(1/Ms) | Error | kdis(1/s) | Error |
| 200 | 0.2969 | 1.14E−08 | 1.51E−09 | 1.92E+04 | 1.35E+03 | 2.19E−04 | 2.45E−05 |
| 100 | 0.2157 | 1.14E−08 | 1.51E−09 | 1.92E+04 | 1.35E+03 | 2.19E−04 | 2.45E−05 |
| 33.3 | 0.1858 | 1.14E−08 | 1.51E−09 | 1.92E+04 | 1.35E+03 | 2.19E−04 | 2.45E−05 |

In some embodiments, an antigen binding molecule (ABM) of the present disclosure binds to P. gingivalis with a Kd of 10⁻⁷ M or less, 5×10⁻⁸ M or less, 2×10⁻⁸ M or less, or about 1×10⁻⁸ M.

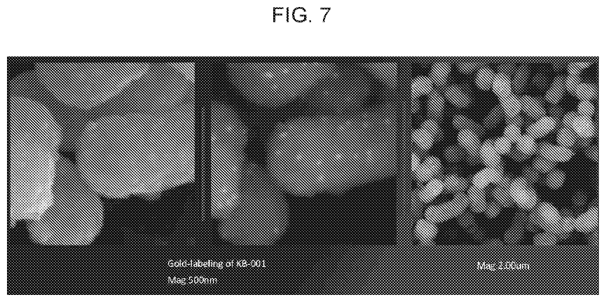

Binding of KB001 to P. gingivalis (W83) was also observed using scanning electron microscopy. The bacteria were labeled with KB001 attached to a gold particles. FIG. 7 shows scanning electron micrographs showing representative images of P. gingivalis without (top panel) and with (bottom panel) filtering to visualize the gold particles. The scanning electron micrographs show approximately 6 individual bacterial cells, and the same view is shown in the top and bottom panels. Direct binding of individual IgG molecules is seen attaching to the cell surface in specific locations on developing/emerging outer membrane blebs/vesicles (OMV). Around 60-80 molecules of the IgG molecules appears bound per bacteria.

Morphological differences in P. gingivalis strains in terms of OMV production and extracellular polymeric substance (EPS) were observed. Clinical isolates were able to produce more OMV and EPS than laboratory strains. KB001 was observed to be binding more to OMV than whole surface. Thus, there exists critical differences among the P. gingivalis strains in terms of OMV and EPS production. The specificity of KB001 may be further defined by testing clinical strains.

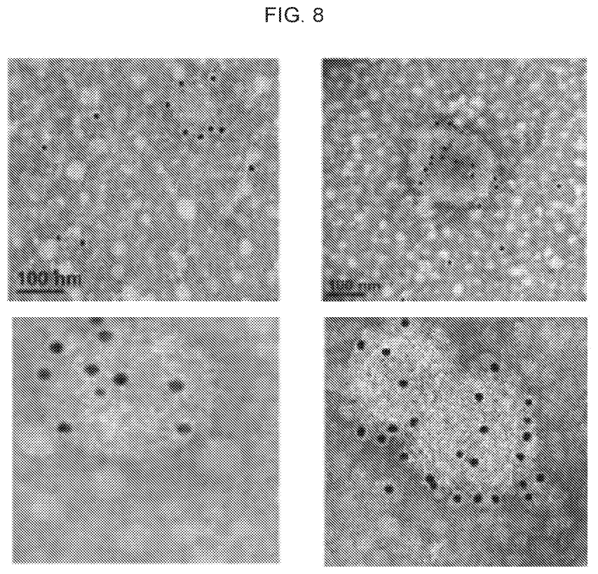

FIG. 8 shows additional electron microscopy images showing binding of KB001 to outer membrane vesicles (OMV) of P. gingivalis, W83. The antibody appears to exhibit strong binding to the OMVs. The size distribution of the OMV ranged from 80-150 nm. KB001 bound to the inner as well outer surface of the OMV bleb.

These blebs are critical for the bacterial survival system as they serve to both feed and/or maintain its energetics, adhesion and biofilm maintenance for the bacteria, and protect it from host defense molecules. In addition, these blebs are considered outer-membrane vesicles, or “microbullets” containing exo-toxins (such as gingipains or LPS) that can flood the systemic circulation, reach the arteries of the heart and large carotid arteries of the neck, thereby increasing the risk of stroke. The outer-membrane vesicles and/or contents thereof can also end up in the brain (see Example 4).

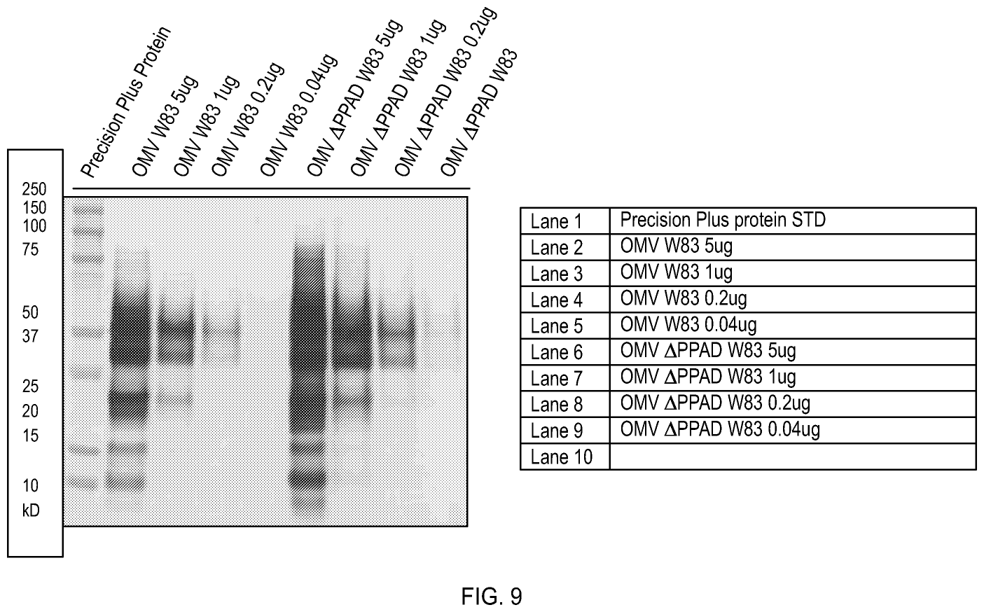

FIG. 9 shows KB001 staining OMV from P. gingivalis strain 33277 and a Peptidylarginine deiminase PPAD C351A 33277 strain in a Western blot demonstrating broad binding activity against different pathogenic strains. PPAD is a virulence factor unique to pathogenic Porphyromonas species, especially P. gingivalis. 100 ul Base samples (conc 500 ug/ml) and 100 μl of NuPAGE loading buffer (novex NP007) with 10% BME (Sigma M-7522) was mixed and heated at 100° C. for 10 min. 5× serial dilutions were made with cold loading buffer. Samples were electrophoresed by using 4-12% Bis-Tris SDS-PAGE (Invitrogen) at 160 v for 60 min.

Subsequently proteins were transferred onto nitrocellulose membrane (Biorad) at 100 v for 60 min, then blocked in 5% milk overnight at R.T. After washing 3×5 min with TTBS (20 mM Tris, 500 mM NaCl, 0.1% Tween-20 pH 8.0), the membrane was incubated with KB001 (1 ug/ml in 10 ml 1% milk) for 2 hrs at R.T. The membrane was then washed 3×5 min in TTBS before probing with secondary antibody anti-mouse (Sigma A4312-1 mL whole molecule alkaline phosphatase 1:10000 in 1% milk) for 2 hr. at room temperature. Membrane was washed 4×5 min with TTBS before developing. Membrane was developed over 5 min using AP-conjugated Substrate kit (Biorad, ref 170643). Molecular mass (Precision Plus Protein Standards, Biorad) is indicated to the left of the membrane.

Without being bound by theory, mechanistically. PPAD activity, in conjunction with Arg-specific gingipains, generates protein fragments with citrullinated C-termini. Such polypeptides are potential de novo epitopes that are key drivers of rheumatoid arthritis. This process could underlie the observed clinical association between rheumatoid arthritis and periodontitis.

In some embodiments, an ABM of the present disclosure binds to outer membrane vesicles (OMV) of P. gingivalis. In some embodiments, the ABM binds to budding or emerging OMV of P. gingivalis.

### Example 4: Specificity of KB-001 Across P.g. Strains

This non-limiting example shows binding of KB001 to phylogenetically diverse strains of P. gingivalis.

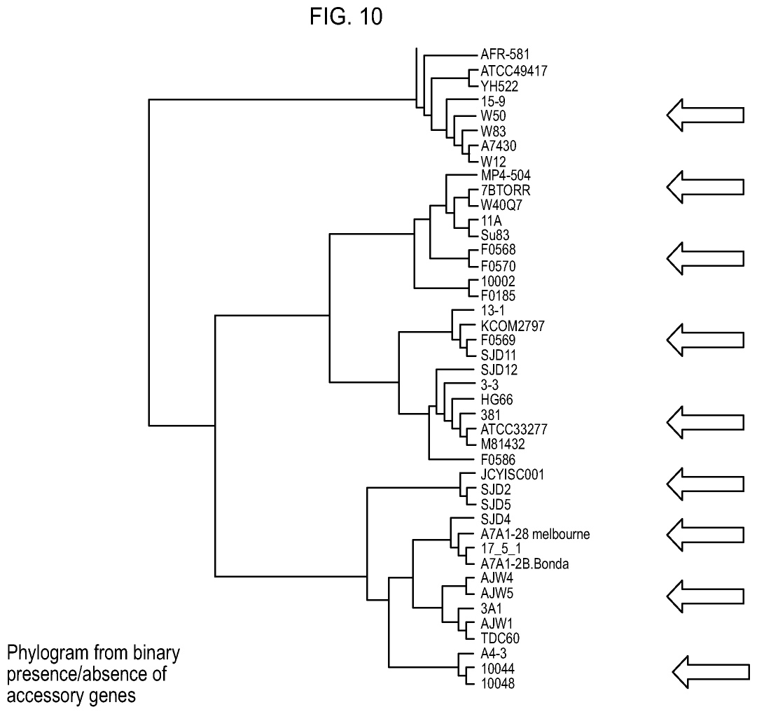

Clinical isolates as well as pathologically significant strains of P. gingivalis were genetically characterized to identify the phylogenetic diversity, using PACBIO sequencing. A distinct phyolgram was generated from the genetic relatedness observations. As show in FIG. 10, a phylogenetic tree of P. gingivalis strains was constructed using binary presence/absence of accessory genes. Using the phyolgram, genetically diverse P. gingivalis strains were identified.

Immuno-electron microscopy of genetically diverse strains of P. gingivalis was done by immunogold labelling to detect specificity of KB001 against P. gingivalis. Ten strains that represent the diversity of strains as determined by comparison of genome sequences (dendrogram, see FIG. 10) were chosen for analysis. The reaction of gold-labeled KB monoclonal antibody with each strain was determined by SEM analysis. The KB-001 antibody was found to bind all genetically diverse strains representing the entire P.g. family.

FIG. 41 shows KB001 binds to P. gingivalis strains W83 and A7436, as well as a clinical isolate. KB001 specifically bound to surface-associated blebs as well as secreted OMVs with the same affinity. The average labeled density of the strains was 50 μm 2. The smallest distance between gold particles (labels) was 0.063 μm, and the largest distance was 0.14 μm. Clinical strains produced a greater number of bleb-like structures on their surface and increased binding by KB001. Without being bound by theory, this may be due to a greater ability of the clinical strains to secrete OMVs. A number of the clinical strains were observed to produce an increase of OMVs and greater binding on the exterior in comparison to surface of the cells.

KB001 recognized 22 laboratory and 105 human clinical isolates and serotypes by immunofluorescence.

### Example 5: Comparison of KB001 Binding Vs 1A1 Binding

This non-limiting example shows the difference in binding characteristics between KB001 and another gingipain monoclonal antibody, 1A1.

When P. gingivalis W83 was immunogold labeled with the respective antibodies, a difference in binding specificity of 1A1 and KB001 was observed (FIG. 11). KB001 was found to binding more to bleb specific regions on the surface of P. gingivalis. In contrast, 1A1 was binding to the general surface. Further, KB001 binding to the W83 was unchanged in dilutions of 1:10, 1:100, 1:1000 tested. Therefore, overall, KB001 has more binding affinity than 1A1.

### Example 6: Loss of KB001 Binding in Pg Knockout Strains

This non-limiting example shows KB001 has reduced or no binding to gingipain knock out strains of P. gingivalis.

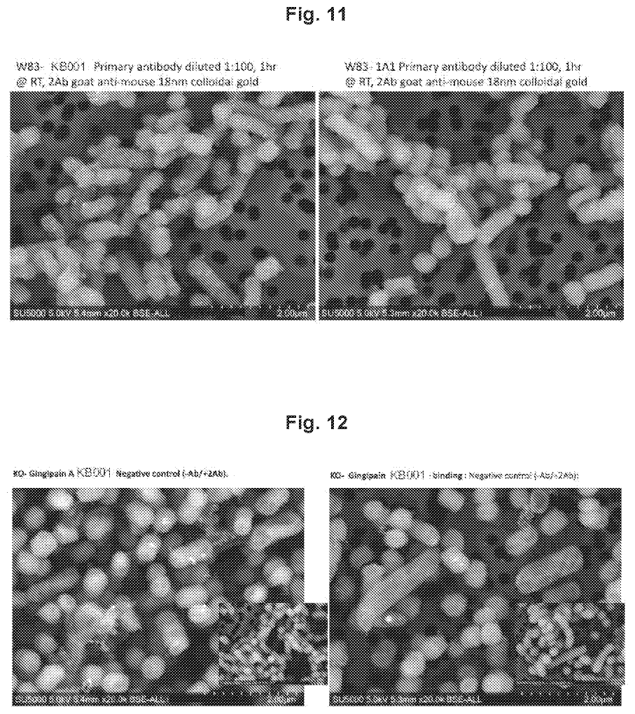

Immunogold staining of gingipain knock out strains (A & B) of P. gingivalis were carried out using KB001. The binding of KB-001 was monitored for two strains: RgpA-/KgP-, and RgpB-/KgP-. It was possible to significantly decrease or result in no binding of the KB-001 antibody to the surface of both gingipain knock out strains in comparison to the W83 strain (FIG. 12). There was decreased or no binding of the antibody to the surface of the gingipain knock out strains in comparison to the W83 strain (a known gingipain rich strain). The minimal binding observed was restricted to the bleb/OMV surface area signifying the potential specificity of KB001 to OMV.

### Example 7: Binding of KB001 to Purified Gingipain

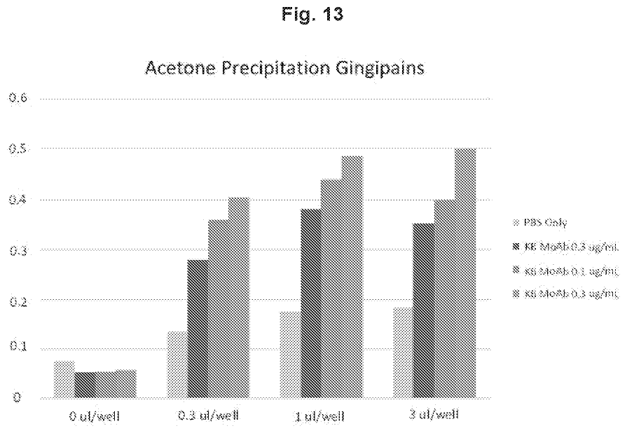

This non-limiting example shows an assay to measure binding of a P. gingivalis gingipain antibody (e.g., KB001) to acetone precipitated gingipain. Plates were coated with 0, 0.3, 1, or 3 μl/well of acetone precipitated gingipain sample and probed with 0, 0.3, 1, and 3 μl/well concentrations of KB001. Crude gingipain was used to coat the wells. Binding was measured by ELISA (FIG. 13) and confirmed the specificity of binding to fully secreted and extruded OMVs from P. gingivalis.

### Example 8: Binding of KB001 Antibody to Targets in Brain Tissue of a Deceased Alzheimer's Disease Patient

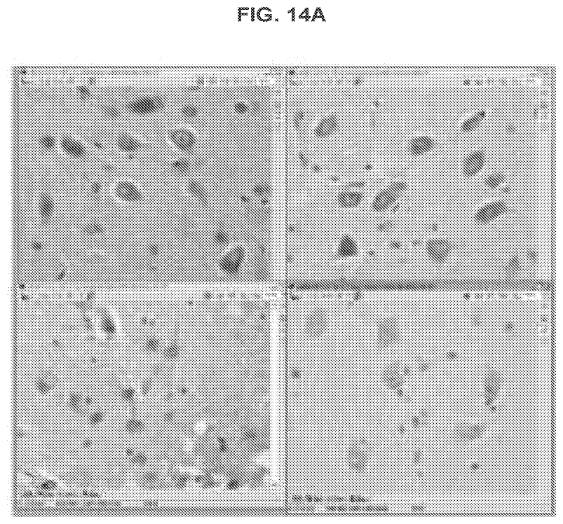

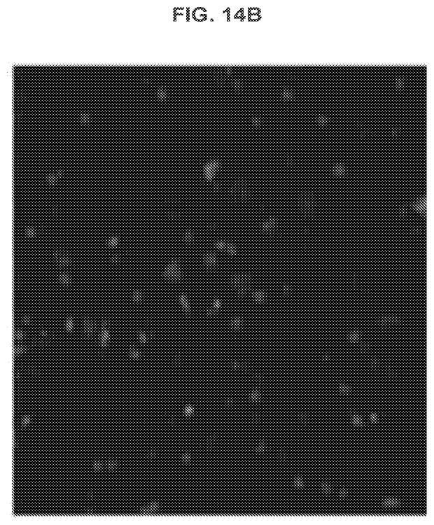

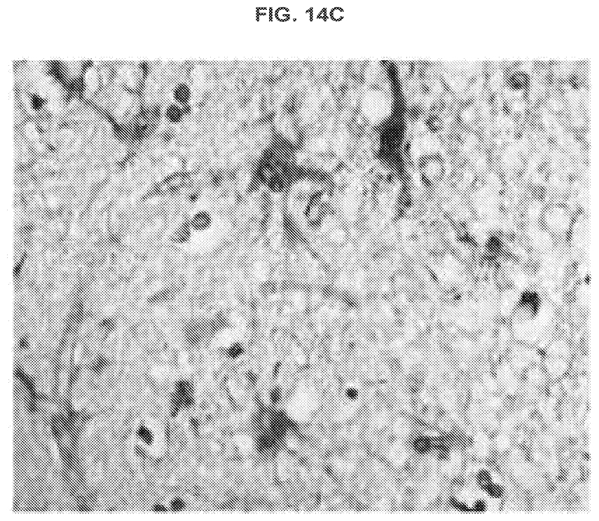

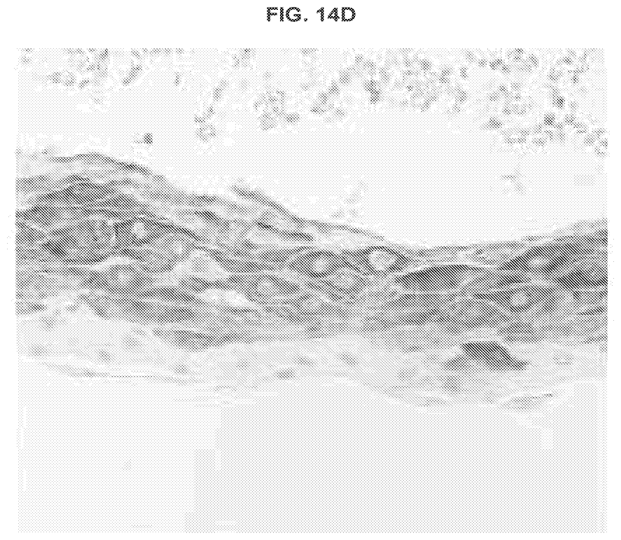

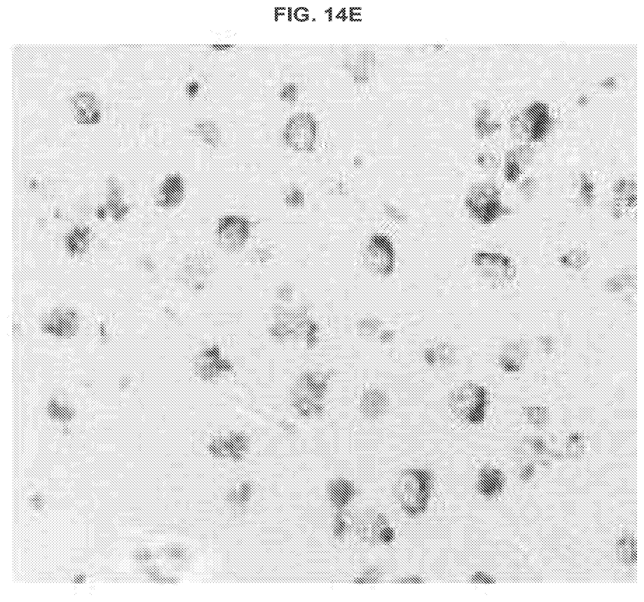

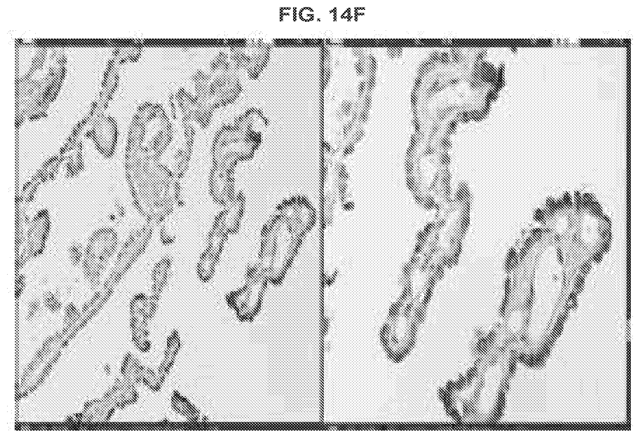

Periodontal disease has been implicated as a risk factor for Alzheimer's disease (AD). Neuropathological characteristics of AD includes accumulation of amyloid-beta (A3), which may be related to an innate immune response to infection. To test the hypothesis that periodontal P. gingivalis infection can induce immune responses in the brain, a brain tissue section from a deceased AD patient was immunohistochemically assayed using KB001. FIG. 14C shows a representative image of staining of the tissue section by KB001. The brown granular staining was observed in hippocampal neurons, microglia and astrocytes, as the antibody bound to gingipain or other P. gingivalis-derived targets in the cells. Thus, KB001 appeared to bind directly to the accumulated exo-toxins in the brain of the AD patient. The antibody labeled neurons, astrocytes and micro-glial cells. FIG. 14A shows further staining of brain tissue sections from an AD patient, using KB001. The staining indicates binding of KB001 to intra-cellular accumulated gingipains located in the brain. FIG. 14E shows IHC staining of the frontal lobe using KB001. These results indicate accumulation of P. gingivalis exo-toxins can occur in an AD patient's brain.

This non-limiting example shows higher sensitivity of KB001 detection of P. gingivalis in tissue samples compared to a PCR-based assay.

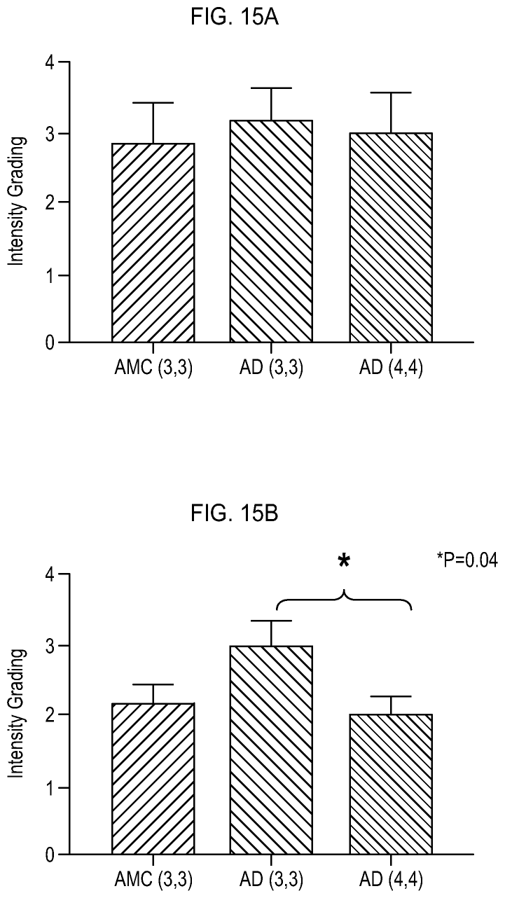

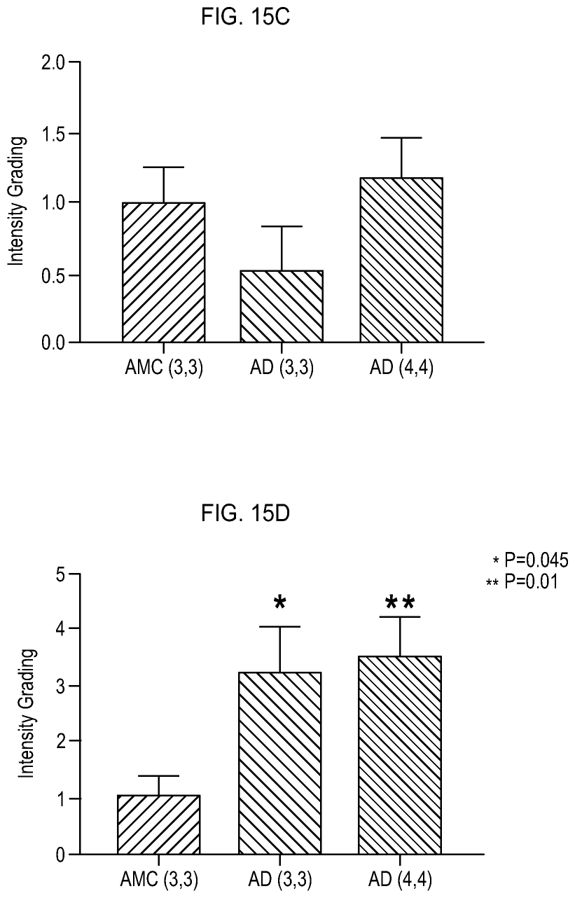

P. gingivalis was carried out using PCR-based liquid hybridization assay of human AD brains and comparative IHC. Forty-six brain tissue samples (frontal and temporal biopsies) from 23 brain specimens (7 AD and 16 AMC) were subjected to PCR-based liquid hybridization assay (PCR-LH) to detect P. gingivalis DNA. Each PCR analysis for Pg DNA used ˜1 microgram of total human DNA extracted from the fresh frozen brain tissue. Since a human genome is approximately three picograms, this represented approximately˜300,000 human cells worth of DNA/assay. Semiquantitative analyses based on the intensity of the autoradiographic signal following PCR-LH to obtain the approximate number of Pg genomic equivalents (copy numbers) for each specimen studied. All samples were negative for P. gingivalis DNA (Table 9.1). FIG. 15D (bottom right panel) shows increased gingipain staining in hippocampus.

To determine Pg genomics equivalents (copy number) per assayed specimen, a series of diluted positive control Pg DNA was isolated and analyzed from pure culture consisting of: 1 μg, 0.5 μg, 100 fg, 20 fg, and 2 fg. These amounts of Pg genomic DNA translate into approximately 500, 250, 50, 10 and 1 genomic equivalents, respectively. 500 genomic equivalents of Pg from an input of one microgram of human DNA corresponds to ˜1 Pg genome/600 human brain cells—similarly if only 10 Pg genomic equivalents from 1 microgram of input DNA that would correspond to 1 Pg genome 30,000 human brain cells.

The densities of immunohistochemical intensity of P. gingipains were assessed relative to none (0) on a scale of 1 to 5 in 7μ sections of temporal lobe/hippocampal area from brains of the age matched control (“AMC”) who were clinically and neuropathologically evaluated by Braak and Braak, and by antibody staging of appropriate region analysis (see Table 8.1 below). Similar assessments were made of analogous areas from brains of patients that were evaluated and determined to be neuropathologically as having met the criteria for a diagnosis of Alzheimer's disease.

TABLE 8.1
Table densitometric comparisons of P. gingipains in Alzheimer
and control brains, segregated by APOE genotypes 3,3 or 4,4

| | | |
|---|---|---|
| AMC 3,3 - | AD 3,3 - | Diagnosis |

| | | | | | |
|---|---|---|---|---|---|
| G03-26 | 1 | C03-54 | 1 | C00-29 | 5 |
| G04-05 | 2 | C05-51 | 4 | C01-80 | 5 |
| G04-21 | 2 | C05-64 | 0 | C07-71 | 5 |
| G05-17 | 0 | C06-35 | 5 | C99-76 | 1 |
| G97-86 | 1 | G04-15 | 5 | G01-78 | 3 |
| G98-114 | 0 | G90-122 | 4 | G03-16 | 2 |

Surprisingly, no significant difference was detected from gingipain antibody staining in the frontal lobe region between control and AD patients. In contrast, AD patient had significantly higher gingipain antibody signal intensity in the hippocampus region.

Staining intensity in the temporal lobe/hippocampal area was measured semi-quantiatively, as shown in FIG. 17B, and results from multiple stained samples are shown in Table 8.2.

TABLE 8.2

| sample_id | specimen_id | diagnosis | location | P. gingivalis detection | IHC result* |
|---|---|---|---|---|---|
| uams_S21-1 | uams_S21 | AD | frontal | Negative | |
| uams_S21-2 | uams_S21 | AD | temporal | Negative | |
| uams_S7-3 | uams_S7 | AD | frontal | Negative | |
| uams_S7-4 | uams_S7 | AD | temporal | Negative | |
| uams_S6-5 | uams_S6 | AD | frontal | Negative | |
| uams_S6-6 | uams_S6 | AD | temporal | Negative | |
| uams_S9-7 | uams_S9 | AD | frontal | Negative | |
| uams_S9-8 | uams_S9 | AD | temporal | Negative | |
| uams_S3-9 | uams_S3 | AD | frontal | Negative | |
| uams_S3-10 | uams_S3 | AD | temporal | Negative | |
| uams_S32-11 | uams_S32 | AD | frontal | Negative | |
| uams_S32-12 | uams_S32 | AD | temporal | Negative | |
| uams_S29-13 | uams_S29 | AD | frontal | Negative | |
| uams_S29-14 | uams_S29 | AD | temporal | Negative | |
| uams_S26-49 | uams_S26 | AMC | frontal | Negative | Positive |
| uams_S26-50 | uams_S26 | AMC | temporal | Negative | Positive |
| uams_S23-51 | uams_S23 | AMC | frontal | Negative | |
| uams_S23-52 | uams_S23 | AMC | temporal | Negative | |
| uams_S12-53 | uams_S12 | AMC | frontal | Negative | |
| uams_S12-54 | uams_S12 | AMC | temporal | Negative | |
| uams_S1-55 | uams_S1 | AMC | frontal | Negative | |
| uams_S1-56 | uams_S1 | AMC | temporal | Negative | |
| uams_S22-57 | uams_S22 | AMC | frontal | Negative | |
| uams_S22-58 | uams_S22 | AMC | temporal | Negative | |
| uams_S14-59 | uams_S14 | AMC | frontal | Negative | Positive |
| uams_S14-60 | uams_S14 | AMC | temporal | Negative | Positive |
| uams_S28-61 | uams_S28 | AMC | frontal | Negative | |
| uams_S28-62 | uams_S28 | AMC | temporal | Negative | |
| uams_S30-63 | uams_S30 | AMC | frontal | Negative | |
| uams_S30-64 | uams_S30 | AMC | temporal | Negative | |
| uams_S13-33 | uams_S13 | AMC | frontal | Negative | |
| uams_S13-34 | uams_S13 | AMC | temporal | Negative | |
| uams_S5-35 | uams_S5 | AMC | frontal | Negative | |
| uams_S5-36 | uams_S5 | AMC | temporal | Negative | |
| uams_S10-37 | uams_S10 | AMC | frontal | Negative | Positive |
| uams_S10-38 | uams_S10 | AMC | temporal | Negative | Positive |
| uams_S11-39 | uams_S11 | AMC | frontal | Negative | |
| uams_S11-40 | uams_S11 | AMC | temporal | Negative | |
| uams_S18-41 | uams_S18 | AMC | frontal | Negative | |
| uams_S18-42 | uams_S18 | AMC | temporal | Negative | |
| uams_S16-43 | uams_S16 | AMC | frontal | Negative | |
| uams_S16-44 | uams_S16 | AMC | temporal | Negative | |
| uams_S19-45 | uams_S19 | AMC | frontal | Negative | |
| uams_S19-46 | uams_S19 | AMC | temporal | Negative | |
| uams_S20-47 | uams_S20 | AMC | frontal | Negative | |
| uams_S20-48 | uams_S20 | AMC | temporal | Negative | |

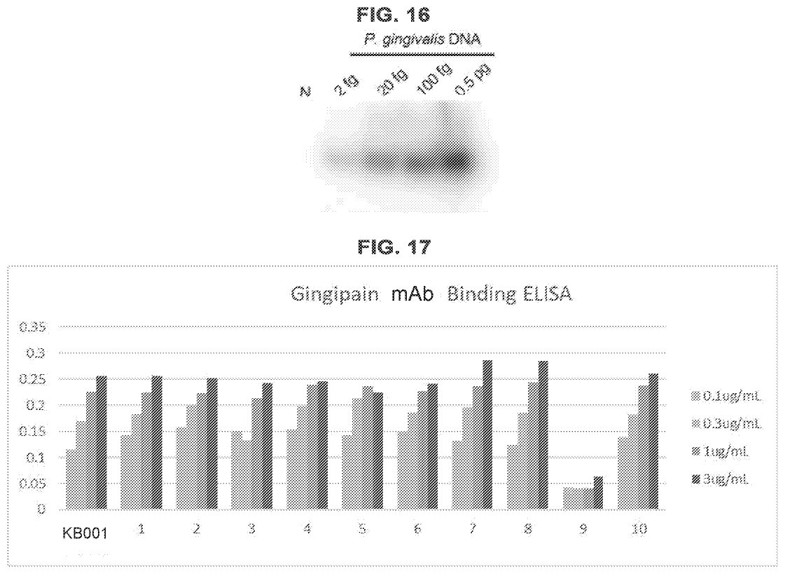

Sensitivity of PCR-based liquid hybridization assay for detection of P. gingivalis was tested. Autoradiography of gel electrophoresis (FIG. 16) shows the PCR-based assay was able to detect 2 fg to 0.5 μg of input purified P. gingivalis genomic DNA. (N: PCR negative control.) Using the PCR-based liquid hybridization assay, all samples were negative for P. gingivalis genomic DNA (Table 9.1). Six samples were positive for KB001 IHC staining.

IHC of 18 hippocampal sections were evaluated and 10 of these were found to be positive (FIG. 14F). As a positive control, KB001 was used to stain gum tissue from a biopsy of a P. gingivalis colonized patient. Brown colored granules are the intra-cellular cytoplasmic localized gingipains as detected with KB001 (FIG. 14D).

### Example 9: Safety/Toxicity Study of KB-001 in Dogs

As disclosed herein, the safety/toxicity profile of KB-001 was assessed in beagle dogs. The test comprised 5 groups, each with 3 males/3 females. Each dog was given a repeat dose sub-gingival or IV application of KB-001 between 0 to 0.33 mg/mL. At day 22 and 43, a necropsy was performed (see Table 3 below).

TABLE 3
Safety./Toxicity study of KB-001 in beagles
Study Design

| | | | | | | Number of Animals per | |
|---|---|---|---|---|---|---|---|
| | | Dose | | Dose | Conc | Necropsy Interval | |
| Group | Treatment | Route | Dose | Volume | (mg/mL) | Day 22 | Day 43 |
| 1 | Vehicle | Gingival | 0 μg | 960 μg | 0 | 3 M/3 F | 3 M/3 F |
| | Control | | | | | | |
| | (sterile saline) | | | | | | |
| 2 | PrevEvent | Gingival | 96 μg | 960 μg | 0.10 | 3 M/3 F | 3 M/3 F |
| 3 | PrevEvent | Gingival | 160 μg | 960 μg | 0.17 | 3 M/3 F | 3 M/3 F |
| 4 | PrevEvent | Gingival | 320 μg | 960 μg | 0.33 | 3 M/3 F | 3 M/3 F |
| 5 | PrevEvent | IV | 320 μg | 1 mL | 0.32 | — | 3 M/3 F |

### Example 10: KB-001 Activity

This non-limiting example shows KB001 prevents processing of HagA by P. gingivalis gingipains.

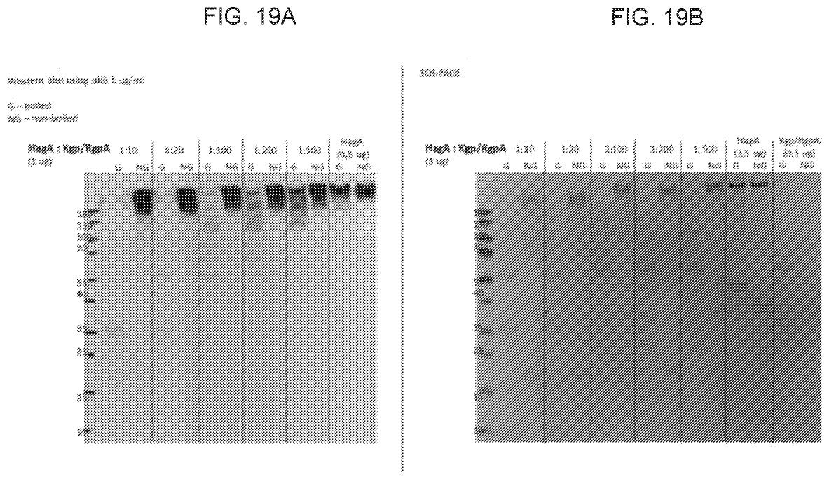

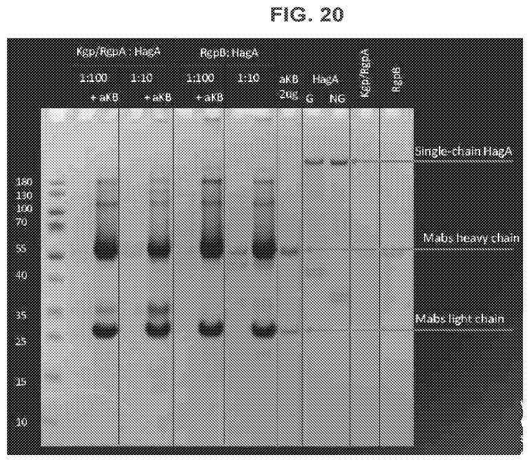

Single chain HagA is processed by gingipains to hemagglutinin/adhesion (HA) domains, which are held together through non-covalent interactions. Mature HagA may assemble on P. gingivalis surface through this process. In FIGS. 19A and 19B, single chain HagA was incubated at the indicated (w:w) ratios with a Kgp/RgpA mixture for 2 hours, and after incubation, boiled or non-boiled samples were resolved by SDS-PAGE. Incubation of single chain HagA with Kgp/RgpA or RgpB generated a complex of the HA domains (FIG. 19B). Without boiling (“NG”), the HA domain complexes were stable in SDS-PAGE (FIG. 20). The individual HA domains were resolved by boiling (“G”). KB001 interfered/blocked full proteolysis of HagA by the gingpain mixture (FIG. 19A).

10× excess of KB001 prevented full proteolysis of HagA by the gingpains (Kgp/RgpA mix or RgpB). Similar results were observed with 100× excess of KB001.

In some embodiments, an ABM of the present disclosure prevents or reduces processing of HagA by P. gingivalis gingipains, e.g., RgpA, RgpB, and/or Kgp. In some embodiments, an ABM of the present disclosure prevents or reduces full proteolysis of HagA by P. gingivalis gingipains, e.g., RgpA, RgpB, and/or Kgp.

### Example 11: Human-Chimeric Antibodies

This non-limiting example shows antigen binding of human-chimeric antibodies derived from KB001, screened and down selected for the best binding as described herein. The antibodies were diluted to 3, 1, 0.3 or 0.1 μg/mL, and binding to gingipain (RgpA) at each dilution of antibody was quantitated by ELISA (FIG. 17). FIG. 17 shows that the antibody binding signal depended on the dilution.

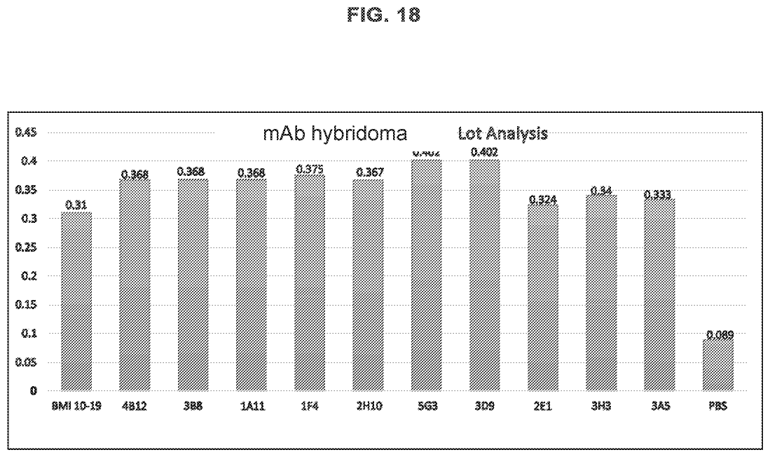

ELISA assay was performed at 0.3 μg/mL of antibody with 6 replicates each. FIG. 18 shows range determination ELISA assay of the 10 antibodies, as described above, against a control standard lot (BMI lot 10-19) at a concentration of 0.3 μg/mL. The best binders were 5G3 and 3D9.

### Example 13: Human-Chimeric Antibodies

This non-limiting example shows the design, generation and production of human-chimeric antibodies to P. gingivalis based on KB001.

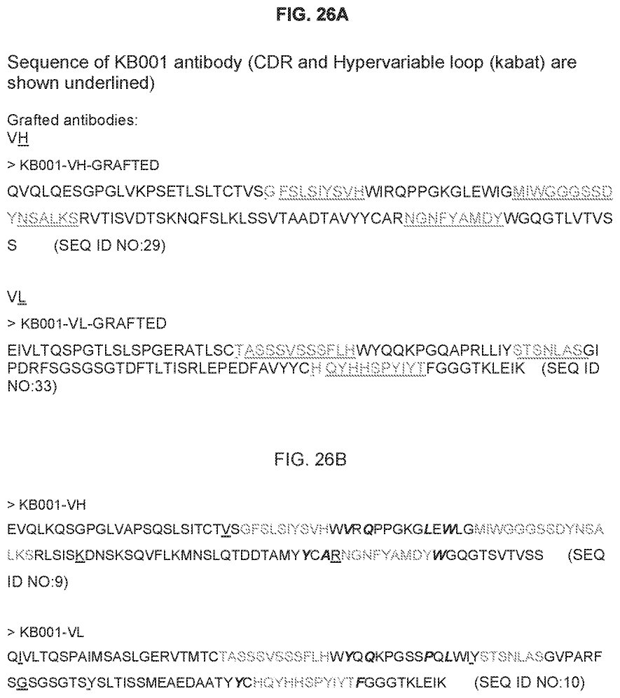

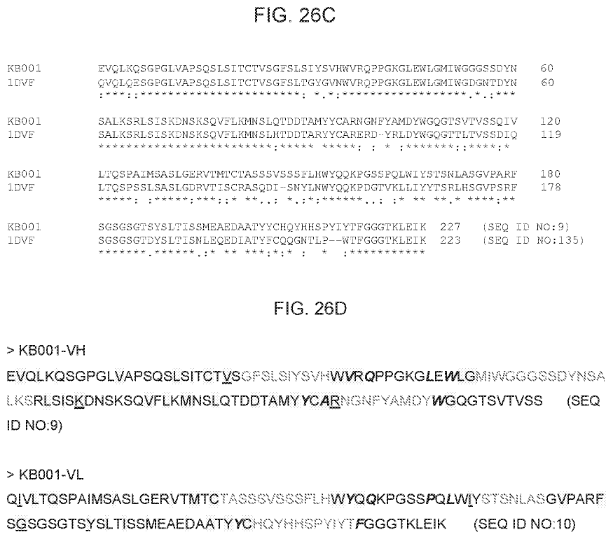

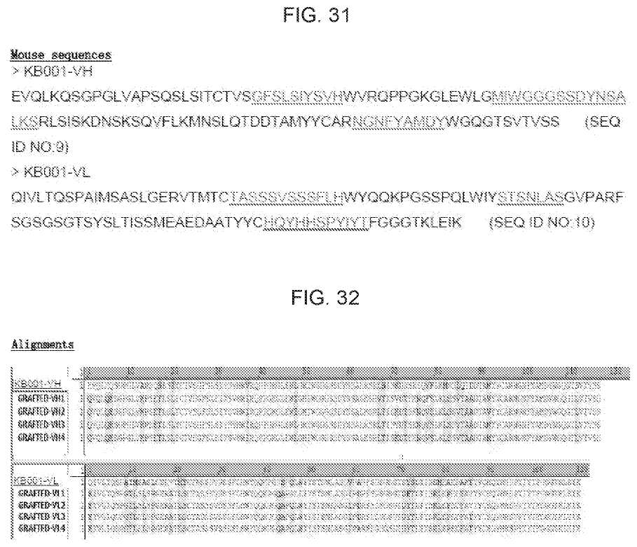

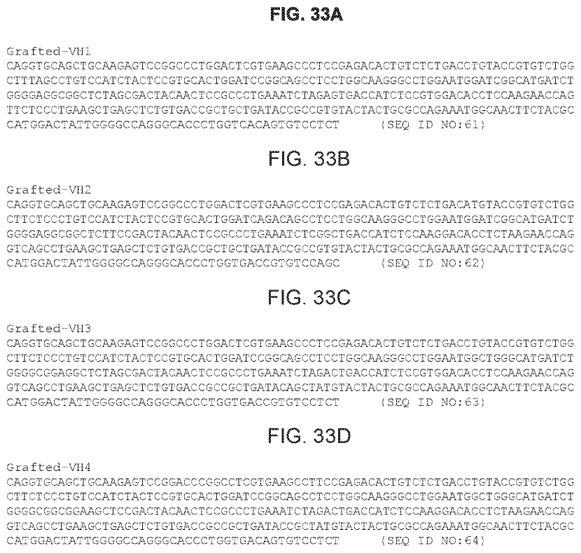

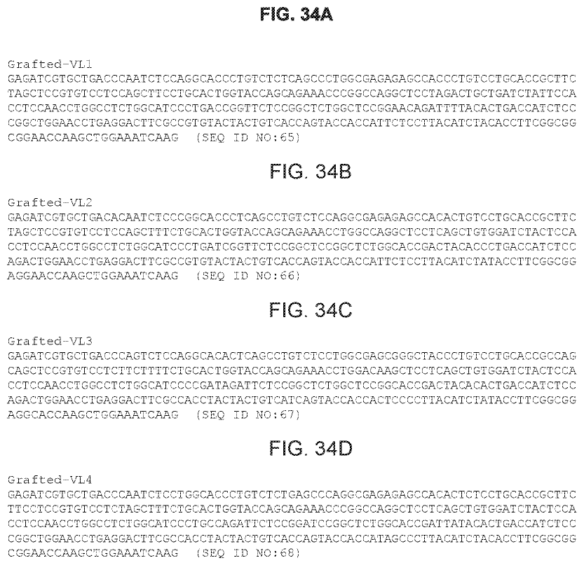

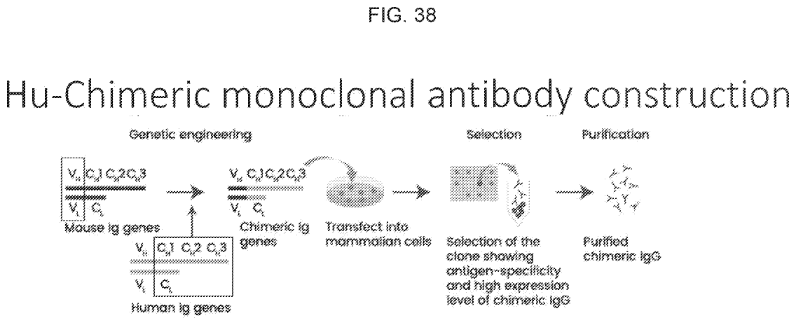

The VH and VL amino acid and corresponding nucleic acid sequences of KB001 are as shown in FIGS. 31, 35A-35B, and 37A-D. The CDRs of the VH and VL of KB001 was grafted onto a human VH and VL framework (FIG. 26A). A schematic design for constructing the humanized chimeric (Hu-Chimeric) antibody is shown in FIG. 38. Non-limiting examples of grafted VH and VL sequences and their alignments to KB001 are given in FIGS. 32-34D. Non-limiting examples of grafted nucleic acid sequences encoding human heavy chain and light chain constant regions of KB001 are given in FIGS. 36A-36B. Back mutations were designed and introduced as follows. The sequences of KB001 antibody were analyzed. Framework region (FR) residues that are believed to be important for the binding activity, e.g., canonical FR residues (underlined) and VH-VL interface residues (bold and italic), of antibody −VH/VL were identified and are shown in FIG. 26B

Homology modeling of KB001 antibody Fv fragments was carried out. KB001 sequences were BLAST searched against PDB_Antibody database for identifying the best templates for Fv fragments and especially for building the domain interface. Structural templatelDVF was selected, identity=66%. Amino acid sequence alignment between KB001 antibody and 1DVF template is shown in FIG. 26C, where ‘|’ is the chain break and * indicates identical amino acid residues in both sequences.

Homology models were built using customized Build Homology Models protocol. Disulfide bridges were specified and linked. Loops were optimized using DOPE method. Based on the homology model of KB001 all framework residues in inner core were highlighted (FIG. 26D). To mutate such residues back to KB001 antibody counterparts can retain inner hydrophobic interaction and reduce potential immunogenicity resulted from back mutation. Residues for back mutating were identified by aligning the VH and VL amino acid sequences of KB001 with the grafted VH and VL sequences, respectively, as shown in FIG. 26E.

FR residues of the grafted antibody were selected for replacement with KB001 antibody Fv equivalent according to the following guideline:
1. FR canonical residues, which do not conform to the canonical structure set, should be selected for priority back mutation;
2. FR residues in the inner core should be selected for priority back mutation;
3. VH-VL interface residues should be selected for priority back mutation;
4. Of all the potential back mutations except the residues in the grafted antibody belonging in all 3 categories aforementioned, the residues that are similar or with same R group in the grafted antibody should be selected for less priority back mutation.

Residues in the grafted antibody that fall in all categories above are different from those of KB001 antibody should be selected for replacement with KB001 antibody counterparts (shown in boxes in FIG. 26E).

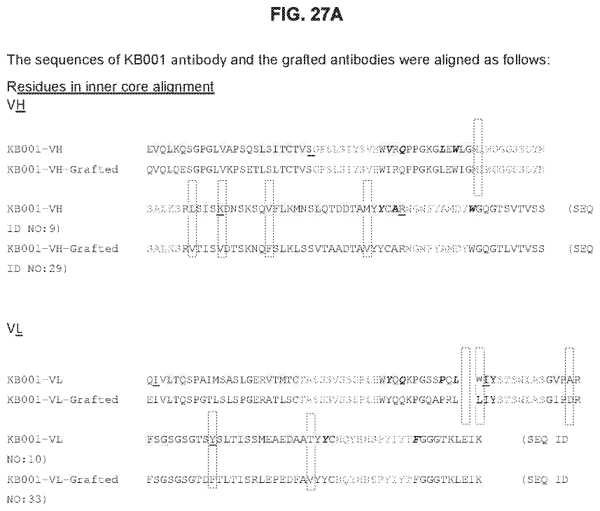

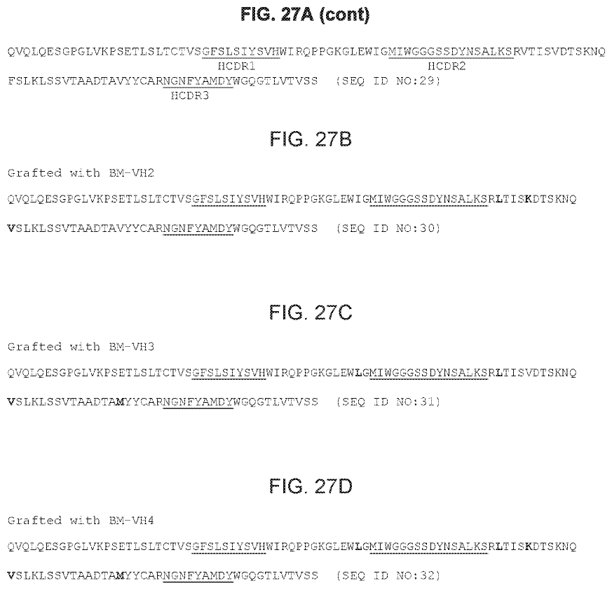

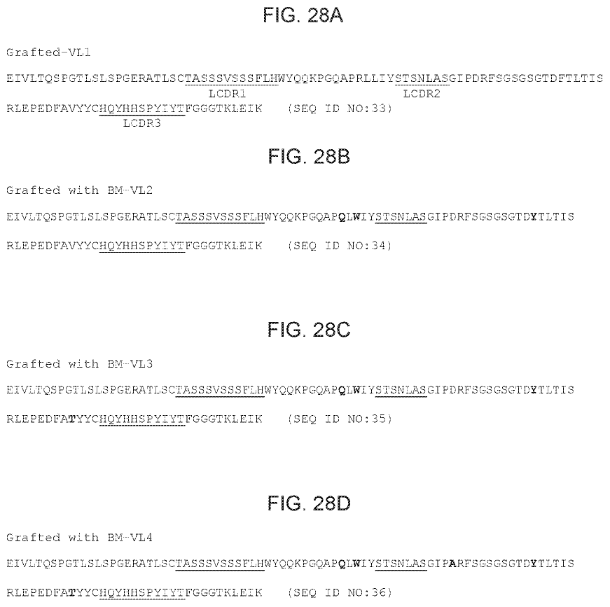

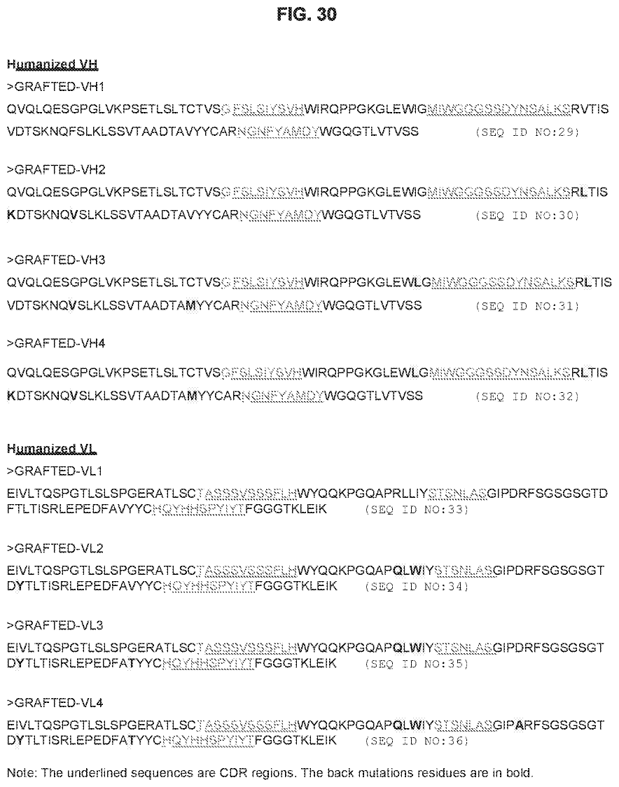

The grafted and back-mutated heavy and light chain variable regions are shown in FIGS. 27A-27D and 28A-28D, respectively, as well as in FIG. 30.

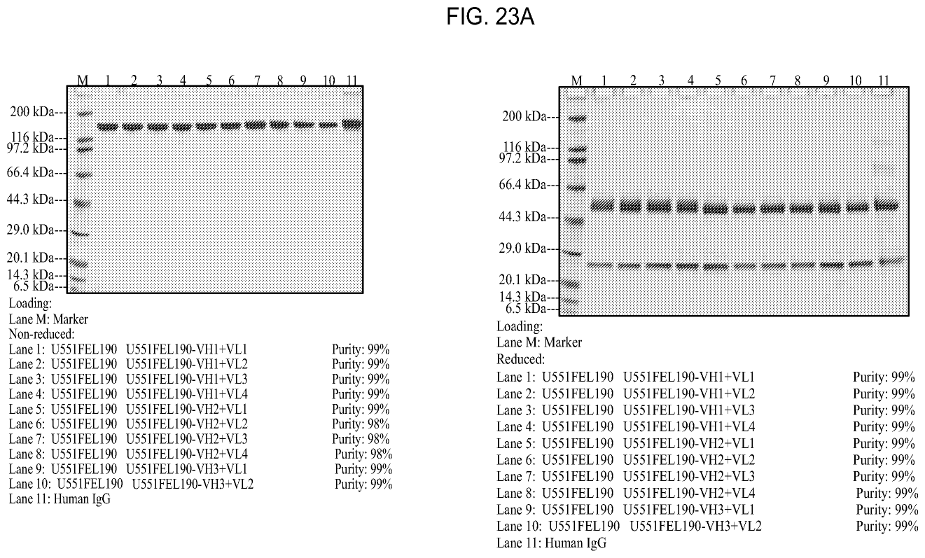

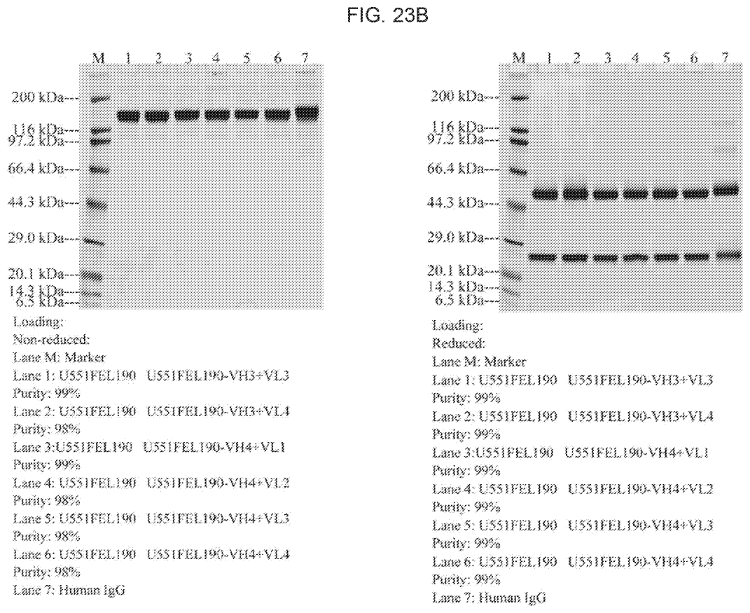

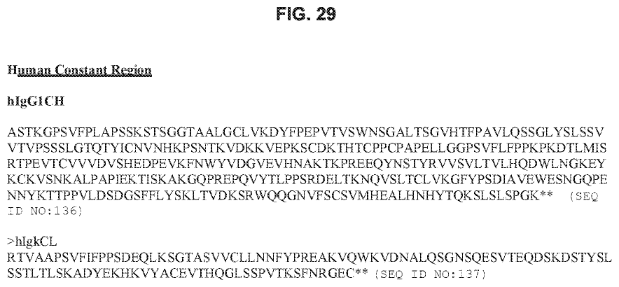

All antibodies included heavy chain and light chain constant regions as shown in FIG. 29 (human IgG1 and human Ig kappa). The following combinations were designed, as shown in Table 13.1, and generated, as shown in FIGS. 23A, 23B, and 47. FIGS. 23A and 23B are images of reduced SDS PAGE gels of individual antibody clones showing heavy and light chains.

TABLE 13.1

| Antibody | VH variant | VL variant |
|---|---|---|
| H1 | VH1 (SEQ ID NO: 29) | VL1 (SEQ ID NO: 33) |
| H2 | VH1 (SEQ ID NO: 29) | VL2 (SEQ ID NO: 34) |
| H3 | VH1 (SEQ ID NO: 29) | VL3 (SEQ ID NO: 35) |
| H4 | VH1 (SEQ ID NO: 29) | VL4 (SEQ ID NO: 36) |
| H5 | VH2 (SEQ ID NO: 30) | VL1 (SEQ ID NO: 33) |
| H6 | VH2 (SEQ ID NO: 30) | VL2 (SEQ ID NO: 34) |
| H7 | VH2 (SEQ ID NO: 30) | VL3 (SEQ ID NO: 35) |
| H8 | VH2 (SEQ ID NO: 30) | VL4 (SEQ ID NO: 36) |
| H9 | VH3 (SEQ ID NO: 31) | VL1 (SEQ ID NO: 33) |
| H10 | VH3 (SEQ ID NO: 31) | VL2 (SEQ ID NO: 34) |
| H11 | VH3 (SEQ ID NO: 31) | VL3 (SEQ ID NO: 35) |
| H12 | VH3 (SEQ ID NO: 31) | VL4 (SEQ ID NO: 36) |
| H13 | VH4 (SEQ ID NO: 32) | VL1 (SEQ ID NO: 33) |
| H14 | VH4 (SEQ ID NO: 32) | VL2 (SEQ ID NO: 34) |
| H15 | VH4 (SEQ ID NO: 32) | VL3 (SEQ ID NO: 35) |
| H16 | VH4 (SEQ ID NO: 32) | VL4 (SEQ ID NO: 36) |

In some embodiments, an ABM of the present disclosure includes a humanized heavy chain variable region (HVR) with one or more back mutations as indicated by rectangular boxes in the VH alignment in FIG. 26E. In some embodiments, an ABM of the present disclosure includes a humanized light chain variable region (LVR) with one or more back mutations as indicated by rectangular boxes in the VL alignment in FIG. 26E. In some embodiments, an ABM of the present disclosure includes a HVR having an amino acid sequences of one of SEQ ID NOS:29-32. In some embodiments, an ABM of the present disclosure includes a LVR having an amino acid sequences of one of SEQ ID NOS:33-36.

### Example 14: Variant Humanized Antibodies

This non-limiting example shows variant humanized antibodies derived from KB001 binding to gingipain, and quantitating binding using ELISA.

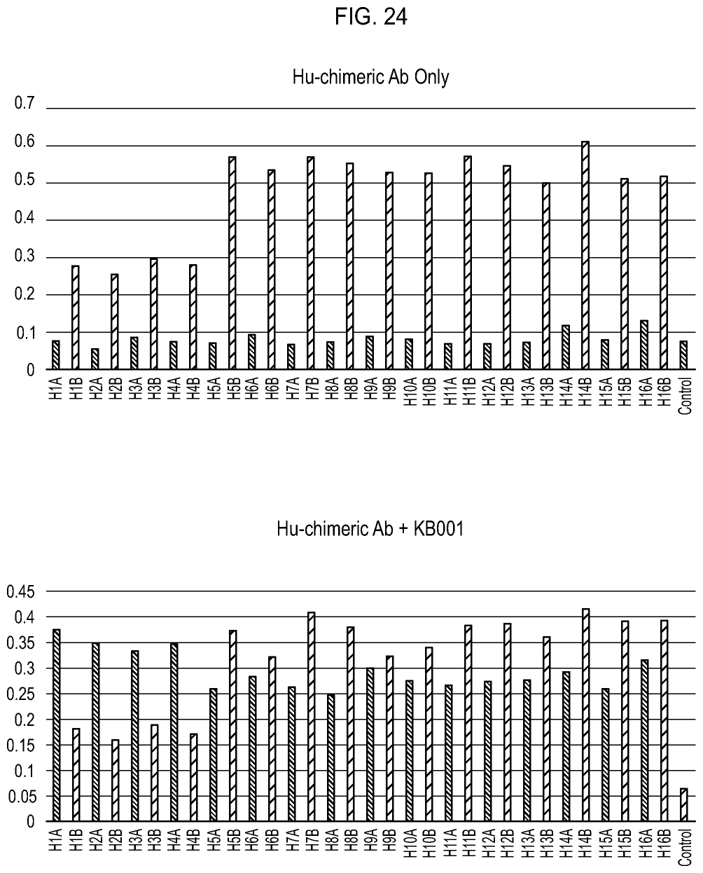

Binding of variant antibodies to gingipain (RgpA) was quantitated by ELISA (FIG. 24). FIG. 24, top panel, shows the signal from HuAb probed with anti-human secondary (bar labeled “B” for each variant) and the signal from the HuAb probed with anti-mouse secondary (bar labeled “A” for each variant). H14, H5, H7 showed the greatest binding, and H11, H1, H2, H3, and H4 showed weaker binding. The low signal for anti-mouse secondary demonstrates that the mouse antibody is specific for mouse IgG and does not react well with human IgG, as expected. FIG. 24, bottom panel, shows the signal from the HuAb+KB001 complex probed with anti-human secondary (bar labeled “B” for each variant) and the signal from the KB001 probed with anti-mouse (bar labeled “A” for each variant), which provides the competitive ELISA data (the lower the bar, the better the competition from HuAb). Here, H14 and H7 demonstrated the most robust binding, while H8 and H14 showed the greatest competition in a 1-hour binding assay. H5, H7, and H15 also exhibited very good competition. The majority of HuAb bind the gingipain antigen well and compete with KB001.

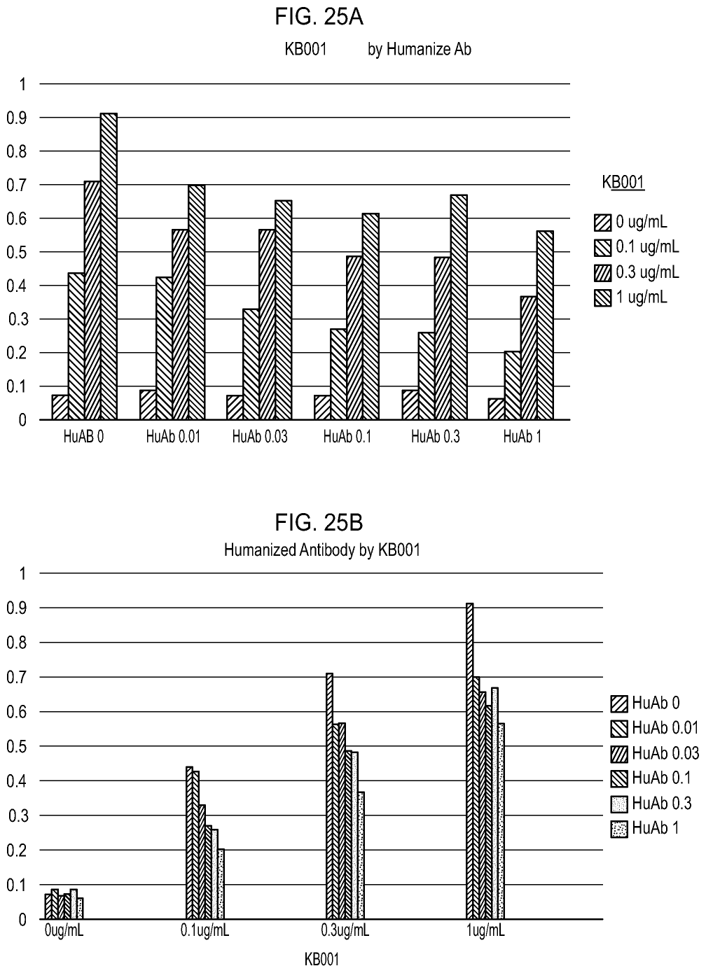

FIGS. 25A and 25B show two presentations of HuAb competition binding assay with KB001 using ELISA. FIG. 25A shows KB001 antibody is increased in competition with six concentrations of HuAb (in μg/mL). FIG. 25B shows the Humanized Ab is increased in competition with four KB001 MoAb concentrations.

These results show antibodies having improved binding affinity compared to KB001 were generated.

### Example 15: Binding Properties of Human-Chimeric Antibodies Using SEM

This non-limiting example shows binding of Hu-Chimeric antibodies using whole P. gingivalis bacteria binding assay.

### Methodology: Scanning Electron Microscopy (SEM)

SEM detection: 1) SE detection
   2) BSE detection

Five out of 16 total Hu-chimeric MAbs were down selected via a ELISA screening binding and competition assays. The selected Hu-chimeric MAbs were H5: VH2+VL1; H7: VH2+VL3; H8: VH2+VL4; H14: VH4+VL2; H15: VH4+VL3. Specimens bound to select Hu-chimeric MAbs were examined with secondary electrons (SE) and backscatter electrons (BSE), and digital micrographs were acquired with a field-emission SEM (SU-5000, Hitachi High Technologies America, Schaumburg, IL, USA) operated at 5 kV.

### Methodology: SEM Fragment Immunolabeling

### Fragment Immunolabeling:

P. gingivalis cells were resuspended into primary fixative containing 4% paraformaldehyde in PBS. Cells were deposited onto poly-L-lysine treated 0.2 μm membrane filters. Filters were incubated onto primary fixative for 30 minutes at room temperature. After fixation, immunogold labeling was performed by exposure of the filters at room temperature as follows: filters were treated with NH4Cl in PBS, rinsed with PBS, incubated in a blocking solution (1% non-fat dry milk, 0.5% cold water fish skin gelatin, 0.01% Tween-20 in PBS) and exposed to the primary antibody fragments that the researcher provide data 1:4000 dilution. Negative control was established by replacing primary antibody with PBS. Filters were washed in PBS and incubated with a 4 nm Colloidal Gold AffiniPure Goat Anti-Human IgG, Fc fragment specific (1:200 dilution; Jackson ImmunoResearch Laboratories, West Grove, PA), washed in PBS, fixed in Trump's fixative (Electron Microscopy Sciences, Hatfield, PA), and water washed. Filters were then enhanced using an HQ Silver Enhancer for 4 minutes (Nanoprobes, Inc., Yaphank, NY) followed by a water wash. After immunogold labeling, the filters were processed for SEM with the aid of a Pelco BioWave laboratory microwave (Ted, Pella, ReddingCA, USA). Filters were dehydrated in a graded ethanol series 25%, 50%, 75%, 95%, 100% and critical point dried (Autosamdri-815, Tousimis, Rockville, MD, USA). Filters were mounted on carbon adhesive tabs on aluminum specimen mounts, and carbon coated (Cressington 328/308R, Ted Pella, Redding, CA, USA). Samples were kept under house vacuum until ready to image.

### SEM Imaging

Specimens were examined with secondary electrons (SE) and backscatter electrons (BSE), and digital micrographs were acquired with a field-emission SEM (SU-5000, Hitachi High Technologies America, Schaumburg, IL, USA) operated at 5 kV.

### Results:

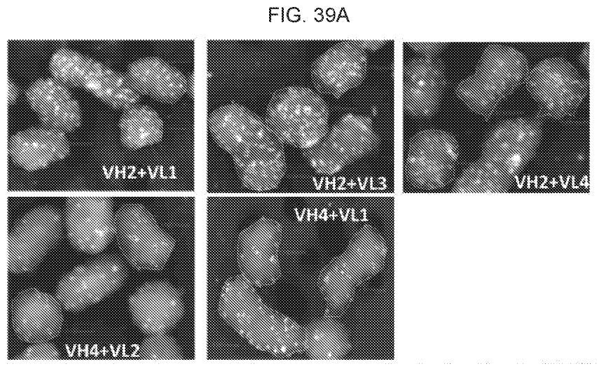

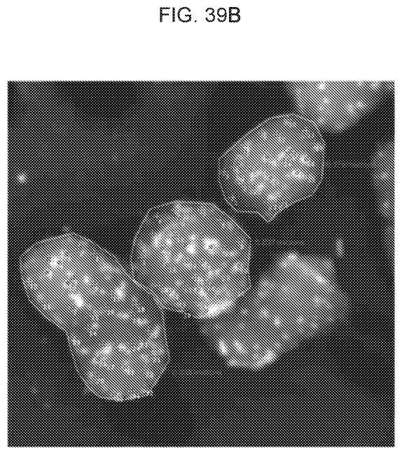

All 5 Hu-chimeric gold labeled Mab fragments demonstrated direct binding to the bacterial surface being located on and associated with emerging/forming outer membrane vesicles (OMVs) (FIG. 39A). The best Hu-chimeric MAbs were H7 And H14. Detailed densitometric measurements were made quantitating the distance and number of bound antibody fragments. FIG. 39B shows magnified, quantitated binding events of H7 (VH2+VL3).

There existed a difference in the binding ability of the human chimeric-antibodies against P. gingivalis (W83). VH4-containing antibodies had a lower binding affinity compared to the VH2-containing antibodies. Among the 5 chimeric antibodies that were compared, VH2+VL3 had the greatest binding in comparison to the other chimeric-antibody combinations.

### Example 16: Binding Properties of Human-Chimeric Antibodies Using SPR

This study was performed to measure the binding affinity of antibodies to HRGPA-6H using Biacore 8K.

TABLE 16.1
Sample Materials

| Samples | MW(KDa) | Concentration(mg/ml) |
|---|---|---|
| HRGPA-6H | 70.5 | 0.85 |
| H14 | 150 | 0.503 |
| H5 | 150 | 0.647 |
| H7 | 150 | 0.515 |
| H8 | 150 | 0.593 |
| KB001 | 150 | 4 |

TABLE 16.2
Instrument and Reagent

| Names | Cat. No. | Lot. No. | Vendor |
|---|---|---|---|
| Biacore T200: | N/A | N/A | GE Healthcare |
| GR18010468 | | | |
| HBS-EP + buffer | BR-1006-69 | 31644 | GE Healthcare |
| Series S Sensor | BR-1005-30 | 10299106 | GE Healthcare |
| Chip CM5 | | | |
| 10 mM sodium | BR-1003-50 | 30789 | GE Healthcare |
| acetate, pH 4.5 | | | |
| Amine coupling kit | BR-1000-50 | 31165 | GE Healthcare |
| Regeneration | Jan. 5, 2021 | Jan. 5, 2021 | Genscript |
| buffer: 10 mM | | | |
| Glycine-HCl pH 1.7 | | | |

Methodology: Immobilization of HRGPA-6H onto CM5 Sensor Chip

The immobilization of HRGPA-6H was performed under 25 degrees Celsius while HBS-EP was used as the running buffer. The sensor chip surface of flow cells 1, 2 were activated by freshly mixed 50 mmol/L N-Hydroxysuccinimide (NHS) and 200 mmol/L 1-ethyl-3-(3-dimethylaminopropyl) carbodiimide hydrochloride (EDC) for 120s (10 μL/min). Afterwards, HRGPA-6H diluted in 10 mmol/L NaAC (pH 4.5) to 4 ug/ml were injected into the flow cell 1.2 to achieve conjugation of appropriate Response Unit respectively. After the amine coupling reaction, the remaining active coupling sites on chip surface were blocked with 120s injection of 1 mol/L ethanolamine hydrochloride.

### Methodology: Affinity Measurement of Antibodies to HRGPA-6H

The assay was performed at 25° C. and the running buffer was HBS-EP+. Diluted antibodies were captured on the sensor chip through Fc capture method. HRGPA-6H was used as the analyte, followed by injecting running buffer as dissociation phase. The running configuration was as listed in TABLE 16.3.

TABLE 16.3
Running configuration
Capture

| Ligand | antibodies |
|---|---|
| Immobilization level(RU) | ~80 |

Association & Dissociation

| | |
|---|---|
| Association contact time(s) | 120 |
| Dissociation contact time(s) | 360 |
| Flow rate(μl/min) | 30 |
| Sample concentrations(nM) | 400, 200, 100, 50, |
| | 25, 12.5, 6.25 |

Surface regeneration

| | |
|---|---|
| Regeneration buffer | 10 mM Glycine-HCl |
| Contact time(s) | 30 |
| Flow rate(μl/min) | 30 |

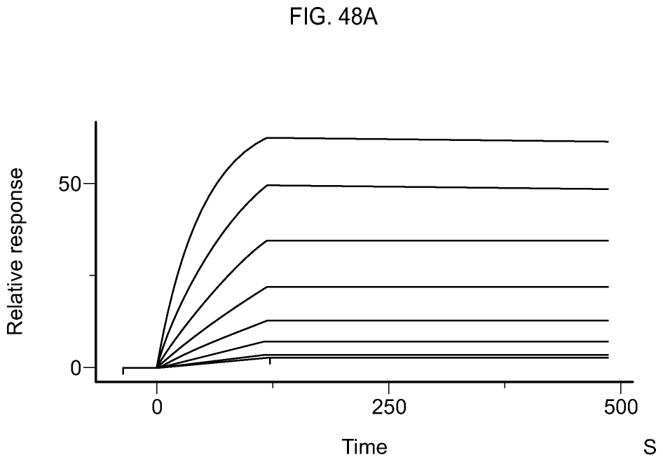

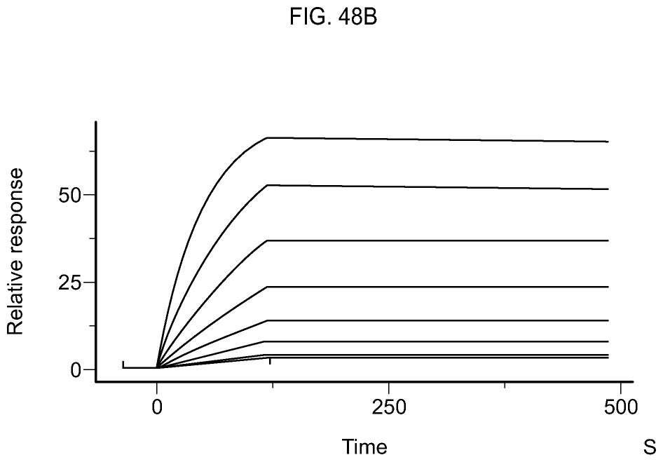

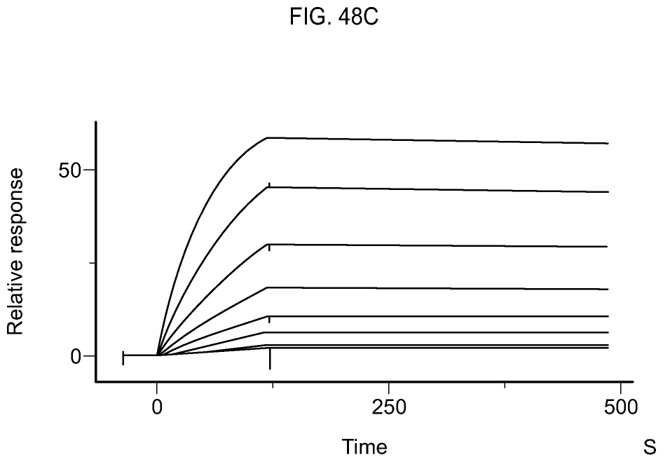

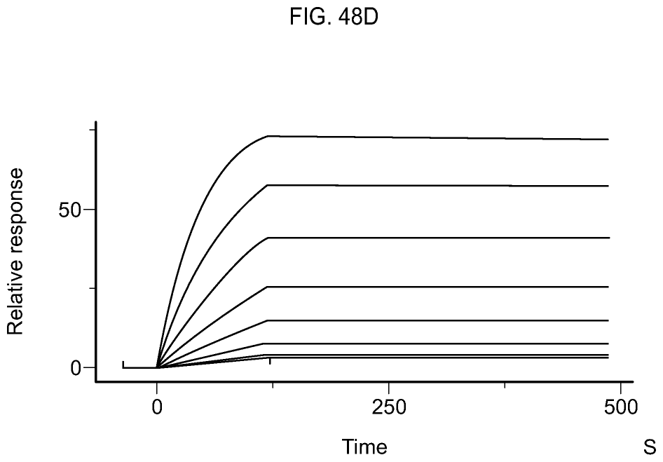

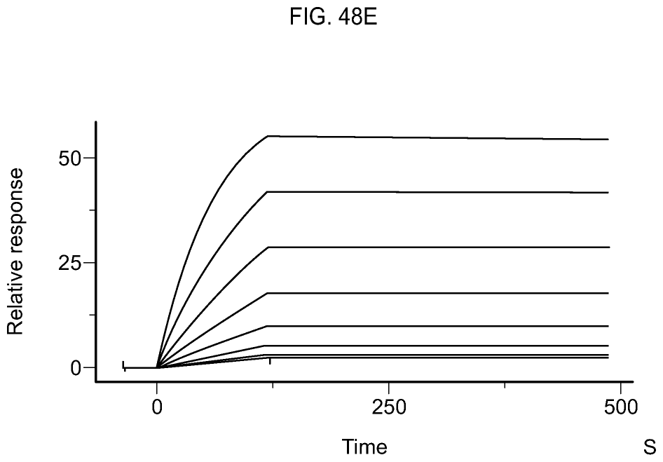

All the data were processed using the Biacore 8K Evaluation software version 1.1. Flow cell 1 and blank injection of buffer in each cycle were used as double reference for Response Units subtraction. The binding kinetic data is given in TABLE 16.4, and the binding sensor-grams are shown in FIGS. 48A-48E. According to the results, the affinity of H7 to HRgpA-6H was stronger than other tested antibodies to HRgpA-6H.

TABLE 16.4
Binding kinetics

| | | ka | kd | KD | Rmax | Chi² |
|---|---|---|---|---|---|---|
| Ligand | Analyte | (1/Ms) | (1/s) | (M) | (RU) | (RU²) |
| HRgpA-6H | H8 | 5.03E+04 | 6.70E−05 | 1.33E−09 | 63.7 | 1.40E−01 |
| HRgpA-6H | H14 | 5.21E+04 | 4.89E−05 | 9.39E−10 | 68.5 | 1.75E−01 |
| HRgpA-6H | KB001 | 4.26E+04 | 8.62E−05 | 2.02E−09 | 62.5 | 1.22E−01 |
| HRgpA-6H | H5 | 5.45E+04 | 4.50E−05 | 8.25E−10 | 74.7 | 1.61E−01 |
| HRgpA-6H | H7 | 4.14E+04 | 1.07E−05 | 2.58E−10 | 57.9 | 5.20E−02 |

Similar binding was assessed with the K222A mutant antibodies against the recombinant Pg protein target/ligand HRgpA-6H (Table 16.5). All four of the K222A mutants were found to have as good or better affinity than their parents. H5 K222A had the overall greatest affinity for HRgpA-6H.

TABLE 16.4
Binding kinetics of K222A antibody variants

| | | | Chi² | ka | kd | KD | Rmax |
|---|---|---|---|---|---|---|---|
| Affinity | Ligand | Analyte | (RU²) | (1/Ms) | (1/s) | (M) | (RU) |
| excellent | HRgpA-6H | H5 K222A | 1.22E−01 | 5.02E+04 | 3.42E−06 | 6.80E−11 | 68 |
| good | HRgpA-6H | H7 K222A | 7.21E−02 | 5.48E+04 | 4.36E−05 | 7.95E−10 | 77.1 |
| good | HRgpA-6H | H8 K222A | 9.25E−02 | 5.80E+04 | 3.30E−05 | 5.70E−10 | 80 |
| moderate | HRgpA-6H | H14 K222A | 7.99E−02 | 5.01E+04 | 6.53E−05 | 1.30E−09 | 68.6 |

### Example 17: Binding Affinity Maturation Through Antibody Mutagenesis

This non-limiting example shows binding of the parental mouse antibody, as well as human chimeric cleavage resistant constructs, to HRGPA-6H. The constructs were made through affinity maturation to enhance the affinity of antibody to HRgpA-6H according to the strategy of PML saturation mutagenesis and FASEBA screening.

### Materials

Amino acid sequences of parental antibody (provided by the client)
Antigen: HRgpA-6H (provided by the client)
Parental antibody: KB001 (provided by the client)
E. coli TG1
Ampicillin stock, 100 mg/ml
2×YT: 1.6% Tryptone, 1.0% Yeast Extract, 0.5% NaCl
IPTG 0.1 mM
Microtiter ELISA plates
Coating buffer: CBS (1.588 g/L Na₂CO₃, 2.928 g/L NaHCO₃)
Blocking buffer: 3% MPBS
Washing buffer: 0.05% PBST
BSA, 10 μg/ml
Tetramethylbenzidine (TMB)
1M HCl
Goat Anti-MOUSE IgG (Fab specific) [HRP]
Goat Anti-Human IgG, F(ab′)₂ [HRP]
Mouse Anti-Human IgG, F(ab′)₂ [HRP]
Goat Anti-Human IgG (H+L) [HRP]
Anti-BSA [HRP]
Biacore 8K (GE Healthcare)
Series S Sensor Chip CM5 (GE Healthcare, Cat. No.: BR-1005-30)

### Methodology: Construction and Production of Parental Fab FASEBA Sample

The DNA sequences encoding the antibody heavy and light chains were synthesized and inserted into FASEBA vector to construct expression plasmids of parental Fab. Then the FASEBA vector was transferred into TG1 competent, and after selecting positive clones for culture, IPTG induced parental Fab expression. The mouse Fab and chimeric Fab were expressed for further validation.

### Methodology: Affinity Measurement of Parental Antibody and Parental Mouse Fab FASEBA Sample

The affinity of parental antibody to antigen protein was determined using a Surface Plasmon Resonance (SPR) biosensor, Biacore 8K (GE Healthcare). The measurements were performed at 25° C. HRGPA-6H was immobilized on the Series S Sensor Chip CM5. KB001-WT-Ab was used as the analyte with association time of 120s and buffer flow was maintained for 360 s for dissociation. The data of dissociation (kd) and association (ka) rate constants were obtained using Biacore 8K evaluation software. The equilibrium dissociation constants (KD) were calculated from the ratio of kd over ka. The affinity of parental mouse Fab FASEBA supernatant to antigen protein was determined using Biacore 8K (GE Healthcare). FASEBA supernatant was captured on the sensor chip. Antigen was used as the analyte with association time of 120s and buffer flow was maintained for 360 s for dissociation. The data of dissociation (kd) and association (ka) rate constants were obtained using Biacore 8K evaluation software.

### Methodology: ELISA Assay of Parental Fab FASEBA Sample

The affinity of parental mouse Fab and chimeric Fab binding to HRGPA-6H was individually determined using ELISA. Microtiter ELISA plates were coated with 10 μg/ml BSA (expression detection) and 2, 1, 0.5, 0.25, 0.125, 0.0625, 0.03125, 0.015625, 0.0078125, 0.0039063 μg/ml antigen protein (binding evaluation) in 100 l CBS at 4° C. overnight, and subsequently incubated with blocking buffer at 37° C. for 1 hour. Then the plates were washed with washing buffer and incubated with diluted 50 ul FASEBA supernatant in 50 ul 0.1% PBST at RT for 2 hours. Next the plates were washed with washing buffer and incubated with 100 μl secondary antibody for 45 minutes. The secondary antibody used Goat Anti-MOUSE IgG (Fab specific) [HRP] for parental mouse Fab and four secondary antibodies (Goat Anti-Human IgG, F(ab′)2 [HRP]; Mouse Anti-Human IgG, F(ab′)2 [HRP]; Goat Anti-Human IgG (H+L) [HRP]; Anti-BSA [HRP]) were used for parental chimeric Fab. After washing, the reaction was developed with 100 l TMB substrate for 10 minutes at room temperature and stopped by adding 50 l of 1 M HCl. The absorbance values were measured at 450 nm using a spectrometer. The HRGPA-6H concentration that OD450 range from 0.5 to 0.8 were selected for subsequent PML library ELISA screening.

### Methodology: Construction of PML Library

According to the parental mouse Fab FASEBA template, totally 65 residues in CDR region were mutate into other 19 desired amino acids using optimal codons for E. coli. DNA oligonucleotide library synthesis was performed on a programmable microarray. The library quality was ensured through NGS and guarantee a minimal coverage of 90%. 44-48 clones were randomly selected from each PML library for expression in E. coli.

### Methodology: FASEBA Screening

44-48 clones were selected from each PML library for expression in 96-deep-well plates. The crude protein secreted in medium was analyzed by ELISA against BSA and HRGPA-6H for the assessment of expression and binding specificity, respectively. Totally 65 PML libraries were tested for binding evaluation and 12 PML libraries were randomly selected for expression detection. Microtiter ELISA plates were coated with 0.0625 μg/ml HRGPA-6H (binding evaluation) and 10 μg/ml BSA (expression detection). The secondary antibody used Goat Anti-MOUSE IgG (Fab specific) [HRP]. The binding ratio was calculated from the mutants OD450 over parental OD450. The mutants that ratio>0.8 were selected for DNA sequencing.

### Results: Affinity Measurement of Parental Antibody and Parental Mouse Fab FASEBA Sample

The affinity of parental antibody with target antigen was measured by Biacore 8K. The result was as shown in Table 17.1. The affinity of parental mouse Fab FASEBA supernatant with target antigen was measured by Biacore 8K. The result were as shown is FIGS. 49-50. Real-time responses were shown, as are the fitting of Biacore experimental data to 1:1 interaction model. According to the curves of non-related FASEBA supernatant (NC) and 2YT medium (Blank), there was non-specific binding for the antigen to chip in low salt buffer and high salt buffer.

TABLE 17.1
Binding kinetics of parental antibody to antigen

| | | ka | kd | KD | Rmax | Chi² |
|---|---|---|---|---|---|---|
| Ligand | Analyte | (1/Ms) | (1/s) | (M) | (RU) | (RU²) |
| HRGPA-6H | KB001-WT-Ab | 4.26E+04 | 8.62E−05 | 2.02E−09 | 62.5 | 1.22E−01 |

### Results: ELISA Assay of Parental Fab FASEBA Sample

The ELISA assay of parental mouse Fab FASEBA was shown in Table 17.2. The concentration of 0.0625 μg/ml HRgpA-6H was selected for further PML library screening. The ELISA assay of parental chimeric Fab FASEBA was shown in Table 17.3. Four secondary antibodies used for parental chimeric Fab FASEBA showed non-specific binding to antigen. The expression validation of parental Fab FASEBA was shown in Table 17.4. The expression level of parental chimeric Fab FASEBA was higher than parental mouse Fab FASEBA.

TABLE 17.2
The ELISA assay between serial diluted antigen
with parental mouse Fab FASEBA sample

| Coating Ag | | | | | | | Parental-Fab |
|---|---|---|---|---|---|---|---|
| concentration | | | | | | | FASEBA |
| (μg/ml) | 2 | 1 | 0.5 | 0.25 | 0.125 | 6.25E−02 | Sample |
| A | 2.035 | 1.941 | 1.757 | 1.584 | 1.169 | 0.755 | Sample #1 |
| B | 1.975 | 2.024 | 1.82 | 1.509 | 1.132 | 0.687 | Sample #2 |

| Coating Ag | | | | | | | Parental-Fab |
|---|---|---|---|---|---|---|---|
| concentration | | | | NC | NC | | FASEBA |
| (μg/ml) | 3.13E−02 | 1.56E−02 | 7.81E−03 | 1 | 2 | Blank | Sample |
| A | 0.429 | 0.245 | 0.167 | 0.065 | 0.078 | 0.071 | Sample #1 |
| B | 0.441 | 0.241 | 0.201 | 0.088 | 0.073 | 0.073 | Sample #2 |

TABLE 17.3
The ELISA assay between serial diluted antigen
with parental chemiric Fab FASEBA sample

| Coating Ag | | | | | | | |
|---|---|---|---|---|---|---|---|
| concentration | | | | | | | |
| (μg/ml) | 2 | 1 | 0.5 | 0.25 | 0.125 | 6.25E−02 | Secondary Ab |
| A | 2.792 | 3.009 | 2.865 | 2.668 | 2.511 | 2.062 | Goat Anti-Human |
| | | | | | | | IgG, F(ab′)₂ |
| B | 3.076 | 2.886 | 2.687 | 2.213 | 1.78 | 1.3 | Anti-BSA (HRP) |
| A | 2.22 | 2.244 | 2.167 | 1.844 | 1.468 | 1.006 | Mouse Anti-Human |
| | | | | | | | IgG, F(ab′)₂ |
| B | 2.274 | 2.328 | 2.315 | 2.066 | 1.891 | 1.414 | Goat Anti-Human |
| | | | | | | | IgG (H + L) |

| Coating Ag | | | | | | | |
|---|---|---|---|---|---|---|---|
| concentration | | | | | | | |
| (μg/ml) | 3.13E−02 | 1.56E−02 | 7.81E−03 | 3.91E−04 | NC | Blank | Secondary Ab |
| A | 1.814 | 1.361 | 0.948 | 0.627 | 0.672 | 1.048 | Goat Anti-Human |
| | | | | | | | IgG, F(ab′)₂ |
| B | 0.776 | 0.522 | 0.3 | 0.165 | 1.088 | 1.853 | Anti-BSA (HRP) |

| Coating Ag | | | | | | | |
|---|---|---|---|---|---|---|---|
| concentration | | | NC | NC | Blank | Blank | |
| (μg/ml) | 3.13E−02 | 1.56E−02 | 1 | 2 | 1 | 2 | Secondary Ab |
| A | 0.59 | 0.36 | 0.552 | 0.288 | 1.13 | 0.992 | Mouse Anti-Human |
| | | | | | | | IgG, F(ab′)₂ |
| B | 1.012 | 0.664 | 1.151 | 0.736 | 1.728 | 1.752 | Goat Anti-Human |
| | | | | | | | IgG (H + L) |

TABLE 17.4
The expression validation of parental Fab FASEBA sample

| | Sample | Sample | NC | NC | Blank | Blank | Parental-Fab |
|---|---|---|---|---|---|---|---|
| | 1 | 2 | 1 | 2 | 1 | 2 | FASEBA Sample |
| A | 1.862 | 1.894 | 3.408 | 3.259 | 0.042 | 0.044 | mouse Fab |
| | | | | | | | FASEBA |
| B | 3.028 | 3.015 | 2.941 | 3.029 | 0.054 | 0.054 | chimeric |
| | | | | | | | Fab FASEBA |

### Results: PML Library Construction

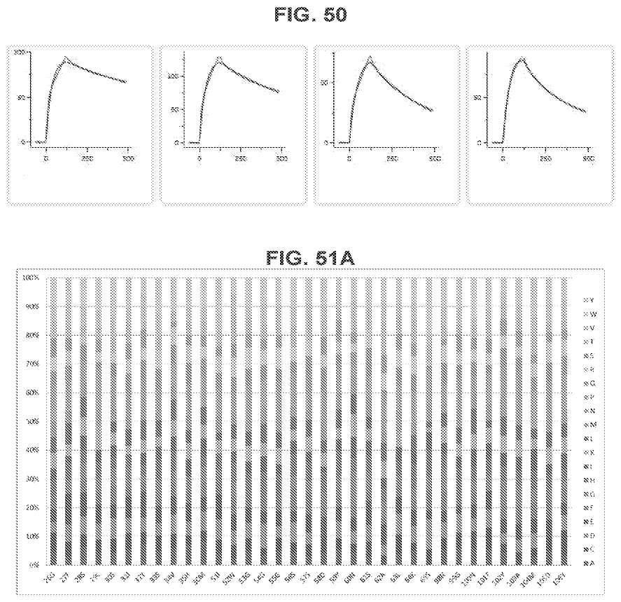

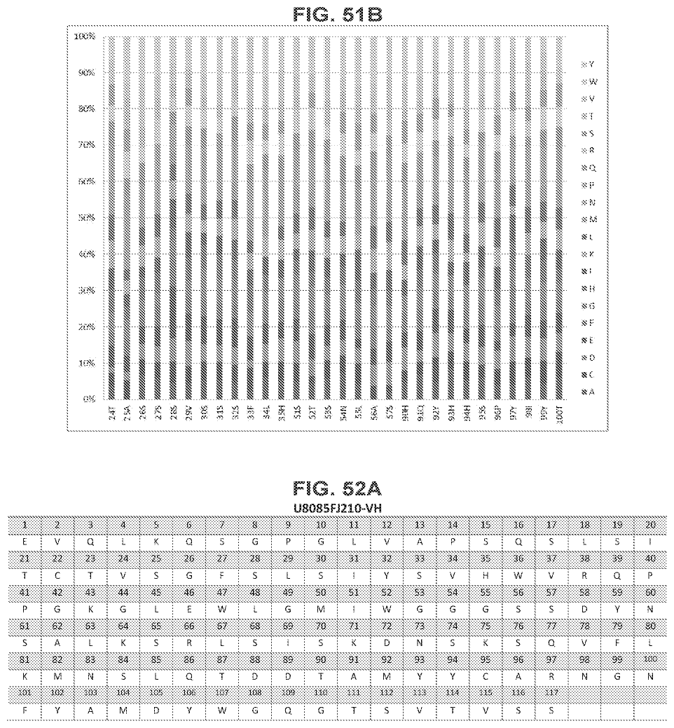

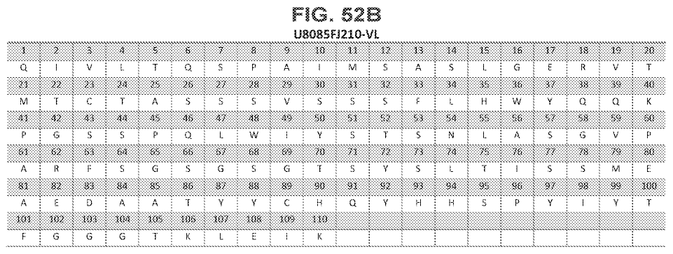

The Precise Mutagenesis Library was synthesized through GenScript advanced oligonucleotide techniques, cloned into U8085FJ210-mouse-Fab-pFASEBA vector as a sub-pool. Each individual PML was generated per residue based on the FASEBA platform with a theoretical diversity at 20. 65 residues in CDR region were selected to mutate (Table 17.5). The library QC was ensured through NGS and results was shown in FIGS. 51A-51B. The parental mouse Fab sequence was as listed in FIGS. 52A-52B.

TABLE 17.5
Residues selected for PML construction

| CDRs | VH-CDR1 | VH-CDR2 | VH-CDR3 | VL-CDR1 | VL-CDR2 | VL-CDR3 |
|---|---|---|---|---|---|---|
| Residue No. | 26-35 | 50-65 | 98-106 | 24-35 | 51-57 | 90-100 |

### Results: FASEBA Screening

From each PML library, more than 44 clones grown and tested for binding activity by ELISA, compared with parental FASEBA supernatant, NC (non-related FASEBA supernatant), blank (2YT medium). The parental was marked in blue and NC was marked in gray. The results were as shown in Tables 17.6-17.7. The ratio was calculated from the mutants OD450 over parental OD450. The mutants that ratio>0.8 were selected for DNA sequencing.

In this non-limiting example, two formats (parental mouse Fab FASEBA and parental chimeric Fab FASEBA) were tested to binding and expression validation. The expression level of parental chimeric Fab FASEBA was higher than parental mouse Fab FASEBA. Due to non-specific binding of HRgpA-6H antigen to chips and four secondary antibodies. From 65 PML libraries, over 2990 individual clones were tested by ELISA. Finally, 802 mutants that binding ratio >0.8 were selected for DNA sequencing.

TABLE 17.6
Fold-change in VH variant binding affinity

| | Sequence Variation | OD450 | |
|---|---|---|---|
| Sample | from WT | nm | Ratio* |
| AHF14445 | S30D | 0.579 | 1.148 |
| AHF14446 | S30D | 0.563 | 1.116 |
| AHF14447 | S30E | 0.567 | 1.124 |
| AHF14448 | S30D | 0.617 | 1.223 |
| AHF14449 | S30N | 0.565 | 1.120 |
| AHF14450 | S61E | 0.603 | 1.222 |
| AHF14451 | S61Q | 0.634 | 1.285 |
| AHF14452 | S61E | 0.567 | 1.149 |
| AHF14453 | S61E | 0.588 | 1.191 |
| AHF14454 | S61D | 0.562 | 1.139 |
| AHF14594 | WT | 0.538 | 1.059 |
| AHF14595 | S28P | 0.545 | 1.009 |
| AHF14596 | WT | 0.568 | 1.052 |
| AHF14597 | S28E | 0.561 | 1.039 |
| AHF14598 | S28E | 0.551 | 1.020 |
| AHF14599 | S30D | 0.528 | 1.047 |
| AHF14600 | S30D | 0.540 | 1.070 |
| AHF14601 | S30D | 0.552 | 1.094 |
| AHF14602 | WT | 0.550 | 1.090 |
| AHF14603 | S30G | 0.533 | 1.056 |
| AHF14604 | S30N | 0.560 | 1.110 |
| AHF14605 | S30Y | 0.530 | 1.051 |
| AHF14606 | S30G | 0.515 | 1.021 |
| AHF14607 | S30A | 0.534 | 1.058 |
| AHF14608 | S30D | 0.547 | 1.084 |
| AHF14609 | WT | 0.531 | 1.053 |
| AHF14610 | I31T | 0.560 | 1.110 |
| AHF14611 | I31V | 0.537 | 1.064 |
| AHF14612 | I31M | 0.522 | 1.035 |
| AHF14613 | I31T | 0.534 | 1.058 |
| AHF14614 | No Sequence | 0.515 | 1.021 |
| AHF14615 | I31V | 0.521 | 1.033 |
| AHF14616 | V34I | 0.891 | 1.081 |
| AHF14617 | WT | 0.924 | 1.121 |
| AHF14618 | V34I | 0.868 | 1.053 |
| AHF14619 | V34I | 0.866 | 1.050 |
| AHF14620 | WT | 0.945 | 1.146 |
| AHF14621 | WT | 0.955 | 1.158 |
| AHF14622 | WT | 0.891 | 1.013 |
| AHF14623 | S56A | 0.891 | 1.013 |
| AHF14624 | WT | 0.898 | 1.021 |
| AHF14625 | S57A | 0.922 | 1.048 |
| AHF14626 | S57V | 0.911 | 1.036 |
| AHF14627 | S57Q | 0.915 | 1.040 |
| AHF14628 | Y59V | 0.830 | 1.049 |
| AHF14629 | S61D | 0.547 | 1.108 |
| AHF14630 | S61T | 0.543 | 1.100 |
| AHF14631 | S61N | 0.537 | 1.088 |
| AHF14632 | S61D | 0.549 | 1.112 |
| AHF14633 | S61E | 0.547 | 1.108 |
| AHF14634 | K64A | 0.890 | 1.076 |
| AHF14635 | K64R | 0.870 | 1.052 |
| AHF14636 | S65T | 0.883 | 1.068 |
| AHF14637 | S65D | 0.866 | 1.047 |
| AHF14638 | S65V | 0.867 | 1.048 |
| AHF14639 | S65D | 0.897 | 1.085 |
| AHF14640 | G99Y | 0.835 | 1.048 |
| AHF14641 | G99S | 0.809 | 1.015 |
| AHF14642 | N100E | 0.886 | 1.046 |
| AHF14643 | A103S | 0.909 | 1.055 |
| AHF14644 | Y106L | 0.791 | 1.029 |
| AHF14645 | Y106T | 0.827 | 1.075 |
| AHF14646 | Y106A | 0.821 | 1.068 |
| AHF14647 | Y106V | 0.795 | 1.034 |
| AHF14648 | Y106R | 0.793 | 1.031 |
| AHF14649 | Y106I | 0.803 | 1.044 |
| AHF14650 | Y106M | 0.794 | 1.033 |
| AHF14651 | Y106M | 0.840 | 1.092 |
| AHF14652 | Y106A | 0.835 | 1.086 |
| AHF14653 | Y106F | 0.815 | 1.060 |
| AHF14654 | Y106V | 0.840 | 1.092 |
| AHF14655 | Y106Q | 0.792 | 1.030 |
| AHF14656 | Y106K | 0.807 | 1.049 |
| AHF14657 | Y106F | 0.856 | 1.113 |
| AHF14658 | Y106S | 0.852 | 1.108 |
| AHF14659 | Y106S | 0.849 | 1.104 |
| AHF14660 | Y106N | 0.792 | 1.030 |
| AHF14661 | Y106W | 0.791 | 1.029 |
| AHF14662 | Y106V | 0.802 | 1.043 |
| AHF14663 | Y106W | 0.809 | 1.052 |
| AHF14664 | Y106W | 0.820 | 1.066 |
| AHF15280 | G26D | 0.470 | 0.925 |
| AHF15281 | G26N | 0.445 | 0.876 |
| AHF15282 | G26D | 0.481 | 0.947 |
| AHF15283 | S28H | 0.511 | 0.946 |
| AHF15284 | S28L | 0.435 | 0.806 |
| AHF15285 | S28G | 0.468 | 0.867 |
| AHF15286 | S28H | 0.526 | 0.974 |
| AHF15287 | S28A | 0.488 | 0.904 |
| AHF15288 | S28P | 0.452 | 0.837 |
| AHF15289 | S28R | 0.458 | 0.848 |
| AHF15290 | S28N | 0.530 | 0.981 |
| AHF15291 | S28L | 0.468 | 0.867 |
| AHF15292 | S28Y | 0.437 | 0.809 |
| AHF15293 | S28G | 0.486 | 0.900 |
| AHF15294 | S28E | 0.494 | 0.915 |
| AHF15295 | S28P | 0.514 | 0.952 |
| AHF15296 | L29P | 0.468 | 0.867 |
| AHF15297 | L29T | 0.502 | 0.930 |
| AHF15298 | L29T | 0.512 | 0.948 |
| AHF15299 | L29V | 0.446 | 0.826 |
| AHF15300 | L29T | 0.501 | 0.928 |
| AHF15301 | S30Q | 0.470 | 0.932 |
| AHF15302 | S30R | 0.481 | 0.953 |
| AHF15303 | S30Y | 0.488 | 0.967 |
| AHF15304 | S30A | 0.499 | 0.989 |
| AHF15305 | S30T | 0.457 | 0.906 |
| AHF15306 | S30K | 0.460 | 0.912 |
| AHF15307 | S30L | 0.468 | 0.928 |
| AHF15308 | S30F | 0.438 | 0.868 |
| AHF15309 | S30K | 0.467 | 0.926 |
| AHF15310 | S30Q | 0.483 | 0.957 |
| AHF15311 | S30K | 0.493 | 0.977 |
| AHF15312 | S30M | 0.437 | 0.866 |
| AHF15313 | S30W | 0.469 | 0.930 |
| AHF15314 | S30H | 0.482 | 0.955 |
| AHF15315 | S30D | 0.448 | 0.888 |
| AHF15316 | S30A | 0.494 | 0.979 |
| AHF15317 | I31Y | 0.449 | 0.890 |
| AHF15318 | I31E | 0.420 | 0.833 |
| AHF15319 | I31F | 0.450 | 0.892 |
| AHF15320 | I31Q | 0.440 | 0.872 |
| AHF15321 | I31Q | 0.439 | 0.870 |
| AHF15322 | I31Y | 0.436 | 0.864 |
| AHF15323 | I31E | 0.458 | 0.908 |
| AHF15324 | I31P | 0.471 | 0.934 |
| AHF15325 | I31L | 0.442 | 0.876 |
| AHF15326 | I31N | 0.429 | 0.850 |
| AHF15327 | I31Y | 0.462 | 0.916 |
| AHF15328 | I31Y | 0.454 | 0.900 |
| AHF15329 | I31E | 0.428 | 0.848 |
| AHF15330 | I31P | 0.428 | 0.848 |
| AHF15331 | I31Y | 0.466 | 0.924 |
| AHF15332 | I31W | 0.459 | 0.910 |
| AHF15333 | I31L | 0.431 | 0.854 |
| AHF15334 | WT | 0.843 | 0.996 |
| AHF15335 | Y32S | 0.717 | 0.847 |
| AHF15336 | Y32S | 0.783 | 0.925 |
| AHF15337 | Y32W | 0.731 | 0.864 |
| AHF15338 | Y32W | 0.718 | 0.848 |
| AHF15339 | Y32S | 0.718 | 0.848 |
| AHF15340 | Y32N | 0.826 | 0.976 |
| AHF15341 | Y32N | 0.775 | 0.916 |
| AHF15342 | Y32W | 0.747 | 0.882 |
| AHF15343 | Y32F | 0.814 | 0.962 |
| AHF15344 | Y32F | 0.807 | 0.953 |
| AHF15345 | V34M | 0.793 | 0.962 |
| AHF15346 | V34M | 0.791 | 0.959 |
| AHF15347 | H35V | 0.706 | 0.856 |
| AHF15348 | WT | 0.733 | 0.830 |
| AHF15349 | I51Q/M92I | 0.722 | 0.817 |
| AHF15350 | I51V | 0.798 | 0.903 |
| AHF15351 | G53A | 0.664 | 0.834 |
| AHF15352 | G53P | 0.754 | 0.947 |
| AHF15353 | G53P | 0.716 | 0.899 |
| AHF15354 | WT | 0.795 | 0.998 |
| AHF15355 | G53S | 0.686 | 0.861 |
| AHF15356 | G53A | 0.741 | 0.930 |
| AHF15357 | G53A | 0.670 | 0.841 |
| AHF15358 | G53A | 0.699 | 0.878 |
| AHF15359 | G55D | 0.697 | 0.866 |
| AHF15360 | G55N | 0.652 | 0.810 |
| AHF15361 | S56G | 0.794 | 0.903 |
| AHF15362 | S56V | 0.761 | 0.865 |
| AHF15363 | S56P | 0.855 | 0.972 |
| AHF15364 | S56T | 0.838 | 0.953 |
| AHF15365 | S56E | 0.772 | 0.878 |
| AHF15366 | S56N | 0.759 | 0.863 |
| AHF15367 | S56L | 0.710 | 0.807 |
| AHF15368 | S56Q | 0.718 | 0.816 |
| AHF15369 | S56P | 0.788 | 0.896 |
| AHF15370 | S56Q | 0.764 | 0.869 |
| AHF15371 | S56G | 0.823 | 0.936 |
| AHF15372 | S56M | 0.809 | 0.920 |
| AHF15373 | S56G | 0.861 | 0.979 |
| AHF15374 | S56A | 0.860 | 0.978 |
| AHF15375 | S56T | 0.844 | 0.960 |
| AHF15376 | Bad Sequence | 0.738 | 0.839 |
| AHF15377 | S56T | 0.853 | 0.970 |
| AHF15378 | S56N | 0.743 | 0.845 |
| AHF15379 | S56P | 0.824 | 0.937 |
| AHF15380 | S56V | 0.775 | 0.881 |
| AHF15381 | S56E | 0.713 | 0.811 |
| AHF15382 | S56G | 0.819 | 0.931 |
| AHF15383 | S56V | 0.777 | 0.883 |
| AHF15384 | S56Q | 0.728 | 0.828 |
| AHF15385 | S56A | 0.820 | 0.932 |
| AHF15386 | S56Q | 0.775 | 0.881 |
| AHF15387 | S56V | 0.760 | 0.864 |
| AHF15388 | S57E | 0.777 | 0.883 |
| AHF15389 | S57R | 0.796 | 0.905 |
| AHF15390 | S57H | 0.800 | 0.910 |
| AHF15391 | S57M | 0.748 | 0.850 |
| AHF15392 | S57P | 0.789 | 0.897 |
| AHF15393 | S57Q | 0.863 | 0.981 |
| AHF15394 | S57P | 0.801 | 0.911 |
| AHF15395 | S57E | 0.781 | 0.888 |
| AHF15396 | S57P | 0.747 | 0.849 |
| AHF15397 | S57E | 0.723 | 0.822 |
| AHF15398 | S57T | 0.843 | 0.958 |
| AHF15399 | S57T | 0.849 | 0.965 |
| AHF15400 | S57E | 0.769 | 0.874 |
| AHF15401 | S57V | 0.869 | 0.988 |
| AHF15402 | S57Q | 0.836 | 0.951 |
| AHF15403 | S57P | 0.724 | 0.823 |
| AHF15404 | WT | 0.785 | 0.992 |
| AHF15405 | D58S | 0.646 | 0.817 |
| AHF15406 | D58G | 0.778 | 0.984 |
| AHF15407 | Y59K | 0.648 | 0.819 |
| AHF15408 | Y59T | 0.744 | 0.941 |
| AHF15409 | Y59L | 0.758 | 0.958 |
| AHF15410 | Y59R | 0.633 | 0.800 |
| AHF15411 | Y59V | 0.790 | 0.999 |
| AHF15412 | Y59M/S57I | 0.701 | 0.886 |
| AHF15413 | Y59L | 0.775 | 0.980 |
| AHF15414 | Y59L | 0.752 | 0.951 |
| AHF15415 | Y59F | 0.668 | 0.845 |
| AHF15416 | Y59L | 0.691 | 0.874 |
| AHF15417 | WT | 0.698 | 0.882 |
| AHF15418 | N60S | 0.452 | 0.916 |
| AHF15419 | N60P | 0.440 | 0.892 |
| AHF15420 | N60T | 0.406 | 0.823 |
| AHF15421 | N60P | 0.428 | 0.867 |
| AHF15422 | WT | 0.476 | 0.965 |
| AHF15423 | N60Q | 0.447 | 0.906 |
| AHF15424 | N60Q | 0.402 | 0.815 |
| AHF15425 | S61W | 0.435 | 0.881 |
| AHF15426 | S61Y | 0.423 | 0.857 |
| AHF15427 | S61R | 0.438 | 0.888 |
| AHF15428 | S61G | 0.460 | 0.932 |
| AHF15429 | S61F | 0.432 | 0.875 |
| AHF15430 | S61W | 0.429 | 0.869 |
| AHF15431 | S61M | 0.493 | 0.999 |
| AHF15432 | S61G | 0.476 | 0.965 |
| AHF15433 | S61C | 0.431 | 0.873 |
| AHF15434 | S61M | 0.479 | 0.971 |
| AHF15435 | S61K | 0.490 | 0.993 |
| AHF15436 | S61H | 0.469 | 0.950 |
| AHF15437 | S61F | 0.473 | 0.958 |
| AHF15438 | S61L | 0.448 | 0.908 |
| AHF15439 | S61D | 0.472 | 0.956 |
| AHF15440 | S61T | 0.447 | 0.906 |
| AHF15441 | A62N | 0.721 | 0.825 |
| AHF15442 | A62N | 0.799 | 0.914 |
| AHF15443 | A62M | 0.770 | 0.881 |
| AHF15444 | A62G | 0.813 | 0.930 |
| AHF15445 | A62Q | 0.737 | 0.843 |
| AHF15446 | A62H | 0.819 | 0.937 |
| AHF15447 | A62V | 0.742 | 0.849 |
| AHF15448 | Bad Sequence | 0.824 | 0.943 |
| AHF15449 | A62V | 0.757 | 0.866 |
| AHF15450 | A62D | 0.853 | 0.976 |
| AHF15451 | A62L | 0.812 | 0.929 |
| AHF15452 | A62D | 0.851 | 0.974 |
| AHF15453 | A62T | 0.839 | 0.960 |
| AHF15454 | A62T | 0.812 | 0.929 |
| AHF15455 | A62F | 0.843 | 0.965 |
| AHF15456 | A62I | 0.702 | 0.803 |
| AHF15457 | Bad Sequence | 0.759 | 0.868 |
| AHF15458 | Bad Sequence | 0.786 | 0.899 |
| AHF15459 | A62R | 0.733 | 0.839 |
| AHF15460 | A62R | 0.847 | 0.969 |
| AHF15461 | A62Q | 0.869 | 0.994 |
| AHF15462 | A62T | 0.782 | 0.895 |
| AHF15463 | A62E | 0.854 | 0.977 |
| AHF15464 | A62F | 0.733 | 0.839 |
| AHF15465 | A62I | 0.808 | 0.924 |
| AHF15466 | A62M | 0.735 | 0.841 |
| AHF15467 | A62R | 0.809 | 0.926 |
| AHF15468 | A62T | 0.827 | 0.946 |
| AHF15469 | A62T | 0.856 | 0.979 |
| AHF15470 | WT | 0.867 | 0.992 |
| AHF15471 | A62H | 0.834 | 0.954 |
| AHF15472 | A62H | 0.844 | 0.966 |
| AHF15473 | A62T | 0.839 | 0.960 |
| AHF15474 | A62G | 0.785 | 0.898 |
| AHF15475 | L63V | 0.784 | 0.897 |
| AHF15476 | L63V | 0.770 | 0.881 |
| AHF15477 | L63Y | 0.836 | 0.957 |
| AHF15478 | L63Y | 0.846 | 0.968 |
| AHF15479 | L63Y | 0.801 | 0.916 |
| AHF15480 | L63H | 0.818 | 0.936 |
| AHF15481 | L63V | 0.778 | 0.890 |
| AHF15482 | L63H | 0.843 | 0.965 |
| AHF15483 | L63H | 0.822 | 0.941 |
| AHF15484 | L63M | 0.800 | 0.915 |
| AHF15485 | L63F | 0.743 | 0.850 |
| AHF15486 | K64I | 0.743 | 0.898 |
| AHF15487 | K64V | 0.715 | 0.865 |
| AHF15488 | K64M | 0.799 | 0.966 |
| AHF15489 | K64G | 0.806 | 0.975 |
| AHF15490 | K64H | 0.752 | 0.909 |
| AHF15491 | K64S | 0.808 | 0.977 |
| AHF15492 | K64V | 0.714 | 0.863 |
| AHF15493 | WT | 0.803 | 0.971 |
| AHF15494 | K64I | 0.756 | 0.914 |
| AHF15495 | K64M | 0.757 | 0.915 |
| AHF15496 | K64M | 0.780 | 0.943 |
| AHF15497 | K64I | 0.715 | 0.865 |
| AHF15498 | K64Y | 0.763 | 0.923 |
| AHF15499 | K64H | 0.788 | 0.953 |
| AHF15500 | K64H | 0.806 | 0.975 |
| AHF15501 | K64H | 0.730 | 0.883 |
| AHF15502 | K64G | 0.811 | 0.981 |
| AHF15503 | K64Q | 0.789 | 0.954 |
| AHF15504 | K64D | 0.781 | 0.944 |
| AHF15505 | K64L | 0.664 | 0.803 |
| AHF15506 | K64M | 0.784 | 0.948 |
| AHF15507 | K64E | 0.789 | 0.954 |
| AHF15508 | S65H | 0.823 | 0.995 |
| AHF15509 | S65Y | 0.724 | 0.875 |
| AHF15510 | S65P | 0.814 | 0.984 |
| AHF15511 | S65R | 0.794 | 0.960 |
| AHF15512 | S65F | 0.740 | 0.895 |
| AHF15513 | S65L | 0.728 | 0.880 |
| AHF15514 | S65G | 0.825 | 0.998 |
| AHF15515 | S65H | 0.767 | 0.927 |
| AHF15516 | S65T | 0.759 | 0.918 |
| AHF15517 | G99F | 0.771 | 0.967 |
| AHF15518 | G99S | 0.769 | 0.965 |
| AHF15519 | G99S | 0.739 | 0.927 |
| AHF15520 | G99N | 0.639 | 0.802 |
| AHF15521 | G99N | 0.698 | 0.876 |
| AHF15522 | G99A | 0.740 | 0.928 |
| AHF15523 | G99S | 0.770 | 0.966 |
| AHF15524 | G99Y | 0.769 | 0.965 |
| AHF15525 | G99C | 0.776 | 0.974 |
| AHF15526 | N100D | 0.820 | 0.968 |
| AHF15527 | N100P | 0.757 | 0.894 |
| AHF15528 | N100I | 0.796 | 0.940 |
| AHF15529 | N100S | 0.799 | 0.943 |
| AHF15530 | N100L | 0.817 | 0.965 |
| AHF15531 | N100A | 0.809 | 0.955 |
| AHF15532 | Bad Sequence | 0.683 | 0.806 |
| AHF15533 | N100D | 0.784 | 0.926 |
| AHF15534 | N100F | 0.779 | 0.920 |
| AHF15535 | N100C | 0.688 | 0.812 |
| AHF15536 | N100G | 0.769 | 0.908 |
| AHF15537 | N100E | 0.823 | 0.972 |
| AHF15538 | N100T | 0.756 | 0.893 |
| AHF15539 | N100L | 0.736 | 0.869 |
| AHF15540 | N100K | 0.801 | 0.946 |
| AHF15541 | N100I | 0.833 | 0.983 |
| AHF15542 | N100L | 0.789 | 0.932 |
| AHF15543 | N100D | 0.841 | 0.993 |
| AHF15544 | N100S | 0.844 | 0.996 |
| AHF15545 | N100R | 0.788 | 0.930 |
| AHF15546 | N100S | 0.805 | 0.950 |
| AHF15547 | N100Q | 0.846 | 0.999 |
| AHF15548 | N100D | 0.842 | 0.994 |
| AHF15549 | N100Y | 0.757 | 0.894 |
| AHF15550 | N100Y | 0.784 | 0.926 |
| AHF15551 | N100G | 0.814 | 0.961 |
| AHF15552 | N100Y | 0.699 | 0.825 |
| AHF15553 | N100K | 0.791 | 0.934 |
| AHF15554 | N100T | 0.783 | 0.924 |
| AHF15555 | F101S | 0.678 | 0.800 |
| AHF15556 | F101Y | 0.690 | 0.815 |
| AHF15557 | F101S | 0.725 | 0.856 |
| AHF15558 | F101W | 0.825 | 0.974 |
| AHF15559 | F101M | 0.755 | 0.891 |
| AHF15560 | F101W | 0.828 | 0.978 |
| AHF15561 | F101Y | 0.718 | 0.848 |
| AHF15562 | F101Y | 0.738 | 0.871 |
| AHF15563 | F101L | 0.681 | 0.804 |
| AHF15564 | F101Y | 0.713 | 0.842 |
| AHF15565 | F101Y | 0.732 | 0.864 |
| AHF15566 | F101W | 0.802 | 0.947 |
| AHF15567 | A103G | 0.815 | 0.945 |
| AHF15568 | Bad Sequence | 0.850 | 0.986 |
| AHF15569 | A103V | 0.849 | 0.985 |
| AHF15570 | A103V | 0.807 | 0.936 |
| AHF15571 | A103G | 0.804 | 0.933 |
| AHF15572 | A103L | 0.783 | 0.908 |
| AHF15573 | A103 | 0.850 | 0.986 |
| AHF15574 | A103M | 0.811 | 0.941 |
| AHF15575 | A103K | 0.723 | 0.839 |
| AHF15576 | A103I | 0.695 | 0.806 |
| AHF15577 | A103G | 0.755 | 0.876 |
| AHF15578 | A103C | 0.770 | 0.893 |
| AHF15579 | A103I | 0.693 | 0.804 |
| AHF15580 | M104L | 0.786 | 0.904 |
| AHF15581 | WT | 0.831 | 0.956 |
| AHF15582 | Y106Q | 0.768 | 0.999 |
| AHF15583 | Y106E | 0.687 | 0.893 |
| AHF15584 | Y106I | 0.730 | 0.949 |
| AHF15585 | Y106E | 0.705 | 0.917 |
| AHF15586 | Y106H | 0.722 | 0.939 |
| AHF15587 | Y106W | 0.733 | 0.953 |
| AHF15588 | Y106D | 0.675 | 0.878 |
| AHF15589 | Y106E | 0.721 | 0.938 |
| AHF15590 | Y106I | 0.728 | 0.947 |
| AHF15591 | Bad Sequence | 0.761 | 0.990 |
| AHF15592 | Y106E | 0.686 | 0.892 |
| AHF15593 | Y106W | 0.748 | 0.973 |
| AHF15594 | Y106I | 0.764 | 0.993 |
| AHF15595 | Y106K | 0.767 | 0.997 |
| AHF15596 | M104V | 0.654 | 0.850 |

*Ratio = OD(Variant)/OD(WT) = fold-change in affinity, as characterized by ELISA

TABLE 17.7
Fold-change in VL variant binding affinity

| | Sequence Variation | OD450 | |
|---|---|---|---|
| Sample | from WT | nm | Ratio* |
| AHF14455 | T100E | 0.674 | 1.287 |
| AHF14456 | T100A | 0.630 | 1.203 |
| =AHF14457 | T100A | 0.636 | 1.215 |
| AHF14458 | T100S | 0.664 | 1.268 |
| AHF14459 | T100K | 0.625 | 1.194 |
| AHF14460 | T100D | 0.645 | 1.232 |
| AHF14461 | T100A | 0.625 | 1.194 |
| AHF14462 | T100H | 0.631 | 1.205 |
| AHF14463 | T100A | 0.631 | 1.205 |
| AHF14464 | T100S | 0.625 | 1.194 |
| AHF14665 | T24N | 0.800 | 1.040 |
| AHF14666 | T24S | 0.822 | 1.069 |
| AHF14667 | T24E | 0.810 | 1.053 |
| AHF14668 | WT | 0.806 | 1.048 |
| AHF14669 | T24S | 0.798 | 1.038 |
| AHF14670 | T24M | 0.797 | 1.036 |
| AHF14671 | T24S | 0.812 | 1.056 |
| AHF14672 | T24K | 0.797 | 1.036 |
| AHF14673 | WT | 0.809 | 1.052 |
| AHF14674 | T24E | 0.852 | 1.108 |
| AHF14675 | T24H | 0.795 | 1.034 |
| AHF14676 | WT | 0.796 | 1.035 |
| AHF14677 | WT | 0.831 | 1.061 |
| AHF14678 | A25F | 0.818 | 1.045 |
| AHF14679 | A25T | 0.823 | 1.051 |
| AHF14680 | WT | 0.835 | 1.066 |
| AHF14681 | A25T | 0.801 | 1.023 |
| AHF14682 | A25F | 0.827 | 1.056 |
| AHF14683 | No Sequence | 0.889 | 1.135 |
| AHF14684 | S26N | 0.823 | 1.051 |
| AHF14685 | WT | 0.809 | 1.033 |
| AHF14686 | S26D | 0.821 | 1.049 |
| AHF14687 | WT | 0.829 | 1.059 |
| AHF14688 | WT | 0.823 | 1.051 |
| AHF14689 | S27K | 0.783 | 1.043 |
| AHF14690 | S27T | 0.783 | 1.043 |
| AHF14691 | S27A | 0.816 | 1.087 |
| AHF14692 | S27A | 0.808 | 1.077 |
| AHF14693 | S28P | 0.808 | 1.077 |
| AHF14694 | S28W | 0.817 | 1.089 |
| AHF14695 | S28K | 0.801 | 1.067 |
| AHF14696 | S28G | 0.818 | 1.090 |
| AHF14697 | S28V | 0.786 | 1.047 |
| AHF14698 | S30F | 0.911 | 1.057 |
| AHF14699 | S30G | 0.916 | 1.063 |
| AHF14700 | S31P | 0.870 | 1.030 |
| AHF14701 | S31E | 0.903 | 1.069 |
| AHF14702 | S31Y | 0.849 | 1.005 |
| AHF14703 | S31G | 0.864 | 1.022 |
| AHF14704 | S31G | 0.899 | 1.064 |
| AHF14705 | S31F | 0.854 | 1.011 |
| AHF14706 | S31P | 0.875 | 1.036 |
| AHF14707 | S31T | 0.859 | 1.017 |
| AHF14708 | S31P | 0.856 | 1.013 |
| AHF14709 | S31N | 0.869 | 1.028 |
| AHF14710 | WT | 0.916 | 1.084 |
| AHF14711 | WT | 0.872 | 1.032 |
| AHF14712 | WT | 0.852 | 1.008 |
| AHF14713 | S51G | 0.915 | 1.046 |
| AHF14714 | T52A | 0.923 | 1.172 |
| AHF14715 | T52A | 0.848 | 1.077 |
| AHF14716 | T52S | 0.833 | 1.058 |
| AHF14717 | T52A | 0.851 | 1.081 |
| AHF14718 | S53N | 0.838 | 1.064 |
| AHF14719 | S53H | 0.827 | 1.050 |
| AHF14720 | S53Q | 0.835 | 1.060 |
| AHF14721 | S53K | 0.841 | 1.068 |
| AHF14722 | N54Q | 0.868 | 1.108 |
| AHF14723 | WT | 0.825 | 1.053 |
| AHF14724 | N54R | 0.876 | 1.118 |
| AHF14725 | WT | 0.828 | 1.057 |
| AHF14726 | N54S | 0.835 | 1.066 |
| AHF14727 | L55N | 0.816 | 1.041 |
| AHF14728 | L55N | 0.815 | 1.040 |
| AHF14729 | L55P | 0.827 | 1.056 |
| AHF14730 | WT | 0.842 | 1.032 |
| AHF14731 | S57N | 0.857 | 1.051 |
| AHF14732 | S57R | 0.845 | 1.036 |
| AHF14733 | S57T | 0.843 | 1.034 |
| AHF14734 | S57K | 0.841 | 1.031 |
| AHF14735 | S57E | 0.843 | 1.034 |
| AHF14736 | S57K | 0.841 | 1.031 |
| AHF14737 | S57P | 0.858 | 1.052 |
| AHF14738 | S57Q | 0.858 | 1.052 |
| AHF14739 | S57G | 0.873 | 1.071 |
| AHF14740 | No Sequence | 0.880 | 1.079 |
| AHF14741 | S57G | 0.825 | 1.012 |
| AHF14742 | S57Q | 0.838 | 1.028 |
| AHF14743 | S57K | 0.840 | 1.030 |
| AHF14744 | S57K | 0.864 | 1.059 |
| AHF14745 | S57R | 0.849 | 1.041 |
| AHF14746 | S57K | 0.846 | 1.037 |
| AHF14747 | S57K | 0.879 | 1.078 |
| AHF14748 | WT | 0.868 | 1.008 |
| AHF14749 | H90W | 0.913 | 1.060 |
| AHF14750 | S95Q | 0.931 | 1.014 |
| AHF14751 | S95Q | 0.936 | 1.019 |
| AHF14752 | S95Q | 0.927 | 1.009 |
| AHF14753 | Y97E | 0.793 | 1.005 |
| AHF14754 | T100Q | 0.586 | 1.119 |
| AHF14755 | T100G | 0.603 | 1.152 |
| AHF14756 | T100Y | 0.546 | 1.043 |
| AHF14757 | T100F | 0.552 | 1.054 |
| AHF14758 | T100R | 0.616 | 1.177 |
| AHF14759 | T100H | 0.597 | 1.140 |
| AHF14760 | T100Q | 0.596 | 1.138 |
| AHF14761 | T100W | 0.585 | 1.117 |
| AHF14762 | T100Q | 0.604 | 1.154 |
| AHF14763 | T100G | 0.597 | 1.140 |
| AHF14764 | T100E | 0.613 | 1.171 |
| AHF14765 | T100M | 0.589 | 1.125 |
| AHF14766 | T100E | 0.621 | 1.186 |
| AHF14767 | T100L | 0.587 | 1.121 |
| AHF14768 | T100A | 0.616 | 1.177 |
| AHF14769 | T100S | 0.602 | 1.150 |
| AHF14770 | T100L | 0.566 | 1.081 |
| AHF15597 | T24I | 0.741 | 0.964 |
| AHF15598 | T24K/R18S | 0.761 | 0.990 |
| AHF15599 | T24L | 0.753 | 0.979 |
| AHF15600 | T24A | 0.757 | 0.984 |
| AHF15601 | T24L | 0.752 | 0.978 |
| AHF15602 | T24Q | 0.765 | 0.995 |
| AHF15603 | T24N/G67D | 0.755 | 0.982 |
| AHF15604 | T24G | 0.686 | 0.892 |
| AHF15605 | T24M | 0.750 | 0.975 |
| AHF15606 | T24K | 0.734 | 0.954 |
| AHF15607 | Bad Sequence | 0.752 | 0.978 |
| AHF15608 | T24N | 0.715 | 0.930 |
| AHF15609 | T24G | 0.735 | 0.956 |
| AHF15610 | T24L | 0.667 | 0.867 |
| AHF15611 | T24S | 0.704 | 0.915 |
| AHF15612 | A25Y | 0.755 | 0.964 |
| AHF15613 | A25F | 0.775 | 0.990 |
| AHF15614 | A25G | 0.668 | 0.853 |
| AHF15615 | A25P | 0.742 | 0.948 |
| AHF15616 | A25Y | 0.757 | 0.967 |
| AHF15617 | A25V | 0.768 | 0.981 |
| AHF15618 | A25E | 0.655 | 0.837 |
| AHF15619 | A25G | 0.699 | 0.893 |
| AHF15620 | A25P | 0.766 | 0.978 |
| AHF15621 | A25G | 0.673 | 0.860 |
| AHF15622 | A25P | 0.749 | 0.957 |
| AHF15623 | S26G | 0.730 | 0.932 |
| AHF15624 | S26N | 0.749 | 0.957 |
| AHF15625 | S26P | 0.644 | 0.822 |
| AHF15626 | S26T | 0.761 | 0.972 |
| AHF15627 | S26P | 0.677 | 0.865 |
| AHF15628 | S27H | 0.664 | 0.885 |
| AHF15629 | S27T | 0.702 | 0.935 |
| AHF15630 | S27N | 0.675 | 0.899 |
| AHF15631 | S27N | 0.724 | 0.965 |
| AHF15632 | S27A | 0.714 | 0.951 |
| AHF15633 | S27K | 0.745 | 0.993 |
| AHF15634 | S27Y | 0.617 | 0.822 |
| AHF15635 | S27T | 0.743 | 0.990 |
| AHF15636 | S27K | 0.697 | 0.929 |
| AHF15637 | S27H | 0.630 | 0.839 |
| AHF15638 | S27L | 0.649 | 0.865 |
| AHF15639 | S27H | 0.726 | 0.967 |
| AHF15640 | S27A | 0.722 | 0.962 |
| AHF15641 | S27V | 0.645 | 0.859 |
| AHF15642 | S27V | 0.646 | 0.861 |
| AHF15643 | Bad Sequence | 0.640 | 0.853 |
| AHF15644 | S27N | 0.640 | 0.853 |
| AHF15645 | S27T | 0.737 | 0.982 |
| AHF15646 | S27W | 0.601 | 0.801 |
| AHF15647 | S27K/S68Y | 0.736 | 0.981 |
| AHF15648 | S27M | 0.709 | 0.945 |
| AHF15649 | S27T | 0.710 | 0.946 |
| AHF15650 | S28F | 0.674 | 0.898 |
| AHF15651 | S28F | 0.721 | 0.961 |
| AHF15652 | S28M | 0.706 | 0.941 |
| AHF15653 | S28T/S12R | 0.747 | 0.995 |
| AHF15654 | S28V | 0.731 | 0.974 |
| AHF15655 | Bad Sequence | 0.716 | 0.954 |
| AHF15656 | S28P | 0.726 | 0.967 |
| AHF15657 | S28W | 0.745 | 0.993 |
| AHF15658 | S28H | 0.719 | 0.958 |
| AHF15659 | S28N | 0.743 | 0.990 |
| AHF15660 | S28F | 0.740 | 0.986 |
| AHF15661 | S28V | 0.717 | 0.955 |
| AHF15662 | S28D | 0.745 | 0.993 |
| AHF15663 | Bad Sequence | 0.690 | 0.919 |
| AHF15664 | Bad Sequence | 0.714 | 0.951 |
| AHF15665 | S28M | 0.675 | 0.899 |
| AHF15666 | S28F | 0.678 | 0.903 |
| AHF15667 | S28D | 0.722 | 0.962 |
| AHF15668 | V29I | 0.853 | 0.990 |
| AHF15669 | V29L | 0.751 | 0.871 |
| AHF15670 | S30H | 0.808 | 0.937 |
| AHF15671 | Bad Sequence | 0.841 | 0.976 |
| AHF15672 | S30G | 0.858 | 0.995 |
| AHF15673 | S30R | 0.778 | 0.903 |
| AHF15674 | S30T | 0.838 | 0.972 |
| AHF15675 | S30E | 0.813 | 0.943 |
| AHF15676 | S30D | 0.702 | 0.814 |
| AHF15677 | S30R | 0.793 | 0.920 |
| AHF15678 | S30W | 0.823 | 0.955 |
| AHF15679 | S30T | 0.817 | 0.948 |
| AHF15680 | S30L | 0.813 | 0.943 |
| AHF15681 | S30D | 0.723 | 0.839 |
| AHF15682 | S30I | 0.860 | 0.998 |
| AHF15683 | S30R | 0.802 | 0.930 |
| AHF15684 | S30M | 0.818 | 0.949 |
| AHF15685 | S30H | 0.747 | 0.867 |
| AHF15686 | S30K | 0.770 | 0.893 |
| AHF15687 | S31F | 0.688 | 0.814 |
| AHF15688 | S31N | 0.791 | 0.936 |
| AHF15689 | S31T | 0.740 | 0.876 |
| AHF15690 | S31Q | 0.830 | 0.982 |
| AHF15691 | Bad Sequence | 0.774 | 0.916 |
| AHF15692 | S31E | 0.798 | 0.944 |
| AHF15693 | S31N | 0.749 | 0.886 |
| AHF15694 | S31F | 0.693 | 0.820 |
| AHF15695 | S31N | 0.789 | 0.934 |
| AHF15696 | S31W | 0.808 | 0.956 |
| AHF15697 | WT | 0.697 | 0.825 |
| AHF15698 | S31D | 0.762 | 0.902 |
| AHF15699 | S31L | 0.742 | 0.878 |
| AHF15700 | S31Q | 0.745 | 0.882 |
| AHF15701 | S31G | 0.824 | 0.975 |
| AHF15702 | S31L | 0.739 | 0.875 |
| AHF15703 | S31N | 0.769 | 0.910 |
| AHF15704 | S31L | 0.745 | 0.882 |
| AHF15705 | S31G | 0.776 | 0.918 |
| AHF15706 | S31T | 0.782 | 0.925 |
| AHF15707 | S31Q | 0.782 | 0.925 |
| AHF15708 | S31L | 0.691 | 0.818 |
| AHF15709 | S32A | 0.748 | 0.885 |
| AHF15710 | S32G | 0.759 | 0.898 |
| AHF15711 | S32A | 0.805 | 0.953 |
| AHF15712 | S32Y | 0.753 | 0.891 |
| AHF15713 | S32P | 0.744 | 0.880 |
| AHF15714 | S32Q | 0.716 | 0.847 |
| AHF15715 | S32W | 0.649 | 0.856 |
| AHF15716 | WT | 0.729 | 0.962 |
| AHF15717 | F33N | 0.640 | 0.844 |
| AHF15718 | F33H | 0.736 | 0.971 |
| AHF15719 | L34V | 0.641 | 0.846 |
| AHF15720 | L34M | 0.736 | 0.971 |
| AHF15721 | L34M | 0.741 | 0.978 |
| AHF15722 | L34V | 0.634 | 0.836 |
| AHF15723 | L34I | 0.670 | 0.884 |
| AHF15724 | WT | 0.710 | 0.811 |
| AHF15725 | S51G | 0.777 | 0.888 |
| AHF15726 | S51G | 0.851 | 0.973 |
| AHF15727 | T52K | 0.681 | 0.865 |
| AHF15728 | T52A | 0.782 | 0.993 |
| AHF15729 | T52R | 0.724 | 0.919 |
| AHF15730 | T52L | 0.769 | 0.977 |
| AHF15731 | T52R | 0.681 | 0.865 |
| AHF15732 | WT | 0.767 | 0.974 |
| AHF15733 | T52R | 0.685 | 0.870 |
| AHF15734 | WT | 0.705 | 0.895 |
| AHF15735 | T52M | 0.662 | 0.841 |
| AHF15736 | S53D | 0.775 | 0.984 |
| AHF15737 | S53C | 0.697 | 0.885 |
| AHF15738 | S53H | 0.777 | 0.987 |
| AHF15739 | S53A | 0.784 | 0.996 |
| AHF15740 | S53R | 0.637 | 0.809 |
| AHF15741 | S53W | 0.727 | 0.923 |
| AHF15742 | S53L | 0.639 | 0.811 |
| AHF15743 | S53Y | 0.776 | 0.985 |
| AHF15744 | S53Y | 0.781 | 0.992 |
| AHF15745 | S53Q | 0.751 | 0.954 |
| AHF15746 | S53E | 0.766 | 0.973 |
| AHF15747 | S53L | 0.636 | 0.808 |
| AHF15748 | S53Q | 0.726 | 0.922 |
| AHF15749 | S53N | 0.707 | 0.898 |
| AHF15750 | S53V | 0.702 | 0.891 |
| AHF15751 | Bad Sequence | 0.668 | 0.853 |
| AHF15752 | N54S | 0.732 | 0.934 |
| AHF15753 | N54I | 0.755 | 0.964 |
| AHF15754 | Bad Sequence | 0.684 | 0.873 |
| AHF15755 | Bad Sequence | 0.746 | 0.952 |
| AHF15756 | N54K | 0.727 | 0.928 |
| AHF15757 | N54W | 0.646 | 0.825 |
| AHF15758 | N54E | 0.744 | 0.950 |
| AHF15759 | N54D | 0.725 | 0.925 |
| AHF15760 | N54I | 0.772 | 0.985 |
| AHF15761 | L34M | 0.714 | 0.911 |
| AHF15762 | N54F | 0.765 | 0.976 |
| AHF15763 | N54I | 0.754 | 0.962 |
| AHF15764 | N54E | 0.740 | 0.944 |
| AHF15765 | N54L | 0.774 | 0.988 |
| AHF15766 | Bad Sequence | 0.766 | 0.978 |
| AHF15767 | N54S | 0.776 | 0.990 |
| AHF15768 | N54A | 0.776 | 0.990 |
| AHF15769 | L55Q | 0.711 | 0.907 |
| AHF15770 | L55S | 0.672 | 0.858 |
| AHF15771 | L55P | 0.764 | 0.975 |
| AHF15772 | L55N | 0.777 | 0.992 |
| AHF15773 | L55T | 0.727 | 0.928 |
| AHF15774 | L55H | 0.695 | 0.887 |
| AHF15775 | L55H | 0.731 | 0.933 |
| AHF15776 | L55Q | 0.692 | 0.883 |
| AHF15777 | L55Q | 0.684 | 0.873 |
| AHF15778 | L55K | 0.769 | 0.981 |
| AHF15779 | L55V | 0.706 | 0.901 |
| AHF15780 | L55P | 0.724 | 0.924 |
| AHF15781 | L55R/S51G | 0.748 | 0.955 |
| AHF15782 | L55Q | 0.662 | 0.845 |
| AHF15783 | L55H | 0.640 | 0.817 |
| AHF15784 | L55S | 0.655 | 0.836 |
| AHF15785 | A56T | 0.731 | 0.896 |
| AHF15786 | A56N | 0.712 | 0.873 |
| AHF15787 | A56Q | 0.705 | 0.865 |
| AHF15788 | A56H | 0.704 | 0.863 |
| AHF15789 | A56R | 0.728 | 0.893 |
| AHF15790 | A56Y | 0.673 | 0.825 |
| AHF15791 | Bad Sequence | 0.733 | 0.899 |
| AHF15792 | A56Y | 0.712 | 0.873 |
| AHF15793 | A56H | 0.699 | 0.857 |
| AHF15794 | A56V | 0.794 | 0.974 |
| AHF15795 | A56Y | 0.726 | 0.890 |
| AHF15796 | A56S | 0.772 | 0.947 |
| AHF15797 | A56H | 0.759 | 0.931 |
| AHF15798 | A56S | 0.751 | 0.921 |
| AHF15799 | A56R | 0.734 | 0.900 |
| AHF15800 | A56H | 0.739 | 0.906 |
| AHF15801 | A56S | 0.774 | 0.949 |
| AHF15802 | A56N | 0.775 | 0.950 |
| AHF15803 | A56P | 0.748 | 0.917 |
| AHF15804 | A56V | 0.795 | 0.975 |
| AHF15805 | A56Y | 0.696 | 0.853 |
| AHF15806 | A56M | 0.802 | 0.983 |
| AHF15807 | A56Y | 0.698 | 0.856 |
| AHF15808 | A56R | 0.751 | 0.921 |
| AHF15809 | A56M | 0.701 | 0.860 |
| AHF15810 | S57A | 0.806 | 0.988 |
| AHF15811 | S57L | 0.781 | 0.958 |
| AHF15812 | S57F | 0.785 | 0.963 |
| AHF15813 | S57F | 0.787 | 0.965 |
| AHF15814 | S57I | 0.791 | 0.970 |
| AHF15815 | S57Q | 0.658 | 0.807 |
| AHF15816 | S57N | 0.813 | 0.997 |
| AHF15817 | S57K | 0.712 | 0.873 |
| AHF15818 | S57W | 0.784 | 0.961 |
| AHF15819 | S57F | 0.797 | 0.977 |
| AHF15820 | S57E | 0.751 | 0.921 |
| AHF15821 | S57R | 0.746 | 0.915 |
| AHF15822 | S57M | 0.745 | 0.914 |
| AHF15823 | H90L | 0.785 | 0.911 |
| AHF15824 | H90W | 0.763 | 0.886 |
| AHF15825 | H90L | 0.694 | 0.806 |
| AHF15826 | Q91M | 0.757 | 0.879 |
| AHF15827 | Q91E | 0.709 | 0.823 |
| AHF15828 | Q91E | 0.694 | 0.806 |
| AHF15829 | Q91E | 0.701 | 0.814 |
| AHF15830 | Y92D | 0.758 | 0.805 |
| AHF15831 | Y92N | 0.773 | 0.821 |
| AHF15832 | H93N | 0.857 | 0.910 |
| AHF15833 | H94Y | 0.865 | 0.942 |
| AHF15834 | H94F | 0.770 | 0.838 |
| AHF15835 | S95F | 0.837 | 0.911 |
| AHF15836 | S95E | 0.855 | 0.931 |
| AHF15837 | S95Q | 0.893 | 0.972 |
| AHF15838 | S95W | 0.804 | 0.875 |
| AHF15839 | S95N | 0.863 | 0.940 |
| AHF15840 | S95W | 0.790 | 0.860 |
| AHF15841 | S95N | 0.884 | 0.962 |
| AHF15842 | S95K | 0.841 | 0.916 |
| AHF15843 | S95A | 0.830 | 0.904 |
| AHF15844 | S95T | 0.871 | 0.948 |
| AHF15845 | S95E | 0.820 | 0.893 |
| AHF15846 | WT | 0.900 | 0.980 |
| AHF15847 | S95I | 0.846 | 0.921 |
| AHF15848 | S95A | 0.872 | 0.949 |
| AHF15849 | S95I | 0.817 | 0.889 |
| AHF15850 | S95K | 0.824 | 0.897 |
| AHF15851 | S95K | 0.813 | 0.885 |
| AHF15852 | S95K | 0.790 | 0.860 |
| AHF15853 | S95H | 0.800 | 0.871 |
| AHF15854 | WT | 0.889 | 0.968 |
| AHF15855 | S95Y | 0.827 | 0.900 |
| AHF15856 | Bad Sequence | 0.884 | 0.962 |
| AHF15857 | S95C | 0.849 | 0.924 |
| AHF15858 | S95I | 0.791 | 0.861 |
| AHF15859 | Y97H | 0.775 | 0.982 |
| AHF15860 | Y97Q | 0.687 | 0.871 |
| AHF15861 | Y97S | 0.633 | 0.802 |
| AHF15862 | Y97R | 0.769 | 0.975 |
| AHF15863 | Y97I | 0.667 | 0.845 |
| AHF15864 | Y97K | 0.654 | 0.829 |
| AHF15865 | Y97F | 0.750 | 0.951 |
| AHF15866 | Y97F | 0.756 | 0.958 |
| AHF15867 | Y97V | 0.662 | 0.839 |
| AHF15868 | Y97L | 0.739 | 0.937 |
| AHF15869 | Bad Sequence | 0.743 | 0.849 |
| AHF15870 | I98L | 0.836 | 0.955 |
| AHF15871 | I98V | 0.832 | 0.951 |
| AHF15872 | I98R | 0.726 | 0.830 |
| AHF15873 | I98R | 0.722 | 0.825 |
| AHF15874 | I98M | 0.729 | 0.833 |
| AHF15875 | I98V | 0.838 | 0.958 |
| AHF15876 | I98V | 0.787 | 0.899 |
| AHF15877 | T100F | 0.494 | 0.944 |
| AHF15878 | T100I | 0.493 | 0.942 |
| AHF15879 | T100P | 0.460 | 0.879 |
| AHF15880 | T100V | 0.461 | 0.881 |
| AHF15881 | T100F | 0.492 | 0.940 |
| AHF15882 | T100F | 0.480 | 0.917 |
| AHF15883 | T100F | 0.517 | 0.988 |
| AHF15884 | T100V | 0.428 | 0.818 |

*Ratio = OD(Variant)/OD(WT) = fold-change in affinity, as characterized by ELISA

1. A human or humanized antigen binding molecule that binds to Porphyromonas gingivalis, wherein the antigen binding molecule comprises:
a heavy chain variable region (HVR) comprising:
a complementarity determining region (HCDR) 1 of a HCDR1 of SEQ ID NO:9 or 37;
a HCDR2 of a HCDR2 of SEQ ID NO:9 or 37; and
a HCDR3 of a HCDR2 of SEQ ID NO:9 or 37; and
a light chain variable region (LVR) comprising:
a complementarity determining region (LCDR) 1 of a LCDR1 of SEQ ID NO:10 or 38;
a LCDR2 of a LCDR2 of SEQ ID NO:10 or 38; and
a LCDR3 of a LCDR2 of SEQ ID NO:10 or 38,
wherein the antigen binding molecule comprises at least one of:
one or more HVR residues selected from L48, L67, K71, V78, and M92, as numbered according to the numbering as provided in SEQ ID NO:37
one or more LVR residues selected from Q46, W48, A61, Y72, and T86, as numbered according to the numbering as provided in SEQ ID NO:38.

2-127. (canceled)

128. A human or humanized antigen binding molecule that binds to Porphyromonas gingivalis.

129. The antigen binding molecule of claim 128, comprising:
(i) a light chain variable region (LVR) having a sequence with at least 80% identity to the sequence of SEQ ID NO: 33;
(ii) a heavy chain variable region (HVR) having a sequence with at least 80% identity to the sequence of SEQ ID NO:30; and/or
(iii) a CH domain having a sequence with at least 80% identity to the sequence of SEQ ID NO: 171.

130. The antigen binding molecule of claim 128, comprising at least one of:
(i) a heavy chain complementarity determining region (HCDR) 1 having a sequence of SEQ ID NO:3;
(ii) a HCDR2 having a sequence of SEQ ID NO:4;
(iii) a HCDR3 having a sequence of SEQ ID NO:5;
(iv) an LCDR1 having a sequence of SEQ ID NO:6;
(v) an LCDR2 having a sequence of SEQ ID NO: 7;
(vi) an LCDR3 having a sequence of SEQ ID NO: 8; and/or
(vii) the CDRs of the 6 CDRs within SEQ ID NO: 1 and 2.

131. The antigen binding molecule of claim 128, wherein the antigen binding molecule binds to at least one of: a HagA protein target, a hemagglutinin protein target, a gingipain protein target, an adhesin protein target, or any combination thereof.

132. The antigen binding molecule of claim 128, wherein the antigen binding molecule neutralizes the activity of at least one of: a HagA protein target, a hemagglutinin protein target, a gingipain protein target, an adhesin protein target, or any combination thereof.

133. The antigen binding molecule of claim 128, wherein the antigen binding molecule is a Fab, a diabody, Fab′, F(ab′)₂, Fv, single-chain antibody, nanobody, domain antibody, bivalent antibody, bispecific antibody, or peptibody.

134. The antigen binding molecule of claim 128, wherein the antibody when administered to a subject orally, topically, intravenously, intradermally, intramuscularly, intracranially, or intercranially.

135. The antigen binding molecule of claim 134, wherein administration to a subject reduces a P. gingivalis infection by at least 80%.

136. The antigen binding molecule of claim 128, wherein the antigen binding molecule is of an IgG isotype.

137. The antigen binding molecule of claim 128, wherein the antigen binding molecule further comprises an alanine at position 222, wherein the change at position 222 of the antibody results in a lower cleavage and inactivation rate of the antigen binding molecule in the presence of a P. gingivalis protease.

138. A nucleic acid encoding a human or humanized antigen binding molecule that binds to Porphyromonas gingivalis.

139. A method of treating or preventing a condition, disorder or disease associated with a P. gingivalis infection, or symptoms thereof, comprising:
identifying a subject in need of treating or preventing a condition, disorder or disease associated with a P. gingivalis infection, or symptoms thereof; and
administering to the subject a therapeutically effective amount of an antigen binding molecule:
a) that binds to Porphyromonas gingivalis, wherein the antigen binding molecule is not KB001;
b) having a heavy chain sequence with at least 80% identity to the sequence of SEQ ID NO: 30, and a light chain sequence with at least 80% identity to the sequence of SEQ ID NO: 33, and/or
c) having a LCDR1, a LCDR2, and a LCDR3 that is a LCDR1, a LCDR2, and a LCDR3 that is within SEQ ID NO: 2 and a HCDR1, a HCDR2, and a HCDR3 that is a HCDR1, a HCDR2, and a HCDR3 that is within SEQ ID NO: 1,
thereby treating or preventing the condition, disorder or disease associated with a P. gingivalis infection, or symptoms thereof.

140. The method of claim 139, wherein the condition, disorder or disease is one or more of: bacterial infection, vascular disease (for example, cardiovascular disease, atherosclerosis, coronary artery disease, myocardial infarction, stroke, and myocardial hypertrophy); systemic disease (for example, type II diabetes, insulin resistance and metabolic syndrome); rheumatoid arthritis; cancer (for example, oral, gastrointestinal, or pancreatic cancer); renal disease, gut microbiome-related disorder (for example, inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (for example, Parkinson's), Peri-Implantitis and/or periodontal disease and/or associated bone loss, cognitive disorders (for example, early, middle, and/or late dementia; Alzheimer's disease); regenerative and stem cell dysfunction; and longevity or age-related disorder and or any future diseases found to be associated with a P. gingivalis infection.

141. The method of claim 139, wherein the condition, disorder, or disease is present in multiple systems, organs, or tissues.

142. The method of claim 139, further comprising administering to the subject at least one other therapeutic agent for treating or preventing the condition, disorder or disease, or symptoms thereof.

143. The method of claim 139, wherein treating or preventing the condition, disorder or disease associated with a P. gingivalis infection results in a decrease inflammation.

144. The method of claim 139, wherein the decrease of inflammation is reduced activity or activation of inflammasomes, reduced cytokine levels, lowered host cell death, reduced proinflammatory mediators, and/or reduced chronic distant site inflammatory atherosclerosis.

145. The method of claim 139, wherein the administering comprises administering the antigen binding domain intravenously, subgingivally, intradermally, subcutaneously, intrathecally, or by nebulization.

146. A method of treating a disorder driven by P. gingivalis comprising:
providing an antibody that binds to a P. gingivalis associated peptide, to a subject,
wherein the antibody is known to function to stop a P. gingivalis infection,
wherein the antibody is a humanized or human antibody, and
wherein position 222 of the antibody has been changed to an alanine;
wherein the change at position 222 of the antibody results in a lower cleavage and inactivation rate of the antibody in the presence of a P. gingivalis protease.

147. A human or humanized antigen binding molecule, comprising:
(i) a light chain variable region (LVR) having a that is the sequence of SEQ ID NO: 33;
(ii) a heavy chain variable region (HVR) having a sequence that is the sequence of SEQ ID NO:30; and
(iii) a CH domain having a sequence with at least 80% identity to the sequence of SEQ ID NO: 171,
wherein position 222 of the human or humanized antigen binding molecule has been changed to an alanine,
wherein the change at position 222 of the human or humanized antigen binding molecule results in a lower cleavage and inactivation rate of the antibody in the presence of a P. gingivalis protease.
